SEQUENCE LISTING (1241 sequences)

SEQ ID NO: 1 - RNA, 36 nt, Artificial Sequence
  Synthetic Construct
guugcaaaacccaagaaauccgucuuucauugacgg

SEQ ID NO: 2 - RNA, 36 nt, Artificial Sequence
  Synthetic Construct
aauagcggcccuaagaaauccgucuuucauugacgg

SEQ ID NO: 3 - RNA, 36 nt, Artificial Sequence
  Synthetic Construct
auuggaacuggcgagaaauccgucuuucauugacgg

SEQ ID NO: 4 - RNA, 36 nt, Artificial Sequence
  Synthetic Construct
ccagcaacaccuaagaaauccgucuuucauugacgg

SEQ ID NO: 5 - RNA, 36 nt, Artificial Sequence
  Synthetic Construct
cggcgcucgaauaggaaauccgucuuucauugacgg

SEQ ID NO: 6 - RNA, 36 nt, Artificial Sequence
  Synthetic Construct
guggcaacaccuaagaaauccgucuuucauugacgg

SEQ ID NO: 7 - RNA, 36 nt, Artificial Sequence
  Synthetic Construct
guugcaacaccuaagaaauccgucuuucauugacgg

SEQ ID NO: 8 - RNA, 36 nt, Artificial Sequence
  Synthetic Construct
guugcaaugccuaagaaauccgucuuucauugacgg

SEQ ID NO: 9 - RNA, 34 nt, Artificial Sequence
  Synthetic Construct
gcaacaccuaagaaauccgucuuucauugacggg

SEQ ID NO: 10 - RNA, 23 nt, Artificial Sequence
  Synthetic Construct
agaaauccgucuuucauugacgg

SEQ ID NO: 11 - DNA, 30 bp, Artificial Sequence
  Synthetic Construct
atataagtggaggcgtcgcgctggcgggca

SEQ ID NO: 12 - DNA, 30 bp, Artificial Sequence
  Synthetic Construct
tattaaacgcgtgcccagccaatcaggaca

SEQ ID NO: 13 - DNA, 30 bp, Artificial Sequence
  Synthetic Construct
aggaatgcccgccagcgcgacgcctccact

SEQ ID NO: 14 - DNA, 30 bp, Artificial Sequence
  Synthetic Construct
tggcctggaggctatccagcgtgagtctct

SEQ ID NO: 15 - DNA, 30 bp, Artificial Sequence
  Synthetic Construct
ctggcctggaggctatccagcgtgagtctc

SEQ ID NO: 16 - DNA, 30 bp, Artificial Sequence
  Synthetic Construct
gctgtgctcgcgctactctctctttctggc

SEQ ID NO: 17 - DNA, 30 bp, Artificial Sequence
  Synthetic Construct
gggccgagatgtctcgctccgtggccttag

SEQ ID NO: 18 - DNA, 30 bp, Artificial Sequence
  Synthetic Construct
ctgaagctgacagcattcgggccgagatgt

SEQ ID NO: 19 - DNA, 30 bp, Artificial Sequence
  Synthetic Construct
aatataagtggaggcgtcgcgctggcgggc

SEQ ID NO: 20 - DNA, 30 bp, Artificial Sequence
  Synthetic Construct
gctgggcacgcgtttaatataagtggaggc

SEQ ID NO: 21 - DNA, 30 bp, Artificial Sequence
  Synthetic Construct
tcacagcccaagatagttaagtggggtaag

SEQ ID NO: 22 - DNA, 30 bp, Artificial Sequence
  Synthetic Construct
agtggggtaagtcttacattcttttgtaag

SEQ ID NO: 23 - DNA, 30 bp, Artificial Sequence
  Synthetic Construct
cattctctgctggatgacgtgagtaaacct

SEQ ID NO: 24 - DNA, 30 bp, Artificial Sequence
  Synthetic Construct
ttttgtaagctgctgaaagttgtgtatgag

SEQ ID NO: 25 - DNA, 30 bp, Artificial Sequence
  Synthetic Construct
gtcacagcccaagatagttaagtggggtaa

SEQ ID NO: 26 - DNA, 30 bp, Artificial Sequence
  Synthetic Construct
cattcttttgtaagctgctgaaagttgtgt

SEQ ID NO: 27 - DNA, 30 bp, Artificial Sequence
  Synthetic Construct
acccccactgaaaaagatgagtatgcctgc

SEQ ID NO: 28 - DNA, 30 bp, Artificial Sequence
  Synthetic Construct
cagcaaggactggtctttctatctcttgta

SEQ ID NO: 29 - DNA, 30 bp, Artificial Sequence
  Synthetic Construct
tatctcttgtactacactgaattcaccccc

SEQ ID NO: 30 - DNA, 30 bp, Artificial Sequence
  Synthetic Construct
ctatctcttgtactacactgaattcacccc

SEQ ID NO: 31 - DNA, 30 bp, Artificial Sequence
  Synthetic Construct
agcaaggactggtctttctatctcttgtac

SEQ ID NO: 32 - DNA, 30 bp, Artificial Sequence
  Synthetic Construct
tgtaagctgctgaaagttgtgtatgagtag

SEQ ID NO: 33 - DNA, 30 bp, Artificial Sequence
  Synthetic Construct
tctttcagcaaggactggtctttctatctc

SEQ ID NO: 34 - DNA, 30 bp, Artificial Sequence
  Synthetic Construct
tactacactgaattcacccccactgaaaaa

SEQ ID NO: 35 - DNA, 30 bp, Artificial Sequence
  Synthetic Construct
gtaagctgctgaaagttgtgtatgagtagt

SEQ ID NO: 36 - DNA, 30 bp, Artificial Sequence
  Synthetic Construct
ccccacttaactatcttgggctgtgacaaa

SEQ ID NO: 37 - DNA, 30 bp, Artificial Sequence
  Synthetic Construct
cagcagcttacaaaagaatgtaagacttac

SEQ ID NO: 38 - DNA, 30 bp, Artificial Sequence
  Synthetic Construct
agcagcttacaaaagaatgtaagacttacc

SEQ ID NO: 39 - DNA, 30 bp, Artificial Sequence
  Synthetic Construct
caaaagaatgtaagacttaccccacttaac

SEQ ID NO: 40 - DNA, 30 bp, Artificial Sequence
  Synthetic Construct
agacttgtctttcagcaaggactggtcttt

SEQ ID NO: 41 - DNA, 30 bp, Artificial Sequence
  Synthetic Construct
actatcttgggctgtgacaaagtcacatgg

SEQ ID NO: 42 - DNA, 30 bp, Artificial Sequence
  Synthetic Construct
ggctgtgacaaagtcacatggttcacacgg

SEQ ID NO: 43 - DNA, 30 bp, Artificial Sequence
  Synthetic Construct
acacggcaggcatactcatctttttcagtg

SEQ ID NO: 44 - DNA, 30 bp, Artificial Sequence
  Synthetic Construct
ttcagtgggggtgaattcagtgtagtacaa

SEQ ID NO: 45 - DNA, 30 bp, Artificial Sequence
  Synthetic Construct
tcagtgggggtgaattcagtgtagtacaag

SEQ ID NO: 46 - DNA, 30 bp, Artificial Sequence
  Synthetic Construct
cagtgggggtgaattcagtgtagtacaaga

SEQ ID NO: 47 - DNA, 30 bp, Artificial Sequence
  Synthetic Construct
agtgggggtgaattcagtgtagtacaagag

SEQ ID NO: 48 - DNA, 30 bp, Artificial Sequence
  Synthetic Construct
agtgtagtacaagagatagaaagaccagtc

SEQ ID NO: 49 - DNA, 30 bp, Artificial Sequence
  Synthetic Construct
ctgaaagacaagtctgaatgctccactttt

SEQ ID NO: 50 - DNA, 30 bp, Artificial Sequence
  Synthetic Construct
taagctgctgaaagttgtgtatgagtagtc

SEQ ID NO: 51 - DNA, 30 bp, Artificial Sequence
  Synthetic Construct
aaaaagtggagcattcagacttgtctttca

SEQ ID NO: 52 - DNA, 30 bp, Artificial Sequence
  Synthetic Construct
catccatccgacattgaagttgacttactg

SEQ ID NO: 53 - DNA, 30 bp, Artificial Sequence
  Synthetic Construct
acttactgaagaatggagagagaattgaaa

SEQ ID NO: 54 - DNA, 30 bp, Artificial Sequence
  Synthetic Construct
atattgccagggtatttcacttggggctaa

SEQ ID NO: 55 - DNA, 30 bp, Artificial Sequence
  Synthetic Construct
gagtacctgaggaatatcgggaaaagacac

SEQ ID NO: 56 - DNA, 30 bp, Artificial Sequence
  Synthetic Construct
ggagtacctgaggaatatcgggaaaagaca

SEQ ID NO: 57 - DNA, 30 bp, Artificial Sequence
  Synthetic Construct
tctgctggatgacgtgagtaaacctgaatc

SEQ ID NO: 58 - DNA, 30 bp, Artificial Sequence
  Synthetic Construct
ccattctctgctggatgacgtgagtaaacc

SEQ ID NO: 59 - DNA, 30 bp, Artificial Sequence
  Synthetic Construct
actttccattctctgctggatgacgtgagt

SEQ ID NO: 60 - DNA, 30 bp, Artificial Sequence
  Synthetic Construct
gactttccattctctgctggatgacgtgag

SEQ ID NO: 61 - DNA, 30 bp, Artificial Sequence
  Synthetic Construct
aggaaatttgactttccattctctgctgga

SEQ ID NO: 62 - DNA, 30 bp, Artificial Sequence
  Synthetic Construct
aatgtcggatggatgaaacccagacacata

SEQ ID NO: 63 - DNA, 30 bp, Artificial Sequence
  Synthetic Construct
agtaagtcaacttcaatgtcggatggatga

SEQ ID NO: 64 - DNA, 30 bp, Artificial Sequence
  Synthetic Construct
ttcagtaagtcaacttcaatgtcggatgga

SEQ ID NO: 65 - DNA, 30 bp, Artificial Sequence
  Synthetic Construct
tctctccattcttcagtaagtcaacttcaa

SEQ ID NO: 66 - DNA, 30 bp, Artificial Sequence
  Synthetic Construct
aattctctctccattcttcagtaagtcaac

SEQ ID NO: 67 - DNA, 30 bp, Artificial Sequence
  Synthetic Construct
ctgaagaatggagagagaattgaaaaagtg

SEQ ID NO: 68 - DNA, 30 bp, Artificial Sequence
  Synthetic Construct
caattctctctccattcttcagtaagtcaa

SEQ ID NO: 69 - DNA, 30 bp, Artificial Sequence
  Synthetic Construct
tcccgatattcctcaggtactccaaagatt

SEQ ID NO: 70 - DNA, 30 bp, Artificial Sequence
  Synthetic Construct
cccgatattcctcaggtactccaaagattc

SEQ ID NO: 71 - DNA, 30 bp, Artificial Sequence
  Synthetic Construct
ccgatattcctcaggtactccaaagattca

SEQ ID NO: 72 - DNA, 30 bp, Artificial Sequence
  Synthetic Construct
ctcaggtactccaaagattcaggtttactc

SEQ ID NO: 73 - DNA, 30 bp, Artificial Sequence
  Synthetic Construct
aggtttactcacgtcatccagcagagaatg

SEQ ID NO: 74 - DNA, 30 bp, Artificial Sequence
  Synthetic Construct
actcacgtcatccagcagagaatggaaagt

SEQ ID NO: 75 - DNA, 30 bp, Artificial Sequence
  Synthetic Construct
ctcacgtcatccagcagagaatggaaagtc

SEQ ID NO: 76 - DNA, 30 bp, Artificial Sequence
  Synthetic Construct
cctgaattgctatgtgtctgggtttcatcc

SEQ ID NO: 77 - DNA, 30 bp, Artificial Sequence
  Synthetic Construct
ctgaattgctatgtgtctgggtttcatcca

SEQ ID NO: 78 - DNA, 30 bp, Artificial Sequence
  Synthetic Construct
ctatgtgtctgggtttcatccatccgacat

SEQ ID NO: 79 - DNA, 30 bp, Artificial Sequence
  Synthetic Construct
ttcaattctctctccattcttcagtaagtc

SEQ ID NO: 80 - DNA, 30 bp, Artificial Sequence
  Synthetic Construct
atccatccgacattgaagttgacttactga

SEQ ID NO: 81 - DNA, 30 bp, Artificial Sequence
  Synthetic Construct
aagttgacttactgaagaatggagagagaa

SEQ ID NO: 82 - DNA, 30 bp, Artificial Sequence
  Synthetic Construct
atgtgtcttttcccgatattcctcaggtac

SEQ ID NO: 83 - DNA, 30 bp, Artificial Sequence
  Synthetic Construct
tcaattctctctccattcttcagtaagtca

SEQ ID NO: 84 - DNA, 30 bp, Artificial Sequence
  Synthetic Construct
atgtcttccttttttttctccactgtcttt

SEQ ID NO: 85 - DNA, 30 bp, Artificial Sequence
  Synthetic Construct
cctccatgatgctgcttacatgtctcgatc

SEQ ID NO: 86 - DNA, 30 bp, Artificial Sequence
  Synthetic Construct
catgtctcgatctatgaaaaagacagtgga

SEQ ID NO: 87 - DNA, 30 bp, Artificial Sequence
  Synthetic Construct
cttttttttctccactgtctttttcataga

SEQ ID NO: 88 - DNA, 30 bp, Artificial Sequence
  Synthetic Construct
tttttctccactgtctttttcatagatcga

SEQ ID NO: 89 - DNA, 30 bp, Artificial Sequence
  Synthetic Construct
ttttctccactgtctttttcatagatcgag

SEQ ID NO: 90 - DNA, 30 bp, Artificial Sequence
  Synthetic Construct
tttctccactgtctttttcatagatcgaga

SEQ ID NO: 91 - DNA, 30 bp, Artificial Sequence
  Synthetic Construct
tctccactgtctttttcatagatcgagaca

SEQ ID NO: 92 - DNA, 30 bp, Artificial Sequence
  Synthetic Construct
ctccactgtctttttcatagatcgagacat

SEQ ID NO: 93 - DNA, 30 bp, Artificial Sequence
  Synthetic Construct
tccactgtctttttcatagatcgagacatg

SEQ ID NO: 94 - DNA, 30 bp, Artificial Sequence
  Synthetic Construct
ttcatagatcgagacatgtaagcagcatca

SEQ ID NO: 95 - DNA, 30 bp, Artificial Sequence
  Synthetic Construct
tcatagatcgagacatgtaagcagcatcat

SEQ ID NO: 96 - DNA, 30 bp, Artificial Sequence
  Synthetic Construct
catagatcgagacatgtaagcagcatcatg

SEQ ID NO: 97 - DNA, 30 bp, Artificial Sequence
  Synthetic Construct
ttctccactgtctttttcatagatcgagac

SEQ ID NO: 98 - DNA, 30 bp, Artificial Sequence
  Synthetic Construct
ttgaccttgagaaaatgtttttgtttcact

SEQ ID NO: 99 - DNA, 30 bp, Artificial Sequence
  Synthetic Construct
atagatcgagacatgtaagcagcatcatgg

SEQ ID NO: 100 - DNA, 30 bp, Artificial Sequence
  Synthetic Construct
ctcaaggtcaaaaacttacctccatgatgc

SEQ ID NO: 101 - DNA, 30 bp, Artificial Sequence
  Synthetic Construct
tctcaaggtcaaaaacttacctccatgatg

SEQ ID NO: 102 - DNA, 30 bp, Artificial Sequence
  Synthetic Construct
tcaaggtcaaaaacttacctccatgatgct

SEQ ID NO: 103 - DNA, 30 bp, Artificial Sequence
  Synthetic Construct
ttgtttcactgtcctgaggactatttatag

SEQ ID NO: 104 - DNA, 30 bp, Artificial Sequence
  Synthetic Construct
tgtttcactgtcctgaggactatttataga

SEQ ID NO: 105 - DNA, 30 bp, Artificial Sequence
  Synthetic Construct
agaaaatgtttttgtttcactgtcctgagg

SEQ ID NO: 106 - DNA, 30 bp, Artificial Sequence
  Synthetic Construct
accttgagaaaatgtttttgtttcactgtc

SEQ ID NO: 107 - DNA, 30 bp, Artificial Sequence
  Synthetic Construct
gaccttgagaaaatgtttttgtttcactgt

SEQ ID NO: 108 - DNA, 30 bp, Artificial Sequence
  Synthetic Construct
tgaccttgagaaaatgtttttgtttcactg

SEQ ID NO: 109 - DNA, 30 bp, Artificial Sequence
  Synthetic Construct
tattaggtattacaagtaatctagaaatga

SEQ ID NO: 110 - DNA, 30 bp, Artificial Sequence
  Synthetic Construct
acattgtattaggtattacaagtaatctag

SEQ ID NO: 111 - DNA, 30 bp, Artificial Sequence
  Synthetic Construct
catagcatttacattgtattaggtattaca

SEQ ID NO: 112 - DNA, 30 bp, Artificial Sequence
  Synthetic Construct
gcatagcatttacattgtattaggtattac

SEQ ID NO: 113 - DNA, 30 bp, Artificial Sequence
  Synthetic Construct
cattgtattaggtattacaagtaatctaga

SEQ ID NO: 114 - DNA, 30 bp, Artificial Sequence
  Synthetic Construct
aacaataacaactatttgcatagcatttac

SEQ ID NO: 115 - DNA, 30 bp, Artificial Sequence
  Synthetic Construct
cttgtcattattccttaaacaataacaact

SEQ ID NO: 116 - DNA, 30 bp, Artificial Sequence
  Synthetic Construct
ttccttaaacaataacaactatttgcatag

SEQ ID NO: 117 - DNA, 30 bp, Artificial Sequence
  Synthetic Construct
tcattattccttaaacaataacaactattt

SEQ ID NO: 118 - DNA, 30 bp, Artificial Sequence
  Synthetic Construct
ttgtcattattccttaaacaataacaacta

SEQ ID NO: 119 - DNA, 30 bp, Artificial Sequence
  Synthetic Construct
tcttgtcattattccttaaacaataacaac

SEQ ID NO: 120 - DNA, 30 bp, Artificial Sequence
  Synthetic Construct
ggtattacaagtaatctagaaatgatttaa

SEQ ID NO: 121 - DNA, 30 bp, Artificial Sequence
  Synthetic Construct
ttcttgtcattattccttaaacaataacaa

SEQ ID NO: 122 - DNA, 30 bp, Artificial Sequence
  Synthetic Construct
cttaaacaataacaactatttgcatagcat

SEQ ID NO: 123 - DNA, 30 bp, Artificial Sequence
  Synthetic Construct
caagtaatctagaaatgatttaaagtatac

SEQ ID NO: 124 - DNA, 30 bp, Artificial Sequence
  Synthetic Construct
cttgctaaaatattaaatccttcagatact

SEQ ID NO: 125 - DNA, 30 bp, Artificial Sequence
  Synthetic Construct
aagtatacaggaggatgtggataggttata

SEQ ID NO: 126 - DNA, 30 bp, Artificial Sequence
  Synthetic Construct
gctccctcttagagtctgcatactcctcat

SEQ ID NO: 127 - DNA, 30 bp, Artificial Sequence
  Synthetic Construct
tgctccctcttagagtctgcatactcctca

SEQ ID NO: 128 - DNA, 30 bp, Artificial Sequence
  Synthetic Construct
agatacttttgctccctcttagagtctgca

SEQ ID NO: 129 - DNA, 30 bp, Artificial Sequence
  Synthetic Construct
aatccttcagatacttttgctccctcttag

SEQ ID NO: 130 - DNA, 30 bp, Artificial Sequence
  Synthetic Construct
ctaaaatattaaatccttcagatacttttg

SEQ ID NO: 131 - DNA, 30 bp, Artificial Sequence
  Synthetic Construct
tttcttgtcattattccttaaacaataaca

SEQ ID NO: 132 - DNA, 30 bp, Artificial Sequence
  Synthetic Construct
aaagtatacaggaggatgtggataggttat

SEQ ID NO: 133 - DNA, 30 bp, Artificial Sequence
  Synthetic Construct
tatatctattccttgctaaaatattaaatc

SEQ ID NO: 134 - DNA, 30 bp, Artificial Sequence
  Synthetic Construct
ggtgtccaagaaggggtcctgaaaccaatc

SEQ ID NO: 135 - DNA, 30 bp, Artificial Sequence
  Synthetic Construct
aatatccatagatttggtgtccaagaaggg

SEQ ID NO: 136 - DNA, 30 bp, Artificial Sequence
  Synthetic Construct
tagaagggacttaaatatccatagatttgg

SEQ ID NO: 137 - DNA, 30 bp, Artificial Sequence
  Synthetic Construct
atagaagggacttaaatatccatagatttg

SEQ ID NO: 138 - DNA, 30 bp, Artificial Sequence
  Synthetic Construct
tatagaagggacttaaatatccatagattt

SEQ ID NO: 139 - DNA, 30 bp, Artificial Sequence
  Synthetic Construct
tatgcaaatactataccattttatagaagg

SEQ ID NO: 140 - DNA, 30 bp, Artificial Sequence
  Synthetic Construct
gtgtccaagaaggggtcctgaaaccaatcc

SEQ ID NO: 141 - DNA, 30 bp, Artificial Sequence
  Synthetic Construct
ttttcttgtcattattccttaaacaataac

SEQ ID NO: 142 - DNA, 30 bp, Artificial Sequence
  Synthetic Construct
aaatctgttatttgtcattcaaagtacagc

SEQ ID NO: 143 - DNA, 30 bp, Artificial Sequence
  Synthetic Construct
ctgagcatgtacagacttttttttcttgtc

SEQ ID NO: 144 - DNA, 30 bp, Artificial Sequence
  Synthetic Construct
ctgaatccacagatgtggagcccctggata

SEQ ID NO: 145 - DNA, 30 bp, Artificial Sequence
  Synthetic Construct
gctgaatccacagatgtggagcccctggat

SEQ ID NO: 146 - DNA, 30 bp, Artificial Sequence
  Synthetic Construct
atccatggtttgctgaatccacagatgtgg

SEQ ID NO: 147 - DNA, 30 bp, Artificial Sequence
  Synthetic Construct
gatccatggtttgctgaatccacagatgtg

SEQ ID NO: 148 - DNA, 30 bp, Artificial Sequence
  Synthetic Construct
tgatccatggtttgctgaatccacagatgt

SEQ ID NO: 149 - DNA, 30 bp, Artificial Sequence
  Synthetic Construct
ttgatccatggtttgctgaatccacagatg

SEQ ID NO: 150 - DNA, 30 bp, Artificial Sequence
  Synthetic Construct
gaatgacaaataacagatttaaaattttca

SEQ ID NO: 151 - DNA, 30 bp, Artificial Sequence
  Synthetic Construct
cccagtgtttttgatccatggtttgctgaa

SEQ ID NO: 152 - DNA, 30 bp, Artificial Sequence
  Synthetic Construct
tccccagtgtttttgatccatggtttgctg

SEQ ID NO: 153 - DNA, 30 bp, Artificial Sequence
  Synthetic Construct
ttccccagtgtttttgatccatggtttgct

SEQ ID NO: 154 - DNA, 30 bp, Artificial Sequence
  Synthetic Construct
tttccccagtgtttttgatccatggtttgc

SEQ ID NO: 155 - DNA, 30 bp, Artificial Sequence
  Synthetic Construct
ttttccccagtgtttttgatccatggtttg

SEQ ID NO: 156 - DNA, 30 bp, Artificial Sequence
  Synthetic Construct
aggaataatgacaagaaaaaaaagtctgta

SEQ ID NO: 157 - DNA, 30 bp, Artificial Sequence
  Synthetic Construct
ctccctcttagagtctgcatactcctcatg

SEQ ID NO: 158 - DNA, 30 bp, Artificial Sequence
  Synthetic Construct
ccccagtgtttttgatccatggtttgctga

SEQ ID NO: 159 - DNA, 30 bp, Artificial Sequence
  Synthetic Construct
tttttcttgtcattattccttaaacaataa

SEQ ID NO: 160 - DNA, 30 bp, Artificial Sequence
  Synthetic Construct
aatgacaaataacagatttaaaattttcaa

SEQ ID NO: 161 - DNA, 29 bp, Artificial Sequence
  Synthetic Construct
aaattttcaaggcatagttttatacctga

SEQ ID NO: 162 - DNA, 30 bp, Artificial Sequence
  Synthetic Construct
actgagcatgtacagacttttttttcttgt

SEQ ID NO: 163 - DNA, 30 bp, Artificial Sequence
  Synthetic Construct
tgtctttactgagcatgtacagactttttt

SEQ ID NO: 164 - DNA, 30 bp, Artificial Sequence
  Synthetic Construct
agcaaaccatggatcaaaaacactggggaa

SEQ ID NO: 165 - DNA, 30 bp, Artificial Sequence
  Synthetic Construct
cgtatccaggggctccacatctgtggattc

SEQ ID NO: 166 - DNA, 30 bp, Artificial Sequence
  Synthetic Construct
aaagtacagcgggccttccgtatccagggg

SEQ ID NO: 167 - DNA, 30 bp, Artificial Sequence
  Synthetic Construct
tcattcaaagtacagcgggccttccgtatc

SEQ ID NO: 168 - DNA, 30 bp, Artificial Sequence
  Synthetic Construct
aaaattttcaaggcatagttttatacctga

SEQ ID NO: 169 - DNA, 30 bp, Artificial Sequence
  Synthetic Construct
gtcattcaaagtacagcgggccttccgtat

SEQ ID NO: 170 - DNA, 30 bp, Artificial Sequence
  Synthetic Construct
aatctgttatttgtcattcaaagtacagcg

SEQ ID NO: 171 - DNA, 30 bp, Artificial Sequence
  Synthetic Construct
taaatctgttatttgtcattcaaagtacag

SEQ ID NO: 172 - DNA, 30 bp, Artificial Sequence
  Synthetic Construct
aaaattttaaatctgttatttgtcattcaa

SEQ ID NO: 173 - DNA, 21 bp, Artificial Sequence
  Synthetic Construct
aaggcatagttttatacctga

SEQ ID NO: 174 - DNA, 22 bp, Artificial Sequence
  Synthetic Construct
caaggcatagttttatacctga

SEQ ID NO: 175 - DNA, 23 bp, Artificial Sequence
  Synthetic Construct
tcaaggcatagttttatacctga

SEQ ID NO: 176 - DNA, 30 bp, Artificial Sequence
  Synthetic Construct
tttgtcattcaaagtacagcgggccttccg

SEQ ID NO: 177 - DNA, 30 bp, Artificial Sequence
  Synthetic Construct
gagtctgcatactcctcatgacctggcccg

SEQ ID NO: 178 - DNA, 30 bp, Artificial Sequence
  Synthetic Construct
actatcttaacaagctttgagtgcaagaga

SEQ ID NO: 179 - DNA, 30 bp, Artificial Sequence
  Synthetic Construct
acttctttgagcatcagattcctaatctgg

SEQ ID NO: 180 - DNA, 30 bp, Artificial Sequence
  Synthetic Construct
tatttctaaattttcccccaaattctaagc

SEQ ID NO: 181 - DNA, 30 bp, Artificial Sequence
  Synthetic Construct
ctaaattttcccccaaattctaagcagagt

SEQ ID NO: 182 - DNA, 30 bp, Artificial Sequence
  Synthetic Construct
taaattttcccccaaattctaagcagagta

SEQ ID NO: 183 - DNA, 30 bp, Artificial Sequence
  Synthetic Construct
tcccccaaattctaagcagagtatgtaaat

SEQ ID NO: 184 - DNA, 30 bp, Artificial Sequence
  Synthetic Construct
cccccaaattctaagcagagtatgtaaatt

SEQ ID NO: 185 - DNA, 30 bp, Artificial Sequence
  Synthetic Construct
ccccaaattctaagcagagtatgtaaattg

SEQ ID NO: 186 - DNA, 30 bp, Artificial Sequence
  Synthetic Construct
taagcagagtatgtaaattggaagttaact

SEQ ID NO: 187 - DNA, 30 bp, Artificial Sequence
  Synthetic Construct
gaagttaacttatgcacgcttaactatctt

SEQ ID NO: 188 - DNA, 30 bp, Artificial Sequence
  Synthetic Construct
acttatgcacgcttaactatcttaacaagc

SEQ ID NO: 189 - DNA, 30 bp, Artificial Sequence
  Synthetic Construct
tgcacgcttaactatcttaacaagctttga

SEQ ID NO: 190 - DNA, 30 bp, Artificial Sequence
  Synthetic Construct
aaggaataatgacaagaaaaaaaagtctgt

SEQ ID NO: 191 - DNA, 30 bp, Artificial Sequence
  Synthetic Construct
acaagctttgagtgcaagagattgaagagt

SEQ ID NO: 192 - DNA, 30 bp, Artificial Sequence
  Synthetic Construct
gagtgcaagagattgaagagttcaaatctg

SEQ ID NO: 193 - DNA, 30 bp, Artificial Sequence
  Synthetic Construct
agtgcaagagattgaagagttcaaatctga

SEQ ID NO: 194 - DNA, 30 bp, Artificial Sequence
  Synthetic Construct
aagagttcaaatctgaccaagatgttgatg

SEQ ID NO: 195 - DNA, 30 bp, Artificial Sequence
  Synthetic Construct
aaatctgaccaagatgttgatgttggataa

SEQ ID NO: 196 - DNA, 30 bp, Artificial Sequence
  Synthetic Construct
atgttggataagagaattctctgctcccca

SEQ ID NO: 197 - DNA, 30 bp, Artificial Sequence
  Synthetic Construct
atccaatccaaatgcggcatcttcaaacct

SEQ ID NO: 198 - DNA, 30 bp, Artificial Sequence
  Synthetic Construct
gaattcatccaatccaaatgcggcatcttc

SEQ ID NO: 199 - DNA, 30 bp, Artificial Sequence
  Synthetic Construct
ggaattcatccaatccaaatgcggcatctt

SEQ ID NO: 200 - DNA, 30 bp, Artificial Sequence
  Synthetic Construct
aaaagcaagcaagcagaatttggaattcat

SEQ ID NO: 201 - DNA, 30 bp, Artificial Sequence
  Synthetic Construct
tgcataaagtgtaagtgtataagcatatca

SEQ ID NO: 202 - DNA, 30 bp, Artificial Sequence
  Synthetic Construct
gtgcataaagtgtaagtgtataagcatatc

SEQ ID NO: 203 - DNA, 30 bp, Artificial Sequence
  Synthetic Construct
caataatcctgtcaattatatttctaaatt

SEQ ID NO: 204 - DNA, 30 bp, Artificial Sequence
  Synthetic Construct
tgtgcataaagtgtaagtgtataagcatat

SEQ ID NO: 205 - DNA, 30 bp, Artificial Sequence
  Synthetic Construct
ttataaccctacattttgtgcataaagtgt

SEQ ID NO: 206 - DNA, 30 bp, Artificial Sequence
  Synthetic Construct
acattattataaccctacattttgtgcata

SEQ ID NO: 207 - DNA, 30 bp, Artificial Sequence
  Synthetic Construct
taaagaagatcatgtccatgttaacattat

SEQ ID NO: 208 - DNA, 30 bp, Artificial Sequence
  Synthetic Construct
ccagccctcctagagctacctgtggagcaa

SEQ ID NO: 209 - DNA, 30 bp, Artificial Sequence
  Synthetic Construct
tctgctccccacctctaagttgccagccct

SEQ ID NO: 210 - DNA, 30 bp, Artificial Sequence
  Synthetic Construct
gataagagaattctctgctccccacctcta

SEQ ID NO: 211 - DNA, 30 bp, Artificial Sequence
  Synthetic Construct
taaccctacattttgtgcataaagtgtaag

SEQ ID NO: 212 - DNA, 30 bp, Artificial Sequence
  Synthetic Construct
ccaataatcctgtcaattatatttctaaat

SEQ ID NO: 213 - DNA, 30 bp, Artificial Sequence
  Synthetic Construct
taacaaatttccaataatcctgtcaattat

SEQ ID NO: 214 - DNA, 30 bp, Artificial Sequence
  Synthetic Construct
attataacaaatttccaataatcctgtcaa

SEQ ID NO: 215 - DNA, 30 bp, Artificial Sequence
  Synthetic Construct
ccctgtttgaaaataaaggggtaatagtgg

SEQ ID NO: 216 - DNA, 30 bp, Artificial Sequence
  Synthetic Construct
aagactgtttccctgtttgaaaataaaggg

SEQ ID NO: 217 - DNA, 30 bp, Artificial Sequence
  Synthetic Construct
ccaagtggaacttgaagactgtttccctgt

SEQ ID NO: 218 - DNA, 30 bp, Artificial Sequence
  Synthetic Construct
accaagtggaacttgaagactgtttccctg

SEQ ID NO: 219 - DNA, 30 bp, Artificial Sequence
  Synthetic Construct
taccaagtggaacttgaagactgtttccct

SEQ ID NO: 220 - DNA, 30 bp, Artificial Sequence
  Synthetic Construct
ttaccaagtggaacttgaagactgtttccc

SEQ ID NO: 221 - DNA, 30 bp, Artificial Sequence
  Synthetic Construct
cctgtttgaaaataaaggggtaatagtggg

SEQ ID NO: 222 - DNA, 30 bp, Artificial Sequence
  Synthetic Construct
tttaccaagtggaacttgaagactgtttcc

SEQ ID NO: 223 - DNA, 30 bp, Artificial Sequence
  Synthetic Construct
cactgtgagccaaactctatatacaagggg

SEQ ID NO: 224 - DNA, 30 bp, Artificial Sequence
  Synthetic Construct
acactgtgagccaaactctatatacaaggg

SEQ ID NO: 225 - DNA, 30 bp, Artificial Sequence
  Synthetic Construct
ctaatctggaaaatgtgaatcactgaggcc

SEQ ID NO: 226 - DNA, 30 bp, Artificial Sequence
  Synthetic Construct
agcatcagattcctaatctggaaaatgtga

SEQ ID NO: 227 - DNA, 30 bp, Artificial Sequence
  Synthetic Construct
gagcatcagattcctaatctggaaaatgtg

SEQ ID NO: 228 - DNA, 30 bp, Artificial Sequence
  Synthetic Construct
tttgagcatcagattcctaatctggaaaat

SEQ ID NO: 229 - DNA, 30 bp, Artificial Sequence
  Synthetic Construct
acattttttaccaagtggaacttgaagact

SEQ ID NO: 230 - DNA, 30 bp, Artificial Sequence
  Synthetic Construct
aacttctttgagcatcagattcctaatctg

SEQ ID NO: 231 - DNA, 30 bp, Artificial Sequence
  Synthetic Construct
gaaaataaaggggtaatagtgggagtgaga

SEQ ID NO: 232 - DNA, 30 bp, Artificial Sequence
  Synthetic Construct
tatgatttatttaacttgtggaacaaaaat

SEQ ID NO: 233 - DNA, 30 bp, Artificial Sequence
  Synthetic Construct
attcattataacaaatttccaataatcctg

SEQ ID NO: 234 - DNA, 30 bp, Artificial Sequence
  Synthetic Construct
cattcattataacaaatttccaataatcct

SEQ ID NO: 235 - DNA, 30 bp, Artificial Sequence
  Synthetic Construct
tatgacaaaatgtttcattcattataacaa

SEQ ID NO: 236 - DNA, 30 bp, Artificial Sequence
  Synthetic Construct
tcaaatgtataagaagtaaatatgaatctt

SEQ ID NO: 237 - DNA, 30 bp, Artificial Sequence
  Synthetic Construct
atcaaatgtataagaagtaaatatgaatct

SEQ ID NO: 238 - DNA, 30 bp, Artificial Sequence
  Synthetic Construct
ctttatcaaatgtataagaagtaaatatga

SEQ ID NO: 239 - DNA, 30 bp, Artificial Sequence
  Synthetic Construct
aaaataaaggggtaatagtgggagtgagat

SEQ ID NO: 240 - DNA, 30 bp, Artificial Sequence
  Synthetic Construct
accacaaccatgccttactttatcaaatgt

SEQ ID NO: 241 - DNA, 30 bp, Artificial Sequence
  Synthetic Construct
acttgtggaacaaaaataaaccagattaac

SEQ ID NO: 242 - DNA, 30 bp, Artificial Sequence
  Synthetic Construct
aacttgtggaacaaaaataaaccagattaa

SEQ ID NO: 243 - DNA, 30 bp, Artificial Sequence
  Synthetic Construct
tttaacttgtggaacaaaaataaaccagat

SEQ ID NO: 244 - DNA, 30 bp, Artificial Sequence
  Synthetic Construct
atttaacttgtggaacaaaaataaaccaga

SEQ ID NO: 245 - DNA, 30 bp, Artificial Sequence
  Synthetic Construct
tgatttatttaacttgtggaacaaaaataa

SEQ ID NO: 246 - DNA, 30 bp, Artificial Sequence
  Synthetic Construct
atgatttatttaacttgtggaacaaaaata

SEQ ID NO: 247 - DNA, 30 bp, Artificial Sequence
  Synthetic Construct
tggaacaaaaataaaccagattaaccacaa

SEQ ID NO: 248 - DNA, 30 bp, Artificial Sequence
  Synthetic Construct
tttaaggaataatgacaagaaaaaaaagtc

SEQ ID NO: 249 - DNA, 30 bp, Artificial Sequence
  Synthetic Construct
attttcaaacagggaaacagtcttcaagtt

SEQ ID NO: 250 - DNA, 30 bp, Artificial Sequence
  Synthetic Construct
ttattgtttaaggaataatgacaagaaaaa

SEQ ID NO: 251 - DNA, 30 bp, Artificial Sequence
  Synthetic Construct
gagtgctgtctccatgtttgatgtatctga

SEQ ID NO: 252 - DNA, 30 bp, Artificial Sequence
  Synthetic Construct
agtgctgtctccatgtttgatgtatctgag

SEQ ID NO: 253 - DNA, 30 bp, Artificial Sequence
  Synthetic Construct
gatgtatctgagcaggttgctccacaggta

SEQ ID NO: 254 - DNA, 30 bp, Artificial Sequence
  Synthetic Construct
atgtatctgagcaggttgctccacaggtag

SEQ ID NO: 255 - DNA, 30 bp, Artificial Sequence
  Synthetic Construct
ctccacaggtagctctaggagggctggcaa

SEQ ID NO: 256 - DNA, 30 bp, Artificial Sequence
  Synthetic Construct
gaggtggggagcagagaattctcttatcca

SEQ ID NO: 257 - DNA, 30 bp, Artificial Sequence
  Synthetic Construct
tcttatccaacatcaacatcttggtcagat

SEQ ID NO: 258 - DNA, 30 bp, Artificial Sequence
  Synthetic Construct
tccaacatcaacatcttggtcagatttgaa

SEQ ID NO: 259 - DNA, 30 bp, Artificial Sequence
  Synthetic Construct
gtcagatttgaactcttcaatctcttgcac

SEQ ID NO: 260 - DNA, 30 bp, Artificial Sequence
  Synthetic Construct
gaactcttcaatctcttgcactcaaagctt

SEQ ID NO: 261 - DNA, 30 bp, Artificial Sequence
  Synthetic Construct
aactcttcaatctcttgcactcaaagcttg

SEQ ID NO: 262 - DNA, 30 bp, Artificial Sequence
  Synthetic Construct
aatctcttgcactcaaagcttgttaagata

SEQ ID NO: 263 - DNA, 30 bp, Artificial Sequence
  Synthetic Construct
cactcaaagcttgttaagatagttaagcgt

SEQ ID NO: 264 - DNA, 30 bp, Artificial Sequence
  Synthetic Construct
ttaagatagttaagcgtgcataagttaact

SEQ ID NO: 265 - DNA, 30 bp, Artificial Sequence
  Synthetic Construct
agatagttaagcgtgcataagttaacttcc

SEQ ID NO: 266 - DNA, 30 bp, Artificial Sequence
  Synthetic Construct
agcgtgcataagttaacttccaatttacat

SEQ ID NO: 267 - DNA, 30 bp, Artificial Sequence
  Synthetic Construct
acttccaatttacatactctgcttagaatt

SEQ ID NO: 268 - DNA, 30 bp, Artificial Sequence
  Synthetic Construct
taatgaatgaaacattttgtcatataagat

SEQ ID NO: 269 - DNA, 30 bp, Artificial Sequence
  Synthetic Construct
ttataatgaatgaaacattttgtcatataa

SEQ ID NO: 270 - DNA, 30 bp, Artificial Sequence
  Synthetic Construct
gttataatgaatgaaacattttgtcatata

SEQ ID NO: 271 - DNA, 30 bp, Artificial Sequence
  Synthetic Construct
gaaatttgttataatgaatgaaacattttg

SEQ ID NO: 272 - DNA, 30 bp, Artificial Sequence
  Synthetic Construct
ttggaaatttgttataatgaatgaaacatt

SEQ ID NO: 273 - DNA, 30 bp, Artificial Sequence
  Synthetic Construct
acaggattattggaaatttgttataatgaa

SEQ ID NO: 274 - DNA, 30 bp, Artificial Sequence
  Synthetic Construct
tactttgagtgctgtctccatgtttgatgt

SEQ ID NO: 275 - DNA, 30 bp, Artificial Sequence
  Synthetic Construct
gaaatataattgacaggattattggaaatt

SEQ ID NO: 276 - DNA, 30 bp, Artificial Sequence
  Synthetic Construct
ggggaaaatttagaaatataattgacagga

SEQ ID NO: 277 - DNA, 30 bp, Artificial Sequence
  Synthetic Construct
gggggaaaatttagaaatataattgacagg

SEQ ID NO: 278 - DNA, 30 bp, Artificial Sequence
  Synthetic Construct
gaatttgggggaaaatttagaaatataatt

SEQ ID NO: 279 - DNA, 30 bp, Artificial Sequence
  Synthetic Construct
catactctgcttagaatttgggggaaaatt

SEQ ID NO: 280 - DNA, 30 bp, Artificial Sequence
  Synthetic Construct
acatactctgcttagaatttgggggaaaat

SEQ ID NO: 281 - DNA, 30 bp, Artificial Sequence
  Synthetic Construct
caatttacatactctgcttagaatttgggg

SEQ ID NO: 282 - DNA, 30 bp, Artificial Sequence
  Synthetic Construct
agaaatataattgacaggattattggaaat

SEQ ID NO: 283 - DNA, 30 bp, Artificial Sequence
  Synthetic Construct
taattctactttgagtgctgtctccatgtt

SEQ ID NO: 284 - DNA, 30 bp, Artificial Sequence
  Synthetic Construct
ataattctactttgagtgctgtctccatgt

SEQ ID NO: 285 - DNA, 30 bp, Artificial Sequence
  Synthetic Construct
tttataattctactttgagtgctgtctcca

SEQ ID NO: 286 - DNA, 30 bp, Artificial Sequence
  Synthetic Construct
aagatgccgcatttggattggatgaattcc

SEQ ID NO: 287 - DNA, 30 bp, Artificial Sequence
  Synthetic Construct
gaagatgccgcatttggattggatgaattc

SEQ ID NO: 288 - DNA, 30 bp, Artificial Sequence
  Synthetic Construct
aggtttgaagatgccgcatttggattggat

SEQ ID NO: 289 - DNA, 30 bp, Artificial Sequence
  Synthetic Construct
caggtttgaagatgccgcatttggattgga

SEQ ID NO: 290 - DNA, 30 bp, Artificial Sequence
  Synthetic Construct
tcaggtttgaagatgccgcatttggattgg

SEQ ID NO: 291 - DNA, 30 bp, Artificial Sequence
  Synthetic Construct
ttttcaggtttgaagatgccgcatttggat

SEQ ID NO: 292 - DNA, 30 bp, Artificial Sequence
  Synthetic Construct
ggattggatgaattccaaattctgcttgct

SEQ ID NO: 293 - DNA, 30 bp, Artificial Sequence
  Synthetic Construct
cttttcaggtttgaagatgccgcatttgga

SEQ ID NO: 294 - DNA, 30 bp, Artificial Sequence
  Synthetic Construct
ttttcttttcaggtttgaagatgccgcatt

SEQ ID NO: 295 - DNA, 30 bp, Artificial Sequence
  Synthetic Construct
cttttcttttcaggtttgaagatgccgcat

SEQ ID NO: 296 - DNA, 30 bp, Artificial Sequence
  Synthetic Construct
tcttttcttttcaggtttgaagatgccgca

SEQ ID NO: 297 - DNA, 30 bp, Artificial Sequence
  Synthetic Construct
ttcttttcttttcaggtttgaagatgccgc

SEQ ID NO: 298 - DNA, 30 bp, Artificial Sequence
  Synthetic Construct
ctaacctttttcttttcttttcaggtttga

SEQ ID NO: 299 - DNA, 30 bp, Artificial Sequence
  Synthetic Construct
attgctaacctttttcttttcttttcaggt

SEQ ID NO: 300 - DNA, 30 bp, Artificial Sequence
  Synthetic Construct
tcttttcaggtttgaagatgccgcatttgg

SEQ ID NO: 301 - DNA, 30 bp, Artificial Sequence
  Synthetic Construct
tgtcatataagattcatatttacttcttat

SEQ ID NO: 302 - DNA, 30 bp, Artificial Sequence
  Synthetic Construct
gattggatgaattccaaattctgcttgctt

SEQ ID NO: 303 - DNA, 30 bp, Artificial Sequence
  Synthetic Construct
caaattctgcttgcttgctttttaatattg

SEQ ID NO: 304 - DNA, 30 bp, Artificial Sequence
  Synthetic Construct
acatggacatgatcttctttataattctac

SEQ ID NO: 305 - DNA, 30 bp, Artificial Sequence
  Synthetic Construct
taataatgttaacatggacatgatcttctt

SEQ ID NO: 306 - DNA, 30 bp, Artificial Sequence
  Synthetic Construct
tgcacaaaatgtagggttataataatgtta

SEQ ID NO: 307 - DNA, 30 bp, Artificial Sequence
  Synthetic Construct
atgcacaaaatgtagggttataataatgtt

SEQ ID NO: 308 - DNA, 30 bp, Artificial Sequence
  Synthetic Construct
cactttatgcacaaaatgtagggttataat

SEQ ID NO: 309 - DNA, 30 bp, Artificial Sequence
  Synthetic Construct
tacacttacactttatgcacaaaatgtagg

SEQ ID NO: 310 - DNA, 30 bp, Artificial Sequence
  Synthetic Construct
gatgaattccaaattctgcttgcttgcttt

SEQ ID NO: 311 - DNA, 30 bp, Artificial Sequence
  Synthetic Construct
atatgcttatacacttacactttatgcaca

SEQ ID NO: 312 - DNA, 30 bp, Artificial Sequence
  Synthetic Construct
aatattgatatgcttatacacttacacttt

SEQ ID NO: 313 - DNA, 30 bp, Artificial Sequence
  Synthetic Construct
taatattgatatgcttatacacttacactt

SEQ ID NO: 314 - DNA, 30 bp, Artificial Sequence
  Synthetic Construct
ttaatattgatatgcttatacacttacact

SEQ ID NO: 315 - DNA, 30 bp, Artificial Sequence
  Synthetic Construct
ctttttaatattgatatgcttatacactta

SEQ ID NO: 316 - DNA, 30 bp, Artificial Sequence
  Synthetic Construct
cttgctttttaatattgatatgcttataca

SEQ ID NO: 317 - DNA, 30 bp, Artificial Sequence
  Synthetic Construct
tgcttgcttgctttttaatattgatatgct

SEQ ID NO: 318 - DNA, 30 bp, Artificial Sequence
  Synthetic Construct
atattgatatgcttatacacttacacttta

SEQ ID NO: 319 - DNA, 30 bp, Artificial Sequence
  Synthetic Construct
ttgtttaaggaataatgacaagaaaaaaaa

SEQ ID NO: 320 - DNA, 30 bp, Artificial Sequence
  Synthetic Construct
gtcatataagattcatatttacttcttata

SEQ ID NO: 321 - DNA, 30 bp, Artificial Sequence
  Synthetic Construct
atatttacttcttatacatttgataaagta

SEQ ID NO: 322 - DNA, 30 bp, Artificial Sequence
  Synthetic Construct
aatggcatagttggggtgacacagctgtct

SEQ ID NO: 323 - DNA, 30 bp, Artificial Sequence
  Synthetic Construct
gggtgacacagctgtctagtgggaggccag

SEQ ID NO: 324 - DNA, 30 bp, Artificial Sequence
  Synthetic Construct
tatattttagccagcgttctttcctgcggg

SEQ ID NO: 325 - DNA, 30 bp, Artificial Sequence
  Synthetic Construct
tagccagcgttctttcctgcgggccaggtc

SEQ ID NO: 326 - DNA, 30 bp, Artificial Sequence
  Synthetic Construct
agccagcgttctttcctgcgggccaggtca

SEQ ID NO: 327 - DNA, 30 bp, Artificial Sequence
  Synthetic Construct
gccagcgttctttcctgcgggccaggtcat

SEQ ID NO: 328 - DNA, 30 bp, Artificial Sequence
  Synthetic Construct
tttcctgcgggccaggtcatgaggagtatg

SEQ ID NO: 329 - DNA, 30 bp, Artificial Sequence
  Synthetic Construct
cctgcgggccaggtcatgaggagtatgcag

SEQ ID NO: 330 - DNA, 30 bp, Artificial Sequence
  Synthetic Construct
ctgcgggccaggtcatgaggagtatgcaga

SEQ ID NO: 331 - DNA, 30 bp, Artificial Sequence
  Synthetic Construct
aatattttagcaaggaatagatatacaatc

SEQ ID NO: 332 - DNA, 30 bp, Artificial Sequence
  Synthetic Construct
atattttagcaaggaatagatatacaatca

SEQ ID NO: 333 - DNA, 30 bp, Artificial Sequence
  Synthetic Construct
tagcaaggaatagatatacaatcatccctt

SEQ ID NO: 334 - DNA, 30 bp, Artificial Sequence
  Synthetic Construct
agcaaggaatagatatacaatcatcccttg

SEQ ID NO: 335 - DNA, 30 bp, Artificial Sequence
  Synthetic Construct
gcaaggaatagatatacaatcatcccttgg

SEQ ID NO: 336 - DNA, 30 bp, Artificial Sequence
  Synthetic Construct
gtctccctgggggattggtttcaggacccc

SEQ ID NO: 337 - DNA, 30 bp, Artificial Sequence
  Synthetic Construct
gtttcaggaccccttcttggacaccaaatc

SEQ ID NO: 338 - DNA, 30 bp, Artificial Sequence
  Synthetic Construct
caggaccccttcttggacaccaaatctatg

SEQ ID NO: 339 - DNA, 30 bp, Artificial Sequence
  Synthetic Construct
taatacctaatacaatgtaaatgctatgca

SEQ ID NO: 340 - DNA, 30 bp, Artificial Sequence
  Synthetic Construct
cttgtaatacctaatacaatgtaaatgcta

SEQ ID NO: 341 - DNA, 30 bp, Artificial Sequence
  Synthetic Construct
tagattacttgtaatacctaatacaatgta

SEQ ID NO: 342 - DNA, 30 bp, Artificial Sequence
  Synthetic Construct
ctagattacttgtaatacctaatacaatgt

SEQ ID NO: 343 - DNA, 30 bp, Artificial Sequence
  Synthetic Construct
aatcatttctagattacttgtaatacctaa

SEQ ID NO: 344 - DNA, 30 bp, Artificial Sequence
  Synthetic Construct
aaatcatttctagattacttgtaataccta

SEQ ID NO: 345 - DNA, 30 bp, Artificial Sequence
  Synthetic Construct
ggaatctgatgctcaaagaagttaaatggc

SEQ ID NO: 346 - DNA, 30 bp, Artificial Sequence
  Synthetic Construct
catataacctatccacatcctcctgtatac

SEQ ID NO: 347 - DNA, 30 bp, Artificial Sequence
  Synthetic Construct
tataaaatggtatagtatttgcatataacc

SEQ ID NO: 348 - DNA, 30 bp, Artificial Sequence
  Synthetic Construct
agtcccttctataaaatggtatagtatttg

SEQ ID NO: 349 - DNA, 30 bp, Artificial Sequence
  Synthetic Construct
aagtcccttctataaaatggtatagtattt

SEQ ID NO: 350 - DNA, 30 bp, Artificial Sequence
  Synthetic Construct
gacaccaaatctatggatatttaagtccct

SEQ ID NO: 351 - DNA, 30 bp, Artificial Sequence
  Synthetic Construct
ttggacaccaaatctatggatatttaagtc

SEQ ID NO: 352 - DNA, 30 bp, Artificial Sequence
  Synthetic Construct
aggaccccttcttggacaccaaatctatgg

SEQ ID NO: 353 - DNA, 30 bp, Artificial Sequence
  Synthetic Construct
gcatataacctatccacatcctcctgtata

SEQ ID NO: 354 - DNA, 30 bp, Artificial Sequence
  Synthetic Construct
cagattaggaatctgatgctcaaagaagtt

SEQ ID NO: 355 - DNA, 30 bp, Artificial Sequence
  Synthetic Construct
ccagattaggaatctgatgctcaaagaagt

SEQ ID NO: 356 - DNA, 30 bp, Artificial Sequence
  Synthetic Construct
tccagattaggaatctgatgctcaaagaag

SEQ ID NO: 357 - DNA, 30 bp, Artificial Sequence
  Synthetic Construct
ttccacaagttaaataaatcataaaacttg

SEQ ID NO: 358 - DNA, 30 bp, Artificial Sequence
  Synthetic Construct
gttccacaagttaaataaatcataaaactt

SEQ ID NO: 359 - DNA, 30 bp, Artificial Sequence
  Synthetic Construct
tgttccacaagttaaataaatcataaaact

SEQ ID NO: 360 - DNA, 30 bp, Artificial Sequence
  Synthetic Construct
ttgttccacaagttaaataaatcataaaac

SEQ ID NO: 361 - DNA, 30 bp, Artificial Sequence
  Synthetic Construct
tttttgttccacaagttaaataaatcataa

SEQ ID NO: 362 - DNA, 30 bp, Artificial Sequence
  Synthetic Construct
atttttgttccacaagttaaataaatcata

SEQ ID NO: 363 - DNA, 30 bp, Artificial Sequence
  Synthetic Construct
cacaagttaaataaatcataaaacttgatg

SEQ ID NO: 364 - DNA, 30 bp, Artificial Sequence
  Synthetic Construct
atctggtttatttttgttccacaagttaaa

SEQ ID NO: 365 - DNA, 30 bp, Artificial Sequence
  Synthetic Construct
ataaagtaaggcatggttgtggttaatctg

SEQ ID NO: 366 - DNA, 30 bp, Artificial Sequence
  Synthetic Construct
gataaagtaaggcatggttgtggttaatct

SEQ ID NO: 367 - DNA, 30 bp, Artificial Sequence
  Synthetic Construct
tacatttgataaagtaaggcatggttgtgg

SEQ ID NO: 368 - DNA, 30 bp, Artificial Sequence
  Synthetic Construct
ttatacatttgataaagtaaggcatggttg

SEQ ID NO: 369 - DNA, 30 bp, Artificial Sequence
  Synthetic Construct
cttcttatacatttgataaagtaaggcatg

SEQ ID NO: 370 - DNA, 30 bp, Artificial Sequence
  Synthetic Construct
acttcttatacatttgataaagtaaggcat

SEQ ID NO: 371 - DNA, 30 bp, Artificial Sequence
  Synthetic Construct
tggttaatctggtttatttttgttccacaa

SEQ ID NO: 372 - DNA, 30 bp, Artificial Sequence
  Synthetic Construct
tcatataagattcatatttacttcttatac

SEQ ID NO: 373 - DNA, 30 bp, Artificial Sequence
  Synthetic Construct
aataaatcataaaacttgatgtgttatctc

SEQ ID NO: 374 - DNA, 30 bp, Artificial Sequence
  Synthetic Construct
tctcttatatctcactcccactattacccc

SEQ ID NO: 375 - DNA, 30 bp, Artificial Sequence
  Synthetic Construct
acattttccagattaggaatctgatgctca

SEQ ID NO: 376 - DNA, 30 bp, Artificial Sequence
  Synthetic Construct
gctcacagtgtaaagggcctcagtgattca

SEQ ID NO: 377 - DNA, 30 bp, Artificial Sequence
  Synthetic Construct
ggctcacagtgtaaagggcctcagtgattc

SEQ ID NO: 378 - DNA, 30 bp, Artificial Sequence
  Synthetic Construct
tatatagagtttggctcacagtgtaaaggg

SEQ ID NO: 379 - DNA, 30 bp, Artificial Sequence
  Synthetic Construct
gtaaaaaatgtgaaccccttgtatatagag

SEQ ID NO: 380 - DNA, 30 bp, Artificial Sequence
  Synthetic Construct
cacttggtaaaaaatgtgaaccccttgtat

SEQ ID NO: 381 - DNA, 30 bp, Artificial Sequence
  Synthetic Construct
atgtgttatctcttatatctcactcccact

SEQ ID NO: 382 - DNA, 30 bp, Artificial Sequence
  Synthetic Construct
aagttccacttggtaaaaaatgtgaacccc

SEQ ID NO: 383 - DNA, 30 bp, Artificial Sequence
  Synthetic Construct
caaacagggaaacagtcttcaagttccact

SEQ ID NO: 384 - DNA, 30 bp, Artificial Sequence
  Synthetic Construct
tcaaacagggaaacagtcttcaagttccac

SEQ ID NO: 385 - DNA, 30 bp, Artificial Sequence
  Synthetic Construct
ttttcaaacagggaaacagtcttcaagttc

SEQ ID NO: 386 - DNA, 30 bp, Artificial Sequence
  Synthetic Construct
aaacctgaaaagaaaagaaaaaggttagca

SEQ ID NO: 387 - DNA, 30 bp, Artificial Sequence
  Synthetic Construct
cccctttattttcaaacagggaaacagtct

SEQ ID NO: 388 - DNA, 30 bp, Artificial Sequence
  Synthetic Construct
tatctcactcccactattacccctttattt

SEQ ID NO: 389 - DNA, 30 bp, Artificial Sequence
  Synthetic Construct
aaacagggaaacagtcttcaagttccactt

SEQ ID NO: 390 - DNA, 30 bp, Artificial Sequence
  Synthetic Construct
gcaatgaatttattttatttggattgcaga

SEQ ID NO: 391 - RNA, 30 nt, Artificial Sequence
  Synthetic Construct
auauaaguggaggcgucgcgcuggcgggca

SEQ ID NO: 392 - RNA, 30 nt, Artificial Sequence
  Synthetic Construct
uauuaaacgcgugcccagccaaucaggaca

SEQ ID NO: 393 - RNA, 30 nt, Artificial Sequence
  Synthetic Construct
aggaaugcccgccagcgcgacgccuccacu

SEQ ID NO: 394 - RNA, 30 nt, Artificial Sequence
  Synthetic Construct
uggccuggaggcuauccagcgugagucucu

SEQ ID NO: 395 - RNA, 30 nt, Artificial Sequence
  Synthetic Construct
cuggccuggaggcuauccagcgugagucuc

SEQ ID NO: 396 - RNA, 30 nt, Artificial Sequence
  Synthetic Construct
gcugugcucgcgcuacucucucuuucuggc

SEQ ID NO: 397 - RNA, 30 nt, Artificial Sequence
  Synthetic Construct
gggccgagaugucucgcuccguggccuuag

SEQ ID NO: 398 - RNA, 30 nt, Artificial Sequence
  Synthetic Construct
cugaagcugacagcauucgggccgagaugu

SEQ ID NO: 399 - RNA, 30 nt, Artificial Sequence
  Synthetic Construct
aauauaaguggaggcgucgcgcuggcgggc

SEQ ID NO: 400 - RNA, 30 nt, Artificial Sequence
  Synthetic Construct
gcugggcacgcguuuaauauaaguggaggc

SEQ ID NO: 401 - RNA, 30 nt, Artificial Sequence
  Synthetic Construct
ucacagcccaagauaguuaagugggguaag

SEQ ID NO: 402 - RNA, 30 nt, Artificial Sequence
  Synthetic Construct
agugggguaagucuuacauucuuuuguaag

SEQ ID NO: 403 - RNA, 30 nt, Artificial Sequence
  Synthetic Construct
cauucucugcuggaugacgugaguaaaccu

SEQ ID NO: 404 - RNA, 30 nt, Artificial Sequence
  Synthetic Construct
uuuuguaagcugcugaaaguuguguaugag

SEQ ID NO: 405 - RNA, 30 nt, Artificial Sequence
  Synthetic Construct
gucacagcccaagauaguuaagugggguaa

SEQ ID NO: 406 - RNA, 30 nt, Artificial Sequence
  Synthetic Construct
cauucuuuuguaagcugcugaaaguugugu

SEQ ID NO: 407 - RNA, 30 nt, Artificial Sequence
  Synthetic Construct
acccccacugaaaaagaugaguaugccugc

SEQ ID NO: 408 - RNA, 30 nt, Artificial Sequence
  Synthetic Construct
cagcaaggacuggucuuucuaucucuugua

SEQ ID NO: 409 - RNA, 30 nt, Artificial Sequence
  Synthetic Construct
uaucucuuguacuacacugaauucaccccc

SEQ ID NO: 410 - RNA, 30 nt, Artificial Sequence
  Synthetic Construct
cuaucucuuguacuacacugaauucacccc

SEQ ID NO: 411 - RNA, 30 nt, Artificial Sequence
  Synthetic Construct
agcaaggacuggucuuucuaucucuuguac

SEQ ID NO: 412 - RNA, 30 nt, Artificial Sequence
  Synthetic Construct
uguaagcugcugaaaguuguguaugaguag

SEQ ID NO: 413 - RNA, 30 nt, Artificial Sequence
  Synthetic Construct
ucuuucagcaaggacuggucuuucuaucuc

SEQ ID NO: 414 - RNA, 30 nt, Artificial Sequence
  Synthetic Construct
uacuacacugaauucacccccacugaaaaa

SEQ ID NO: 415 - RNA, 30 nt, Artificial Sequence
  Synthetic Construct
guaagcugcugaaaguuguguaugaguagu

SEQ ID NO: 416 - RNA, 30 nt, Artificial Sequence
  Synthetic Construct
ccccacuuaacuaucuugggcugugacaaa

SEQ ID NO: 417 - RNA, 30 nt, Artificial Sequence
  Synthetic Construct
cagcagcuuacaaaagaauguaagacuuac

SEQ ID NO: 418 - RNA, 30 nt, Artificial Sequence
  Synthetic Construct
agcagcuuacaaaagaauguaagacuuacc

SEQ ID NO: 419 - RNA, 30 nt, Artificial Sequence
  Synthetic Construct
caaaagaauguaagacuuaccccacuuaac

SEQ ID NO: 420 - RNA, 30 nt, Artificial Sequence
  Synthetic Construct
agacuugucuuucagcaaggacuggucuuu

SEQ ID NO: 421 - RNA, 30 nt, Artificial Sequence
  Synthetic Construct
acuaucuugggcugugacaaagucacaugg

SEQ ID NO: 422 - RNA, 30 nt, Artificial Sequence
  Synthetic Construct
ggcugugacaaagucacaugguucacacgg

SEQ ID NO: 423 - RNA, 30 nt, Artificial Sequence
  Synthetic Construct
acacggcaggcauacucaucuuuuucagug

SEQ ID NO: 424 - RNA, 30 nt, Artificial Sequence
  Synthetic Construct
uucagugggggugaauucaguguaguacaa

SEQ ID NO: 425 - RNA, 30 nt, Artificial Sequence
  Synthetic Construct
ucagugggggugaauucaguguaguacaag

SEQ ID NO: 426 - RNA, 30 nt, Artificial Sequence
  Synthetic Construct
cagugggggugaauucaguguaguacaaga

SEQ ID NO: 427 - RNA, 30 nt, Artificial Sequence
  Synthetic Construct
agugggggugaauucaguguaguacaagag

SEQ ID NO: 428 - RNA, 30 nt, Artificial Sequence
  Synthetic Construct
aguguaguacaagagauagaaagaccaguc

SEQ ID NO: 429 - RNA, 30 nt, Artificial Sequence
  Synthetic Construct
cugaaagacaagucugaaugcuccacuuuu

SEQ ID NO: 430 - RNA, 30 nt, Artificial Sequence
  Synthetic Construct
uaagcugcugaaaguuguguaugaguaguc

SEQ ID NO: 431 - RNA, 30 nt, Artificial Sequence
  Synthetic Construct
aaaaaguggagcauucagacuugucuuuca

SEQ ID NO: 432 - RNA, 30 nt, Artificial Sequence
  Synthetic Construct
cauccauccgacauugaaguugacuuacug

SEQ ID NO: 433 - RNA, 30 nt, Artificial Sequence
  Synthetic Construct
acuuacugaagaauggagagagaauugaaa

SEQ ID NO: 434 - RNA, 30 nt, Artificial Sequence
  Synthetic Construct
auauugccaggguauuucacuuggggcuaa

SEQ ID NO: 435 - RNA, 30 nt, Artificial Sequence
  Synthetic Construct
gaguaccugaggaauaucgggaaaagacac

SEQ ID NO: 436 - RNA, 30 nt, Artificial Sequence
  Synthetic Construct
ggaguaccugaggaauaucgggaaaagaca

SEQ ID NO: 437 - RNA, 30 nt, Artificial Sequence
  Synthetic Construct
ucugcuggaugacgugaguaaaccugaauc

SEQ ID NO: 438 - RNA, 30 nt, Artificial Sequence
  Synthetic Construct
ccauucucugcuggaugacgugaguaaacc

SEQ ID NO: 439 - RNA, 30 nt, Artificial Sequence
  Synthetic Construct
acuuuccauucucugcuggaugacgugagu

SEQ ID NO: 440 - RNA, 30 nt, Artificial Sequence
  Synthetic Construct
gacuuuccauucucugcuggaugacgugag

SEQ ID NO: 441 - RNA, 30 nt, Artificial Sequence
  Synthetic Construct
aggaaauuugacuuuccauucucugcugga

SEQ ID NO: 442 - RNA, 30 nt, Artificial Sequence
  Synthetic Construct
aaugucggauggaugaaacccagacacaua

SEQ ID NO: 443 - RNA, 30 nt, Artificial Sequence
  Synthetic Construct
aguaagucaacuucaaugucggauggauga

SEQ ID NO: 444 - RNA, 30 nt, Artificial Sequence
  Synthetic Construct
uucaguaagucaacuucaaugucggaugga

SEQ ID NO: 445 - RNA, 30 nt, Artificial Sequence
  Synthetic Construct
ucucuccauucuucaguaagucaacuucaa

SEQ ID NO: 446 - RNA, 30 nt, Artificial Sequence
  Synthetic Construct
aauucucucuccauucuucaguaagucaac

SEQ ID NO: 447 - RNA, 30 nt, Artificial Sequence
  Synthetic Construct
cugaagaauggagagagaauugaaaaagug

SEQ ID NO: 448 - RNA, 30 nt, Artificial Sequence
  Synthetic Construct
caauucucucuccauucuucaguaagucaa

SEQ ID NO: 449 - RNA, 30 nt, Artificial Sequence
  Synthetic Construct
ucccgauauuccucagguacuccaaagauu

SEQ ID NO: 450 - RNA, 30 nt, Artificial Sequence
  Synthetic Construct
cccgauauuccucagguacuccaaagauuc

SEQ ID NO: 451 - RNA, 30 nt, Artificial Sequence
  Synthetic Construct
ccgauauuccucagguacuccaaagauuca

SEQ ID NO: 452 - RNA, 30 nt, Artificial Sequence
  Synthetic Construct
cucagguacuccaaagauucagguuuacuc

SEQ ID NO: 453 - RNA, 30 nt, Artificial Sequence
  Synthetic Construct
agguuuacucacgucauccagcagagaaug

SEQ ID NO: 454 - RNA, 30 nt, Artificial Sequence
  Synthetic Construct
acucacgucauccagcagagaauggaaagu

SEQ ID NO: 455 - RNA, 30 nt, Artificial Sequence
  Synthetic Construct
cucacgucauccagcagagaauggaaaguc

SEQ ID NO: 456 - RNA, 30 nt, Artificial Sequence
  Synthetic Construct
ccugaauugcuaugugucuggguuucaucc

SEQ ID NO: 457 - RNA, 30 nt, Artificial Sequence
  Synthetic Construct
cugaauugcuaugugucuggguuucaucca

SEQ ID NO: 458 - RNA, 30 nt, Artificial Sequence
  Synthetic Construct
cuaugugucuggguuucauccauccgacau

SEQ ID NO: 459 - RNA, 30 nt, Artificial Sequence
  Synthetic Construct
uucaauucucucuccauucuucaguaaguc

SEQ ID NO: 460 - RNA, 30 nt, Artificial Sequence
  Synthetic Construct
auccauccgacauugaaguugacuuacuga

SEQ ID NO: 461 - RNA, 30 nt, Artificial Sequence
  Synthetic Construct
aaguugacuuacugaagaauggagagagaa

SEQ ID NO: 462 - RNA, 30 nt, Artificial Sequence
  Synthetic Construct
augugucuuuucccgauauuccucagguac

SEQ ID NO: 463 - RNA, 30 nt, Artificial Sequence
  Synthetic Construct
ucaauucucucuccauucuucaguaaguca

SEQ ID NO: 464 - RNA, 30 nt, Artificial Sequence
  Synthetic Construct
augucuuccuuuuuuuucuccacugucuuu

SEQ ID NO: 465 - RNA, 30 nt, Artificial Sequence
  Synthetic Construct
ccuccaugaugcugcuuacaugucucgauc

SEQ ID NO: 466 - RNA, 30 nt, Artificial Sequence
  Synthetic Construct
caugucucgaucuaugaaaaagacagugga

SEQ ID NO: 467 - RNA, 30 nt, Artificial Sequence
  Synthetic Construct
cuuuuuuuucuccacugucuuuuucauaga

SEQ ID NO: 468 - RNA, 30 nt, Artificial Sequence
  Synthetic Construct
uuuuucuccacugucuuuuucauagaucga

SEQ ID NO: 469 - RNA, 30 nt, Artificial Sequence
  Synthetic Construct
uuuucuccacugucuuuuucauagaucgag

SEQ ID NO: 470 - RNA, 30 nt, Artificial Sequence
  Synthetic Construct
uuucuccacugucuuuuucauagaucgaga

SEQ ID NO: 471 - RNA, 30 nt, Artificial Sequence
  Synthetic Construct
ucuccacugucuuuuucauagaucgagaca

SEQ ID NO: 472 - RNA, 30 nt, Artificial Sequence
  Synthetic Construct
cuccacugucuuuuucauagaucgagacau

SEQ ID NO: 473 - RNA, 30 nt, Artificial Sequence
  Synthetic Construct
uccacugucuuuuucauagaucgagacaug

SEQ ID NO: 474 - RNA, 30 nt, Artificial Sequence
  Synthetic Construct
uucauagaucgagacauguaagcagcauca

SEQ ID NO: 475 - RNA, 30 nt, Artificial Sequence
  Synthetic Construct
ucauagaucgagacauguaagcagcaucau

SEQ ID NO: 476 - RNA, 30 nt, Artificial Sequence
  Synthetic Construct
cauagaucgagacauguaagcagcaucaug

SEQ ID NO: 477 - RNA, 30 nt, Artificial Sequence
  Synthetic Construct
uucuccacugucuuuuucauagaucgagac

SEQ ID NO: 478 - RNA, 30 nt, Artificial Sequence
  Synthetic Construct
uugaccuugagaaaauguuuuuguuucacu

SEQ ID NO: 479 - RNA, 30 nt, Artificial Sequence
  Synthetic Construct
auagaucgagacauguaagcagcaucaugg

SEQ ID NO: 480 - RNA, 30 nt, Artificial Sequence
  Synthetic Construct
cucaaggucaaaaacuuaccuccaugaugc

SEQ ID NO: 481 - RNA, 30 nt, Artificial Sequence
  Synthetic Construct
ucucaaggucaaaaacuuaccuccaugaug

SEQ ID NO: 482 - RNA, 30 nt, Artificial Sequence
  Synthetic Construct
ucaaggucaaaaacuuaccuccaugaugcu

SEQ ID NO: 483 - RNA, 30 nt, Artificial Sequence
  Synthetic Construct
uuguuucacuguccugaggacuauuuauag

SEQ ID NO: 484 - RNA, 30 nt, Artificial Sequence
  Synthetic Construct
uguuucacuguccugaggacuauuuauaga

SEQ ID NO: 485 - RNA, 30 nt, Artificial Sequence
  Synthetic Construct
agaaaauguuuuuguuucacuguccugagg

SEQ ID NO: 486 - RNA, 30 nt, Artificial Sequence
  Synthetic Construct
accuugagaaaauguuuuuguuucacuguc

SEQ ID NO: 487 - RNA, 30 nt, Artificial Sequence
  Synthetic Construct
gaccuugagaaaauguuuuuguuucacugu

SEQ ID NO: 488 - RNA, 30 nt, Artificial Sequence
  Synthetic Construct
ugaccuugagaaaauguuuuuguuucacug

SEQ ID NO: 489 - RNA, 30 nt, Artificial Sequence
  Synthetic Construct
uauuagguauuacaaguaaucuagaaauga

SEQ ID NO: 490 - RNA, 30 nt, Artificial Sequence
  Synthetic Construct
acauuguauuagguauuacaaguaaucuag

SEQ ID NO: 491 - RNA, 30 nt, Artificial Sequence
  Synthetic Construct
cauagcauuuacauuguauuagguauuaca

SEQ ID NO: 492 - RNA, 30 nt, Artificial Sequence
  Synthetic Construct
gcauagcauuuacauuguauuagguauuac

SEQ ID NO: 493 - RNA, 30 nt, Artificial Sequence
  Synthetic Construct
cauuguauuagguauuacaaguaaucuaga

SEQ ID NO: 494 - RNA, 30 nt, Artificial Sequence
  Synthetic Construct
aacaauaacaacuauuugcauagcauuuac

SEQ ID NO: 495 - RNA, 30 nt, Artificial Sequence
  Synthetic Construct
cuugucauuauuccuuaaacaauaacaacu

SEQ ID NO: 496 - RNA, 30 nt, Artificial Sequence
  Synthetic Construct
uuccuuaaacaauaacaacuauuugcauag

SEQ ID NO: 497 - RNA, 30 nt, Artificial Sequence
  Synthetic Construct
ucauuauuccuuaaacaauaacaacuauuu

SEQ ID NO: 498 - RNA, 30 nt, Artificial Sequence
  Synthetic Construct
uugucauuauuccuuaaacaauaacaacua

SEQ ID NO: 499 - RNA, 30 nt, Artificial Sequence
  Synthetic Construct
ucuugucauuauuccuuaaacaauaacaac

SEQ ID NO: 500 - RNA, 30 nt, Artificial Sequence
  Synthetic Construct
gguauuacaaguaaucuagaaaugauuuaa

SEQ ID NO: 501 - RNA, 30 nt, Artificial Sequence
  Synthetic Construct
uucuugucauuauuccuuaaacaauaacaa

SEQ ID NO: 502 - RNA, 30 nt, Artificial Sequence
  Synthetic Construct
cuuaaacaauaacaacuauuugcauagcau

SEQ ID NO: 503 - RNA, 30 nt, Artificial Sequence
  Synthetic Construct
caaguaaucuagaaaugauuuaaaguauac

SEQ ID NO: 504 - RNA, 30 nt, Artificial Sequence
  Synthetic Construct
cuugcuaaaauauuaaauccuucagauacu

SEQ ID NO: 505 - RNA, 30 nt, Artificial Sequence
  Synthetic Construct
aaguauacaggaggauguggauagguuaua

SEQ ID NO: 506 - RNA, 30 nt, Artificial Sequence
  Synthetic Construct
gcucccucuuagagucugcauacuccucau

SEQ ID NO: 507 - RNA, 30 nt, Artificial Sequence
  Synthetic Construct
ugcucccucuuagagucugcauacuccuca

SEQ ID NO: 508 - RNA, 30 nt, Artificial Sequence
  Synthetic Construct
agauacuuuugcucccucuuagagucugca

SEQ ID NO: 509 - RNA, 30 nt, Artificial Sequence
  Synthetic Construct
aauccuucagauacuuuugcucccucuuag

SEQ ID NO: 510 - RNA, 30 nt, Artificial Sequence
  Synthetic Construct
cuaaaauauuaaauccuucagauacuuuug

SEQ ID NO: 511 - RNA, 30 nt, Artificial Sequence
  Synthetic Construct
uuucuugucauuauuccuuaaacaauaaca

SEQ ID NO: 512 - RNA, 30 nt, Artificial Sequence
  Synthetic Construct
aaaguauacaggaggauguggauagguuau

SEQ ID NO: 513 - RNA, 30 nt, Artificial Sequence
  Synthetic Construct
uauaucuauuccuugcuaaaauauuaaauc

SEQ ID NO: 514 - RNA, 30 nt, Artificial Sequence
  Synthetic Construct
gguguccaagaagggguccugaaaccaauc

SEQ ID NO: 515 - RNA, 30 nt, Artificial Sequence
  Synthetic Construct
aauauccauagauuugguguccaagaaggg

SEQ ID NO: 516 - RNA, 30 nt, Artificial Sequence
  Synthetic Construct
uagaagggacuuaaauauccauagauuugg

SEQ ID NO: 517 - RNA, 30 nt, Artificial Sequence
  Synthetic Construct
auagaagggacuuaaauauccauagauuug

SEQ ID NO: 518 - RNA, 30 nt, Artificial Sequence
  Synthetic Construct
uauagaagggacuuaaauauccauagauuu

SEQ ID NO: 519 - RNA, 30 nt, Artificial Sequence
  Synthetic Construct
uaugcaaauacuauaccauuuuauagaagg

SEQ ID NO: 520 - RNA, 30 nt, Artificial Sequence
  Synthetic Construct
guguccaagaagggguccugaaaccaaucc

SEQ ID NO: 521 - RNA, 30 nt, Artificial Sequence
  Synthetic Construct
uuuucuugucauuauuccuuaaacaauaac

SEQ ID NO: 522 - RNA, 30 nt, Artificial Sequence
  Synthetic Construct
aaaucuguuauuugucauucaaaguacagc

SEQ ID NO: 523 - RNA, 30 nt, Artificial Sequence
  Synthetic Construct
cugagcauguacagacuuuuuuuucuuguc

SEQ ID NO: 524 - RNA, 30 nt, Artificial Sequence
  Synthetic Construct
cugaauccacagauguggagccccuggaua

SEQ ID NO: 525 - RNA, 30 nt, Artificial Sequence
  Synthetic Construct
gcugaauccacagauguggagccccuggau

SEQ ID NO: 526 - RNA, 30 nt, Artificial Sequence
  Synthetic Construct
auccaugguuugcugaauccacagaugugg

SEQ ID NO: 527 - RNA, 30 nt, Artificial Sequence
  Synthetic Construct
gauccaugguuugcugaauccacagaugug

SEQ ID NO: 528 - RNA, 30 nt, Artificial Sequence
  Synthetic Construct
ugauccaugguuugcugaauccacagaugu

SEQ ID NO: 529 - RNA, 30 nt, Artificial Sequence
  Synthetic Construct
uugauccaugguuugcugaauccacagaug

SEQ ID NO: 530 - RNA, 30 nt, Artificial Sequence
  Synthetic Construct
gaaugacaaauaacagauuuaaaauuuuca

SEQ ID NO: 531 - RNA, 30 nt, Artificial Sequence
  Synthetic Construct
cccaguguuuuugauccaugguuugcugaa

SEQ ID NO: 532 - RNA, 30 nt, Artificial Sequence
  Synthetic Construct
uccccaguguuuuugauccaugguuugcug

SEQ ID NO: 533 - RNA, 30 nt, Artificial Sequence
  Synthetic Construct
uuccccaguguuuuugauccaugguuugcu

SEQ ID NO: 534 - RNA, 30 nt, Artificial Sequence
  Synthetic Construct
uuuccccaguguuuuugauccaugguuugc

SEQ ID NO: 535 - RNA, 30 nt, Artificial Sequence
  Synthetic Construct
uuuuccccaguguuuuugauccaugguuug

SEQ ID NO: 536 - RNA, 30 nt, Artificial Sequence
  Synthetic Construct
aggaauaaugacaagaaaaaaaagucugua

SEQ ID NO: 537 - RNA, 30 nt, Artificial Sequence
  Synthetic Construct
cucccucuuagagucugcauacuccucaug

SEQ ID NO: 538 - RNA, 30 nt, Artificial Sequence
  Synthetic Construct
ccccaguguuuuugauccaugguuugcuga

SEQ ID NO: 539 - RNA, 30 nt, Artificial Sequence
  Synthetic Construct
uuuuucuugucauuauuccuuaaacaauaa

SEQ ID NO: 540 - RNA, 30 nt, Artificial Sequence
  Synthetic Construct
aaugacaaauaacagauuuaaaauuuucaa

SEQ ID NO: 541 - RNA, 29 nt, Artificial Sequence
  Synthetic Construct
aaauuuucaaggcauaguuuuauaccuga

SEQ ID NO: 542 - RNA, 30 nt, Artificial Sequence
  Synthetic Construct
acugagcauguacagacuuuuuuuucuugu

SEQ ID NO: 543 - RNA, 30 nt, Artificial Sequence
  Synthetic Construct
ugucuuuacugagcauguacagacuuuuuu

SEQ ID NO: 544 - RNA, 30 nt, Artificial Sequence
  Synthetic Construct
agcaaaccauggaucaaaaacacuggggaa

SEQ ID NO: 545 - RNA, 30 nt, Artificial Sequence
  Synthetic Construct
cguauccaggggcuccacaucuguggauuc

SEQ ID NO: 546 - RNA, 30 nt, Artificial Sequence
  Synthetic Construct
aaaguacagcgggccuuccguauccagggg

SEQ ID NO: 547 - RNA, 30 nt, Artificial Sequence
  Synthetic Construct
ucauucaaaguacagcgggccuuccguauc

SEQ ID NO: 548 - RNA, 30 nt, Artificial Sequence
  Synthetic Construct
aaaauuuucaaggcauaguuuuauaccuga

SEQ ID NO: 549 - RNA, 30 nt, Artificial Sequence
  Synthetic Construct
gucauucaaaguacagcgggccuuccguau

SEQ ID NO: 550 - RNA, 30 nt, Artificial Sequence
  Synthetic Construct
aaucuguuauuugucauucaaaguacagcg

SEQ ID NO: 551 - RNA, 30 nt, Artificial Sequence
  Synthetic Construct
uaaaucuguuauuugucauucaaaguacag

SEQ ID NO: 552 - RNA, 30 nt, Artificial Sequence
  Synthetic Construct
aaaauuuuaaaucuguuauuugucauucaa

SEQ ID NO: 553 - RNA, 21 nt, Artificial Sequence
  Synthetic Construct
aaggcauaguuuuauaccuga

SEQ ID NO: 554 - RNA, 22 nt, Artificial Sequence
  Synthetic Construct
caaggcauaguuuuauaccuga

SEQ ID NO: 555 - RNA, 23 nt, Artificial Sequence
  Synthetic Construct
ucaaggcauaguuuuauaccuga

SEQ ID NO: 556 - RNA, 30 nt, Artificial Sequence
  Synthetic Construct
uuugucauucaaaguacagcgggccuuccg

SEQ ID NO: 557 - RNA, 30 nt, Artificial Sequence
  Synthetic Construct
gagucugcauacuccucaugaccuggcccg

SEQ ID NO: 558 - RNA, 30 nt, Artificial Sequence
  Synthetic Construct
acuaucuuaacaagcuuugagugcaagaga

SEQ ID NO: 559 - RNA, 30 nt, Artificial Sequence
  Synthetic Construct
acuucuuugagcaucagauuccuaaucugg

SEQ ID NO: 560 - RNA, 30 nt, Artificial Sequence
  Synthetic Construct
uauuucuaaauuuucccccaaauucuaagc

SEQ ID NO: 561 - RNA, 30 nt, Artificial Sequence
  Synthetic Construct
cuaaauuuucccccaaauucuaagcagagu

SEQ ID NO: 562 - RNA, 30 nt, Artificial Sequence
  Synthetic Construct
uaaauuuucccccaaauucuaagcagagua

SEQ ID NO: 563 - RNA, 30 nt, Artificial Sequence
  Synthetic Construct
ucccccaaauucuaagcagaguauguaaau

SEQ ID NO: 564 - RNA, 30 nt, Artificial Sequence
  Synthetic Construct
cccccaaauucuaagcagaguauguaaauu

SEQ ID NO: 565 - RNA, 30 nt, Artificial Sequence
  Synthetic Construct
ccccaaauucuaagcagaguauguaaauug

SEQ ID NO: 566 - RNA, 30 nt, Artificial Sequence
  Synthetic Construct
uaagcagaguauguaaauuggaaguuaacu

SEQ ID NO: 567 - RNA, 30 nt, Artificial Sequence
  Synthetic Construct
gaaguuaacuuaugcacgcuuaacuaucuu

SEQ ID NO: 568 - RNA, 30 nt, Artificial Sequence
  Synthetic Construct
acuuaugcacgcuuaacuaucuuaacaagc

SEQ ID NO: 569 - RNA, 30 nt, Artificial Sequence
  Synthetic Construct
ugcacgcuuaacuaucuuaacaagcuuuga

SEQ ID NO: 570 - RNA, 30 nt, Artificial Sequence
  Synthetic Construct
aaggaauaaugacaagaaaaaaaagucugu

SEQ ID NO: 571 - RNA, 30 nt, Artificial Sequence
  Synthetic Construct
acaagcuuugagugcaagagauugaagagu

SEQ ID NO: 572 - RNA, 30 nt, Artificial Sequence
  Synthetic Construct
gagugcaagagauugaagaguucaaaucug

SEQ ID NO: 573 - RNA, 30 nt, Artificial Sequence
  Synthetic Construct
agugcaagagauugaagaguucaaaucuga

SEQ ID NO: 574 - RNA, 30 nt, Artificial Sequence
  Synthetic Construct
aagaguucaaaucugaccaagauguugaug

SEQ ID NO: 575 - RNA, 30 nt, Artificial Sequence
  Synthetic Construct
aaaucugaccaagauguugauguuggauaa

SEQ ID NO: 576 - RNA, 30 nt, Artificial Sequence
  Synthetic Construct
auguuggauaagagaauucucugcucccca

SEQ ID NO: 577 - RNA, 30 nt, Artificial Sequence
  Synthetic Construct
auccaauccaaaugcggcaucuucaaaccu

SEQ ID NO: 578 - RNA, 30 nt, Artificial Sequence
  Synthetic Construct
gaauucauccaauccaaaugcggcaucuuc

SEQ ID NO: 579 - RNA, 30 nt, Artificial Sequence
  Synthetic Construct
ggaauucauccaauccaaaugcggcaucuu

SEQ ID NO: 580 - RNA, 30 nt, Artificial Sequence
  Synthetic Construct
aaaagcaagcaagcagaauuuggaauucau

SEQ ID NO: 581 - RNA, 30 nt, Artificial Sequence
  Synthetic Construct
ugcauaaaguguaaguguauaagcauauca

SEQ ID NO: 582 - RNA, 30 nt, Artificial Sequence
  Synthetic Construct
gugcauaaaguguaaguguauaagcauauc

SEQ ID NO: 583 - RNA, 30 nt, Artificial Sequence
  Synthetic Construct
caauaauccugucaauuauauuucuaaauu

SEQ ID NO: 584 - RNA, 30 nt, Artificial Sequence
  Synthetic Construct
ugugcauaaaguguaaguguauaagcauau

SEQ ID NO: 585 - RNA, 30 nt, Artificial Sequence
  Synthetic Construct
uuauaacccuacauuuugugcauaaagugu

SEQ ID NO: 586 - RNA, 30 nt, Artificial Sequence
  Synthetic Construct
acauuauuauaacccuacauuuugugcaua

SEQ ID NO: 587 - RNA, 30 nt, Artificial Sequence
  Synthetic Construct
uaaagaagaucauguccauguuaacauuau

SEQ ID NO: 588 - RNA, 30 nt, Artificial Sequence
  Synthetic Construct
ccagcccuccuagagcuaccuguggagcaa

SEQ ID NO: 589 - RNA, 30 nt, Artificial Sequence
  Synthetic Construct
ucugcuccccaccucuaaguugccagcccu

SEQ ID NO: 590 - RNA, 30 nt, Artificial Sequence
  Synthetic Construct
gauaagagaauucucugcuccccaccucua

SEQ ID NO: 591 - RNA, 30 nt, Artificial Sequence
  Synthetic Construct
uaacccuacauuuugugcauaaaguguaag

SEQ ID NO: 592 - RNA, 30 nt, Artificial Sequence
  Synthetic Construct
ccaauaauccugucaauuauauuucuaaau

SEQ ID NO: 593 - RNA, 30 nt, Artificial Sequence
  Synthetic Construct
uaacaaauuuccaauaauccugucaauuau

SEQ ID NO: 594 - RNA, 30 nt, Artificial Sequence
  Synthetic Construct
auuauaacaaauuuccaauaauccugucaa

SEQ ID NO: 595 - RNA, 30 nt, Artificial Sequence
  Synthetic Construct
cccuguuugaaaauaaagggguaauagugg

SEQ ID NO: 596 - RNA, 30 nt, Artificial Sequence
  Synthetic Construct
aagacuguuucccuguuugaaaauaaaggg

SEQ ID NO: 597 - RNA, 30 nt, Artificial Sequence
  Synthetic Construct
ccaaguggaacuugaagacuguuucccugu

SEQ ID NO: 598 - RNA, 30 nt, Artificial Sequence
  Synthetic Construct
accaaguggaacuugaagacuguuucccug

SEQ ID NO: 599 - RNA, 30 nt, Artificial Sequence
  Synthetic Construct
uaccaaguggaacuugaagacuguuucccu

SEQ ID NO: 600 - RNA, 30 nt, Artificial Sequence
  Synthetic Construct
uuaccaaguggaacuugaagacuguuuccc

SEQ ID NO: 601 - RNA, 30 nt, Artificial Sequence
  Synthetic Construct
ccuguuugaaaauaaagggguaauaguggg

SEQ ID NO: 602 - RNA, 30 nt, Artificial Sequence
  Synthetic Construct
uuuaccaaguggaacuugaagacuguuucc

SEQ ID NO: 603 - RNA, 30 nt, Artificial Sequence
  Synthetic Construct
cacugugagccaaacucuauauacaagggg

SEQ ID NO: 604 - RNA, 30 nt, Artificial Sequence
  Synthetic Construct
acacugugagccaaacucuauauacaaggg

SEQ ID NO: 605 - RNA, 30 nt, Artificial Sequence
  Synthetic Construct
cuaaucuggaaaaugugaaucacugaggcc

SEQ ID NO: 606 - RNA, 30 nt, Artificial Sequence
  Synthetic Construct
agcaucagauuccuaaucuggaaaauguga

SEQ ID NO: 607 - RNA, 30 nt, Artificial Sequence
  Synthetic Construct
gagcaucagauuccuaaucuggaaaaugug

SEQ ID NO: 608 - RNA, 30 nt, Artificial Sequence
  Synthetic Construct
uuugagcaucagauuccuaaucuggaaaau

SEQ ID NO: 609 - RNA, 30 nt, Artificial Sequence
  Synthetic Construct
acauuuuuuaccaaguggaacuugaagacu

SEQ ID NO: 610 - RNA, 30 nt, Artificial Sequence
  Synthetic Construct
aacuucuuugagcaucagauuccuaaucug

SEQ ID NO: 611 - RNA, 30 nt, Artificial Sequence
  Synthetic Construct
gaaaauaaagggguaauagugggagugaga

SEQ ID NO: 612 - RNA, 30 nt, Artificial Sequence
  Synthetic Construct
uaugauuuauuuaacuuguggaacaaaaau

SEQ ID NO: 613 - RNA, 30 nt, Artificial Sequence
  Synthetic Construct
auucauuauaacaaauuuccaauaauccug

SEQ ID NO: 614 - RNA, 30 nt, Artificial Sequence
  Synthetic Construct
cauucauuauaacaaauuuccaauaauccu

SEQ ID NO: 615 - RNA, 30 nt, Artificial Sequence
  Synthetic Construct
uaugacaaaauguuucauucauuauaacaa

SEQ ID NO: 616 - RNA, 30 nt, Artificial Sequence
  Synthetic Construct
ucaaauguauaagaaguaaauaugaaucuu

SEQ ID NO: 617 - RNA, 30 nt, Artificial Sequence
  Synthetic Construct
aucaaauguauaagaaguaaauaugaaucu

SEQ ID NO: 618 - RNA, 30 nt, Artificial Sequence
  Synthetic Construct
cuuuaucaaauguauaagaaguaaauauga

SEQ ID NO: 619 - RNA, 30 nt, Artificial Sequence
  Synthetic Construct
aaaauaaagggguaauagugggagugagau

SEQ ID NO: 620 - RNA, 30 nt, Artificial Sequence
  Synthetic Construct
accacaaccaugccuuacuuuaucaaaugu

SEQ ID NO: 621 - RNA, 30 nt, Artificial Sequence
  Synthetic Construct
acuuguggaacaaaaauaaaccagauuaac

SEQ ID NO: 622 - RNA, 30 nt, Artificial Sequence
  Synthetic Construct
aacuuguggaacaaaaauaaaccagauuaa

SEQ ID NO: 623 - RNA, 30 nt, Artificial Sequence
  Synthetic Construct
uuuaacuuguggaacaaaaauaaaccagau

SEQ ID NO: 624 - RNA, 30 nt, Artificial Sequence
  Synthetic Construct
auuuaacuuguggaacaaaaauaaaccaga

SEQ ID NO: 625 - RNA, 30 nt, Artificial Sequence
  Synthetic Construct
ugauuuauuuaacuuguggaacaaaaauaa

SEQ ID NO: 626 - RNA, 30 nt, Artificial Sequence
  Synthetic Construct
augauuuauuuaacuuguggaacaaaaaua

SEQ ID NO: 627 - RNA, 30 nt, Artificial Sequence
  Synthetic Construct
uggaacaaaaauaaaccagauuaaccacaa

SEQ ID NO: 628 - RNA, 30 nt, Artificial Sequence
  Synthetic Construct
uuuaaggaauaaugacaagaaaaaaaaguc

SEQ ID NO: 629 - RNA, 30 nt, Artificial Sequence
  Synthetic Construct
auuuucaaacagggaaacagucuucaaguu

SEQ ID NO: 630 - RNA, 30 nt, Artificial Sequence
  Synthetic Construct
uuauuguuuaaggaauaaugacaagaaaaa

SEQ ID NO: 631 - RNA, 30 nt, Artificial Sequence
  Synthetic Construct
gagugcugucuccauguuugauguaucuga

SEQ ID NO: 632 - RNA, 30 nt, Artificial Sequence
  Synthetic Construct
agugcugucuccauguuugauguaucugag

SEQ ID NO: 633 - RNA, 30 nt, Artificial Sequence
  Synthetic Construct
gauguaucugagcagguugcuccacaggua

SEQ ID NO: 634 - RNA, 30 nt, Artificial Sequence
  Synthetic Construct
auguaucugagcagguugcuccacagguag

SEQ ID NO: 635 - RNA, 30 nt, Artificial Sequence
  Synthetic Construct
cuccacagguagcucuaggagggcuggcaa

SEQ ID NO: 636 - RNA, 30 nt, Artificial Sequence
  Synthetic Construct
gagguggggagcagagaauucucuuaucca

SEQ ID NO: 637 - RNA, 30 nt, Artificial Sequence
  Synthetic Construct
ucuuauccaacaucaacaucuuggucagau

SEQ ID NO: 638 - RNA, 30 nt, Artificial Sequence
  Synthetic Construct
uccaacaucaacaucuuggucagauuugaa

SEQ ID NO: 639 - RNA, 30 nt, Artificial Sequence
  Synthetic Construct
gucagauuugaacucuucaaucucuugcac

SEQ ID NO: 640 - RNA, 30 nt, Artificial Sequence
  Synthetic Construct
gaacucuucaaucucuugcacucaaagcuu

SEQ ID NO: 641 - RNA, 30 nt, Artificial Sequence
  Synthetic Construct
aacucuucaaucucuugcacucaaagcuug

SEQ ID NO: 642 - RNA, 30 nt, Artificial Sequence
  Synthetic Construct
aaucucuugcacucaaagcuuguuaagaua

SEQ ID NO: 643 - RNA, 30 nt, Artificial Sequence
  Synthetic Construct
cacucaaagcuuguuaagauaguuaagcgu

SEQ ID NO: 644 - RNA, 30 nt, Artificial Sequence
  Synthetic Construct
uuaagauaguuaagcgugcauaaguuaacu

SEQ ID NO: 645 - RNA, 30 nt, Artificial Sequence
  Synthetic Construct
agauaguuaagcgugcauaaguuaacuucc

SEQ ID NO: 646 - RNA, 30 nt, Artificial Sequence
  Synthetic Construct
agcgugcauaaguuaacuuccaauuuacau

SEQ ID NO: 647 - RNA, 30 nt, Artificial Sequence
  Synthetic Construct
acuuccaauuuacauacucugcuuagaauu

SEQ ID NO: 648 - RNA, 30 nt, Artificial Sequence
  Synthetic Construct
uaaugaaugaaacauuuugucauauaagau

SEQ ID NO: 649 - RNA, 30 nt, Artificial Sequence
  Synthetic Construct
uuauaaugaaugaaacauuuugucauauaa

SEQ ID NO: 650 - RNA, 30 nt, Artificial Sequence
  Synthetic Construct
guuauaaugaaugaaacauuuugucauaua

SEQ ID NO: 651 - RNA, 30 nt, Artificial Sequence
  Synthetic Construct
gaaauuuguuauaaugaaugaaacauuuug

SEQ ID NO: 652 - RNA, 30 nt, Artificial Sequence
  Synthetic Construct
uuggaaauuuguuauaaugaaugaaacauu

SEQ ID NO: 653 - RNA, 30 nt, Artificial Sequence
  Synthetic Construct
acaggauuauuggaaauuuguuauaaugaa

SEQ ID NO: 654 - RNA, 30 nt, Artificial Sequence
  Synthetic Construct
uacuuugagugcugucuccauguuugaugu

SEQ ID NO: 655 - RNA, 30 nt, Artificial Sequence
  Synthetic Construct
gaaauauaauugacaggauuauuggaaauu

SEQ ID NO: 656 - RNA, 30 nt, Artificial Sequence
  Synthetic Construct
ggggaaaauuuagaaauauaauugacagga

SEQ ID NO: 657 - RNA, 30 nt, Artificial Sequence
  Synthetic Construct
gggggaaaauuuagaaauauaauugacagg

SEQ ID NO: 658 - RNA, 30 nt, Artificial Sequence
  Synthetic Construct
gaauuugggggaaaauuuagaaauauaauu

SEQ ID NO: 659 - RNA, 30 nt, Artificial Sequence
  Synthetic Construct
cauacucugcuuagaauuugggggaaaauu

SEQ ID NO: 660 - RNA, 30 nt, Artificial Sequence
  Synthetic Construct
acauacucugcuuagaauuugggggaaaau

SEQ ID NO: 661 - RNA, 30 nt, Artificial Sequence
  Synthetic Construct
caauuuacauacucugcuuagaauuugggg

SEQ ID NO: 662 - RNA, 30 nt, Artificial Sequence
  Synthetic Construct
agaaauauaauugacaggauuauuggaaau

SEQ ID NO: 663 - RNA, 30 nt, Artificial Sequence
  Synthetic Construct
uaauucuacuuugagugcugucuccauguu

SEQ ID NO: 664 - RNA, 30 nt, Artificial Sequence
  Synthetic Construct
auaauucuacuuugagugcugucuccaugu

SEQ ID NO: 665 - RNA, 30 nt, Artificial Sequence
  Synthetic Construct
uuuauaauucuacuuugagugcugucucca

SEQ ID NO: 666 - RNA, 30 nt, Artificial Sequence
  Synthetic Construct
aagaugccgcauuuggauuggaugaauucc

SEQ ID NO: 667 - RNA, 30 nt, Artificial Sequence
  Synthetic Construct
gaagaugccgcauuuggauuggaugaauuc

SEQ ID NO: 668 - RNA, 30 nt, Artificial Sequence
  Synthetic Construct
agguuugaagaugccgcauuuggauuggau

SEQ ID NO: 669 - RNA, 30 nt, Artificial Sequence
  Synthetic Construct
cagguuugaagaugccgcauuuggauugga

SEQ ID NO: 670 - RNA, 30 nt, Artificial Sequence
  Synthetic Construct
ucagguuugaagaugccgcauuuggauugg

SEQ ID NO: 671 - RNA, 30 nt, Artificial Sequence
  Synthetic Construct
uuuucagguuugaagaugccgcauuuggau

SEQ ID NO: 672 - RNA, 30 nt, Artificial Sequence
  Synthetic Construct
ggauuggaugaauuccaaauucugcuugcu

SEQ ID NO: 673 - RNA, 30 nt, Artificial Sequence
  Synthetic Construct
cuuuucagguuugaagaugccgcauuugga

SEQ ID NO: 674 - RNA, 30 nt, Artificial Sequence
  Synthetic Construct
uuuucuuuucagguuugaagaugccgcauu

SEQ ID NO: 675 - RNA, 30 nt, Artificial Sequence
  Synthetic Construct
cuuuucuuuucagguuugaagaugccgcau

SEQ ID NO: 676 - RNA, 30 nt, Artificial Sequence
  Synthetic Construct
ucuuuucuuuucagguuugaagaugccgca

SEQ ID NO: 677 - RNA, 30 nt, Artificial Sequence
  Synthetic Construct
uucuuuucuuuucagguuugaagaugccgc

SEQ ID NO: 678 - RNA, 30 nt, Artificial Sequence
  Synthetic Construct
cuaaccuuuuucuuuucuuuucagguuuga

SEQ ID NO: 679 - RNA, 30 nt, Artificial Sequence
  Synthetic Construct
auugcuaaccuuuuucuuuucuuuucaggu

SEQ ID NO: 680 - RNA, 30 nt, Artificial Sequence
  Synthetic Construct
ucuuuucagguuugaagaugccgcauuugg

SEQ ID NO: 681 - RNA, 30 nt, Artificial Sequence
  Synthetic Construct
ugucauauaagauucauauuuacuucuuau

SEQ ID NO: 682 - RNA, 30 nt, Artificial Sequence
  Synthetic Construct
gauuggaugaauuccaaauucugcuugcuu

SEQ ID NO: 683 - RNA, 30 nt, Artificial Sequence
  Synthetic Construct
caaauucugcuugcuugcuuuuuaauauug

SEQ ID NO: 684 - RNA, 30 nt, Artificial Sequence
  Synthetic Construct
acauggacaugaucuucuuuauaauucuac

SEQ ID NO: 685 - RNA, 30 nt, Artificial Sequence
  Synthetic Construct
uaauaauguuaacauggacaugaucuucuu

SEQ ID NO: 686 - RNA, 30 nt, Artificial Sequence
  Synthetic Construct
ugcacaaaauguaggguuauaauaauguua

SEQ ID NO: 687 - RNA, 30 nt, Artificial Sequence
  Synthetic Construct
augcacaaaauguaggguuauaauaauguu

SEQ ID NO: 688 - RNA, 30 nt, Artificial Sequence
  Synthetic Construct
cacuuuaugcacaaaauguaggguuauaau

SEQ ID NO: 689 - RNA, 30 nt, Artificial Sequence
  Synthetic Construct
uacacuuacacuuuaugcacaaaauguagg

SEQ ID NO: 690 - RNA, 30 nt, Artificial Sequence
  Synthetic Construct
gaugaauuccaaauucugcuugcuugcuuu

SEQ ID NO: 691 - RNA, 30 nt, Artificial Sequence
  Synthetic Construct
auaugcuuauacacuuacacuuuaugcaca

SEQ ID NO: 692 - RNA, 30 nt, Artificial Sequence
  Synthetic Construct
aauauugauaugcuuauacacuuacacuuu

SEQ ID NO: 693 - RNA, 30 nt, Artificial Sequence
  Synthetic Construct
uaauauugauaugcuuauacacuuacacuu

SEQ ID NO: 694 - RNA, 30 nt, Artificial Sequence
  Synthetic Construct
uuaauauugauaugcuuauacacuuacacu

SEQ ID NO: 695 - RNA, 30 nt, Artificial Sequence
  Synthetic Construct
cuuuuuaauauugauaugcuuauacacuua

SEQ ID NO: 696 - RNA, 30 nt, Artificial Sequence
  Synthetic Construct
cuugcuuuuuaauauugauaugcuuauaca

SEQ ID NO: 697 - RNA, 30 nt, Artificial Sequence
  Synthetic Construct
ugcuugcuugcuuuuuaauauugauaugcu

SEQ ID NO: 698 - RNA, 30 nt, Artificial Sequence
  Synthetic Construct
auauugauaugcuuauacacuuacacuuua

SEQ ID NO: 699 - RNA, 30 nt, Artificial Sequence
  Synthetic Construct
uuguuuaaggaauaaugacaagaaaaaaaa

SEQ ID NO: 700 - RNA, 30 nt, Artificial Sequence
  Synthetic Construct
gucauauaagauucauauuuacuucuuaua

SEQ ID NO: 701 - RNA, 30 nt, Artificial Sequence
  Synthetic Construct
auauuuacuucuuauacauuugauaaagua

SEQ ID NO: 702 - RNA, 30 nt, Artificial Sequence
  Synthetic Construct
aauggcauaguuggggugacacagcugucu

SEQ ID NO: 703 - RNA, 30 nt, Artificial Sequence
  Synthetic Construct
gggugacacagcugucuagugggaggccag

SEQ ID NO: 704 - RNA, 30 nt, Artificial Sequence
  Synthetic Construct
uauauuuuagccagcguucuuuccugcggg

SEQ ID NO: 705 - RNA, 30 nt, Artificial Sequence
  Synthetic Construct
uagccagcguucuuuccugcgggccagguc

SEQ ID NO: 706 - RNA, 30 nt, Artificial Sequence
  Synthetic Construct
agccagcguucuuuccugcgggccagguca

SEQ ID NO: 707 - RNA, 30 nt, Artificial Sequence
  Synthetic Construct
gccagcguucuuuccugcgggccaggucau

SEQ ID NO: 708 - RNA, 30 nt, Artificial Sequence
  Synthetic Construct
uuuccugcgggccaggucaugaggaguaug

SEQ ID NO: 709 - RNA, 30 nt, Artificial Sequence
  Synthetic Construct
ccugcgggccaggucaugaggaguaugcag

SEQ ID NO: 710 - RNA, 30 nt, Artificial Sequence
  Synthetic Construct
cugcgggccaggucaugaggaguaugcaga

SEQ ID NO: 711 - RNA, 30 nt, Artificial Sequence
  Synthetic Construct
aauauuuuagcaaggaauagauauacaauc

SEQ ID NO: 712 - RNA, 30 nt, Artificial Sequence
  Synthetic Construct
auauuuuagcaaggaauagauauacaauca

SEQ ID NO: 713 - RNA, 30 nt, Artificial Sequence
  Synthetic Construct
uagcaaggaauagauauacaaucaucccuu

SEQ ID NO: 714 - RNA, 30 nt, Artificial Sequence
  Synthetic Construct
agcaaggaauagauauacaaucaucccuug

SEQ ID NO: 715 - RNA, 30 nt, Artificial Sequence
  Synthetic Construct
gcaaggaauagauauacaaucaucccuugg

SEQ ID NO: 716 - RNA, 30 nt, Artificial Sequence
  Synthetic Construct
gucucccugggggauugguuucaggacccc

SEQ ID NO: 717 - RNA, 30 nt, Artificial Sequence
  Synthetic Construct
guuucaggaccccuucuuggacaccaaauc

SEQ ID NO: 718 - RNA, 30 nt, Artificial Sequence
  Synthetic Construct
caggaccccuucuuggacaccaaaucuaug

SEQ ID NO: 719 - RNA, 30 nt, Artificial Sequence
  Synthetic Construct
uaauaccuaauacaauguaaaugcuaugca

SEQ ID NO: 720 - RNA, 30 nt, Artificial Sequence
  Synthetic Construct
cuuguaauaccuaauacaauguaaaugcua

SEQ ID NO: 721 - RNA, 30 nt, Artificial Sequence
  Synthetic Construct
uagauuacuuguaauaccuaauacaaugua

SEQ ID NO: 722 - RNA, 30 nt, Artificial Sequence
  Synthetic Construct
cuagauuacuuguaauaccuaauacaaugu

SEQ ID NO: 723 - RNA, 30 nt, Artificial Sequence
  Synthetic Construct
aaucauuucuagauuacuuguaauaccuaa

SEQ ID NO: 724 - RNA, 30 nt, Artificial Sequence
  Synthetic Construct
aaaucauuucuagauuacuuguaauaccua

SEQ ID NO: 725 - RNA, 30 nt, Artificial Sequence
  Synthetic Construct
ggaaucugaugcucaaagaaguuaaauggc

SEQ ID NO: 726 - RNA, 30 nt, Artificial Sequence
  Synthetic Construct
cauauaaccuauccacauccuccuguauac

SEQ ID NO: 727 - RNA, 30 nt, Artificial Sequence
  Synthetic Construct
uauaaaaugguauaguauuugcauauaacc

SEQ ID NO: 728 - RNA, 30 nt, Artificial Sequence
  Synthetic Construct
agucccuucuauaaaaugguauaguauuug

SEQ ID NO: 729 - RNA, 30 nt, Artificial Sequence
  Synthetic Construct
aagucccuucuauaaaaugguauaguauuu

SEQ ID NO: 730 - RNA, 30 nt, Artificial Sequence
  Synthetic Construct
gacaccaaaucuauggauauuuaagucccu

SEQ ID NO: 731 - RNA, 30 nt, Artificial Sequence
  Synthetic Construct
uuggacaccaaaucuauggauauuuaaguc

SEQ ID NO: 732 - RNA, 30 nt, Artificial Sequence
  Synthetic Construct
aggaccccuucuuggacaccaaaucuaugg

SEQ ID NO: 733 - RNA, 30 nt, Artificial Sequence
  Synthetic Construct
gcauauaaccuauccacauccuccuguaua

SEQ ID NO: 734 - RNA, 30 nt, Artificial Sequence
  Synthetic Construct
cagauuaggaaucugaugcucaaagaaguu

SEQ ID NO: 735 - RNA, 30 nt, Artificial Sequence
  Synthetic Construct
ccagauuaggaaucugaugcucaaagaagu

SEQ ID NO: 736 - RNA, 30 nt, Artificial Sequence
  Synthetic Construct
uccagauuaggaaucugaugcucaaagaag

SEQ ID NO: 737 - RNA, 30 nt, Artificial Sequence
  Synthetic Construct
uuccacaaguuaaauaaaucauaaaacuug

SEQ ID NO: 738 - RNA, 30 nt, Artificial Sequence
  Synthetic Construct
guuccacaaguuaaauaaaucauaaaacuu

SEQ ID NO: 739 - RNA, 30 nt, Artificial Sequence
  Synthetic Construct
uguuccacaaguuaaauaaaucauaaaacu

SEQ ID NO: 740 - RNA, 30 nt, Artificial Sequence
  Synthetic Construct
uuguuccacaaguuaaauaaaucauaaaac

SEQ ID NO: 741 - RNA, 30 nt, Artificial Sequence
  Synthetic Construct
uuuuuguuccacaaguuaaauaaaucauaa

SEQ ID NO: 742 - RNA, 30 nt, Artificial Sequence
  Synthetic Construct
auuuuuguuccacaaguuaaauaaaucaua

SEQ ID NO: 743 - RNA, 30 nt, Artificial Sequence
  Synthetic Construct
cacaaguuaaauaaaucauaaaacuugaug

SEQ ID NO: 744 - RNA, 30 nt, Artificial Sequence
  Synthetic Construct
aucugguuuauuuuuguuccacaaguuaaa

SEQ ID NO: 745 - RNA, 30 nt, Artificial Sequence
  Synthetic Construct
auaaaguaaggcaugguugugguuaaucug

SEQ ID NO: 746 - RNA, 30 nt, Artificial Sequence
  Synthetic Construct
gauaaaguaaggcaugguugugguuaaucu

SEQ ID NO: 747 - RNA, 30 nt, Artificial Sequence
  Synthetic Construct
uacauuugauaaaguaaggcaugguugugg

SEQ ID NO: 748 - RNA, 30 nt, Artificial Sequence
  Synthetic Construct
uuauacauuugauaaaguaaggcaugguug

SEQ ID NO: 749 - RNA, 30 nt, Artificial Sequence
  Synthetic Construct
cuucuuauacauuugauaaaguaaggcaug

SEQ ID NO: 750 - RNA, 30 nt, Artificial Sequence
  Synthetic Construct
acuucuuauacauuugauaaaguaaggcau

SEQ ID NO: 751 - RNA, 30 nt, Artificial Sequence
  Synthetic Construct
ugguuaaucugguuuauuuuuguuccacaa

SEQ ID NO: 752 - RNA, 30 nt, Artificial Sequence
  Synthetic Construct
ucauauaagauucauauuuacuucuuauac

SEQ ID NO: 753 - RNA, 30 nt, Artificial Sequence
  Synthetic Construct
aauaaaucauaaaacuugauguguuaucuc

SEQ ID NO: 754 - RNA, 30 nt, Artificial Sequence
  Synthetic Construct
ucucuuauaucucacucccacuauuacccc

SEQ ID NO: 755 - RNA, 30 nt, Artificial Sequence
  Synthetic Construct
acauuuuccagauuaggaaucugaugcuca

SEQ ID NO: 756 - RNA, 30 nt, Artificial Sequence
  Synthetic Construct
gcucacaguguaaagggccucagugauuca

SEQ ID NO: 757 - RNA, 30 nt, Artificial Sequence
  Synthetic Construct
ggcucacaguguaaagggccucagugauuc

SEQ ID NO: 758 - RNA, 30 nt, Artificial Sequence
  Synthetic Construct
uauauagaguuuggcucacaguguaaaggg

SEQ ID NO: 759 - RNA, 30 nt, Artificial Sequence
  Synthetic Construct
guaaaaaaugugaaccccuuguauauagag

SEQ ID NO: 760 - RNA, 30 nt, Artificial Sequence
  Synthetic Construct
cacuugguaaaaaaugugaaccccuuguau

SEQ ID NO: 761 - RNA, 30 nt, Artificial Sequence
  Synthetic Construct
auguguuaucucuuauaucucacucccacu

SEQ ID NO: 762 - RNA, 30 nt, Artificial Sequence
  Synthetic Construct
aaguuccacuugguaaaaaaugugaacccc

SEQ ID NO: 763 - RNA, 30 nt, Artificial Sequence
  Synthetic Construct
caaacagggaaacagucuucaaguuccacu

SEQ ID NO: 764 - RNA, 30 nt, Artificial Sequence
  Synthetic Construct
ucaaacagggaaacagucuucaaguuccac

SEQ ID NO: 765 - RNA, 30 nt, Artificial Sequence
  Synthetic Construct
uuuucaaacagggaaacagucuucaaguuc

SEQ ID NO: 766 - RNA, 30 nt, Artificial Sequence
  Synthetic Construct
aaaccugaaaagaaaagaaaaagguuagca

SEQ ID NO: 767 - RNA, 30 nt, Artificial Sequence
  Synthetic Construct
ccccuuuauuuucaaacagggaaacagucu

SEQ ID NO: 768 - RNA, 30 nt, Artificial Sequence
  Synthetic Construct
uaucucacucccacuauuaccccuuuauuu

SEQ ID NO: 769 - RNA, 30 nt, Artificial Sequence
  Synthetic Construct
aaacagggaaacagucuucaaguuccacuu

SEQ ID NO: 770 - RNA, 30 nt, Artificial Sequence
  Synthetic Construct
gcaaugaauuuauuuuauuuggauugcaga

SEQ ID NO: 771 - DNA, 3162 bp, Artificial Sequence
  Synthetic Construct
atgagcagcgcgatcaaaagctacaagagcgttctgcgtccgaacgagcgtaagaaccaactgctgaaaagcaccattcagtgcctggaagacggtagcgcgttctttttcaagatgctgcaaggcctgtttggtggcatcaccccggagattgttcgtttcagcaccgaacaggagaaacagcaacaggatatcgcgctgtggtgcgcggttaactggttccgtccggtgagccaagacagcctgacccacaccattgcgagcgataacctggtggagaagtttgaggaatactatggtggcaccgcgagcgacgcgatcaaacagtacttcagcgcgagcattggcgaaagctactattggaacgactgccgtcaacagtactatgatctgtgccgtgagctgggtgttgaggtgagcgacctgacccatgatctggagatcctgtgccgtgaaaagtgcctggcggttgcgaccgagagcaaccagaacaacagcatcattagcgttctgtttggcaccggcgaaaaagaggaccgtagcgtgaaactgcgtatcaccaagaaaattctggaggcgatcagcaacctgaaagaaatcccgaagaacgttgcgccgattcaagagatcattctgaacgtggcgaaagcgaccaaggaaaccttccgtcaggtgtatgcgggtaacctgggtgcgccgagcaccctggagaaatttatcgcgaaggacggccaaaaagagttcgatctgaagaaactgcagaccgacctgaagaaagttattcgtggtaaaagcaaggagcgtgattggtgctgccaggaagagctgcgtagctacgtggagcaaaacaccatccagtatgacctgtgggcgtggggcgaaatgttcaacaaagcgcacaccgcgctgaaaatcaagagcacccgtaactacaactttgcgaagcaacgtctggaacagttcaaagagattcagagcctgaacaacctgctggttgtgaagaagctgaacgactttttcgatagcgaatttttcagcggcgaggaaacctacaccatctgcgttcaccatctgggtggcaaggacctgagcaaactgtataaggcgtgggaggatgatccggcggacccggaaaacgcgattgtggttctgtgcgacgatctgaaaaacaactttaagaaagagccgatccgtaacattctgcgttacatcttcaccattcgtcaagaatgcagcgcgcaggacatcctggcggcggcgaagtacaaccaacagctggatcgttataaaagccaaaaggcgaacccgagcgttctgggtaaccagggctttacctggaccaacgcggtgatcctgccggagaaggcgcagcgtaacgaccgtccgaacagcctggatctgcgtatttggctgtacctgaaactgcgtcacccggacggtcgttggaagaaacaccatatcccgttctacgatacccgtttcttccaagaaatttatgcggcgggcaacagcccggttgacacctgccagtttcgtaccccgcgtttcggttatcacctgccgaaactgaccgatcagaccgcgatccgtgttaacaagaaacatgtgaaagcggcgaagaccgaggcgcgtattcgtctggcgatccaacagggcaccctgccggtgagcaacctgaagatcaccgaaattagcgcgaccatcaacagcaaaggtcaagtgcgtattccggttaagtttgacgtgggtcgtcaaaaaggcaccctgcagatcggtgaccgtttctgcggctacgatcaaaaccagaccgcgagccacgcgtatagcctgtgggaagtggttaaagagggtcaataccataaagagctgggctgctttgttcgtttcatcagcagcggtgacatcgtgagcattaccgagaaccgtggcaaccaatttgatcagctgagctatgaaggtctggcgtacccgcaatatgcggactggcgtaagaaagcgagcaagttcgtgagcctgtggcagatcaccaagaaaaacaagaaaaaggaaatcgtgaccgttgaagcgaaagagaagtttgacgcgatctgcaagtaccagccgcgtctgtataaattcaacaaggagtacgcgtatctgctgcgtgatattgttcgtggcaaaagcctggtggaactgcaacagattcgtcaagagatctttcgtttcattgaacaggactgcggtgttacccgtctgggcagcctgagcctgagcaccctggaaaccgtgaaagcggttaagggtatcatttacagctattttagcaccgcgctgaacgcgagcaagaacaacccgatcagcgacgaacagcgtaaagagtttgatccggaactgttcgcgctgctggaaaagctggagctgattcgtacccgtaaaaagaaacaaaaagtggaacgtatcgcgaacagcctgattcagacctgcctggagaacaacatcaagttcattcgtggtgaaggcgacctgagcaccaccaacaacgcgaccaagaaaaaggcgaacagccgtagcatggattggttggcgcgtggtgtttttaacaaaatccgtcaactggcgccgatgcacaacattaccctgttcggttgcggcagcctgtacaccagccaccaggacccgctggtgcatcgtaacccggataaagcgatgaagtgccgttgggcggcgatcccggttaaggacattggcgattgggtgctgcgtaagctgagccaaaacctgcgtgcgaaaaacatcggcaccggcgagtactatcaccaaggtgttaaagagttcctgagccattatgaactgcaggacctggaggaagagctgctgaagtggcgtagcgatcgtaaaagcaacattccgtgctgggtgctgcagaaccgtctggcggagaagctgggcaacaaagaagcggtggtttacatcccggttcgtggtggccgtatttattttgcgacccacaaggtggcgaccggtgcggtgagcatcgttttcgaccaaaaacaagtgtgggtttgcaacgcggatcatgttgcggcggcgaacatcgcgctgaccgtgaagggtattggcgaacaaagcagcgacgaagagaacccggatggtagccgtatcaaactgcagctgaccagc

SEQ ID NO: 772 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTS

SEQ ID NO: 773 - DNA, 7692 bp, Homo sapiens
acttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatgtctcgctccgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagcctaaccagggcttttgcgggagcgcatgccttttggctgtaattcgtgcatttttttttaagaaaaacgcctgccttctgcgtgagattctccagagcaaactgggcggcatgggccctgtggtcttttcgtacagagggcttcctctttggctctttgcctggttgtttccaagatgtactgtgcctcttactttcggttttgaaaacatgagggggttgggcgtggtagcttacgcctgtaatcccagcacttagggaggccgaggcgggaggatggcttgaggtccgtagttgagaccagcctggccaacatggtgaagcctggtctctacaaaaaataataacaaaaattagccgggtgtggtggctcgtgcctgtggtcccagctgctccggtggctgaggcgggaggatctcttgagcttaggcttttgagctatcatggcgccagtgcactccagcgtgggcaacagagcgagaccctgtctctcaaaaaagaaaaaaaaaaaaaaagaaagagaaaagaaaagaaagaaagaagtgaaggtttgtcagtcaggggagctgtaaaaccattaataaagataatccaagatggttaccaagactgttgaggacgccagagatcttgagcactttctaagtacctggcaatacactaagcgcgctcaccttttcctctggcaaaacatgatcgaaagcagaatgttttgatcatgagaaaattgcatttaatttgaatacaatttatttacaacataaaggataatgtatatatcaccaccattactggtatttgctggttatgttagatgtcattttaaaaaataacaatctgatatttaaaaaaaaatcttattttgaaaatttccaaagtaatacatgccatgcatagaccatttctggaagataccacaagaaacatgtaatgatgattgcctctgaaggtctattttcctcctctgacctgtgtgtgggttttgtttttgttttactgtgggcataaattaatttttcagttaagttttggaagcttaaataactctccaaaagtcataaagccagtaactggttgagcccaaattcaaacccagcctgtctgatacttgtcctcttcttagaaaagattacagtgatgctctcacaaaatcttgccgccttccctcaaacagagagttccaggcaggatgaatctgtgctctgatccctgaggcatttaatatgttcttattattagaagctcagatgcaaagagctctcttagcttttaatgttatgaaaaaaatcaggtcttcattagattccccaatccacctcttgatggggctagtagcctttccttaatgatagggtgtttctagagagatatatctggtcaaggtggcctggtactcctccttctccccacagcctcccagacaaggaggagtagctgccttttagtgatcatgtaccctgaatataagtgtatttaaaagaattttatacacatatatttagtgtcaatctgtatatttagtagcactaacacttctcttcattttcaatgaaaaatatagagtttataatattttcttcccacttccccatggatggtctagtcatgcctctcattttggaaagtactgtttctgaaacattaggcaatatattcccaacctggctagtttacagcaatcacctgtggatgctaattaaaacgcaaatcccactgtcacatgcattactccatttgatcataatggaaagtatgttctgtcccatttgccatagtcctcacctatccctgttgtattttatcgggtccaactcaaccatttaaggtatttgccagctcttgtatgcatttaggttttgtttctttgttttttagctcatgaaattaggtacaaagtcagagaggggtctggcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcagggaaagaagaatcctacagggtcatgttcccttctcctgtggagtggcatgaagaaggtgtatggccccaggtatggccatattactgaccctctacagagagggcaaaggaactgccagtatggtattgcaggataaaggcaggtggttacccacattacctgcaaggctttgatctttcttctgccatttccacattggacatctctgctgaggagagaaaatgaaccactcttttcctttgtataatgttgttttattcttcagacagaagagaggagttatacagctctgcagacatcccattcctgtatggggactgtgtttgcctcttagaggttcccaggccactagaggagataaagggaaacagattgttataacttgatataatgatactataatagatgtaactacaaggagctccagaagcaagagagagggaggaacttggacttctctgcatctttagttggagtccaaaggcttttcaatgaaattctactgcccagggtacattgatgctgaaaccccattcaaatctcctgttatattctagaacagggaattgatttgggagagcatcaggaaggtggatgatctgcccagtcacactgttagtaaattgtagagccaggacctgaactctaatatagtcatgtgttacttaatgacggggacatgttctgagaaatgcttacacaaacctaggtgttgtagcctactacacgcataggctacatggtatagcctattgctcctagactacaaacctgtacagcctgttactgtactgaatactgtgggcagttgtaacacaatggtaagtatttgtgtatctaaacatagaagttgcagtaaaaatatgctattttaatcttatgagaccactgtcatatatacagtccatcattgaccaaaacatcatatcagcattttttcttctaagattttgggagcaccaaagggatacactaacaggatatactctttataatgggtttggagaactgtctgcagctacttcttttaaaaaggtgatctacacagtagaaattagacaagtttggtaatgagatctgcaatccaaataaaataaattcattgctaacctttttcttttcttttcaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctcttatatctcactcccactattacccctttattttcaaacagggaaacagtcttcaagttccacttggtaaaaaatgtgaaccccttgtatatagagtttggctcacagtgtaaagggcctcagtgattcacattttccagattaggaatctgatgctcaaagaagttaaatggcatagttggggtgacacagctgtctagtgggaggccagccttctatattttagccagcgttctttcctgcgggccaggtcatgaggagtatgcagactctaagagggagcaaaagtatctgaaggatttaatattttagcaaggaatagatatacaatcatcccttggtctccctgggggattggtttcaggaccccttcttggacaccaaatctatggatatttaagtcccttctataaaatggtatagtatttgcatataacctatccacatcctcctgtatactttaaatcatttctagattacttgtaatacctaatacaatgtaaatgctatgcaaatagttgttattgtttaaggaataatgacaagaaaaaaaagtctgtacatgctcagtaaagacacaaccatccctttttttccccagtgtttttgatccatggtttgctgaatccacagatgtggagcccctggatacggaaggcccgctgtactttgaatgacaaataacagatttaaaattttcaaggcatagttttatacctga

SEQ ID NO: 774 - DNA, 197 bp, Homo sapiens
gattggctgggcacgcgtttaatataagtggaggcgtcgcgctggcgggcattcctgaagctgacagcattcgggccgagatgtctcgctccgtggccttagctgtgctcgcgctactctctctttctggcctggaggctatccagcgtgagtctctcctaccctcccgctctggtccttcctctcccgctctgcac

SEQ ID NO: 775 - DNA, 379 bp, Homo sapiens
aagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtc

SEQ ID NO: 776 - DNA, 128 bp, Homo sapiens
aaagtaaaacttaatgtcttccttttttttctccactgtctttttcatagatcgagacatgtaagcagcatcatggaggtaagtttttgaccttgagaaaatgtttttgtttcactgtcctgaggact

SEQ ID NO: 777 - DNA, 1321 bp, Homo sapiens
gcaatccaaataaaataaattcattgctaacctttttcttttcttttcaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctcttatatctcactcccactattacccctttattttcaaacagggaaacagtcttcaagttccacttggtaaaaaatgtgaaccccttgtatatagagtttggctcacagtgtaaagggcctcagtgattcacattttccagattaggaatctgatgctcaaagaagttaaatggcatagttggggtgacacagctgtctagtgggaggccagccttctatattttagccagcgttctttcctgcgggccaggtcatgaggagtatgcagactctaagagggagcaaaagtatctgaaggatttaatattttagcaaggaatagatatacaatcatcccttggtctccctgggggattggtttcaggaccccttcttggacaccaaatctatggatatttaagtcccttctataaaatggtatagtatttgcatataacctatccacatcctcctgtatactttaaatcatttctagattacttgtaatacctaatacaatgtaaatgctatgcaaatagttgttattgtttaaggaataatgacaagaaaaaaaagtctgtacatgctcagtaaagacacaaccatccctttttttccccagtgtttttgatccatggtttgctgaatccacagatgtggagcccctggatacggaaggcccgctgtactttgaatgacaaataacagatttaaaattttcaaggcatagttttatacctga

SEQ ID NO: 778 - RNA, 43 nt, Artificial Sequence
  Synthetic Construct
agaaauccgucuuucauugacggaaugucggauggaugaaacc

SEQ ID NO: 779 - RNA, 43 nt, Artificial Sequence
  Synthetic Construct
agaaauccgucuuucauugacggcuaucucuuguacuacacug

SEQ ID NO: 780 - RNA, 43 nt, Artificial Sequence
  Synthetic Construct
agaaauccgucuuucauugacggacacggcaggcauacucauc

SEQ ID NO: 781 - RNA, 43 nt, Artificial Sequence
  Synthetic Construct
agaaauccgucuuucauugacgggucacagcccaagauaguua

SEQ ID NO: 782 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGRWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 783 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 784 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGGRIKLQLTS

SEQ ID NO: 785 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 786 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLATMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 787 - DNA, 3222 bp, Artificial Sequence
  Synthetic Construct
atggcttccatctctaggccatacggcaccaagctgcgaccggacgcacggaagaaggagatgctcgataagttctttaatacactgactaagggtcagcgcgtgttcgcagacctggccctgtgcatctatggctccctgaccctggagatggccaagtctctggagccagaaagtgattcagaactggtgtgcgctattgggtggtttcggctggtggacaagaccatctggtccaaggatggcatcaagcaggagaatctggtgaaacagtacgaagcctattccggaaaggaggcttctgaagtggtcaaaacatacctgaacagccccagctccgacaagtacgtgtggatcgattgcaggcagaaattcctgaggtttcagcgcgagctcggcactcgcaacctgtccgaggacttcgaatgtatgctctttgaacagtacattagactgaccaagggcgagatcgaagggtatgccgctatttcaaatatgttcggaaacggcgagaaggaagaccggagcaagaaaagaatgtacgctacacggatgaaagattggctggaggcaaacgaaaatatcacttgggagcagtatagagaggccctgaagaaccagctgaatgctaaaaacctggagcaggttgtggccaattacaaggggaacgctggcggggcagaccccttctttaagtatagcttctccaaagagggaatggtgagcaagaaagaacatgcacagcagctcgacaagttcaaaaccgtcctgaagaacaaagcccgggacctgaattttccaaacaaggagaagctgaagcagtacctggaggccgaaatcggcattccggtcgacgctaacgtgtactcccagatgttctctaacggggtgagtgaggtccagcctaagaccacacggaatatgtcttttagtaacgagaaactggatctgctcactgaactgaaggacctgaacaagggcgatgggttcgagtacgccagagaagtgctgaacgggttctttgactccgagctccacactaccgaggataagtttaatatcacctctaggtacctgggaggcgacaaatcaaaccgcctgagcaaactctataagatctggaagaaagagggtgtggactgcgaggaaggcattcagcagttctgtgaagccgtcaaagataagatgggccagatccccattcgaaatgtgctgaagtacctgtggcagttccgggagacagtcagtgccgaggattttgaagcagccgctaaggctaaccatctggaggaaaagatcagccgggtgaaagcccacccaatcgtgattagcaataggtactgggcttttgggacttccgcactggtgggaaacattatgcccgcagacaagaggcatcagggagagtatgccggtcagaatttcaaaatgtggctggaggctgaactgcactacgatggcaagaaagcaaagcaccatctgcctttttataacgcccgcttctttgaggaagtgtactgctatcacccctctgtcgccgagatcactcctttcaaaaccaagcagtttggctgtgaaatcgggaaggacattccagattacgtgagcgtcgctctgaaggacaatccgtataagaaagcaaccaaacgaatcctgcgtgcaatctacaatcccgtcgccaacacaactggcgttgataagaccacaaactgcagcttcatgatcaaacgcgagaatgacgaatataagctggtcatcaaccgaaaaatttccgtggatcggcctaagagaatcgaagtgggcaggacaattatggggtacgaccgcaatcagacagctagcgatacttattggattggccggctggtgccacctggaacccggggcgcataccgcatcggagagtggagcgtccagtatattaagtccgggcctgtcctgtctagtactcagggagttaacaattccactaccgaccagctggtgtacaacggcatgccatcaagctccgagcggttcaaggcctggaagaaagccagaatggcttttatccgaaaactcattcgtcagctgaatgacgagggactggaatctaagggtcaggattatatccccgagaacccttctagtttcgatgtgcggggcgaaaccctgtacgtctttaacagtaattatctgaaggccctggtgagcaaacacagaaaggccaagaaacctgttgaggggatcctggacgagattgaagcctggacatctaaagacaaggattcatgcagcctgatgcggctgagcagcctgagcgatgcttccatgcagggaatcgccagcctgaagagtctgattaacagctacttcaacaagaatggctgtaaaaccatcgaggacaaagaaaagtttaatcccgtgctgtatgccaagctggttgaggtggaacagcggagaacaaacaagcggtctgagaaagtgggaagaatcgcaggtagtctggagcagctggccctgctgaacggggttgaggtggtcatcggcgaagctgacctgggggaggtcgaaaaaggaaagagtaagaaacagaattcacggaacatggattggtgcgcaaagcaggtggcacagcggctggagtacaaactggccttccatggaatcggttactttggagtgaaccccatgtataccagccaccaggaccctttcgaacataggcgcgtggctgatcacatcgtcatgcgagcacgttttgaggaagtcaacgtggagaacattgccgaatggcacgtgcgaaatttctcaaactacctgcgtgcagacagcggcactgggctgtactataagcaggccaccatggacttcctgaaacattacggtctggaggaacacgctgagggcctggaaaataagaaaatcaagttctatgactttagaaagatcctggaggataaaaacctgacaagcgtgatcattccaaagaggggcgggcgcatctacatggccaccaacccagtgacatccgactctaccccgattacatacgccggcaagacttataataggtgtaacgctgatgaggtggcagccgctaatatcgttatttctgtgctggctccccgcagtaagaaaaacgaggaacaggacgatatccctctgattaccaagaaagccgagagtaagtcaccaccgaaagaccggaagagatcaaaaacaagccagctgcctcagaaa

SEQ ID NO: 788 - RNA, 36 nt, Artificial Sequence
  Synthetic Construct
ucucaacgauagucagacauguguccucagugacac

SEQ ID NO: 789 - RNA, 36 nt, Artificial Sequence
  Synthetic Construct
uuuuaacaacacucaggcauguguccacagugacac

SEQ ID NO: 790 - RNA, 36 nt, Artificial Sequence
  Synthetic Construct
uugaacggauacucagacauguguuuccagugacac

SEQ ID NO: 791 - RNA, 36 nt, Artificial Sequence
  Synthetic Construct
ugcccucaauagucagauguguguccacagugacac

SEQ ID NO: 792 - RNA, 36 nt, Artificial Sequence
  Synthetic Construct
ucucaaugauacuuagauacguguccucagugacac

SEQ ID NO: 793 - RNA, 36 nt, Artificial Sequence
  Synthetic Construct
ucucaaugauacucagacauguguccccagugacac

SEQ ID NO: 794 - RNA, 36 nt, Artificial Sequence
  Synthetic Construct
ucucaaugauacuaagacauguguccucagugacac

SEQ ID NO: 795 - RNA, 36 nt, Artificial Sequence
  Synthetic Construct
ucucaacuauacucagacauguguccucagugacac

SEQ ID NO: 796 - RNA, 36 nt, Artificial Sequence
  Synthetic Construct
ucucaacgauacucagacauguguccucagugacac

SEQ ID NO: 797 - RNA, 36 nt, Artificial Sequence
  Synthetic Construct
ucucaacgauacuaagauauguguccucagcgacac

SEQ ID NO: 798 - RNA, 36 nt, Artificial Sequence
  Synthetic Construct
ucucaacgauacuaagauauguguccccagugacac

SEQ ID NO: 799 - RNA, 36 nt, Artificial Sequence
  Synthetic Construct
ucucaacgauacuaagauauguguccacagugacac

SEQ ID NO: 800 - RNA, 36 nt, Artificial Sequence
  Synthetic Construct
ucucaacaauacucagacauguguccccagugacac

SEQ ID NO: 801 - RNA, 36 nt, Artificial Sequence
  Synthetic Construct
ucucaacaauacuaaggcauguguccccagugaccc

SEQ ID NO: 802 - RNA, 36 nt, Artificial Sequence
  Synthetic Construct
ucucaaagauacucagacacguguccccagugacac

SEQ ID NO: 803 - RNA, 36 nt, Artificial Sequence
  Synthetic Construct
ucucaaaaauacucagacauguguccucagugacac

SEQ ID NO: 804 - RNA, 36 nt, Artificial Sequence
  Synthetic Construct
gcgaaacaacagucagacauguguccccagugacac

SEQ ID NO: 805 - RNA, 36 nt, Artificial Sequence
  Synthetic Construct
ccucaacgauauuaagacauguguccgcagugacac

SEQ ID NO: 806 - RNA, 22 nt, Artificial Sequence
  Synthetic Construct
agacauguguccucagugacac

SEQ ID NO: 807 - RNA, 36 nt, Artificial Sequence
  Synthetic Construct
guuggaaugacuaauuuuugugcccaccguuggcac

SEQ ID NO: 808 - RNA, 24 nt, Artificial Sequence
  Synthetic Construct
aauuuuugugcccaucguuggcac

SEQ ID NO: 809 - RNA, 23 nt, Artificial Sequence
  Synthetic Construct
auuuuugugcccaucguuggcac

SEQ ID NO: 810 - RNA, 36 nt, Artificial Sequence
  Synthetic Construct
cuagcaaugaccuaauaguguguccuuaguugacau

SEQ ID NO: 811 - RNA, 36 nt, Artificial Sequence
  Synthetic Construct
ccuacaauaccuaagaaauccguccuaaguugacgg

SEQ ID NO: 812 - RNA, 22 nt, Artificial Sequence
  Synthetic Construct
auaguguguccuuaguugacau

SEQ ID NO: 813 - RNA, 100 nt, Artificial Sequence
  Synthetic Construct
  misc_feature (1)..(4): The linkages between nucleotides at positions 1, 2, 3, and 4 are phosphorothioate linkages
  modified_base (1)..(2): gm
  modified_base (3)..(3): cm
  misc_feature (97)..(100): The linkages between nucleotides at positions 97, 98, 99, and 100 are phosphorothioate linkages
  modified_base (97)..(99): um
ggccgagaugucucgcuccgguuuuagagcuagaaauagcaaguuaaaauaaggcuaguccguuaucaacuugaaaaaguggcaccgagucggugcuuuu

SEQ ID NO: 814 - protein, 1074 aa, Artificial Sequence
  Synthetic Construct
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLEAELHYDGKKAKHHLPFYNARFFEEVYCYHPSVAEITPFKTKQFGCEIGKDIPDYVSVALKDNPYKKATKRILRAIYNPVANTTGVDKTTNCSFMIKRENDEYKLVINRKISVDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNEEQDDIPLITKKAESKSPPKDRKRSKTSQLPQK

SEQ ID NO: 815 - protein, 1074 aa, Artificial Sequence
  Synthetic Construct
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLEAELHYDGKKAKHHLPFYNARFFEEVYCYHPSVAEITPFKTKQFGCEIGKDIPDYVSVALKDNPYKKATKRILRAIYNPVANTTGVDKTTNCSFMIKRENDEYKLVINRKISRDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNREQDDIPLITKKAESKSPPKDRKRSKTSQLPQK

SEQ ID NO: 816 - protein, 1074 aa, Artificial Sequence
  Synthetic Construct
MASISRPYGTKLRPDARKKEMLDKFFNTLTKGQRVFADLALCIYGSLTLEMAKSLEPESDSELVCAIGWFRLVDKTIWSKDGIKQENLVKQYEAYSGKEASEVVKTYLNSPSSDKYVWIDCRQKFLRFQRELGTRNLSEDFECMLFEQYIRLTKGEIEGYAAISNMFGNGEKEDRSKKRMYATRMKDWLEANENITWEQYREALKNQLNAKNLEQVVANYKGNAGGADPFFKYSFSKEGMVSKKEHAQQLDKFKTVLKNKARDLNFPNKEKLKQYLEAEIGIPVDANVYSQMFSNGVSEVQPKTTRNMSFSNEKLDLLTELKDLNKGDGFEYAREVLNGFFDSELHTTEDKFNITSRYLGGDKSNRLSKLYKIWKKEGVDCEEGIQQFCEAVKDKMGQIPIRNVLKYLWQFRETVSAEDFEAAAKANHLEEKISRVKAHPIVISNRYWAFGTSALVGNIMPADKRHQGEYAGQNFKMWLRAELHYDGKKAKHHLPFYNARFFEEVYCYHPSVAEITPFKTKQFGCEIGKDIPDYVSVALKDNPYKKATKRILRAIYNPVANTTRVDKTTNCSFMIKRENDEYKLVINRKISRDRPKRIEVGRTIMGYDRNQTASDTYWIGRLVPPGTRGAYRIGEWSVQYIKSGPVLSSTQGVNNSTTDQLVYNGMPSSSERFKAWKKARMAFIRKLIRQLNDEGLESKGQDYIPENPSSFDVRGETLYVFNSNYLKALVSKHRKAKKPVEGILDEIEAWTSKDKDSCSLMRLSSLSDASMQGIASLKSLINSYFNKNGCKTIEDKEKFNPVLYAKLVEVEQRRTNKRSEKVGRIAGSLEQLALLNGVEVVIGEADLGEVEKGKSKKQNSRNMDWCAKQVAQRLEYKLAFHGIGYFGVNPMYTSHQDPFEHRRVADHIVMRARFEEVNVENIAEWHVRNFSNYLRADSGTGLYYKQATMDFLKHYGLEEHAEGLENKKIKFYDFRKILEDKNLTSVIIPKRGGRIYMATNPVTSDSTPITYAGKTYNRCNADEVAAANIVISVLAPRSKKNREQDDIPLITKKAESKSPPKDRKRSKTSQLPQK

SEQ ID NO: 817 - protein, 1093 aa, Artificial Sequence
  Synthetic Construct
MSNKEKNASETRKAYTTKMIPRSHDRMKLLGNFMDYLMDGTPIFFELWNQFGGGIDRDIISGTANKDKISDDLLLAVNWFKVMPINSKPQGVSPSNLANLFQQYSGSEPDIQAQEYFASNFDTEKHQWKDMRVEYERLLAELQLSRSDMHHDLKLMYKEKCIGLSLSTAHYITSVMFGTGAKNNRQTKHQFYSKVIQLLEESTQINSVEQLASIILKAGDCDSYRKLRIRCSRKGATPSILKIVQDYELGTNHDDEVNVPSLIANLKEKLGRFEYECEWKCMEKIKAFLASKVGPYYLGSYSAMLENALSPIKGMTTKNCKFVLKQIDAKNDIKYENEPFGKIVEGFFDSPYFESDTNVKWVLHPHHIGESNIKTLWEDLNAIHSKYEEDIASLSEDKKEKRIKVYQGDVCQTINTYCEEVGKEAKTPLVQLLRYLYSRKDDIAVDKIIDGITFLSKKHKVEKQKINPVIQKYPSFNFGNNSKLLGKIISPKDKLKHNLKCNRNQVDNYIWIEIKVLNTKTMRWEKHHYALSSTRFLEEVYYPATSENPPDALAARFRTKTNGYEGKPALSAEQIEQIRSAPVGLRKVKKRQMRLEAARQQNLLPRYTWGKDFNINICKRGNNFEVTLATKVKKKKEKNYKVVLGYDANIVRKNTYAAIEAHANGDGVIDYNDLPVKPIESGFVTVESQVRDKSYDQLSYNGVKLLYCKPHVESRRSFLEKYRNGTMKDNRGNNIQIDFMKDFEAIADDETSLYYFNMKYCKLLQSSIRNHSSQAKEYREEIFELLRDGKLSVLKLSSLSNLSFVMFKVAKSLIGTYFGHLLKKPKNSKSDVKAPPITDEDKQKADPEMFALRLALEEKRLNKVKSKKEVIANKIVAKALELRDKYGPVLIKGENISDTTKKGKKSSTNSFLMDWLARGVANKVKEMVMMHQGLEFVEVNPNFTSHQDPFVHKNPENTFRARYSRCTPSELTEKNRKEILSFLSDKPSKRPTNAYYNEGAMAFLATYGLKKNDVLGVSLEKFKQIMANILHQRSEDQLLFPSRGGMFYLATYKLDADATSVNWNGKQFWVCNADLVAAYNVGLVDIQKDFKKK

SEQ ID NO: 818 - protein, 1098 aa, Artificial Sequence
  Synthetic Construct
MSISNNNILPYNPKLLPDDRKHKMLVDTFNQLDLIRNNLHDMIIALYGALKYDNIKQFASKEKPHISADALCSINWFRLVKTNERKPAIESNQIISKFIQYSGHTPDKYALSHITGNHEPSHKWIDCREYAINYARIMHLSFSQFQDLATACLNCKILILNGTLTSSWAWGANSALFGGSDKENFSVKAKILNSFIENLKDEMNTTKFQVVEKVCQQIGSSDAADLFDLYRSTVKDGNRGPATGRNPKVMNLFSQDGEISSEQREDFIESFQKVMQEKNSKQIIPHLDKLKYHLVKQSGLYDIYSWAAAIKNANSTIVASNSSNLNTILNKTEKQQTFEELRKDEKIVACSKILLSVNDTLPEDLHYNPSTSNLGKNLDVFFDLLNENSVHTIENKEEKNKIVKECVNQYMEECKGLNKPPMPVLLTFISDYAHKHQAQDFLSAAKMNFIDLKIKSIKVVPTVHGSSPYTWISNLSKKNKDGKMIRTPNSSLIGWIIPPEEIHDQKFAGQNPIIWAVLRVYCNNKWEMHHFPFSDSRFFTEVYAYKPNLPYLPGGENRSKRFGYRHSTNLSNESRQILLDKSKYAKANKSVLRCMENMTHNVVFDPKTSLNIRIKTDKNNSPVLDDKGRITFVMQINHRILEKYNNTKIEIGDRILAYDQNQSENHTYAILQRTEEGSHAHQFNGWYVRVLETGKVTSIVQGLSGPIDQLNYDGMPVTSHKFNCWQADRSAFVSQFASLKISETETFDEAYQAINAQGAYTWNLFYLRILRKALRVCHMENINQFREEILAISKNRLSPMSLGSLSQNSLKMIRAFKSIINCYMSRMSFVDELQKKEGDLELHTIMRLTDNKLNDKRVEKINRASSFLTNKAHSMGCKMIVGESDLPVADSKTSKKQNVDRMDWCARALSHKVEYACKLMGLAYRGIPAYMSSHQDPLVHLVESKRSVLRPRFVVADKSDVKQHHLDNLRRMLNSKTKVGTAVYYREAVELMCEELGIHKTDMAKGKVSLSDFVDKFIGEKAIFPQRGGRFYMSTKRLTTGAKLICYSGSDVWLSDADEIAAINIGMFVVCDQTGAFKKKKKEKLDDEECDILPFRPM

SEQ ID NO: 819 - DNA, 20 bp, Artificial Sequence
  Synthetic Construct
ctgaagctgacagcattcgg

SEQ ID NO: 820 - DNA, 20 bp, Artificial Sequence
  Synthetic Construct
ctggcctggaggctatccag

SEQ ID NO: 821 - DNA, 20 bp, Artificial Sequence
  Synthetic Construct
tggcctggaggctatccagc

SEQ ID NO: 822 - DNA, 20 bp, Artificial Sequence
  Synthetic Construct
ctctcccgctctgcaccctc

SEQ ID NO: 823 - DNA, 20 bp, Artificial Sequence
  Synthetic Construct
ccttctccaagttctccttg

SEQ ID NO: 824 - DNA, 20 bp, Artificial Sequence
  Synthetic Construct
tccaagttctccttggtggc

SEQ ID NO: 825 - DNA, 20 bp, Artificial Sequence
  Synthetic Construct
tccttggtggcccgccgtgg

SEQ ID NO: 826 - DNA, 20 bp, Artificial Sequence
  Synthetic Construct
gtggcccgccgtggggctag

SEQ ID NO: 827 - DNA, 20 bp, Artificial Sequence
  Synthetic Construct
cccctttcggcggggagcag

SEQ ID NO: 828 - DNA, 20 bp, Artificial Sequence
  Synthetic Construct
cggcggggagcaggggagac

SEQ ID NO: 829 - DNA, 20 bp, Artificial Sequence
  Synthetic Construct
ggcggggagcaggggagacc

SEQ ID NO: 830 - DNA, 20 bp, Artificial Sequence
  Synthetic Construct
ggcctacggcgacgggaggg

SEQ ID NO: 831 - DNA, 20 bp, Artificial Sequence
  Synthetic Construct
gcctacggcgacgggagggt

SEQ ID NO: 832 - DNA, 20 bp, Artificial Sequence
  Synthetic Construct
agggcgtcgataagcgtcag

SEQ ID NO: 833 - DNA, 20 bp, Artificial Sequence
  Synthetic Construct
gggcgtcgataagcgtcaga

SEQ ID NO: 834 - DNA, 20 bp, Artificial Sequence
  Synthetic Construct
ggggagggtttctcttccgc

SEQ ID NO: 835 - DNA, 20 bp, Artificial Sequence
  Synthetic Construct
ctcttccgctctttcgcggg

SEQ ID NO: 836 - DNA, 20 bp, Artificial Sequence
  Synthetic Construct
tcttccgctctttcgcgggg

SEQ ID NO: 837 - DNA, 20 bp, Artificial Sequence
  Synthetic Construct
cgctctttcgcggggcctct

SEQ ID NO: 838 - DNA, 20 bp, Artificial Sequence
  Synthetic Construct
cgcggggcctctggctcccc

SEQ ID NO: 839 - DNA, 20 bp, Artificial Sequence
  Synthetic Construct
gcggggcctctggctccccc

SEQ ID NO: 840 - DNA, 20 bp, Artificial Sequence
  Synthetic Construct
gtgaacgcgtggaggggcgc

SEQ ID NO: 841 - DNA, 20 bp, Artificial Sequence
  Synthetic Construct
tgaacgcgtggaggggcgct

SEQ ID NO: 842 - DNA, 20 bp, Artificial Sequence
  Synthetic Construct
gggtctgggggaggcgtcgc

SEQ ID NO: 843 - DNA, 20 bp, Artificial Sequence
  Synthetic Construct
cccgggcgacgcctccccca

SEQ ID NO: 844 - DNA, 20 bp, Artificial Sequence
  Synthetic Construct
acaaacctcagcgccgcgcc

SEQ ID NO: 845 - DNA, 20 bp, Artificial Sequence
  Synthetic Construct
gggacgagcctacccgtccc

SEQ ID NO: 846 - DNA, 20 bp, Artificial Sequence
  Synthetic Construct
ggacgagcctacccgtcccc

SEQ ID NO: 847 - DNA, 20 bp, Artificial Sequence
  Synthetic Construct
tcgacgccctaaactttgtc

SEQ ID NO: 848 - DNA, 20 bp, Artificial Sequence
  Synthetic Construct
gtcccgaccctcccgtcgcc

SEQ ID NO: 849 - DNA, 20 bp, Artificial Sequence
  Synthetic Construct
tcccgaccctcccgtcgccg

SEQ ID NO: 850 - DNA, 20 bp, Artificial Sequence
  Synthetic Construct
cccactcccaggccaccccg

SEQ ID NO: 851 - DNA, 20 bp, Artificial Sequence
  Synthetic Construct
cccgagatccagccctggac

SEQ ID NO: 852 - DNA, 20 bp, Artificial Sequence
  Synthetic Construct
gagaagggaagtcacggagc

SEQ ID NO: 853 - DNA, 20 bp, Artificial Sequence
  Synthetic Construct
aggaatgcccgccagcgcga

SEQ ID NO: 854 - DNA, 20 bp, Artificial Sequence
  Synthetic Construct
tgagggaaagataccaagtc

SEQ ID NO: 855 - DNA, 20 bp, Artificial Sequence
  Synthetic Construct
attcttcaaaatggaggtgg

SEQ ID NO: 856 - DNA, 20 bp, Artificial Sequence
  Synthetic Construct
ttcttcaaaatggaggtggc

SEQ ID NO: 857 - DNA, 20 bp, Artificial Sequence
  Synthetic Construct
ttcaaaatggaggtggcttg

SEQ ID NO: 858 - DNA, 20 bp, Artificial Sequence
  Synthetic Construct
aaaatggaggtggcttgttg

SEQ ID NO: 859 - DNA, 20 bp, Artificial Sequence
  Synthetic Construct
ttgggaaggtggaagctcat

SEQ ID NO: 860 - DNA, 20 bp, Artificial Sequence
  Synthetic Construct
ggaaggtggaagctcatttg

SEQ ID NO: 861 - DNA, 20 bp, Artificial Sequence
  Synthetic Construct
ggccagagtggaaatggaat

SEQ ID NO: 862 - DNA, 20 bp, Artificial Sequence
  Synthetic Construct
gccagagtggaaatggaatt

SEQ ID NO: 863 - DNA, 20 bp, Artificial Sequence
  Synthetic Construct
ggagaaatcgatgaccaaat

SEQ ID NO: 864 - DNA, 20 bp, Artificial Sequence
  Synthetic Construct
gtgcctgatatagcttgaca

SEQ ID NO: 865 - DNA, 20 bp, Artificial Sequence
  Synthetic Construct
acaccaagttagccccaagt

SEQ ID NO: 866 - DNA, 20 bp, Artificial Sequence
  Synthetic Construct
gccccaagtgaaataccctg

SEQ ID NO: 867 - DNA, 20 bp, Artificial Sequence
  Synthetic Construct
atgtgtcttttcccgatatt

SEQ ID NO: 868 - DNA, 20 bp, Artificial Sequence
  Synthetic Construct
agtggggtaagtcttacatt

SEQ ID NO: 869 - DNA, 20 bp, Artificial Sequence
  Synthetic Construct
cattcttttgtaagctgctg

SEQ ID NO: 870 - DNA, 20 bp, Artificial Sequence
  Synthetic Construct
ttttgtaagctgctgaaagt

SEQ ID NO: 871 - DNA, 20 bp, Artificial Sequence
  Synthetic Construct
tgtaagctgctgaaagttgt

SEQ ID NO: 872 - DNA, 20 bp, Artificial Sequence
  Synthetic Construct
gtaagctgctgaaagttgtg

SEQ ID NO: 873 - DNA, 20 bp, Artificial Sequence
  Synthetic Construct
taagctgctgaaagttgtgt

SEQ ID NO: 874 - DNA, 20 bp, Artificial Sequence
  Synthetic Construct
tgtatgagtagtcatatcat

SEQ ID NO: 875 - DNA, 20 bp, Artificial Sequence
  Synthetic Construct
gatataaaaaaggtctatgg

SEQ ID NO: 876 - DNA, 20 bp, Artificial Sequence
  Synthetic Construct
atataaaaaaggtctatggc

SEQ ID NO: 877 - DNA, 20 bp, Artificial Sequence
  Synthetic Construct
ggattgtcagggaatgttct

SEQ ID NO: 878 - DNA, 20 bp, Artificial Sequence
  Synthetic Construct
tcagggaatgttcttaaaga

SEQ ID NO: 879 - DNA, 20 bp, Artificial Sequence
  Synthetic Construct
ttaaagatcagattagtggc

SEQ ID NO: 880 - DNA, 20 bp, Artificial Sequence
  Synthetic Construct
aagatcagattagtggcacc

SEQ ID NO: 881 - DNA, 20 bp, Artificial Sequence
  Synthetic Construct
gtggcacctgctgagatact

SEQ ID NO: 882 - DNA, 20 bp, Artificial Sequence
  Synthetic Construct
ctgaaccagtagtttccctg

SEQ ID NO: 883 - DNA, 20 bp, Artificial Sequence
  Synthetic Construct
tgaaccagtagtttccctgc

SEQ ID NO: 884 - DNA, 20 bp, Artificial Sequence
  Synthetic Construct
ccctgcagttgagcagggag

SEQ ID NO: 885 - DNA, 20 bp, Artificial Sequence
  Synthetic Construct
cctgcagttgagcagggagc

SEQ ID NO: 886 - DNA, 20 bp, Artificial Sequence
  Synthetic Construct
agcagggagcagcagcagca

SEQ ID NO: 887 - DNA, 20 bp, Artificial Sequence
  Synthetic Construct
cacaaatacatatacactct

SEQ ID NO: 888 - DNA, 20 bp, Artificial Sequence
  Synthetic Construct
acacttcttacctactggct

SEQ ID NO: 889 - DNA, 20 bp, Artificial Sequence
  Synthetic Construct
ttacctactggcttcctcta

SEQ ID NO: 890 - DNA, 20 bp, Artificial Sequence
  Synthetic Construct
cctactggcttcctctagct

SEQ ID NO: 891 - DNA, 20 bp, Artificial Sequence
  Synthetic Construct
ctctagcttttgtggcagct

SEQ ID NO: 892 - DNA, 20 bp, Artificial Sequence
  Synthetic Construct
tgtggcagcttcaggtatat

SEQ ID NO: 893 - DNA, 20 bp, Artificial Sequence
  Synthetic Construct
gtggcagcttcaggtatatt

SEQ ID NO: 894 - DNA, 20 bp, Artificial Sequence
  Synthetic Construct
tggcagcttcaggtatattt

SEQ ID NO: 895 - DNA, 20 bp, Artificial Sequence
  Synthetic Construct
aggtatatttagcactgaac

SEQ ID NO: 896 - DNA, 20 bp, Artificial Sequence
  Synthetic Construct
agcactgaacgaacatctca

SEQ ID NO: 897 - DNA, 20 bp, Artificial Sequence
  Synthetic Construct
gcactgaacgaacatctcaa

SEQ ID NO: 898 - DNA, 20 bp, Artificial Sequence
  Synthetic Construct
gtttgtaagtcctgctgtcc

SEQ ID NO: 899 - DNA, 20 bp, Artificial Sequence
  Synthetic Construct
tttgtaagtcctgctgtcct

SEQ ID NO: 900 - DNA, 20 bp, Artificial Sequence
  Synthetic Construct
gtaagtcctgctgtcctagc

SEQ ID NO: 901 - DNA, 20 bp, Artificial Sequence
  Synthetic Construct
taagtcctgctgtcctagca

SEQ ID NO: 902 - DNA, 20 bp, Artificial Sequence
  Synthetic Construct
tccagtactttctggctgga

SEQ ID NO: 903 - DNA, 20 bp, Artificial Sequence
  Synthetic Construct
ctggctggattggtatctga

SEQ ID NO: 904 - DNA, 20 bp, Artificial Sequence
  Synthetic Construct
tggctggattggtatctgag

SEQ ID NO: 905 - DNA, 20 bp, Artificial Sequence
  Synthetic Construct
gtatctgaggctagtaggaa

SEQ ID NO: 906 - DNA, 20 bp, Artificial Sequence
  Synthetic Construct
ttcctgctgggtagctctaa

SEQ ID NO: 907 - DNA, 20 bp, Artificial Sequence
  Synthetic Construct
ctgctgggtagctctaaaca

SEQ ID NO: 908 - DNA, 20 bp, Artificial Sequence
  Synthetic Construct
atgggtaggaacagcagcct

SEQ ID NO: 909 - DNA, 20 bp, Artificial Sequence
  Synthetic Construct
tgccagccttatttctaacc

SEQ ID NO: 910 - DNA, 20 bp, Artificial Sequence
  Synthetic Construct
tttctaaccattttagacat

SEQ ID NO: 911 - DNA, 20 bp, Artificial Sequence
  Synthetic Construct
ctaaccattttagacatttg

SEQ ID NO: 912 - DNA, 20 bp, Artificial Sequence
  Synthetic Construct
taaccattttagacatttgt

SEQ ID NO: 913 - DNA, 20 bp, Artificial Sequence
  Synthetic Construct
tagacatttgttagtacatg

SEQ ID NO: 914 - DNA, 20 bp, Artificial Sequence
  Synthetic Construct
agacatttgttagtacatgg

SEQ ID NO: 915 - DNA, 20 bp, Artificial Sequence
  Synthetic Construct
gacatttgttagtacatggt

SEQ ID NO: 916 - DNA, 20 bp, Artificial Sequence
  Synthetic Construct
gttagtacatggtattttaa

SEQ ID NO: 917 - DNA, 20 bp, Artificial Sequence
  Synthetic Construct
ttagtacatggtattttaaa

SEQ ID NO: 918 - DNA, 20 bp, Artificial Sequence
  Synthetic Construct
gtacatggtattttaaaagt

SEQ ID NO: 919 - DNA, 20 bp, Artificial Sequence
  Synthetic Construct
taaaagtaaaacttaatgtc

SEQ ID NO: 920 - DNA, 20 bp, Artificial Sequence
  Synthetic Construct
aaaagtaaaacttaatgtct

SEQ ID NO: 921 - DNA, 20 bp, Artificial Sequence
  Synthetic Construct
aaagtaaaacttaatgtctt

SEQ ID NO: 922 - DNA, 20 bp, Artificial Sequence
  Synthetic Construct
atgtcttccttttttttctc

SEQ ID NO: 923 - DNA, 20 bp, Artificial Sequence
  Synthetic Construct
cttttttttctccactgtct

SEQ ID NO: 924 - DNA, 20 bp, Artificial Sequence
  Synthetic Construct
tttttctccactgtcttttt

SEQ ID NO: 925 - DNA, 20 bp, Artificial Sequence
  Synthetic Construct
ttttctccactgtctttttc

SEQ ID NO: 926 - DNA, 20 bp, Artificial Sequence
  Synthetic Construct
ttcatagatcgagacatgta

SEQ ID NO: 927 - DNA, 20 bp, Artificial Sequence
  Synthetic Construct
tcatagatcgagacatgtaa

SEQ ID NO: 928 - DNA, 20 bp, Artificial Sequence
  Synthetic Construct
catagatcgagacatgtaag

SEQ ID NO: 929 - DNA, 20 bp, Artificial Sequence
  Synthetic Construct
atagatcgagacatgtaagc

SEQ ID NO: 930 - DNA, 20 bp, Artificial Sequence
  Synthetic Construct
ttgaccttgagaaaatgttt

SEQ ID NO: 931 - DNA, 20 bp, Artificial Sequence
  Synthetic Construct
tgaccttgagaaaatgtttt

SEQ ID NO: 932 - DNA, 20 bp, Artificial Sequence
  Synthetic Construct
gaccttgagaaaatgttttt

SEQ ID NO: 933 - DNA, 20 bp, Artificial Sequence
  Synthetic Construct
accttgagaaaatgtttttg

SEQ ID NO: 934 - DNA, 20 bp, Artificial Sequence
  Synthetic Construct
agaaaatgtttttgtttcac

SEQ ID NO: 935 - DNA, 20 bp, Artificial Sequence
  Synthetic Construct
ttgtttcactgtcctgagga

SEQ ID NO: 936 - DNA, 20 bp, Artificial Sequence
  Synthetic Construct
tgtttcactgtcctgaggac

SEQ ID NO: 937 - DNA, 20 bp, Artificial Sequence
  Synthetic Construct
gtttcactgtcctgaggact

SEQ ID NO: 938 - DNA, 20 bp, Artificial Sequence
  Synthetic Construct
tttcactgtcctgaggacta

SEQ ID NO: 939 - DNA, 20 bp, Artificial Sequence
  Synthetic Construct
cactgtcctgaggactattt

SEQ ID NO: 940 - DNA, 20 bp, Artificial Sequence
  Synthetic Construct
actgtcctgaggactattta

SEQ ID NO: 941 - DNA, 20 bp, Artificial Sequence
  Synthetic Construct
atagacagctctaacatgat

SEQ ID NO: 942 - DNA, 20 bp, Artificial Sequence
  Synthetic Construct
tagacagctctaacatgata

SEQ ID NO: 943 - DNA, 20 bp, Artificial Sequence
  Synthetic Construct
tcatgttagagctgtctata

SEQ ID NO: 944 - DNA, 20 bp, Artificial Sequence
  Synthetic Construct
gagctgtctataaatagtcc

SEQ ID NO: 945 - DNA, 20 bp, Artificial Sequence
  Synthetic Construct
tctcaaggtcaaaaacttac

SEQ ID NO: 946 - DNA, 20 bp, Artificial Sequence
  Synthetic Construct
ctcaaggtcaaaaacttacc

SEQ ID NO: 947 - DNA, 20 bp, Artificial Sequence
  Synthetic Construct
tcaaggtcaaaaacttacct

SEQ ID NO: 948 - DNA, 20 bp, Artificial Sequence
  Synthetic Construct
catgtctcgatctatgaaaa

SEQ ID NO: 949 - DNA, 20 bp, Artificial Sequence
  Synthetic Construct
agttttacttttaaaatacc

SEQ ID NO: 950 - DNA, 20 bp, Artificial Sequence
  Synthetic Construct
tacttttaaaataccatgta

SEQ ID NO: 951 - DNA, 20 bp, Artificial Sequence
  Synthetic Construct
acttttaaaataccatgtac

SEQ ID NO: 952 - DNA, 20 bp, Artificial Sequence
  Synthetic Construct
cttttaaaataccatgtact

SEQ ID NO: 953 - DNA, 20 bp, Artificial Sequence
  Synthetic Construct
taaaataccatgtactaaca

SEQ ID NO: 954 - DNA, 20 bp, Artificial Sequence
  Synthetic Construct
aaaataccatgtactaacaa

SEQ ID NO: 955 - DNA, 20 bp, Artificial Sequence
  Synthetic Construct
aaataccatgtactaacaaa

SEQ ID NO: 956 - DNA, 20 bp, Artificial Sequence
  Synthetic Construct
gaaataaggctggcagaata

SEQ ID NO: 957 - DNA, 20 bp, Artificial Sequence
  Synthetic Construct
ctacccatgaatacattgtt

SEQ ID NO: 958 - DNA, 20 bp, Artificial Sequence
  Synthetic Construct
tttagagctacccagcagga

SEQ ID NO: 959 - DNA, 20 bp, Artificial Sequence
  Synthetic Construct
agagctacccagcaggaaca

SEQ ID NO: 960 - DNA, 20 bp, Artificial Sequence
  Synthetic Construct
gagctacccagcaggaacaa

SEQ ID NO: 961 - DNA, 20 bp, Artificial Sequence
  Synthetic Construct
ctactagcctcagataccaa

SEQ ID NO: 962 - DNA, 20 bp, Artificial Sequence
  Synthetic Construct
taggatgctaggacagcagg

SEQ ID NO: 963 - DNA, 20 bp, Artificial Sequence
  Synthetic Construct
caaacaaaggcctatacctt

SEQ ID NO: 964 - DNA, 20 bp, Artificial Sequence
  Synthetic Construct
ttgagatgttcgttcagtgc

SEQ ID NO: 965 - DNA, 20 bp, Artificial Sequence
  Synthetic Construct
agatgttcgttcagtgctaa

SEQ ID NO: 966 - DNA, 20 bp, Artificial Sequence
  Synthetic Construct
gttcagtgctaaatatacct

SEQ ID NO: 967 - DNA, 20 bp, Artificial Sequence
  Synthetic Construct
agtgctaaatatacctgaag

SEQ ID NO: 968 - DNA, 20 bp, Artificial Sequence
  Synthetic Construct
agagtgtatatgtatttgtg

SEQ ID NO: 969 - DNA, 20 bp, Artificial Sequence
  Synthetic Construct
gtgcaagtgctgctgctgct

SEQ ID NO: 970 - DNA, 20 bp, Artificial Sequence
  Synthetic Construct
tgcaagtgctgctgctgctc

SEQ ID NO: 971 - DNA, 20 bp, Artificial Sequence
  Synthetic Construct
agaaaccatgctgtgcatca

SEQ ID NO: 972 - DNA, 20 bp, Artificial Sequence
  Synthetic Construct
aagaacattccctgacaatc

SEQ ID NO: 973 - DNA, 20 bp, Artificial Sequence
  Synthetic Construct
agaacattccctgacaatcc

SEQ ID NO: 974 - DNA, 20 bp, Artificial Sequence
  Synthetic Construct
cctgacaatcccaatatgca

SEQ ID NO: 975 - DNA, 20 bp, Artificial Sequence
  Synthetic Construct
tttatatcagatgggatggg

SEQ ID NO: 976 - DNA, 20 bp, Artificial Sequence
  Synthetic Construct
atatcagatgggatgggact

SEQ ID NO: 977 - DNA, 20 bp, Artificial Sequence
  Synthetic Construct
tatcagatgggatgggactc

SEQ ID NO: 978 - DNA, 20 bp, Artificial Sequence
  Synthetic Construct
agggtagtatggccatagac

SEQ ID NO: 979 - DNA, 20 bp, Artificial Sequence
  Synthetic Construct
tttatatcaaagcagcttta

SEQ ID NO: 980 - DNA, 20 bp, Artificial Sequence
  Synthetic Construct
ttatatcaaagcagctttat

SEQ ID NO: 981 - DNA, 20 bp, Artificial Sequence
  Synthetic Construct
tatatcaaagcagctttatg

SEQ ID NO: 982 - DNA, 20 bp, Artificial Sequence
  Synthetic Construct
atatcaaagcagctttatga

SEQ ID NO: 983 - DNA, 20 bp, Artificial Sequence
  Synthetic Construct
tatcaaagcagctttatgat

SEQ ID NO: 984 - DNA, 20 bp, Artificial Sequence
  Synthetic Construct
atgatatgactactcataca

SEQ ID NO: 985 - DNA, 20 bp, Artificial Sequence
  Synthetic Construct
tgatatgactactcatacac

SEQ ID NO: 986 - DNA, 20 bp, Artificial Sequence
  Synthetic Construct
cagcagcttacaaaagaatg

SEQ ID NO: 987 - DNA, 20 bp, Artificial Sequence
  Synthetic Construct
agcagcttacaaaagaatgt

SEQ ID NO: 988 - DNA, 20 bp, Artificial Sequence
  Synthetic Construct
ggagtacctgaggaatatcg

SEQ ID NO: 989 - DNA, 20 bp, Artificial Sequence
  Synthetic Construct
gagtacctgaggaatatcgg

SEQ ID NO: 990 - DNA, 20 bp, Artificial Sequence
  Synthetic Construct
atattgccagggtatttcac

SEQ ID NO: 991 - DNA, 20 bp, Artificial Sequence
  Synthetic Construct
ccagggtatttcacttgggg

SEQ ID NO: 992 - DNA, 20 bp, Artificial Sequence
  Synthetic Construct
cacttggggctaacttggtg

SEQ ID NO: 993 - DNA, 20 bp, Artificial Sequence
  Synthetic Construct
acttggggctaacttggtgt

SEQ ID NO: 994 - DNA, 20 bp, Artificial Sequence
  Synthetic Construct
gggctaacttggtgtcaagc

SEQ ID NO: 995 - DNA, 20 bp, Artificial Sequence
  Synthetic Construct
gtgtcaagctatatcaggca

SEQ ID NO: 996 - DNA, 20 bp, Artificial Sequence
  Synthetic Construct
acatttggtcatcgatttct

SEQ ID NO: 997 - DNA, 20 bp, Artificial Sequence
  Synthetic Construct
catttggtcatcgatttctc

SEQ ID NO: 998 - DNA, 20 bp, Artificial Sequence
  Synthetic Construct
ggtcatcgatttctcccaat

SEQ ID NO: 999 - DNA, 20 bp, Artificial Sequence
  Synthetic Construct
gtcatcgatttctcccaatt

SEQ ID NO: 1000 - DNA, 20 bp, Artificial Sequence
  Synthetic Construct
ctcccaattccatttccact

SEQ ID NO: 1001 - DNA, 20 bp, Artificial Sequence
  Synthetic Construct
tcccaattccatttccactc

SEQ ID NO: 1002 - DNA, 20 bp, Artificial Sequence
  Synthetic Construct
catttccactctggccaaat

SEQ ID NO: 1003 - DNA, 20 bp, Artificial Sequence
  Synthetic Construct
ccactctggccaaatgagct

SEQ ID NO: 1004 - DNA, 20 bp, Artificial Sequence
  Synthetic Construct
cactctggccaaatgagctt

SEQ ID NO: 1005 - DNA, 20 bp, Artificial Sequence
  Synthetic Construct
caccttcccaacaagccacc

SEQ ID NO: 1006 - DNA, 20 bp, Artificial Sequence
  Synthetic Construct
ccaacaagccacctccattt

SEQ ID NO: 1007 - DNA, 20 bp, Artificial Sequence
  Synthetic Construct
tgaagaataaaccgtgactt

SEQ ID NO: 1008 - DNA, 20 bp, Artificial Sequence
  Synthetic Construct
gaagaataaaccgtgacttg

SEQ ID NO: 1009 - DNA, 20 bp, Artificial Sequence
  Synthetic Construct
aagaataaaccgtgacttgg

SEQ ID NO: 1010 - DNA, 20 bp, Artificial Sequence
  Synthetic Construct
gtatctttccctcataattc

SEQ ID NO: 1011 - DNA, 20 bp, Artificial Sequence
  Synthetic Construct
ccctcataattcctctatac

SEQ ID NO: 1012 - DNA, 20 bp, Artificial Sequence
  Synthetic Construct
cctcataattcctctataca

SEQ ID NO: 1013 - DNA, 20 bp, Artificial Sequence
  Synthetic Construct
ctctatacatgccttttttg

SEQ ID NO: 1014 - DNA, 20 bp, Artificial Sequence
  Synthetic Construct
tttgttttttttctagcaga

SEQ ID NO: 1015 - DNA, 20 bp, Artificial Sequence
  Synthetic Construct
ttgttttttttctagcagat

SEQ ID NO: 1016 - DNA, 20 bp, Artificial Sequence
  Synthetic Construct
tgttttttttctagcagatt

SEQ ID NO: 1017 - DNA, 20 bp, Artificial Sequence
  Synthetic Construct
gttttttttctagcagattt

SEQ ID NO: 1018 - DNA, 20 bp, Artificial Sequence
  Synthetic Construct
ttttttttctagcagatttc

SEQ ID NO: 1019 - RNA, 20 nt, Artificial Sequence
  Synthetic Construct
cugaagcugacagcauucgg

SEQ ID NO: 1020 - RNA, 20 nt, Artificial Sequence
  Synthetic Construct
cuggccuggaggcuauccag

SEQ ID NO: 1021 - RNA, 20 nt, Artificial Sequence
  Synthetic Construct
uggccuggaggcuauccagc

SEQ ID NO: 1022 - RNA, 20 nt, Artificial Sequence
  Synthetic Construct
cucucccgcucugcacccuc

SEQ ID NO: 1023 - RNA, 20 nt, Artificial Sequence
  Synthetic Construct
ccuucuccaaguucuccuug

SEQ ID NO: 1024 - RNA, 20 nt, Artificial Sequence
  Synthetic Construct
uccaaguucuccuugguggc

SEQ ID NO: 1025 - RNA, 20 nt, Artificial Sequence
  Synthetic Construct
uccuugguggcccgccgugg

SEQ ID NO: 1026 - RNA, 20 nt, Artificial Sequence
  Synthetic Construct
guggcccgccguggggcuag

SEQ ID NO: 1027 - RNA, 20 nt, Artificial Sequence
  Synthetic Construct
ccccuuucggcggggagcag

SEQ ID NO: 1028 - RNA, 20 nt, Artificial Sequence
  Synthetic Construct
cggcggggagcaggggagac

SEQ ID NO: 1029 - RNA, 20 nt, Artificial Sequence
  Synthetic Construct
ggcggggagcaggggagacc

SEQ ID NO: 1030 - RNA, 20 nt, Artificial Sequence
  Synthetic Construct
ggccuacggcgacgggaggg

SEQ ID NO: 1031 - RNA, 20 nt, Artificial Sequence
  Synthetic Construct
gccuacggcgacgggagggu

SEQ ID NO: 1032 - RNA, 20 nt, Artificial Sequence
  Synthetic Construct
agggcgucgauaagcgucag

SEQ ID NO: 1033 - RNA, 20 nt, Artificial Sequence
  Synthetic Construct
gggcgucgauaagcgucaga

SEQ ID NO: 1034 - RNA, 20 nt, Artificial Sequence
  Synthetic Construct
ggggaggguuucucuuccgc

SEQ ID NO: 1035 - RNA, 20 nt, Artificial Sequence
  Synthetic Construct
cucuuccgcucuuucgcggg

SEQ ID NO: 1036 - RNA, 20 nt, Artificial Sequence
  Synthetic Construct
ucuuccgcucuuucgcgggg

SEQ ID NO: 1037 - RNA, 20 nt, Artificial Sequence
  Synthetic Construct
cgcucuuucgcggggccucu

SEQ ID NO: 1038 - RNA, 20 nt, Artificial Sequence
  Synthetic Construct
cgcggggccucuggcucccc

SEQ ID NO: 1039 - RNA, 20 nt, Artificial Sequence
  Synthetic Construct
gcggggccucuggcuccccc

SEQ ID NO: 1040 - RNA, 20 nt, Artificial Sequence
  Synthetic Construct
gugaacgcguggaggggcgc

SEQ ID NO: 1041 - RNA, 20 nt, Artificial Sequence
  Synthetic Construct
ugaacgcguggaggggcgcu

SEQ ID NO: 1042 - RNA, 20 nt, Artificial Sequence
  Synthetic Construct
gggucugggggaggcgucgc

SEQ ID NO: 1043 - RNA, 20 nt, Artificial Sequence
  Synthetic Construct
cccgggcgacgccuccccca

SEQ ID NO: 1044 - RNA, 20 nt, Artificial Sequence
  Synthetic Construct
acaaaccucagcgccgcgcc

SEQ ID NO: 1045 - RNA, 20 nt, Artificial Sequence
  Synthetic Construct
gggacgagccuacccguccc

SEQ ID NO: 1046 - RNA, 20 nt, Artificial Sequence
  Synthetic Construct
ggacgagccuacccgucccc

SEQ ID NO: 1047 - RNA, 20 nt, Artificial Sequence
  Synthetic Construct
ucgacgcccuaaacuuuguc

SEQ ID NO: 1048 - RNA, 20 nt, Artificial Sequence
  Synthetic Construct
gucccgacccucccgucgcc

SEQ ID NO: 1049 - RNA, 20 nt, Artificial Sequence
  Synthetic Construct
ucccgacccucccgucgccg

SEQ ID NO: 1050 - RNA, 20 nt, Artificial Sequence
  Synthetic Construct
cccacucccaggccaccccg

SEQ ID NO: 1051 - RNA, 20 nt, Artificial Sequence
  Synthetic Construct
cccgagauccagcccuggac

SEQ ID NO: 1052 - RNA, 20 nt, Artificial Sequence
  Synthetic Construct
gagaagggaagucacggagc

SEQ ID NO: 1053 - RNA, 20 nt, Artificial Sequence
  Synthetic Construct
aggaaugcccgccagcgcga

SEQ ID NO: 1054 - RNA, 20 nt, Artificial Sequence
  Synthetic Construct
ugagggaaagauaccaaguc

SEQ ID NO: 1055 - RNA, 20 nt, Artificial Sequence
  Synthetic Construct
auucuucaaaauggaggugg

SEQ ID NO: 1056 - RNA, 20 nt, Artificial Sequence
  Synthetic Construct
uucuucaaaauggagguggc

SEQ ID NO: 1057 - RNA, 20 nt, Artificial Sequence
  Synthetic Construct
uucaaaauggagguggcuug

SEQ ID NO: 1058 - RNA, 20 nt, Artificial Sequence
  Synthetic Construct
aaaauggagguggcuuguug

SEQ ID NO: 1059 - RNA, 20 nt, Artificial Sequence
  Synthetic Construct
uugggaagguggaagcucau

SEQ ID NO: 1060 - RNA, 20 nt, Artificial Sequence
  Synthetic Construct
ggaagguggaagcucauuug

SEQ ID NO: 1061 - RNA, 20 nt, Artificial Sequence
  Synthetic Construct
ggccagaguggaaauggaau

SEQ ID NO: 1062 - RNA, 20 nt, Artificial Sequence
  Synthetic Construct
gccagaguggaaauggaauu

SEQ ID NO: 1063 - RNA, 20 nt, Artificial Sequence
  Synthetic Construct
ggagaaaucgaugaccaaau

SEQ ID NO: 1064 - RNA, 20 nt, Artificial Sequence
  Synthetic Construct
gugccugauauagcuugaca

SEQ ID NO: 1065 - RNA, 20 nt, Artificial Sequence
  Synthetic Construct
acaccaaguuagccccaagu

SEQ ID NO: 1066 - RNA, 20 nt, Artificial Sequence
  Synthetic Construct
gccccaagugaaauacccug

SEQ ID NO: 1067 - RNA, 20 nt, Artificial Sequence
  Synthetic Construct
augugucuuuucccgauauu

SEQ ID NO: 1068 - RNA, 20 nt, Artificial Sequence
  Synthetic Construct
agugggguaagucuuacauu

SEQ ID NO: 1069 - RNA, 20 nt, Artificial Sequence
  Synthetic Construct
cauucuuuuguaagcugcug

SEQ ID NO: 1070 - RNA, 20 nt, Artificial Sequence
  Synthetic Construct
uuuuguaagcugcugaaagu

SEQ ID NO: 1071 - RNA, 20 nt, Artificial Sequence
  Synthetic Construct
uguaagcugcugaaaguugu

SEQ ID NO: 1072 - RNA, 20 nt, Artificial Sequence
  Synthetic Construct
guaagcugcugaaaguugug

SEQ ID NO: 1073 - RNA, 20 nt, Artificial Sequence
  Synthetic Construct
uaagcugcugaaaguugugu

SEQ ID NO: 1074 - RNA, 20 nt, Artificial Sequence
  Synthetic Construct
uguaugaguagucauaucau

SEQ ID NO: 1075 - RNA, 20 nt, Artificial Sequence
  Synthetic Construct
gauauaaaaaaggucuaugg

SEQ ID NO: 1076 - RNA, 20 nt, Artificial Sequence
  Synthetic Construct
auauaaaaaaggucuauggc

SEQ ID NO: 1077 - RNA, 20 nt, Artificial Sequence
  Synthetic Construct
ggauugucagggaauguucu

SEQ ID NO: 1078 - RNA, 20 nt, Artificial Sequence
  Synthetic Construct
ucagggaauguucuuaaaga

SEQ ID NO: 1079 - RNA, 20 nt, Artificial Sequence
  Synthetic Construct
uuaaagaucagauuaguggc

SEQ ID NO: 1080 - RNA, 20 nt, Artificial Sequence
  Synthetic Construct
aagaucagauuaguggcacc

SEQ ID NO: 1081 - RNA, 20 nt, Artificial Sequence
  Synthetic Construct
guggcaccugcugagauacu

SEQ ID NO: 1082 - RNA, 20 nt, Artificial Sequence
  Synthetic Construct
cugaaccaguaguuucccug

SEQ ID NO: 1083 - RNA, 20 nt, Artificial Sequence
  Synthetic Construct
ugaaccaguaguuucccugc

SEQ ID NO: 1084 - RNA, 20 nt, Artificial Sequence
  Synthetic Construct
cccugcaguugagcagggag

SEQ ID NO: 1085 - RNA, 20 nt, Artificial Sequence
  Synthetic Construct
ccugcaguugagcagggagc

SEQ ID NO: 1086 - RNA, 20 nt, Artificial Sequence
  Synthetic Construct
agcagggagcagcagcagca

SEQ ID NO: 1087 - RNA, 20 nt, Artificial Sequence
  Synthetic Construct
cacaaauacauauacacucu

SEQ ID NO: 1088 - RNA, 20 nt, Artificial Sequence
  Synthetic Construct
acacuucuuaccuacuggcu

SEQ ID NO: 1089 - RNA, 20 nt, Artificial Sequence
  Synthetic Construct
uuaccuacuggcuuccucua

SEQ ID NO: 1090 - RNA, 20 nt, Artificial Sequence
  Synthetic Construct
ccuacuggcuuccucuagcu

SEQ ID NO: 1091 - RNA, 20 nt, Artificial Sequence
  Synthetic Construct
cucuagcuuuuguggcagcu

SEQ ID NO: 1092 - RNA, 20 nt, Artificial Sequence
  Synthetic Construct
uguggcagcuucagguauau

SEQ ID NO: 1093 - RNA, 20 nt, Artificial Sequence
  Synthetic Construct
guggcagcuucagguauauu

SEQ ID NO: 1094 - RNA, 20 nt, Artificial Sequence
  Synthetic Construct
uggcagcuucagguauauuu

SEQ ID NO: 1095 - RNA, 20 nt, Artificial Sequence
  Synthetic Construct
agguauauuuagcacugaac

SEQ ID NO: 1096 - RNA, 20 nt, Artificial Sequence
  Synthetic Construct
agcacugaacgaacaucuca

SEQ ID NO: 1097 - RNA, 20 nt, Artificial Sequence
  Synthetic Construct
gcacugaacgaacaucucaa

SEQ ID NO: 1098 - RNA, 20 nt, Artificial Sequence
  Synthetic Construct
guuuguaaguccugcugucc

SEQ ID NO: 1099 - RNA, 20 nt, Artificial Sequence
  Synthetic Construct
uuuguaaguccugcuguccu

SEQ ID NO: 1100 - RNA, 20 nt, Artificial Sequence
  Synthetic Construct
guaaguccugcuguccuagc

SEQ ID NO: 1101 - RNA, 20 nt, Artificial Sequence
  Synthetic Construct
uaaguccugcuguccuagca

SEQ ID NO: 1102 - RNA, 20 nt, Artificial Sequence
  Synthetic Construct
uccaguacuuucuggcugga

SEQ ID NO: 1103 - RNA, 20 nt, Artificial Sequence
  Synthetic Construct
cuggcuggauugguaucuga

SEQ ID NO: 1104 - RNA, 20 nt, Artificial Sequence
  Synthetic Construct
uggcuggauugguaucugag

SEQ ID NO: 1105 - RNA, 20 nt, Artificial Sequence
  Synthetic Construct
guaucugaggcuaguaggaa

SEQ ID NO: 1106 - RNA, 20 nt, Artificial Sequence
  Synthetic Construct
uuccugcuggguagcucuaa

SEQ ID NO: 1107 - RNA, 20 nt, Artificial Sequence
  Synthetic Construct
cugcuggguagcucuaaaca

SEQ ID NO: 1108 - RNA, 20 nt, Artificial Sequence
  Synthetic Construct
auggguaggaacagcagccu

SEQ ID NO: 1109 - RNA, 20 nt, Artificial Sequence
  Synthetic Construct
ugccagccuuauuucuaacc

SEQ ID NO: 1110 - RNA, 20 nt, Artificial Sequence
  Synthetic Construct
uuucuaaccauuuuagacau

SEQ ID NO: 1111 - RNA, 20 nt, Artificial Sequence
  Synthetic Construct
cuaaccauuuuagacauuug

SEQ ID NO: 1112 - RNA, 20 nt, Artificial Sequence
  Synthetic Construct
uaaccauuuuagacauuugu

SEQ ID NO: 1113 - RNA, 20 nt, Artificial Sequence
  Synthetic Construct
uagacauuuguuaguacaug

SEQ ID NO: 1114 - RNA, 20 nt, Artificial Sequence
  Synthetic Construct
agacauuuguuaguacaugg

SEQ ID NO: 1115 - RNA, 20 nt, Artificial Sequence
  Synthetic Construct
gacauuuguuaguacauggu

SEQ ID NO: 1116 - RNA, 20 nt, Artificial Sequence
  Synthetic Construct
guuaguacaugguauuuuaa

SEQ ID NO: 1117 - RNA, 20 nt, Artificial Sequence
  Synthetic Construct
uuaguacaugguauuuuaaa

SEQ ID NO: 1118 - RNA, 20 nt, Artificial Sequence
  Synthetic Construct
guacaugguauuuuaaaagu

SEQ ID NO: 1119 - RNA, 20 nt, Artificial Sequence
  Synthetic Construct
uaaaaguaaaacuuaauguc

SEQ ID NO: 1120 - RNA, 20 nt, Artificial Sequence
  Synthetic Construct
aaaaguaaaacuuaaugucu

SEQ ID NO: 1121 - RNA, 20 nt, Artificial Sequence
  Synthetic Construct
aaaguaaaacuuaaugucuu

SEQ ID NO: 1122 - RNA, 20 nt, Artificial Sequence
  Synthetic Construct
augucuuccuuuuuuuucuc

SEQ ID NO: 1123 - RNA, 20 nt, Artificial Sequence
  Synthetic Construct
cuuuuuuuucuccacugucu

SEQ ID NO: 1124 - RNA, 20 nt, Artificial Sequence
  Synthetic Construct
uuuuucuccacugucuuuuu

SEQ ID NO: 1125 - RNA, 20 nt, Artificial Sequence
  Synthetic Construct
uuuucuccacugucuuuuuc

SEQ ID NO: 1126 - RNA, 20 nt, Artificial Sequence
  Synthetic Construct
uucauagaucgagacaugua

SEQ ID NO: 1127 - RNA, 20 nt, Artificial Sequence
  Synthetic Construct
ucauagaucgagacauguaa

SEQ ID NO: 1128 - RNA, 20 nt, Artificial Sequence
  Synthetic Construct
cauagaucgagacauguaag

SEQ ID NO: 1129 - RNA, 20 nt, Artificial Sequence
  Synthetic Construct
auagaucgagacauguaagc

SEQ ID NO: 1130 - RNA, 20 nt, Artificial Sequence
  Synthetic Construct
uugaccuugagaaaauguuu

SEQ ID NO: 1131 - RNA, 20 nt, Artificial Sequence
  Synthetic Construct
ugaccuugagaaaauguuuu

SEQ ID NO: 1132 - RNA, 20 nt, Artificial Sequence
  Synthetic Construct
gaccuugagaaaauguuuuu

SEQ ID NO: 1133 - RNA, 20 nt, Artificial Sequence
  Synthetic Construct
accuugagaaaauguuuuug

SEQ ID NO: 1134 - RNA, 20 nt, Artificial Sequence
  Synthetic Construct
agaaaauguuuuuguuucac

SEQ ID NO: 1135 - RNA, 20 nt, Artificial Sequence
  Synthetic Construct
uuguuucacuguccugagga

SEQ ID NO: 1136 - RNA, 20 nt, Artificial Sequence
  Synthetic Construct
uguuucacuguccugaggac

SEQ ID NO: 1137 - RNA, 20 nt, Artificial Sequence
  Synthetic Construct
guuucacuguccugaggacu

SEQ ID NO: 1138 - RNA, 20 nt, Artificial Sequence
  Synthetic Construct
uuucacuguccugaggacua

SEQ ID NO: 1139 - RNA, 20 nt, Artificial Sequence
  Synthetic Construct
cacuguccugaggacuauuu

SEQ ID NO: 1140 - RNA, 20 nt, Artificial Sequence
  Synthetic Construct
acuguccugaggacuauuua

SEQ ID NO: 1141 - RNA, 20 nt, Artificial Sequence
  Synthetic Construct
auagacagcucuaacaugau

SEQ ID NO: 1142 - RNA, 20 nt, Artificial Sequence
  Synthetic Construct
uagacagcucuaacaugaua

SEQ ID NO: 1143 - RNA, 20 nt, Artificial Sequence
  Synthetic Construct
ucauguuagagcugucuaua

SEQ ID NO: 1144 - RNA, 20 nt, Artificial Sequence
  Synthetic Construct
gagcugucuauaaauagucc

SEQ ID NO: 1145 - RNA, 20 nt, Artificial Sequence
  Synthetic Construct
ucucaaggucaaaaacuuac

SEQ ID NO: 1146 - RNA, 20 nt, Artificial Sequence
  Synthetic Construct
cucaaggucaaaaacuuacc

SEQ ID NO: 1147 - RNA, 20 nt, Artificial Sequence
  Synthetic Construct
ucaaggucaaaaacuuaccu

SEQ ID NO: 1148 - RNA, 20 nt, Artificial Sequence
  Synthetic Construct
caugucucgaucuaugaaaa

SEQ ID NO: 1149 - RNA, 20 nt, Artificial Sequence
  Synthetic Construct
aguuuuacuuuuaaaauacc

SEQ ID NO: 1150 - RNA, 20 nt, Artificial Sequence
  Synthetic Construct
uacuuuuaaaauaccaugua

SEQ ID NO: 1151 - RNA, 20 nt, Artificial Sequence
  Synthetic Construct
acuuuuaaaauaccauguac

SEQ ID NO: 1152 - RNA, 20 nt, Artificial Sequence
  Synthetic Construct
cuuuuaaaauaccauguacu

SEQ ID NO: 1153 - RNA, 20 nt, Artificial Sequence
  Synthetic Construct
uaaaauaccauguacuaaca

SEQ ID NO: 1154 - RNA, 20 nt, Artificial Sequence
  Synthetic Construct
aaaauaccauguacuaacaa

SEQ ID NO: 1155 - RNA, 20 nt, Artificial Sequence
  Synthetic Construct
aaauaccauguacuaacaaa

SEQ ID NO: 1156 - RNA, 20 nt, Artificial Sequence
  Synthetic Construct
gaaauaaggcuggcagaaua

SEQ ID NO: 1157 - RNA, 20 nt, Artificial Sequence
  Synthetic Construct
cuacccaugaauacauuguu

SEQ ID NO: 1158 - RNA, 20 nt, Artificial Sequence
  Synthetic Construct
uuuagagcuacccagcagga

SEQ ID NO: 1159 - RNA, 20 nt, Artificial Sequence
  Synthetic Construct
agagcuacccagcaggaaca

SEQ ID NO: 1160 - RNA, 20 nt, Artificial Sequence
  Synthetic Construct
gagcuacccagcaggaacaa

SEQ ID NO: 1161 - RNA, 20 nt, Artificial Sequence
  Synthetic Construct
cuacuagccucagauaccaa

SEQ ID NO: 1162 - RNA, 20 nt, Artificial Sequence
  Synthetic Construct
uaggaugcuaggacagcagg

SEQ ID NO: 1163 - RNA, 20 nt, Artificial Sequence
  Synthetic Construct
caaacaaaggccuauaccuu

SEQ ID NO: 1164 - RNA, 20 nt, Artificial Sequence
  Synthetic Construct
uugagauguucguucagugc

SEQ ID NO: 1165 - RNA, 20 nt, Artificial Sequence
  Synthetic Construct
agauguucguucagugcuaa

SEQ ID NO: 1166 - RNA, 20 nt, Artificial Sequence
  Synthetic Construct
guucagugcuaaauauaccu

SEQ ID NO: 1167 - RNA, 20 nt, Artificial Sequence
  Synthetic Construct
agugcuaaauauaccugaag

SEQ ID NO: 1168 - RNA, 20 nt, Artificial Sequence
  Synthetic Construct
agaguguauauguauuugug

SEQ ID NO: 1169 - RNA, 20 nt, Artificial Sequence
  Synthetic Construct
gugcaagugcugcugcugcu

SEQ ID NO: 1170 - RNA, 20 nt, Artificial Sequence
  Synthetic Construct
ugcaagugcugcugcugcuc

SEQ ID NO: 1171 - RNA, 20 nt, Artificial Sequence
  Synthetic Construct
agaaaccaugcugugcauca

SEQ ID NO: 1172 - RNA, 20 nt, Artificial Sequence
  Synthetic Construct
aagaacauucccugacaauc

SEQ ID NO: 1173 - RNA, 20 nt, Artificial Sequence
  Synthetic Construct
agaacauucccugacaaucc

SEQ ID NO: 1174 - RNA, 20 nt, Artificial Sequence
  Synthetic Construct
ccugacaaucccaauaugca

SEQ ID NO: 1175 - RNA, 20 nt, Artificial Sequence
  Synthetic Construct
uuuauaucagaugggauggg

SEQ ID NO: 1176 - RNA, 20 nt, Artificial Sequence
  Synthetic Construct
auaucagaugggaugggacu

SEQ ID NO: 1177 - RNA, 20 nt, Artificial Sequence
  Synthetic Construct
uaucagaugggaugggacuc

SEQ ID NO: 1178 - RNA, 20 nt, Artificial Sequence
  Synthetic Construct
aggguaguauggccauagac

SEQ ID NO: 1179 - RNA, 20 nt, Artificial Sequence
  Synthetic Construct
uuuauaucaaagcagcuuua

SEQ ID NO: 1180 - RNA, 20 nt, Artificial Sequence
  Synthetic Construct
uuauaucaaagcagcuuuau

SEQ ID NO: 1181 - RNA, 20 nt, Artificial Sequence
  Synthetic Construct
uauaucaaagcagcuuuaug

SEQ ID NO: 1182 - RNA, 20 nt, Artificial Sequence
  Synthetic Construct
auaucaaagcagcuuuauga

SEQ ID NO: 1183 - RNA, 20 nt, Artificial Sequence
  Synthetic Construct
uaucaaagcagcuuuaugau

SEQ ID NO: 1184 - RNA, 20 nt, Artificial Sequence
  Synthetic Construct
augauaugacuacucauaca

SEQ ID NO: 1185 - RNA, 20 nt, Artificial Sequence
  Synthetic Construct
ugauaugacuacucauacac

SEQ ID NO: 1186 - RNA, 20 nt, Artificial Sequence
  Synthetic Construct
cagcagcuuacaaaagaaug

SEQ ID NO: 1187 - RNA, 20 nt, Artificial Sequence
  Synthetic Construct
agcagcuuacaaaagaaugu

SEQ ID NO: 1188 - RNA, 20 nt, Artificial Sequence
  Synthetic Construct
ggaguaccugaggaauaucg

SEQ ID NO: 1189 - RNA, 20 nt, Artificial Sequence
  Synthetic Construct
gaguaccugaggaauaucgg

SEQ ID NO: 1190 - RNA, 20 nt, Artificial Sequence
  Synthetic Construct
auauugccaggguauuucac

SEQ ID NO: 1191 - RNA, 20 nt, Artificial Sequence
  Synthetic Construct
ccaggguauuucacuugggg

SEQ ID NO: 1192 - RNA, 20 nt, Artificial Sequence
  Synthetic Construct
cacuuggggcuaacuuggug

SEQ ID NO: 1193 - RNA, 20 nt, Artificial Sequence
  Synthetic Construct
acuuggggcuaacuuggugu

SEQ ID NO: 1194 - RNA, 20 nt, Artificial Sequence
  Synthetic Construct
gggcuaacuuggugucaagc

SEQ ID NO: 1195 - RNA, 20 nt, Artificial Sequence
  Synthetic Construct
gugucaagcuauaucaggca

SEQ ID NO: 1196 - RNA, 20 nt, Artificial Sequence
  Synthetic Construct
acauuuggucaucgauuucu

SEQ ID NO: 1197 - RNA, 20 nt, Artificial Sequence
  Synthetic Construct
cauuuggucaucgauuucuc

SEQ ID NO: 1198 - RNA, 20 nt, Artificial Sequence
  Synthetic Construct
ggucaucgauuucucccaau

SEQ ID NO: 1199 - RNA, 20 nt, Artificial Sequence
  Synthetic Construct
gucaucgauuucucccaauu

SEQ ID NO: 1200 - RNA, 20 nt, Artificial Sequence
  Synthetic Construct
cucccaauuccauuuccacu

SEQ ID NO: 1201 - RNA, 20 nt, Artificial Sequence
  Synthetic Construct
ucccaauuccauuuccacuc

SEQ ID NO: 1202 - RNA, 20 nt, Artificial Sequence
  Synthetic Construct
cauuuccacucuggccaaau

SEQ ID NO: 1203 - RNA, 20 nt, Artificial Sequence
  Synthetic Construct
ccacucuggccaaaugagcu

SEQ ID NO: 1204 - RNA, 20 nt, Artificial Sequence
  Synthetic Construct
cacucuggccaaaugagcuu

SEQ ID NO: 1205 - RNA, 20 nt, Artificial Sequence
  Synthetic Construct
caccuucccaacaagccacc

SEQ ID NO: 1206 - RNA, 20 nt, Artificial Sequence
  Synthetic Construct
ccaacaagccaccuccauuu

SEQ ID NO: 1207 - RNA, 20 nt, Artificial Sequence
  Synthetic Construct
ugaagaauaaaccgugacuu

SEQ ID NO: 1208 - RNA, 20 nt, Artificial Sequence
  Synthetic Construct
gaagaauaaaccgugacuug

SEQ ID NO: 1209 - RNA, 20 nt, Artificial Sequence
  Synthetic Construct
aagaauaaaccgugacuugg

SEQ ID NO: 1210 - RNA, 20 nt, Artificial Sequence
  Synthetic Construct
guaucuuucccucauaauuc

SEQ ID NO: 1211 - RNA, 20 nt, Artificial Sequence
  Synthetic Construct
cccucauaauuccucuauac

SEQ ID NO: 1212 - RNA, 20 nt, Artificial Sequence
  Synthetic Construct
ccucauaauuccucuauaca

SEQ ID NO: 1213 - RNA, 20 nt, Artificial Sequence
  Synthetic Construct
cucuauacaugccuuuuuug

SEQ ID NO: 1214 - RNA, 20 nt, Artificial Sequence
  Synthetic Construct
uuuguuuuuuuucuagcaga

SEQ ID NO: 1215 - RNA, 20 nt, Artificial Sequence
  Synthetic Construct
uuguuuuuuuucuagcagau

SEQ ID NO: 1216 - RNA, 20 nt, Artificial Sequence
  Synthetic Construct
uguuuuuuuucuagcagauu

SEQ ID NO: 1217 - RNA, 20 nt, Artificial Sequence
  Synthetic Construct
guuuuuuuucuagcagauuu

SEQ ID NO: 1218 - RNA, 20 nt, Artificial Sequence
  Synthetic Construct
uuuuuuuucuagcagauuuc

SEQ ID NO: 1219 - DNA, 3809 bp, Artificial Sequence
  Synthetic Construct
gtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagcctaaccagggcttttgcgggagcgcatgccttttggctgtaattcgtgcatttttttttaagaaaaacgcctgccttctgcgtgagattctccagagcaaactgggcggcatgggccctgtggtcttttcgtacagagggcttcctctttggctctttgcctggttgtttccaagatgtactgtgcctcttactttcggttttgaaaacatgagggggttgggcgtggtagcttacgcctgtaatcccagcacttagggaggccgaggcgggaggatggcttgaggtccgtagttgagaccagcctggccaacatggtgaagcctggtctctacaaaaaataataacaaaaattagccgggtgtggtggctcgtgcctgtggtcccagctgctccggtggctgaggcgggaggatctcttgagcttaggcttttgagctatcatggcgccagtgcactccagcgtgggcaacagagcgagaccctgtctctcaaaaaagaaaaaaaaaaaaaaagaaagagaaaagaaaagaaagaaagaagtgaaggtttgtcagtcaggggagctgtaaaaccattaataaagataatccaagatggttaccaagactgttgaggacgccagagatcttgagcactttctaagtacctggcaatacactaagcgcgctcaccttttcctctggcaaaacatgatcgaaagcagaatgttttgatcatgagaaaattgcatttaatttgaatacaatttatttacaacataaaggataatgtatatatcaccaccattactggtatttgctggttatgttagatgtcattttaaaaaataacaatctgatatttaaaaaaaaatcttattttgaaaatttccaaagtaatacatgccatgcatagaccatttctggaagataccacaagaaacatgtaatgatgattgcctctgaaggtctattttcctcctctgacctgtgtgtgggttttgtttttgttttactgtgggcataaattaatttttcagttaagttttggaagcttaaataactctccaaaagtcataaagccagtaactggttgagcccaaattcaaacccagcctgtctgatacttgtcctcttcttagaaaagattacagtgatgctctcacaaaatcttgccgccttccctcaaacagagagttccaggcaggatgaatctgtgctctgatccctgaggcatttaatatgttcttattattagaagctcagatgcaaagagctctcttagcttttaatgttatgaaaaaaatcaggtcttcattagattccccaatccacctcttgatggggctagtagcctttccttaatgatagggtgtttctagagagatatatctggtcaaggtggcctggtactcctccttctccccacagcctcccagacaaggaggagtagctgccttttagtgatcatgtaccctgaatataagtgtatttaaaagaattttatacacatatatttagtgtcaatctgtatatttagtagcactaacacttctcttcattttcaatgaaaaatatagagtttataatattttcttcccacttccccatggatggtctagtcatgcctctcattttggaaagtactgtttctgaaacattaggcaatatattcccaacctggctagtttacagcaatcacctgtggatgctaattaaaacgcaaatcccactgtcacatgcattactccatttgatcataatggaaagtatgttctgtcccatttgccatagtcctcacctatccctgttgtattttatcgggtccaactcaaccatttaaggtatttgccagctcttgtatgcatttaggttttgtttctttgttttttagctcatgaaattaggtacaaagtcagagaggggtctggcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcag

SEQ ID NO: 1220 - DNA, 627 bp, Artificial Sequence
  Synthetic Construct
gtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatag

SEQ ID NO: 1221 - DNA, 1250 bp, Artificial Sequence
  Synthetic Construct
gtaagtttttgaccttgagaaaatgtttttgtttcactgtcctgaggactatttatagacagctctaacatgataaccctcactatgtggagaacattgacagagtaacattttagcagggaaagaagaatcctacagggtcatgttcccttctcctgtggagtggcatgaagaaggtgtatggccccaggtatggccatattactgaccctctacagagagggcaaaggaactgccagtatggtattgcaggataaaggcaggtggttacccacattacctgcaaggctttgatctttcttctgccatttccacattggacatctctgctgaggagagaaaatgaaccactcttttcctttgtataatgttgttttattcttcagacagaagagaggagttatacagctctgcagacatcccattcctgtatggggactgtgtttgcctcttagaggttcccaggccactagaggagataaagggaaacagattgttataacttgatataatgatactataatagatgtaactacaaggagctccagaagcaagagagagggaggaacttggacttctctgcatctttagttggagtccaaaggcttttcaatgaaattctactgcccagggtacattgatgctgaaaccccattcaaatctcctgttatattctagaacagggaattgatttgggagagcatcaggaaggtggatgatctgcccagtcacactgttagtaaattgtagagccaggacctgaactctaatatagtcatgtgttacttaatgacggggacatgttctgagaaatgcttacacaaacctaggtgttgtagcctactacacgcataggctacatggtatagcctattgctcctagactacaaacctgtacagcctgttactgtactgaatactgtgggcagttgtaacacaatggtaagtatttgtgtatctaaacatagaagttgcagtaaaaatatgctattttaatcttatgagaccactgtcatatatacagtccatcattgaccaaaacatcatatcagcattttttcttctaagattttgggagcaccaaagggatacactaacaggatatactctttataatgggtttggagaactgtctgcagctacttcttttaaaaaggtgatctacacagtagaaattagacaagtttggtaatgagatctgcaatccaaataaaataaattcattgctaacctttttcttttcttttcag

SEQ ID NO: 1222 - RNA, 43 nt, Artificial Sequence
  Synthetic Construct
agaaauccgucuuucauugacggaggaaugcccgccagcgcga

SEQ ID NO: 1223 - RNA, 43 nt, Artificial Sequence
  Synthetic Construct
agaaauccgucuuucauugacggcuggccuggaggcuauccag

SEQ ID NO: 1224 - RNA, 43 nt, Artificial Sequence
  Synthetic Construct
agaaauccgucuuucauugacggucccgauauuccucagguac

SEQ ID NO: 1225 - RNA, 43 nt, Artificial Sequence
  Synthetic Construct
agaaauccgucuuucauugacggggaguaccugaggaauaucg

SEQ ID NO: 1226 - RNA, 43 nt, Artificial Sequence
  Synthetic Construct
agaaauccgucuuucauugacggccauucucugcuggaugacg

SEQ ID NO: 1227 - RNA, 43 nt, Artificial Sequence
  Synthetic Construct
agaaauccgucuuucauugacggaguaagucaacuucaauguc

SEQ ID NO: 1228 - RNA, 43 nt, Artificial Sequence
  Synthetic Construct
agaaauccgucuuucauugacgguucaauucucucuccauucu

SEQ ID NO: 1229 - RNA, 43 nt, Artificial Sequence
  Synthetic Construct
agaaauccgucuuucauugacggcagcaaggacuggucuuucu

SEQ ID NO: 1230 - RNA, 43 nt, Artificial Sequence
  Synthetic Construct
agaaauccgucuuucauugacgguucagugggggugaauucag

SEQ ID NO: 1231 - DNA, 20 bp, Artificial Sequence
  Synthetic Construct
aggaatgcccgccagcgcga

SEQ ID NO: 1232 - DNA, 20 bp, Artificial Sequence
  Synthetic Construct
ctggcctggaggctatccag

SEQ ID NO: 1233 - DNA, 20 bp, Artificial Sequence
  Synthetic Construct
tcccgatattcctcaggtac

SEQ ID NO: 1234 - DNA, 20 bp, Artificial Sequence
  Synthetic Construct
ggagtacctgaggaatatcg

SEQ ID NO: 1235 - DNA, 20 bp, Artificial Sequence
  Synthetic Construct
ccattctctgctggatgacg

SEQ ID NO: 1236 - DNA, 20 bp, Artificial Sequence
  Synthetic Construct
aatgtcggatggatgaaacc

SEQ ID NO: 1237 - DNA, 20 bp, Artificial Sequence
  Synthetic Construct
agtaagtcaacttcaatgtc

SEQ ID NO: 1238 - DNA, 20 bp, Artificial Sequence
  Synthetic Construct
ttcaattctctctccattct

SEQ ID NO: 1239 - DNA, 20 bp, Artificial Sequence
  Synthetic Construct
cagcaaggactggtctttct

SEQ ID NO: 1240 - DNA, 20 bp, Artificial Sequence
  Synthetic Construct
ctatctcttgtactacactg

SEQ ID NO: 1241 - DNA, 20 bp, Artificial Sequence
  Synthetic Construct
ttcagtgggggtgaattcag

(19) United States
(12) Patent Application Publication
(10) Pub. No.: US 2023/0399639 A1
(43) Pub. Date: Dec. 14, 2023

(54) COMPOSITIONS COMPRISING AN RNA GUIDE TARGETING B2M AND USES THEREOF

(71) Applicant: Arbor Biotechnologies, Inc., Cambridge, MA (US)

(72) Inventors: Quinton Norman Wessells, Cambridge, MA (US); Jeffrey Raymond Haswell, Needham, MA (US); Tia Marie DiTommaso, Newton, MA (US); Noah Michael Jakimo, Cambridge, MA (US)

(73) Assignee: Arbor Biotechnologies, Inc., Cambridge, MA (US)

(21) Appl. No.: 18/034,609

(22) PCT Filed: Oct. 29, 2021
(86) PCT No.: PCT/US2021/057398

§ 371 Date: Apr. 28, 2023

Related U.S. Application Data
(60) Provisional application No. 63/252,719, filed on Oct. 6, 2021.
(60) Provisional application No. 63/107,869, filed on Oct. 30, 2020.

(51) Int. Cl. C12N 15/11; C12N 9/22; C12N 15/90

(52) U.S. Cl. CPC C12N 15/11; C12N 9/22; C12N 15/907; C12N 15/111; C12N 2310/20

(57) ABSTRACT

The present invention relates to compositions comprising RNA guides targeting B2M, processes for characterizing the compositions, cells comprising the compositions, and methods of using the compositions.

### SEQUENCE LISTING

The instant application contains a Sequence Listing which has been submitted electronically in ASCII format and is hereby incorporated by reference in its entirety. Said ASCII copy, created on Oct. 29, 2021, is named 51451-014WO3_Sequence_Listing_10_29_21_ST25, and is 369,037 bytes in size.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY OF THE INVENTION

It is against the above background that the present invention provides certain advantages and advancements over the prior art. Although this invention disclosed herein is not limited to specific advantages or functionalities, the invention provides a composition comprising an RNA guide, wherein the RNA guide comprises (i) a spacer sequence that is substantially complementary to a target sequence within a B2M gene and (ii) a direct repeat sequence; wherein the target sequence is adjacent to a protospacer adjacent motif (PAM) comprising the sequence 5′-NTTN-3′.

In one aspect of the composition, the target sequence is within exon 1, exon 2, exon 3, exon 4, intron 1, intron 2, or intron 3 of the B2M gene.

In another aspect of the composition, the B2M gene comprises the sequence of SEQ ID NO: 773, the reverse complement of SEQ ID NO: 773, a variant of SEQ ID NO: 773, or the reverse complement of a variant of SEQ ID NO: 773.

In another aspect of the composition, the spacer sequence comprises: a. nucleotide 1 through nucleotide 16 of a sequence that is at least 90% identical to a sequence of any one of SEQ ID NOs: 391-770 or 1019-1218; b. nucleotide 1 through nucleotide 17 of a sequence that is at least 90% identical to a sequence of any one of SEQ ID NOs: 391-770 or 1019-1218; c. nucleotide 1 through nucleotide 18 of a sequence that is at least 90% identical to a sequence of any one of SEQ ID NOs: 391-770 or 1019-1218; d. nucleotide 1 through nucleotide 19 of a sequence that is at least 90% identical to a sequence of any one of SEQ ID NOs: 391-770 or 1019-1218; e. nucleotide 1 through nucleotide 20 of a sequence that is at least 90% identical to a sequence of any one of SEQ ID NOs: 391-770 or 1019-1218; f. nucleotide 1 through nucleotide 21 of a sequence that is at least 90% identical to a sequence of any one of SEQ ID NOs: 391-770; g. nucleotide 1 through nucleotide 22 of a sequence that is at least 90% identical to a sequence of any one of SEQ ID NOs: 391-552 and 554-770; h. nucleotide 1 through nucleotide 23 of a sequence that is at least 90% identical to a sequence of any one of SEQ ID NOs: 391-552 and 555-770; i. nucleotide 1 through nucleotide 24 of a sequence that is at least 90% identical to a sequence of any one of SEQ ID NOs: 391-552 and 556-770; j. nucleotide 1 through nucleotide 25 of a sequence that is at least 90% identical to a sequence of any one of SEQ ID NOs: 391-552 and 556-770; k. nucleotide 1 through nucleotide 26 of a sequence that is at least 90% identical to a sequence of any one of SEQ ID NOs: 391-552 and 556-770; 1. nucleotide 1 through nucleotide 27 of a sequence that is at least 90% identical to a sequence of any one of SEQ ID NOs: 391-552 and 556-770; m. nucleotide 1 through nucleotide 28 of a sequence that is at least 90% identical to a sequence of any one of SEQ ID NOs: 391-552 and 556-770; n. nucleotide 1 through nucleotide 29 of a sequence that is at least 90% identical to a sequence of any one of SEQ ID NOs: 391-552 and 556-770; or o. nucleotide 1 through nucleotide 30 of a sequence that is at least 90% identical to a sequence of any one of SEQ ID NOs: 391-540, 542-552, and 556-770.

In another aspect of the composition, the spacer sequence comprises: a. nucleotide 1 through nucleotide 16 of any one of SEQ ID NOs: 391-770 or 1019-1218; b. nucleotide 1 through nucleotide 17 of any one of SEQ ID NOs: 391-770 or 1019-1218; c. nucleotide 1 through nucleotide 18 of any one of SEQ ID NOs: 391-770 or 1019-1218; d. nucleotide 1 through nucleotide 19 of any one of SEQ ID NOs: 391-770 or 1019-1218; e. nucleotide 1 through nucleotide 20 of any one of SEQ ID NOs: 391-770 or 1019-1218; f. nucleotide 1 through nucleotide 21 of any one of SEQ ID NOs: 391-770; g. nucleotide 1 through nucleotide 22 of any one of SEQ ID NOs: 391-552 and 554-770; h. nucleotide 1 through nucleotide 23 of any one of SEQ ID NOs: 391-552 and 555-770; i. nucleotide 1 through nucleotide 24 of any one of SEQ ID NOs: 391-552 and 556-770; j. nucleotide 1 through nucleotide 25 of any one of SEQ ID NOs: 391-552 and 556-770; k. nucleotide 1 through nucleotide 26 of any one of SEQ ID NOs: 391-552 and 556-770; 1. nucleotide 1 through nucleotide 27 of any one of SEQ ID NOs: 391-552 and 556-770; m. nucleotide 1 through nucleotide 28 of any one of SEQ ID NOs: 391-552 and 556-770; n. nucleotide 1 through nucleotide 29 of any one of SEQ ID NOs: 391-552 and 556-770; or o. nucleotide 1 through nucleotide 30 of any one of SEQ ID NOs: 391-540, 542-552, and 556-770.

In another aspect of the composition, the direct repeat comprises: a. nucleotide 1 through nucleotide 36 of a sequence that is at least 90% identical to a sequence of any one of SEQ ID NOs: 1-8; b. nucleotide 2 through nucleotide 36 of a sequence that is at least 90% identical to a sequence of any one of SEQ ID NOs: 1-8; c. nucleotide 3 through nucleotide 36 of a sequence that is at least 90% identical to a sequence of any one of SEQ ID NOs: 1-8; d. nucleotide 4 through nucleotide 36 of a sequence that is at least 90% identical to a sequence of any one of SEQ ID NOs: 1-8; e. nucleotide 5 through nucleotide 36 of a sequence that is at least 90% identical to a sequence of any one of SEQ ID NOs: 1-8; f. nucleotide 6 through nucleotide 36 of a sequence that is at least 90% identical to a sequence of any one of SEQ ID NOs: 1-8; g. nucleotide 7 through nucleotide 36 of a sequence that is at least 90% identical to a sequence of any one of SEQ ID NOs: 1-8; h. nucleotide 8 through nucleotide 36 of a sequence that is at least 90% identical to a sequence of any one of SEQ ID NOs: 1-8; i. nucleotide 9 through nucleotide 36 of a sequence that is at least 90% identical to a sequence of any one of SEQ ID NOs: 1-8; j. nucleotide 10 through nucleotide 36 of a sequence that is at least 90% identical to a sequence of any one of SEQ ID NOs: 1-8; k. nucleotide 11 through nucleotide 36 of a sequence that is at least 90% identical to a sequence of any one of SEQ ID NOs: 1-8; 1. nucleotide 12 through nucleotide 36 of a sequence that is at least 90% identical to a sequence of any one of SEQ ID NOs: 1-8; m. nucleotide 13 through nucleotide 36 of a sequence that is at least 90% identical to a sequence of any one of SEQ ID NOs: 1-8; n. nucleotide 14 through nucleotide 36 of a sequence that is at least 90% identical to a sequence of any one of SEQ ID NOs: 1-8; o. nucleotide 1 through nucleotide 34 of a sequence that is at least 90% identical to a sequence of SEQ ID NO: 9; p. nucleotide 2 through nucleotide 34 of a sequence that is at least 90% identical to a sequence of SEQ ID NO: 9; q. nucleotide 3 through nucleotide 34 of a sequence that is at least 90% identical to a sequence of SEQ ID NO: 9; r. nucleotide 4 through nucleotide 34 of a sequence that is at least 90% identical to a sequence of SEQ ID NO: 9; s. nucleotide 5 through nucleotide 34 of a sequence that is at least 90% identical to a sequence of SEQ ID NO: 9; t. nucleotide 6 through nucleotide 34 of a sequence that is at least 90% identical to a sequence of SEQ ID NO: 9; u. nucleotide 7 through nucleotide 34 of a sequence that is at least 90% identical to a sequence of SEQ ID NO: 9; v. nucleotide 8 through nucleotide 34 of a sequence that is at least 90% identical to a sequence of SEQ ID NO: 9; w. nucleotide 9 through nucleotide 34 of a sequence that is at least 90% identical to a sequence of SEQ ID NO: 9; x. nucleotide 10 through nucleotide 34 of a sequence that is at least 90% identical to a sequence of SEQ ID NO: 9; y. nucleotide 11 through nucleotide 34 of a sequence that is at least 90% identical to a sequence of SEQ ID NO: 9; z. nucleotide 12 through nucleotide 34 of a sequence that is at least 90% identical to a sequence of SEQ ID NO: 9; or aa. a sequence that is at least 90% identical to a sequence of SEQ ID NO: 10 or a portion thereof.

In another aspect of the composition, the direct repeat comprises: a. nucleotide 1 through nucleotide 36 of any one of SEQ ID NOs: 1-8; b. nucleotide 2 through nucleotide 36 of any one of SEQ ID NOs: 1-8; c. nucleotide 3 through nucleotide 36 of any one of SEQ ID NOs: 1-8; d. nucleotide 4 through nucleotide 36 of any one of SEQ ID NOs: 1-8; e. nucleotide 5 through nucleotide 36 of any one of SEQ ID NOs: 1-8; f. nucleotide 6 through nucleotide 36 of any one of SEQ ID NOs: 1-8; g. nucleotide 7 through nucleotide 36 of any one of SEQ ID NOs: 1-8; h. nucleotide 8 through nucleotide 36 of any one of SEQ ID NOs: 1-8; i. nucleotide 9 through nucleotide 36 of any one of SEQ ID NOs: 1-8; j. nucleotide 10 through nucleotide 36 of any one of SEQ ID NOs: 1-8; k. nucleotide 11 through nucleotide 36 of any one of SEQ ID NOs: 1-8; 1. nucleotide 12 through nucleotide 36 of any one of SEQ ID NOs: 1-8; m. nucleotide 13 through nucleotide 36 of any one of SEQ ID NOs: 1-8; n. nucleotide 14 through nucleotide 36 of any one of SEQ ID NOs: 1-8; o. nucleotide 1 through nucleotide 34 of SEQ ID NO: 9; p. nucleotide 2 through nucleotide 34 of SEQ ID NO: 9; q. nucleotide 3 through nucleotide 34 of SEQ ID NO: 9; r. nucleotide 4 through nucleotide 34 of SEQ ID NO: 9; s. nucleotide 5 through nucleotide 34 of SEQ ID NO: 9; t. nucleotide 6 through nucleotide 34 of SEQ ID NO: 9; u. nucleotide 7 through nucleotide 34 of SEQ ID NO: 9; v. nucleotide 8 through nucleotide 34 of SEQ ID NO: 9; w. nucleotide 9 through nucleotide 34 of SEQ ID NO: 9; x. nucleotide 10 through nucleotide 34 of SEQ ID NO: 9; y. nucleotide 11 through nucleotide 34 of SEQ ID NO: 9; z. nucleotide 12 through nucleotide 34 of SEQ ID NO: 9; or aa. SEQ ID NO: 10 or a portion thereof.

In another aspect of the composition, the direct repeat comprises: a. nucleotide 1 through nucleotide 36 of a sequence that is at least 90% identical to a sequence of any one of SEQ ID NOs: 788-805; b. nucleotide 2 through nucleotide 36 of a sequence that is at least 90% identical to a sequence of any one of SEQ ID NOs: 788-805; c. nucleotide 3 through nucleotide 36 of a sequence that is at least 90% identical to a sequence of any one of SEQ ID NOs: 788-805; d. nucleotide 4 through nucleotide 36 of a sequence that is at least 90% identical to a sequence of any one of SEQ ID NOs: 788-805; e. nucleotide 5 through nucleotide 36 of a sequence that is at least 90% identical to a sequence of any one of SEQ ID NOs: 788-805; f. nucleotide 6 through nucleotide 36 of a sequence that is at least 90% identical to a sequence of any one of SEQ ID NOs: 788-805; g. nucleotide 7 through nucleotide 36 of a sequence that is at least 90% identical to a sequence of any one of SEQ ID NOs: 788-805; h. nucleotide 8 through nucleotide 36 of a sequence that is at least 90% identical to a sequence of any one of SEQ ID NOs: 788-805; i. nucleotide 9 through nucleotide 36 of a sequence that is at least 90% identical to a sequence of any one of SEQ ID NOs: 788-805; j. nucleotide 10 through nucleotide 36 of a sequence that is at least 90% identical to a sequence of any one of SEQ ID NOs: 788-805; k. nucleotide 11 through nucleotide 36 of a sequence that is at least 90% identical to a sequence of any one of SEQ ID NOs: 788-805; 1. nucleotide 12 through nucleotide 36 of a sequence that is at least 90% identical to a sequence of any one of SEQ ID NOs: 788-805; m. nucleotide 13 through nucleotide 36 of a sequence that is at least 90% identical to a sequence of any one of SEQ ID NOs: 788-805; n. nucleotide 14 through nucleotide 36 of a sequence that is at least 90% identical to a sequence of any one of SEQ ID NOs: 788-805; or o. a sequence that is at least 90% identical to a sequence of SEQ ID NO: 806 or a portion thereof.

In another aspect of the composition, the direct repeat comprises: a. nucleotide 1 through nucleotide 36 of any one of SEQ ID NOs: 788-805; b. nucleotide 2 through nucleotide 36 of any one of SEQ ID NOs: 788-805; c. nucleotide 3 through nucleotide 36 of any one of SEQ ID NOs: 788-805; d. nucleotide 4 through nucleotide 36 of any one of SEQ ID NOs: 788-805; e. nucleotide 5 through nucleotide 36 of any one of SEQ ID NOs: 788-805; f. nucleotide 6 through nucleotide 36 of any one of SEQ ID NOs: 788-805; g. nucleotide 7 through nucleotide 36 of any one of SEQ ID NOs: 788-805; h. nucleotide 8 through nucleotide 36 of any one of SEQ ID NOs: 788-805; i. nucleotide 9 through nucleotide 36 of any one of SEQ ID NOs: 788-805; j. nucleotide 10 through nucleotide 36 of any one of SEQ ID NOs: 788-805; k. nucleotide 11 through nucleotide 36 of any one of SEQ ID NOs: 788-805; 1. nucleotide 12 through nucleotide 36 of any one of SEQ ID NOs: 788-805; m. nucleotide 13 through nucleotide 36 of any one of SEQ ID NOs: 788-805; n. nucleotide 14 through nucleotide 36 of any one of SEQ ID NOs: 788-805; or o. SEQ ID NO: 806 or a portion thereof.

In another aspect of the composition, the direct repeat comprises: a. nucleotide 1 through nucleotide 36 of a sequence that is at least 90% identical to SEQ ID NO: 807; b. nucleotide 2 through nucleotide 36 of a sequence that is at least 90% identical to SEQ ID NO: 807; c. nucleotide 3 through nucleotide 36 of a sequence that is at least 90% identical to SEQ ID NO: 807; d. nucleotide 4 through nucleotide 36 of a sequence that is at least 90% identical to SEQ ID NO: 807; e. nucleotide 5 through nucleotide 36 of a sequence that is at least 90% identical to SEQ ID NO: 807; f. nucleotide 6 through nucleotide 36 of a sequence that is at least 90% identical to SEQ ID NO: 807; g. nucleotide 7 through nucleotide 36 of a sequence that is at least 90% identical to SEQ ID NO: 807; h. nucleotide 8 through nucleotide 36 of a sequence that is at least 90% identical to SEQ ID NO: 807; i. nucleotide 9 through nucleotide 36 of a sequence that is at least 90% identical to SEQ ID NO: 807; j. nucleotide 10 through nucleotide 36 of a sequence that is at least 90% identical to SEQ ID NO: 807; k. nucleotide 11 through nucleotide 36 of a sequence that is at least 90% identical to SEQ ID NO: 807; 1. nucleotide 12 through nucleotide 36 of a sequence that is at least 90% identical to SEQ ID NO: 807; m. nucleotide 13 through nucleotide 36 of a sequence that is at least 90% identical to SEQ ID NO: 807; n. nucleotide 14 through nucleotide 36 of a sequence that is at least 90% identical to SEQ ID NO: 807; or o. a sequence that is at least 90% identical to a sequence of SEQ ID NO: 808 or SEQ ID NO: 809 or a portion thereof.

In another aspect of the composition, the direct repeat comprises: a. nucleotide 1 through nucleotide 36 of SEQ ID NO: 807; b. nucleotide 2 through nucleotide 36 of SEQ ID NO: 807; c. nucleotide 3 through nucleotide 36 of SEQ ID NO: 807; d. nucleotide 4 through nucleotide 36 of SEQ ID NO: 807; e. nucleotide 5 through nucleotide 36 of SEQ ID NO: 807; f. nucleotide 6 through nucleotide 36 of SEQ ID NO: 807; g. nucleotide 7 through nucleotide 36 of SEQ ID NO: 807; h. nucleotide 8 through nucleotide 36 of SEQ ID NO: 807; i. nucleotide 9 through nucleotide 36 of SEQ ID NO: 807; j. nucleotide 10 through nucleotide 36 of SEQ ID NO: 807; k. nucleotide 11 through nucleotide 36 of SEQ ID NO: 807; 1. nucleotide 12 through nucleotide 36 of SEQ ID NO: 807; m. nucleotide 13 through nucleotide 36 of SEQ ID NO: 807; n. nucleotide 14 through nucleotide 36 of SEQ ID NO: 807; or o. SEQ ID NO: 808 or SEQ ID NO: 809 or a portion thereof.

In another aspect of the composition, the direct repeat comprises: a. nucleotide 1 through nucleotide 36 of a sequence that is at least 90% identical to a sequence of SEQ ID NO: 810 or SEQ ID NO: 811; b. nucleotide 2 through nucleotide 36 of a sequence that is at least 90% identical to a sequence of SEQ ID NO: 810 or SEQ ID NO: 811; c. nucleotide 3 through nucleotide 36 of a sequence that is at least 90% identical to a sequence of SEQ ID NO: 810 or SEQ ID NO: 811; d. nucleotide 4 through nucleotide 36 of a sequence that is at least 90% identical to a sequence of SEQ ID NO: 810 or SEQ ID NO: 811; e. nucleotide 5 through nucleotide 36 of a sequence that is at least 90% identical to a sequence of SEQ ID NO: 810 or SEQ ID NO: 811; f. nucleotide 6 through nucleotide 36 of a sequence that is at least 90% identical to a sequence of SEQ ID NO: 810 or SEQ ID NO: 811; g. nucleotide 7 through nucleotide 36 of a sequence that is at least 90% identical to a sequence of SEQ ID NO: 810 or SEQ ID NO: 811; h. nucleotide 8 through nucleotide 36 of a sequence that is at least 90% identical to a sequence of SEQ ID NO: 810 or SEQ ID NO: 811; i. nucleotide 9 through nucleotide 36 of a sequence that is at least 90% identical to a sequence of SEQ ID NO: 810 or SEQ ID NO: 811; j. nucleotide 10 through nucleotide 36 of a sequence that is at least 90% identical to a sequence of SEQ ID NO: 810 or SEQ ID NO: 811; k. nucleotide 11 through nucleotide 36 of a sequence that is at least 90% identical to a sequence of SEQ ID NO: 810 or SEQ ID NO: 811; 1. nucleotide 12 through nucleotide 36 of a sequence that is at least 90% identical to a sequence of SEQ ID NO: 810 or SEQ ID NO: 811; m. nucleotide 13 through nucleotide 36 of a sequence that is at least 90% identical to a sequence of SEQ ID NO: 810 or SEQ ID NO: 811; n. nucleotide 14 through nucleotide 36 of a sequence that is at least 90% identical to a sequence of SEQ ID NO: 810 or SEQ ID NO: 811; o. nucleotide 15 through nucleotide 36 of a sequence that is at least 90% identical to a sequence of SEQ ID NO: 810 or SEQ ID NO: 811; or p. a sequence that is at least 90% identical to a sequence of SEQ ID NO: 812 or a portion thereof.

In another aspect of the composition, the direct repeat comprises: a. nucleotide 1 through nucleotide 36 of SEQ ID NO: 810 or SEQ ID NO: 811; b. nucleotide 2 through nucleotide 36 of SEQ ID NO: 810 or SEQ ID NO: 811; c. nucleotide 3 through nucleotide 36 of SEQ ID NO: 810 or SEQ ID NO: 811; d. nucleotide 4 through nucleotide 36 of SEQ ID NO: 810 or SEQ ID NO: 811; e. nucleotide 5 through nucleotide 36 of SEQ ID NO: 810 or SEQ ID NO: 811; f. nucleotide 6 through nucleotide 36 of SEQ ID NO: 810 or SEQ ID NO: 811; g. nucleotide 7 through nucleotide 36 of SEQ ID NO: 810 or SEQ ID NO: 811; h. nucleotide 8 through nucleotide 36 of SEQ ID NO: 810 or SEQ ID NO: 811; i. nucleotide 9 through nucleotide 36 of SEQ ID NO: 810 or SEQ ID NO: 811; j. nucleotide 10 through nucleotide 36 of SEQ ID NO: 810 or SEQ ID NO: 811; k. nucleotide 11 through nucleotide 36 of SEQ ID NO: 810 or SEQ ID NO: 811; 1. nucleotide 12 through nucleotide 36 of SEQ ID NO: 810 or SEQ ID NO: 811; m. nucleotide 13 through nucleotide 36 of SEQ ID NO: 810 or SEQ ID NO: 811; n. nucleotide 14 through nucleotide 36 of SEQ ID NO: 810 or SEQ ID NO: 811; o. nucleotide 15 through nucleotide 36 of SEQ ID NO: 810 or SEQ ID NO: 811; or p. SEQ ID NO: 812 or a portion thereof.

In another aspect of the composition, the spacer sequence is substantially complementary to the complement of a sequence of any one of SEQ ID NOs: 11-390 or 819-1018.

In another aspect of the composition, the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In another aspect of the composition, the target sequence is immediately adjacent to the PAM sequence.

In another aspect of the composition, the composition further comprises a Cas12i polypeptide.

In another aspect of the composition, the Cas12i polypeptide is: a. a Cas12i2 polypeptide comprising a sequence that is at least 90% identical to the sequence of SEQ ID NO: 772, SEQ ID NO: 782, SEQ ID NO: 783, SEQ ID NO: 784, SEQ ID NO: 785, or SEQ ID NO: 786; b. a Cas12i4 polypeptide comprising a sequence that is at least 90% identical to the sequence of SEQ ID NO: 814, SEQ ID NO: 815, or SEQ ID NO: 816; c. a Cas12i1 polypeptide comprising a sequence that is at least 90% identical to the sequence of SEQ ID NO: 817; or d. a Cas12i3 polypeptide comprising a sequence that is at least 90% identical to the sequence of SEQ ID NO: 818.

In another aspect of the composition, the Cas12i polypeptide is: a. a Cas12i2 polypeptide comprising a sequence of SEQ ID NO: 772, SEQ ID NO: 782, SEQ ID NO: 783, SEQ ID NO: 784, SEQ ID NO: 785, or SEQ ID NO: 786; b. a Cas12i4 polypeptide comprising a sequence of SEQ ID NO: 814, SEQ ID NO: 815, or SEQ ID NO: 816; c. a Cas12i1 polypeptide comprising a sequence of SEQ ID NO: 817; or d. a Cas12i3 polypeptide comprising a sequence of SEQ ID NO: 818.

In another aspect of the composition, the RNA guide and the Cas12i polypeptide form a ribonucleoprotein complex.

In another aspect of the composition, the ribonucleoprotein complex binds a target nucleic acid.

In another aspect of the composition, the composition is present within a cell.

In another aspect of the composition, the RNA guide and the Cas12i polypeptide are encoded in a vector, e.g., expression vector. In another aspect of the composition, the RNA guide and the Cas12i polypeptide are encoded in a single vector or the RNA guide is encoded in a first vector and the Cas12i polypeptide is encoded in a second vector.

The invention further provides a vector system comprising one or more vectors encoding an RNA guide disclosed herein and a Cas12i polypeptide. In an embodiment, the vector system comprises a first vector encoding an RNA guide disclosed herein and a second vector encoding a Cas12i polypeptide. The vectors may be expression vectors.

The invention further provides a composition comprising an RNA guide and a Cas12i polypeptide, wherein the RNA guide comprises (i) a spacer sequence that is substantially complementary to a target sequence within a B2M gene and (ii) a direct repeat sequence.

In one aspect of the composition, the target sequence is within exon 1, exon 2, exon 3, exon 4, intron 1, intron 2, or intron 3 of the B2M gene.

In another aspect of the composition, the B2M gene comprises the sequence of SEQ ID NO: 773, the reverse complement of SEQ ID NO: 773, a variant of the sequence of SEQ ID NO: 773, or the reverse complement of a variant of SEQ ID NO: 773.

In another aspect of the composition, the spacer sequence comprises: a. nucleotide 1 through nucleotide 16 of a sequence that is at least 90% identical to a sequence of any one of SEQ ID NOs: 391-770 or 1019-1218; b. nucleotide 1 through nucleotide 17 of a sequence that is at least 90% identical to a sequence of any one of SEQ ID NOs: 391-770 or 1019-1218; c. nucleotide 1 through nucleotide 18 of a sequence that is at least 90% identical to a sequence of any one of SEQ ID NOs: 391-770 or 1019-1218; d. nucleotide 1 through nucleotide 19 of a sequence that is at least 90% identical to a sequence of any one of SEQ ID NOs: 391-770 or 1019-1218; e. nucleotide 1 through nucleotide 20 of a sequence that is at least 90% identical to a sequence of any one of SEQ ID NOs: 391-770 or 1019-1218; f. nucleotide 1 through nucleotide 21 of a sequence that is at least 90% identical to a sequence of any one of SEQ ID NOs: 391-770; g. nucleotide 1 through nucleotide 22 of a sequence that is at least 90% identical to a sequence of any one of SEQ ID NOs: 391-552 and 554-770; h. nucleotide 1 through nucleotide 23 of a sequence that is at least 90% identical to a sequence of any one of SEQ ID NOs: 391-552 and 555-770; i. nucleotide 1 through nucleotide 24 of a sequence that is at least 90% identical to a sequence of any one of SEQ ID NOs: 391-552 and 556-770; j. nucleotide 1 through nucleotide 25 of a sequence that is at least 90% identical to a sequence of any one of SEQ ID NOs: 391-552 and 556-770; k. nucleotide 1 through nucleotide 26 of a sequence that is at least 90% identical to a sequence of any one of SEQ ID NOs: 391-552 and 556-770; 1. nucleotide 1 through nucleotide 27 of a sequence that is at least 90% identical to a sequence of any one of SEQ ID NOs: 391-552 and 556-770; m. nucleotide 1 through nucleotide 28 of a sequence that is at least 90% identical to a sequence of any one of SEQ ID NOs: 391-552 and 556-770; n. nucleotide 1 through nucleotide 29 of a sequence that is at least 90% identical to a sequence of any one of SEQ ID NOs: 391-552 and 556-770; or o. nucleotide 1 through nucleotide 30 of a sequence that is at least 90% identical to a sequence of any one of SEQ ID NOs: 391-540, 542-552, and 556-770.

In another aspect of the composition, the spacer sequence comprises: a. nucleotide 1 through nucleotide 16 of any one of SEQ ID NOs: 391-770 or 1019-1218; b. nucleotide 1 through nucleotide 17 of any one of SEQ ID NOs: 391-770 or 1019-1218; c. nucleotide 1 through nucleotide 18 of any one of SEQ ID NOs: 391-770 or 1019-1218; d. nucleotide 1 through nucleotide 19 of any one of SEQ ID NOs: 391-770 or 1019-1218; e. nucleotide 1 through nucleotide 20 of any one of SEQ ID NOs: 391-770 or 1019-1218; f. nucleotide 1 through nucleotide 21 of any one of SEQ ID NOs: 391-770; g. nucleotide 1 through nucleotide 22 of any one of SEQ ID NOs: 391-552 and 554-770; h. nucleotide 1 through nucleotide 23 of any one of SEQ ID NOs: 391-552 and 555-770; i. nucleotide 1 through nucleotide 24 of any one of SEQ ID NOs: 391-552 and 556-770; j. nucleotide 1 through nucleotide 25 of any one of SEQ ID NOs: 391-552 and 556-770; k. nucleotide 1 through nucleotide 26 of any one of SEQ ID NOs: 391-552 and 556-770; 1. nucleotide 1 through nucleotide 27 of any one of SEQ ID NOs: 391-552 and 556-770; m. nucleotide 1 through nucleotide 28 of any one of SEQ ID NOs: 391-552 and 556-770; n. nucleotide 1 through nucleotide 29 of any one of SEQ ID NOs: 391-552 and 556-770; or o. nucleotide 1 through nucleotide 30 of any one of SEQ ID NOs: 391-540, 542-552, and 556-770.

In another aspect of the composition, the direct repeat comprises: a. nucleotide 1 through nucleotide 36 of a sequence that is at least 90% identical to a sequence of any one of SEQ ID NOs: 1-8; b. nucleotide 2 through nucleotide 36 of a sequence that is at least 90% identical to a sequence of any one of SEQ ID NOs: 1-8; c. nucleotide 3 through nucleotide 36 of a sequence that is at least 90% identical to a sequence of any one of SEQ ID NOs: 1-8; d. nucleotide 4 through nucleotide 36 of a sequence that is at least 90% identical to a sequence of any one of SEQ ID NOs: 1-8; e. nucleotide 5 through nucleotide 36 of a sequence that is at least 90% identical to a sequence of any one of SEQ ID NOs: 1-8; f. nucleotide 6 through nucleotide 36 of a sequence that is at least 90% identical to a sequence of any one of SEQ ID NOs: 1-8; g. nucleotide 7 through nucleotide 36 of a sequence that is at least 90% identical to a sequence of any one of SEQ ID NOs: 1-8; h. nucleotide 8 through nucleotide 36 of a sequence that is at least 90% identical to a sequence of any one of SEQ ID NOs: 1-8; i. nucleotide 9 through nucleotide 36 of a sequence that is at least 90% identical to a sequence of any one of SEQ ID NOs: 1-8; j. nucleotide 10 through nucleotide 36 of a sequence that is at least 90% identical to a sequence of any one of SEQ ID NOs: 1-8; k. nucleotide 11 through nucleotide 36 of a sequence that is at least 90% identical to a sequence of any one of SEQ ID NOs: 1-8; 1. nucleotide 12 through nucleotide 36 of a sequence that is at least 90% identical to a sequence of any one of SEQ ID NOs: 1-8; m. nucleotide 13 through nucleotide 36 of a sequence that is at least 90% identical to a sequence of any one of SEQ ID NOs: 1-8; n. nucleotide 14 through nucleotide 36 of a sequence that is at least 90% identical to a sequence of any one of SEQ ID NOs: 1-8; o. nucleotide 1 through nucleotide 34 of a sequence that is at least 90% identical to a sequence of SEQ ID NO: 9; p. nucleotide 2 through nucleotide 34 of a sequence that is at least 90% identical to a sequence of SEQ ID NO: 9; q. nucleotide 3 through nucleotide 34 of a sequence that is at least 90% identical to a sequence of SEQ ID NO: 9; r. nucleotide 4 through nucleotide 34 of a sequence that is at least 90% identical to a sequence of SEQ ID NO: 9; s. nucleotide 5 through nucleotide 34 of a sequence that is at least 90% identical to a sequence of SEQ ID NO: 9; t. nucleotide 6 through nucleotide 34 of a sequence that is at least 90% identical to a sequence of SEQ ID NO: 9; u. nucleotide 7 through nucleotide 34 of a sequence that is at least 90% identical to a sequence of SEQ ID NO: 9; v. nucleotide 8 through nucleotide 34 of a sequence that is at least 90% identical to a sequence of SEQ ID NO: 9; w. nucleotide 9 through nucleotide 34 of a sequence that is at least 90% identical to a sequence of SEQ ID NO: 9; x. nucleotide 10 through nucleotide 34 of a sequence that is at least 90% identical to a sequence of SEQ ID NO: 9; y. nucleotide 11 through nucleotide 34 of a sequence that is at least 90% identical to a sequence of SEQ ID NO: 9; z. nucleotide 12 through nucleotide 34 of a sequence that is at least 90% identical to a sequence of SEQ ID NO: 9; or aa. a sequence that is at least 90% identical to a sequence of SEQ ID NO: 10 or a portion thereof.

In another aspect of the composition, the direct repeat comprises: a. nucleotide 1 through nucleotide 36 of any one of SEQ ID NOs: 1-8; b. nucleotide 2 through nucleotide 36 of any one of SEQ ID NOs: 1-8; c. nucleotide 3 through nucleotide 36 of any one of SEQ ID NOs: 1-8; d. nucleotide 4 through nucleotide 36 of any one of SEQ ID NOs: 1-8; e. nucleotide 5 through nucleotide 36 of any one of SEQ ID NOs: 1-8; f. nucleotide 6 through nucleotide 36 of any one of SEQ ID NOs: 1-8; g. nucleotide 7 through nucleotide 36 of any one of SEQ ID NOs: 1-8; h. nucleotide 8 through nucleotide 36 of any one of SEQ ID NOs: 1-8; i. nucleotide 9 through nucleotide 36 of any one of SEQ ID NOs: 1-8; j. nucleotide 10 through nucleotide 36 of any one of SEQ ID NOs: 1-8; k. nucleotide 11 through nucleotide 36 of any one of SEQ ID NOs: 1-8; 1. nucleotide 12 through nucleotide 36 of any one of SEQ ID NOs: 1-8; m. nucleotide 13 through nucleotide 36 of any one of SEQ ID NOs: 1-8; n. nucleotide 14 through nucleotide 36 of any one of SEQ ID NOs: 1-8; o. nucleotide 1 through nucleotide 34 of SEQ ID NO: 9; p. nucleotide 2 through nucleotide 34 of SEQ ID NO: 9; q. nucleotide 3 through nucleotide 34 of SEQ ID NO: 9; r. nucleotide 4 through nucleotide 34 of SEQ ID NO: 9; s. nucleotide 5 through nucleotide 34 of SEQ ID NO: 9; t. nucleotide 6 through nucleotide 34 of SEQ ID NO: 9; u. nucleotide 7 through nucleotide 34 of SEQ ID NO: 9; v. nucleotide 8 through nucleotide 34 of SEQ ID NO: 9; w. nucleotide 9 through nucleotide 34 of SEQ ID NO: 9; x. nucleotide 10 through nucleotide 34 of SEQ ID NO: 9; y. nucleotide 11 through nucleotide 34 of SEQ ID NO: 9; z. nucleotide 12 through nucleotide 34 of SEQ ID NO: 9; or aa. SEQ ID NO: 10 or a portion thereof.

In another aspect of the composition, the direct repeat comprises: a. nucleotide 1 through nucleotide 36 of a sequence that is at least 90% identical to a sequence of any one of SEQ ID NOs: 788-805; b. nucleotide 2 through nucleotide 36 of a sequence that is at least 90% identical to a sequence of any one of SEQ ID NOs: 788-805; c. nucleotide 3 through nucleotide 36 of a sequence that is at least 90% identical to a sequence of any one of SEQ ID NOs: 788-805; d. nucleotide 4 through nucleotide 36 of a sequence that is at least 90% identical to a sequence of any one of SEQ ID NOs: 788-805; e. nucleotide 5 through nucleotide 36 of a sequence that is at least 90% identical to a sequence of any one of SEQ ID NOs: 788-805; f. nucleotide 6 through nucleotide 36 of a sequence that is at least 90% identical to a sequence of any one of SEQ ID NOs: 788-805; g. nucleotide 7 through nucleotide 36 of a sequence that is at least 90% identical to a sequence of any one of SEQ ID NOs: 788-805; h. nucleotide 8 through nucleotide 36 of a sequence that is at least 90% identical to a sequence of any one of SEQ ID NOs: 788-805; i. nucleotide 9 through nucleotide 36 of a sequence that is at least 90% identical to a sequence of any one of SEQ ID NOs: 788-805; j. nucleotide 10 through nucleotide 36 of a sequence that is at least 90% identical to a sequence of any one of SEQ ID NOs: 788-805; k. nucleotide 11 through nucleotide 36 of a sequence that is at least 90% identical to a sequence of any one of SEQ ID NOs: 788-805; 1. nucleotide 12 through nucleotide 36 of a sequence that is at least 90% identical to a sequence of any one of SEQ ID NOs: 788-805; m. nucleotide 13 through nucleotide 36 of a sequence that is at least 90% identical to a sequence of any one of SEQ ID NOs: 788-805; n. nucleotide 14 through nucleotide 36 of a sequence that is at least 90% identical to a sequence of any one of SEQ ID NOs: 788-805; or o. a sequence that is at least 90% identical to a sequence of SEQ ID NO: 806 or a portion thereof.

In another aspect of the composition, the direct repeat comprises: a. nucleotide 1 through nucleotide 36 of any one of SEQ ID NOs: 788-805; b. nucleotide 2 through nucleotide 36 of any one of SEQ ID NOs: 788-805; c. nucleotide 3 through nucleotide 36 of any one of SEQ ID NOs: 788-805; d. nucleotide 4 through nucleotide 36 of any one of SEQ ID NOs: 788-805; e. nucleotide 5 through nucleotide 36 of any one of SEQ ID NOs: 788-805; f. nucleotide 6 through nucleotide 36 of any one of SEQ ID NOs: 788-805; g. nucleotide 7 through nucleotide 36 of any one of SEQ ID NOs: 788-805; h. nucleotide 8 through nucleotide 36 of any one of SEQ ID NOs: 788-805; i. nucleotide 9 through nucleotide 36 of any one of SEQ ID NOs: 788-805; j. nucleotide 10 through nucleotide 36 of any one of SEQ ID NOs: 788-805; k. nucleotide 11 through nucleotide 36 of any one of SEQ ID NOs: 788-805; 1. nucleotide 12 through nucleotide 36 of any one of SEQ ID NOs: 788-805; m. nucleotide 13 through nucleotide 36 of any one of SEQ ID NOs: 788-805; n. nucleotide 14 through nucleotide 36 of any one of SEQ ID NOs: 788-805; or o. SEQ ID NO: 806 or a portion thereof.

In another aspect of the composition, the direct repeat comprises: a. nucleotide 1 through nucleotide 36 of a sequence that is at least 90% identical to SEQ ID NO: 807; b. nucleotide 2 through nucleotide 36 of a sequence that is at least 90% identical to SEQ ID NO: 807; c. nucleotide 3 through nucleotide 36 of a sequence that is at least 90% identical to SEQ ID NO: 807; d. nucleotide 4 through nucleotide 36 of a sequence that is at least 90% identical to SEQ ID NO: 807; e. nucleotide 5 through nucleotide 36 of a sequence that is at least 90% identical to SEQ ID NO: 807; f. nucleotide 6 through nucleotide 36 of a sequence that is at least 90% identical to SEQ ID NO: 807; g. nucleotide 7 through nucleotide 36 of a sequence that is at least 90% identical to SEQ ID NO: 807; h. nucleotide 8 through nucleotide 36 of a sequence that is at least 90% identical to SEQ ID NO: 807; i. nucleotide 9 through nucleotide 36 of a sequence that is at least 90% identical to SEQ ID NO: 807; j. nucleotide 10 through nucleotide 36 of a sequence that is at least 90% identical to SEQ ID NO: 807; k. nucleotide 11 through nucleotide 36 of a sequence that is at least 90% identical to SEQ ID NO: 807; 1. nucleotide 12 through nucleotide 36 of a sequence that is at least 90% identical to SEQ ID NO: 807; m. nucleotide 13 through nucleotide 36 of a sequence that is at least 90% identical to SEQ ID NO: 807; n. nucleotide 14 through nucleotide 36 of a sequence that is at least 90% identical to SEQ ID NO: 807; or o. a sequence that is at least 90% identical to a sequence of SEQ ID NO: 808 or SEQ ID NO: 809 or a portion thereof.

In another aspect of the composition, the direct repeat comprises: a. nucleotide 1 through nucleotide 36 of SEQ ID NO: 807; b. nucleotide 2 through nucleotide 36 of SEQ ID NO: 807; c. nucleotide 3 through nucleotide 36 of SEQ ID NO: 807; d. nucleotide 4 through nucleotide 36 of SEQ ID NO: 807; e. nucleotide 5 through nucleotide 36 of SEQ ID NO: 807; f. nucleotide 6 through nucleotide 36 of SEQ ID NO: 807; g. nucleotide 7 through nucleotide 36 of SEQ ID NO: 807; h. nucleotide 8 through nucleotide 36 of SEQ ID NO: 807; i. nucleotide 9 through nucleotide 36 of SEQ ID NO: 807; j. nucleotide 10 through nucleotide 36 of SEQ ID NO: 807; k. nucleotide 11 through nucleotide 36 of SEQ ID NO: 807; 1. nucleotide 12 through nucleotide 36 of SEQ ID NO: 807; m. nucleotide 13 through nucleotide 36 of SEQ ID NO: 807; n. nucleotide 14 through nucleotide 36 of SEQ ID NO: 807; or o. SEQ ID NO: 808 or SEQ ID NO: 809 or a portion thereof.

In another aspect of the composition, the direct repeat comprises: a. nucleotide 1 through nucleotide 36 of a sequence that is at least 90% identical to a sequence of SEQ ID NO: 810 or SEQ ID NO: 811; b. nucleotide 2 through nucleotide 36 of a sequence that is at least 90% identical to a sequence of SEQ ID NO: 810 or SEQ ID NO: 811; c. nucleotide 3 through nucleotide 36 of a sequence that is at least 90% identical to a sequence of SEQ ID NO: 810 or SEQ ID NO: 811; d. nucleotide 4 through nucleotide 36 of a sequence that is at least 90% identical to a sequence of SEQ ID NO: 810 or SEQ ID NO: 811; e. nucleotide 5 through nucleotide 36 of a sequence that is at least 90% identical to a sequence of SEQ ID NO: 810 or SEQ ID NO: 811; f. nucleotide 6 through nucleotide 36 of a sequence that is at least 90% identical to a sequence of SEQ ID NO: 810 or SEQ ID NO: 811; g. nucleotide 7 through nucleotide 36 of a sequence that is at least 90% identical to a sequence of SEQ ID NO: 810 or SEQ ID NO: 811; h. nucleotide 8 through nucleotide 36 of a sequence that is at least 90% identical to a sequence of SEQ ID NO: 810 or SEQ ID NO: 811; i. nucleotide 9 through nucleotide 36 of a sequence that is at least 90% identical to a sequence of SEQ ID NO: 810 or SEQ ID NO: 811; j. nucleotide 10 through nucleotide 36 of a sequence that is at least 90% identical to a sequence of SEQ ID NO: 810 or SEQ ID NO: 811; k. nucleotide 11 through nucleotide 36 of a sequence that is at least 90% identical to a sequence of SEQ ID NO: 810 or SEQ ID NO: 811; 1. nucleotide 12 through nucleotide 36 of a sequence that is at least 90% identical to a sequence of SEQ ID NO: 810 or SEQ ID NO: 811; m. nucleotide 13 through nucleotide 36 of a sequence that is at least 90% identical to a sequence of SEQ ID NO: 810 or SEQ ID NO: 811; n. nucleotide 14 through nucleotide 36 of a sequence that is at least 90% identical to a sequence of SEQ ID NO: 810 or SEQ ID NO: 811; o. nucleotide 15 through nucleotide 36 of a sequence that is at least 90% identical to a sequence of SEQ ID NO: 810 or SEQ ID NO: 811; or p. a sequence that is at least 90% identical to a sequence of SEQ ID NO: 812 or a portion thereof.

In another aspect of the composition, the direct repeat comprises: a. nucleotide 1 through nucleotide 36 of SEQ ID NO: 810 or SEQ ID NO: 811; b. nucleotide 2 through nucleotide 36 of SEQ ID NO: 810 or SEQ ID NO: 811; c. nucleotide 3 through nucleotide 36 of SEQ ID NO: 810 or SEQ ID NO: 811; d. nucleotide 4 through nucleotide 36 of SEQ ID NO: 810 or SEQ ID NO: 811; e. nucleotide 5 through nucleotide 36 of SEQ ID NO: 810 or SEQ ID NO: 811; f. nucleotide 6 through nucleotide 36 of SEQ ID NO: 810 or SEQ ID NO: 811; g. nucleotide 7 through nucleotide 36 of SEQ ID NO: 810 or SEQ ID NO: 811; h. nucleotide 8 through nucleotide 36 of SEQ ID NO: 810 or SEQ ID NO: 811; i. nucleotide 9 through nucleotide 36 of SEQ ID NO: 810 or SEQ ID NO: 811; j. nucleotide 10 through nucleotide 36 of SEQ ID NO: 810 or SEQ ID NO: 811; k. nucleotide 11 through nucleotide 36 of SEQ ID NO: 810 or SEQ ID NO: 811; 1. nucleotide 12 through nucleotide 36 of SEQ ID NO: 810 or SEQ ID NO: 811; m. nucleotide 13 through nucleotide 36 of SEQ ID NO: 810 or SEQ ID NO: 811; n. nucleotide 14 through nucleotide 36 of SEQ ID NO: 810 or SEQ ID NO: 811; o. nucleotide 15 through nucleotide 36 of SEQ ID NO: 810 or SEQ ID NO: 811; or p. SEQ ID NO: 812 or a portion thereof.

In another aspect of the composition, the spacer sequence is substantially complementary to the complement of a sequence of any one of SEQ ID NOs: 11-390 or 819-1018.

In another aspect of the composition, the target sequence is adjacent to a protospacer adjacent motif (PAM) comprising the sequence 5′-NTTN-3′.

In another aspect of the composition, the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In another aspect of the composition, the target sequence is immediately adjacent to the PAM sequence.

In another aspect of the composition, the target sequence is within 1, 2, 3, 4, or 5 nucleotides of the PAM sequence.

In another aspect of the composition, the Cas12i polypeptide is: a. a Cas12i2 polypeptide comprising a sequence that is at least 90% identical to the sequence of SEQ ID NO: 772, SEQ ID NO: 782, SEQ ID NO: 783, SEQ ID NO: 784, SEQ ID NO: 785, or SEQ ID NO: 786; b. a Cas12i4 polypeptide comprising a sequence that is at least 90% identical to the sequence of SEQ ID NO: 814, SEQ ID NO: 815, or SEQ ID NO: 816; c. a Cas12i1 polypeptide comprising a sequence that is at least 90% identical to the sequence of SEQ ID NO: 817; or d. a Cas12i3 polypeptide comprising a sequence that is at least 90% identical to the sequence of SEQ ID NO: 818.

In another aspect of the composition, the Cas12i polypeptide is: a. a Cas12i2 polypeptide comprising a sequence of SEQ ID NO: 772, SEQ ID NO: 782, SEQ ID NO: 783, SEQ ID NO: 784, SEQ ID NO: 785, or SEQ ID NO: 786; b. a Cas12i4 polypeptide comprising a sequence of SEQ ID NO: 814, SEQ ID NO: 815, or SEQ ID NO: 816; c. a Cas12i1 polypeptide comprising a sequence of SEQ ID NO: 817; or d. a Cas12i3 polypeptide comprising a sequence of SEQ ID NO: 818.

In another aspect of the composition, the RNA guide and the Cas12i polypeptide form a ribonucleoprotein complex.

In another aspect of the composition, the ribonucleoprotein complex binds a target nucleic acid.

In another aspect of the composition, the composition is present within a cell.

In another aspect of the composition, the RNA guide and the Cas12i polypeptide are encoded in a vector, e.g., expression vector. In another aspect of the composition, the RNA guide and the Cas12i polypeptide are encoded in a single vector or the RNA guide is encoded in a first vector and the Cas12i polypeptide is encoded in a second vector.

The invention further provides a vector system comprising one or more vectors encoding an RNA guide disclosed herein and a Cas12i polypeptide. In an embodiment, the vector system comprises a first vector encoding an RNA guide disclosed herein and a second vector encoding a Cas12i polypeptide. The vectors may be expression vectors.

In another aspect of the composition, the RNA guide does not consist of the sequence of:

(SEQ ID NO: 778)
AGAAAUCCGUCUUUCAUUGACGGAAUGUCGGAUGGAUGAAACC;
(SEQ ID NO: 779)
AGAAAUCCGUCUUUCAUUGACGGCUAUCUCUUGUACUACACUG;
(SEQ ID NO: 780)
AGAAAUCCGUCUUUCAUUGACGGACACGGCAGGCAUACUCAUC;
or
(SEQ ID NO: 781)
AGAAAUCCGUCUUUCAUUGACGGGUCACAGCCCAAGAUAGUUA.

The invention yet further provides an RNA guide comprising (i) a spacer sequence that is substantially complementary to a target sequence within a B2M gene and (ii) a direct repeat sequence.

In one aspect of the RNA guide, the target sequence is within exon 1, exon 2, exon 3, exon 4, intron 1, intron 2, or intron 3 of the B2M gene.

In another aspect of the RNA guide, the B2M gene comprises the sequence of SEQ ID NO: 773, the reverse complement of SEQ ID NO: 773, a variant of the sequence of SEQ ID NO: 773, or the reverse complement of a variant of SEQ ID NO: 773.

In another aspect of the RNA guide, the spacer sequence comprises: a. nucleotide 1 through nucleotide 16 of a sequence that is at least 90% identical to a sequence of any one of SEQ ID NOs: 391-770 or 1019-1218; b. nucleotide 1 through nucleotide 17 of a sequence that is at least 90% identical to a sequence of any one of SEQ ID NOs: 391-770 or 1019-1218; c. nucleotide 1 through nucleotide 18 of a sequence that is at least 90% identical to a sequence of any one of SEQ ID NOs: 391-770 or 1019-1218; d. nucleotide 1 through nucleotide 19 of a sequence that is at least 90% identical to a sequence of any one of SEQ ID NOs: 391-770 or 1019-1218; e. nucleotide 1 through nucleotide 20 of a sequence that is at least 90% identical to a sequence of any one of SEQ ID NOs: 391-770 or 1019-1218; f. nucleotide 1 through nucleotide 21 of a sequence that is at least 90% identical to a sequence of any one of SEQ ID NOs: 391-770; g. nucleotide 1 through nucleotide 22 of a sequence that is at least 90% identical to a sequence of any one of SEQ ID NOs: 391-552 and 554-770; h. nucleotide 1 through nucleotide 23 of a sequence that is at least 90% identical to a sequence of any one of SEQ ID NOs: 391-552 and 555-770; i. nucleotide 1 through nucleotide 24 of a sequence that is at least 90% identical to a sequence of any one of SEQ ID NOs: 391-552 and 556-770; j. nucleotide 1 through nucleotide 25 of a sequence that is at least 90% identical to a sequence of any one of SEQ ID NOs: 391-552 and 556-770; k. nucleotide 1 through nucleotide 26 of a sequence that is at least 90% identical to a sequence of any one of SEQ ID NOs: 391-552 and 556-770; 1. nucleotide 1 through nucleotide 27 of a sequence that is at least 90% identical to a sequence of any one of SEQ ID NOs: 391-552 and 556-770; m. nucleotide 1 through nucleotide 28 of a sequence that is at least 90% identical to a sequence of any one of SEQ ID NOs: 391-552 and 556-770; n. nucleotide 1 through nucleotide 29 of a sequence that is at least 90% identical to a sequence of any one of SEQ ID NOs: 391-552 and 556-770; or o. nucleotide 1 through nucleotide 30 of a sequence that is at least 90% identical to a sequence of any one of SEQ ID NOs: 391-540, 542-552, and 556-770.

In another aspect of the RNA guide, the spacer sequence comprises: a. nucleotide 1 through nucleotide 16 of any one of SEQ ID NOs: 391-770 or 1019-1218; b. nucleotide 1 through nucleotide 17 of any one of SEQ ID NOs: 391-770 or 1019-1218; c. nucleotide 1 through nucleotide 18 of any one of SEQ ID NOs: 391-770 or 1019-1218; d. nucleotide 1 through nucleotide 19 of any one of SEQ ID NOs: 391-770 or 1019-1218; e. nucleotide 1 through nucleotide 20 of any one of SEQ ID NOs: 391-770 or 1019-1218; f. nucleotide 1 through nucleotide 21 of any one of SEQ ID NOs: 391-770; g. nucleotide 1 through nucleotide 22 of any one of SEQ ID NOs: 391-552 and 554-770; h. nucleotide 1 through nucleotide 23 of any one of SEQ ID NOs: 391-552 and 555-770; i. nucleotide 1 through nucleotide 24 of any one of SEQ ID NOs: 391-552 and 556-770; j. nucleotide 1 through nucleotide 25 of any one of SEQ ID NOs: 391-552 and 556-770; k. nucleotide 1 through nucleotide 26 of any one of SEQ ID NOs: 391-552 and 556-770; 1. nucleotide 1 through nucleotide 27 of any one of SEQ ID NOs: 391-552 and 556-770; m. nucleotide 1 through nucleotide 28 of any one of SEQ ID NOs: 391-552 and 556-770; n. nucleotide 1 through nucleotide 29 of any one of SEQ ID NOs: 391-552 and 556-770; or o. nucleotide 1 through nucleotide 30 of any one of SEQ ID NOs: 391-540, 542-552, and 556-770.

In another aspect of the RNA guide, the direct repeat comprises: a. nucleotide 1 through nucleotide 36 of a sequence that is at least 90% identical to a sequence of any one of SEQ ID NOs: 1-8; b. nucleotide 2 through nucleotide 36 of a sequence that is at least 90% identical to a sequence of any one of SEQ ID NOs: 1-8; c. nucleotide 3 through nucleotide 36 of a sequence that is at least 90% identical to a sequence of any one of SEQ ID NOs: 1-8; d. nucleotide 4 through nucleotide 36 of a sequence that is at least 90% identical to a sequence of any one of SEQ ID NOs: 1-8; e. nucleotide 5 through nucleotide 36 of a sequence that is at least 90% identical to a sequence of any one of SEQ ID NOs: 1-8; f. nucleotide 6 through nucleotide 36 of a sequence that is at least 90% identical to a sequence of any one of SEQ ID NOs: 1-8; g. nucleotide 7 through nucleotide 36 of a sequence that is at least 90% identical to a sequence of any one of SEQ ID NOs: 1-8; h. nucleotide 8 through nucleotide 36 of a sequence that is at least 90% identical to a sequence of any one of SEQ ID NOs: 1-8; i. nucleotide 9 through nucleotide 36 of a sequence that is at least 90% identical to a sequence of any one of SEQ ID NOs: 1-8; j. nucleotide 10 through nucleotide 36 of a sequence that is at least 90% identical to a sequence of any one of SEQ ID NOs: 1-8; k. nucleotide 11 through nucleotide 36 of a sequence that is at least 90% identical to a sequence of any one of SEQ ID NOs: 1-8; l. nucleotide 12 through nucleotide 36 of a sequence that is at least 90% identical to a sequence of any one of SEQ ID NOs: 1-8; m. nucleotide 13 through nucleotide 36 of a sequence that is at least 90% identical to a sequence of any one of SEQ ID NOs: 1-8; n. nucleotide 14 through nucleotide 36 of a sequence that is at least 90% identical to a sequence of any one of SEQ ID NOs: 1-8; o. nucleotide 1 through nucleotide 34 of a sequence that is at least 90% identical to a sequence of SEQ ID NO: 9; p. nucleotide 2 through nucleotide 34 of a sequence that is at least 90% identical to a sequence of SEQ ID NO: 9; q. nucleotide 3 through nucleotide 34 of a sequence that is at least 90% identical to a sequence of SEQ ID NO: 9; r. nucleotide 4 through nucleotide 34 of a sequence that is at least 90% identical to a sequence of SEQ ID NO: 9; s. nucleotide 5 through nucleotide 34 of a sequence that is at least 90% identical to a sequence of SEQ ID NO: 9; t. nucleotide 6 through nucleotide 34 of a sequence that is at least 90% identical to a sequence of SEQ ID NO: 9; u. nucleotide 7 through nucleotide 34 of a sequence that is at least 90% identical to a sequence of SEQ ID NO: 9; v. nucleotide 8 through nucleotide 34 of a sequence that is at least 90% identical to a sequence of SEQ ID NO: 9; w. nucleotide 9 through nucleotide 34 of a sequence that is at least 90% identical to a sequence of SEQ ID NO: 9; x. nucleotide 10 through nucleotide 34 of a sequence that is at least 90% identical to a sequence of SEQ ID NO: 9; y. nucleotide 11 through nucleotide 34 of a sequence that is at least 90% identical to a sequence of SEQ ID NO: 9; z. nucleotide 12 through nucleotide 34 of a sequence that is at least 90% identical to a sequence of SEQ ID NO: 9; or aa. a sequence that is at least 90% identical to a sequence of SEQ ID NO: 10 or a portion thereof.

In another aspect of the RNA guide, the direct repeat comprises: a. nucleotide 1 through nucleotide 36 of any one of SEQ ID NOs: 1-8; b. nucleotide 2 through nucleotide 36 of any one of SEQ ID NOs: 1-8; c. nucleotide 3 through nucleotide 36 of any one of SEQ ID NOs: 1-8; d. nucleotide 4 through nucleotide 36 of any one of SEQ ID NOs: 1-8; e. nucleotide 5 through nucleotide 36 of any one of SEQ ID NOs: 1-8; f. nucleotide 6 through nucleotide 36 of any one of SEQ ID NOs: 1-8; g. nucleotide 7 through nucleotide 36 of any one of SEQ ID NOs: 1-8; h. nucleotide 8 through nucleotide 36 of any one of SEQ ID NOs: 1-8; i. nucleotide 9 through nucleotide 36 of any one of SEQ ID NOs: 1-8; j. nucleotide 10 through nucleotide 36 of any one of SEQ ID NOs: 1-8; k. nucleotide 11 through nucleotide 36 of any one of SEQ ID NOs: 1-8; l. nucleotide 12 through nucleotide 36 of any one of SEQ ID NOs: 1-8; m. nucleotide 13 through nucleotide 36 of any one of SEQ ID NOs: 1-8; n. nucleotide 14 through nucleotide 36 of any one of SEQ ID NOs: 1-8; o. nucleotide 1 through nucleotide 34 of SEQ ID NO: 9; p. nucleotide 2 through nucleotide 34 of SEQ ID NO: 9; q. nucleotide 3 through nucleotide 34 of SEQ ID NO: 9; r. nucleotide 4 through nucleotide 34 of SEQ ID NO: 9; s. nucleotide 5 through nucleotide 34 of SEQ ID NO: 9; t. nucleotide 6 through nucleotide 34 of SEQ ID NO: 9; u. nucleotide 7 through nucleotide 34 of SEQ ID NO: 9; v. nucleotide 8 through nucleotide 34 of SEQ ID NO: 9; w. nucleotide 9 through nucleotide 34 of SEQ ID NO: 9; x. nucleotide 10 through nucleotide 34 of SEQ ID NO: 9; y. nucleotide 11 through nucleotide 34 of SEQ ID NO: 9; z. nucleotide 12 through nucleotide 34 of SEQ ID NO: 9; or aa. SEQ ID NO: 10 or a portion thereof.

In another aspect of the RNA guide, the direct repeat comprises: a. nucleotide 1 through nucleotide 36 of a sequence that is at least 90% identical to a sequence of any one of SEQ ID NOs: 788-805; b. nucleotide 2 through nucleotide 36 of a sequence that is at least 90% identical to a sequence of any one of SEQ ID NOs: 788-805; c. nucleotide 3 through nucleotide 36 of a sequence that is at least 90% identical to a sequence of any one of SEQ ID NOs: 788-805; d. nucleotide 4 through nucleotide 36 of a sequence that is at least 90% identical to a sequence of any one of SEQ ID NOs: 788-805; e. nucleotide 5 through nucleotide 36 of a sequence that is at least 90% identical to a sequence of any one of SEQ ID NOs: 788-805; f. nucleotide 6 through nucleotide 36 of a sequence that is at least 90% identical to a sequence of any one of SEQ ID NOs: 788-805; g. nucleotide 7 through nucleotide 36 of a sequence that is at least 90% identical to a sequence of any one of SEQ ID NOs: 788-805; h. nucleotide 8 through nucleotide 36 of a sequence that is at least 90% identical to a sequence of any one of SEQ ID NOs: 788-805; i. nucleotide 9 through nucleotide 36 of a sequence that is at least 90% identical to a sequence of any one of SEQ ID NOs: 788-805; j. nucleotide 10 through nucleotide 36 of a sequence that is at least 90% identical to a sequence of any one of SEQ ID NOs: 788-805; k. nucleotide 11 through nucleotide 36 of a sequence that is at least 90% identical to a sequence of any one of SEQ ID NOs: 788-805; l. nucleotide 12 through nucleotide 36 of a sequence that is at least 90% identical to a sequence of any one of SEQ ID NOs: 788-805; m. nucleotide 13 through nucleotide 36 of a sequence that is at least 90% identical to a sequence of any one of SEQ ID NOs: 788-805; n. nucleotide 14 through nucleotide 36 of a sequence that is at least 90% identical to a sequence of any one of SEQ ID NOs: 788-805; or o. a sequence that is at least 90% identical to a sequence of SEQ ID NO: 806 or a portion thereof.

In another aspect of the RNA guide, the direct repeat comprises: a. nucleotide 1 through nucleotide 36 of any one of SEQ ID NOs: 788-805; b. nucleotide 2 through nucleotide 36 of any one of SEQ ID NOs: 788-805; c. nucleotide 3 through nucleotide 36 of any one of SEQ ID NOs: 788-805; d. nucleotide 4 through nucleotide 36 of any one of SEQ ID NOs: 788-805; e. nucleotide 5 through nucleotide 36 of any one of SEQ ID NOs: 788-805; f. nucleotide 6 through nucleotide 36 of any one of SEQ ID NOs: 788-805; g. nucleotide 7 through nucleotide 36 of any one of SEQ ID NOs: 788-805; h. nucleotide 8 through nucleotide 36 of any one of SEQ ID NOs: 788-805; i. nucleotide 9 through nucleotide 36 of any one of SEQ ID NOs: 788-805; j. nucleotide 10 through nucleotide 36 of any one of SEQ ID NOs: 788-805; k. nucleotide 11 through nucleotide 36 of any one of SEQ ID NOs: 788-805; l. nucleotide 12 through nucleotide 36 of any one of SEQ ID NOs: 788-805; m. nucleotide 13 through nucleotide 36 of any one of SEQ ID NOs: 788-805; n. nucleotide 14 through nucleotide 36 of any one of SEQ ID NOs: 788-805; or o. SEQ ID NO: 806 or a portion thereof.

In another aspect of the RNA guide, the direct repeat comprises: a. nucleotide 1 through nucleotide 36 of a sequence that is at least 90% identical to SEQ ID NO: 807; b. nucleotide 2 through nucleotide 36 of a sequence that is at least 90% identical to SEQ ID NO: 807; c. nucleotide 3 through nucleotide 36 of a sequence that is at least 90% identical to SEQ ID NO: 807; d. nucleotide 4 through nucleotide 36 of a sequence that is at least 90% identical to SEQ ID NO: 807; e. nucleotide 5 through nucleotide 36 of a sequence that is at least 90% identical to SEQ ID NO: 807; f. nucleotide 6 through nucleotide 36 of a sequence that is at least 90% identical to SEQ ID NO: 807; g. nucleotide 7 through nucleotide 36 of a sequence that is at least 90% identical to SEQ ID NO: 807; h. nucleotide 8 through nucleotide 36 of a sequence that is at least 90% identical to SEQ ID NO: 807; i. nucleotide 9 through nucleotide 36 of a sequence that is at least 90% identical to SEQ ID NO: 807; j. nucleotide 10 through nucleotide 36 of a sequence that is at least 90% identical to SEQ ID NO: 807; k. nucleotide 11 through nucleotide 36 of a sequence that is at least 90% identical to SEQ ID NO: 807; l. nucleotide 12 through nucleotide 36 of a sequence that is at least 90% identical to SEQ ID NO: 807; m. nucleotide 13 through nucleotide 36 of a sequence that is at least 90% identical to SEQ ID NO: 807; n. nucleotide 14 through nucleotide 36 of a sequence that is at least 90% identical to SEQ ID NO: 807; or o. a sequence that is at least 90% identical to a sequence of SEQ ID NO: 808 or SEQ ID NO: 809 or a portion thereof.

In another aspect of the RNA guide, the direct repeat comprises: a. nucleotide 1 through nucleotide 36 of SEQ ID NO: 807; b. nucleotide 2 through nucleotide 36 of SEQ ID NO: 807; c. nucleotide 3 through nucleotide 36 of SEQ ID NO: 807; d. nucleotide 4 through nucleotide 36 of SEQ ID NO: 807; e. nucleotide 5 through nucleotide 36 of SEQ ID NO: 807; f. nucleotide 6 through nucleotide 36 of SEQ ID NO: 807; g. nucleotide 7 through nucleotide 36 of SEQ ID NO: 807; h. nucleotide 8 through nucleotide 36 of SEQ ID NO: 807; i. nucleotide 9 through nucleotide 36 of SEQ ID NO: 807; j. nucleotide 10 through nucleotide 36 of SEQ ID NO: 807; k. nucleotide 11 through nucleotide 36 of SEQ ID NO: 807; l. nucleotide 12 through nucleotide 36 of SEQ ID NO: 807; m. nucleotide 13 through nucleotide 36 of SEQ ID NO: 807; n. nucleotide 14 through nucleotide 36 of SEQ ID NO: 807; or o. SEQ ID NO: 808 or SEQ ID NO: 809 or a portion thereof.

In another aspect of the RNA guide, the direct repeat comprises: a. nucleotide 1 through nucleotide 36 of a sequence that is at least 90% identical to a sequence of SEQ ID NO: 810 or SEQ ID NO: 811; b. nucleotide 2 through nucleotide 36 of a sequence that is at least 90% identical to a sequence of SEQ ID NO: 810 or SEQ ID NO: 811; c. nucleotide 3 through nucleotide 36 of a sequence that is at least 90% identical to a sequence of SEQ ID NO: 810 or SEQ ID NO: 811; d. nucleotide 4 through nucleotide 36 of a sequence that is at least 90% identical to a sequence of SEQ ID NO: 810 or SEQ ID NO: 811; e. nucleotide 5 through nucleotide 36 of a sequence that is at least 90% identical to a sequence of SEQ ID NO: 810 or SEQ ID NO: 811; f. nucleotide 6 through nucleotide 36 of a sequence that is at least 90% identical to a sequence of SEQ ID NO: 810 or SEQ ID NO: 811; g. nucleotide 7 through nucleotide 36 of a sequence that is at least 90% identical to a sequence of SEQ ID NO: 810 or SEQ ID NO: 811; h. nucleotide 8 through nucleotide 36 of a sequence that is at least 90% identical to a sequence of SEQ ID NO: 810 or SEQ ID NO: 811; i. nucleotide 9 through nucleotide 36 of a sequence that is at least 90% identical to a sequence of SEQ ID NO: 810 or SEQ ID NO: 811; j. nucleotide 10 through nucleotide 36 of a sequence that is at least 90% identical to a sequence of SEQ ID NO: 810 or SEQ ID NO: 811; k. nucleotide 11 through nucleotide 36 of a sequence that is at least 90% identical to a sequence of SEQ ID NO: 810 or SEQ ID NO: 811; l. nucleotide 12 through nucleotide 36 of a sequence that is at least 90% identical to a sequence of SEQ ID NO: 810 or SEQ ID NO: 811; m. nucleotide 13 through nucleotide 36 of a sequence that is at least 90% identical to a sequence of SEQ ID NO: 810 or SEQ ID NO: 811; n. nucleotide 14 through nucleotide 36 of a sequence that is at least 90% identical to a sequence of SEQ ID NO: 810 or SEQ ID NO: 811; o. nucleotide 15 through nucleotide 36 of a sequence that is at least 90% identical to a sequence of SEQ ID NO: 810 or SEQ ID NO: 811; or p. a sequence that is at least 90% identical to a sequence of SEQ ID NO: 812 or a portion thereof.

In another aspect of the RNA guide, the direct repeat comprises: a. nucleotide 1 through nucleotide 36 of SEQ ID NO: 810 or SEQ ID NO: 811; b. nucleotide 2 through nucleotide 36 of SEQ ID NO: 810 or SEQ ID NO: 811; c. nucleotide 3 through nucleotide 36 of SEQ ID NO: 810 or SEQ ID NO: 811; d. nucleotide 4 through nucleotide 36 of SEQ ID NO: 810 or SEQ ID NO: 811; e. nucleotide 5 through nucleotide 36 of SEQ ID NO: 810 or SEQ ID NO: 811; f. nucleotide 6 through nucleotide 36 of SEQ ID NO: 810 or SEQ ID NO: 811; g. nucleotide 7 through nucleotide 36 of SEQ ID NO: 810 or SEQ ID NO: 811; h. nucleotide 8 through nucleotide 36 of SEQ ID NO: 810 or SEQ ID NO: 811; i. nucleotide 9 through nucleotide 36 of SEQ ID NO: 810 or SEQ ID NO: 811; j. nucleotide 10 through nucleotide 36 of SEQ ID NO: 810 or SEQ ID NO: 811; k. nucleotide 11 through nucleotide 36 of SEQ ID NO: 810 or SEQ ID NO: 811; l. nucleotide 12 through nucleotide 36 of SEQ ID NO: 810 or SEQ ID NO: 811; m. nucleotide 13 through nucleotide 36 of SEQ ID NO: 810 or SEQ ID NO: 811; n. nucleotide 14 through nucleotide 36 of SEQ ID NO: 810 or SEQ ID NO: 811; o. nucleotide 15 through nucleotide 36 of SEQ ID NO: 810 or SEQ ID NO: 811; or p. SEQ ID NO: 812 or a portion thereof.

In another aspect of the RNA guide, the spacer sequence is substantially complementary to the complement of a sequence of any one of SEQ ID NOs: 11-390 or 819-1018.

In another aspect of the RNA guide, the target sequence is adjacent to a protospacer adjacent motif (PAM) comprising the sequence 5′-NTTN-3′, wherein N is any nucleotide.

In another aspect of the RNA guide, the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In another aspect of the RNA guide, the target sequence is immediately adjacent to the PAM sequence.

In another aspect of the RNA guide, the target sequence is within 1, 2, 3, 4, or 5 nucleotides of the PAM sequence.

In another aspect of the RNA guide, the RNA guide does not consist of the sequence of:

(SEQ ID NO: 778)
AGAAAUCCGUCUUUCAUUGACGGAAUGUCGGAUGGAUGAAACC;
(SEQ ID NO: 779)
AGAAAUCCGUCUUUCAUUGACGGCUAUCUCUUGUACUACACUG;
(SEQ ID NO: 780)
AGAAAUCCGUCUUUCAUUGACGGACACGGCAGGCAUACUCAUC;
or
(SEQ ID NO: 781)
AGAAAUCCGUCUUUCAUUGACGGGUCACAGCCCAAGAUAGUUA.

The invention yet further provides a nucleic acid encoding an RNA guide as described herein.

The invention yet further provides a vector comprising such an RNA guide as described herein.

The invention yet further provides a cell comprising a composition, an RNA guide, a nucleic acid, or a vector as described herein.

In one aspect of the cell, the cell is a eukaryotic cell, an animal cell, a mammalian cell, a human cell, a primary cell, a cell line, a stem cell, or a T cell.

The invention yet further provides a kit comprising a composition, an RNA guide, a nucleic acid, or a vector as described herein.

The invention yet further provides a method of editing a B2M sequence, the method comprising contacting a B2M sequence with a composition or an RNA guide as described herein. In an embodiment, the method is carried out in vitro. In an embodiment, the method is carried out ex vivo.

In one aspect of the method, the B2M sequence is in a cell.

In one aspect of the method, the composition or the RNA guide induces a deletion in the B2M sequence.

In one aspect of the method, the deletion is adjacent to a 5′-NTTN-3′ sequence, wherein N is any nucleotide.

In one aspect of the method, the deletion is downstream of the 5′-NTTN-3′ sequence.

In one aspect of the method, the deletion is up to about 40 nucleotides in length.

In one aspect of the method, the deletion is from about 4 nucleotides to 40 nucleotides in length.

In one aspect of the method, the deletion is from about 4 nucleotides to 25 nucleotides in length.

In one aspect of the method, the deletion is from about 10 nucleotides to 25 nucleotides in length.

In one aspect of the method, the deletion is from about 10 nucleotides to 15 nucleotides in length.

In one aspect of the method, the deletion starts within about 5 nucleotides to about 15 nucleotides of the 5′-NTTN-3′ sequence.

In one aspect of the method, the deletion starts within about 5 nucleotides to about 10 nucleotides of the 5′-NTTN-3′ sequence.

In one aspect of the method, the deletion starts within about 10 nucleotides to about 15 nucleotides of the 5′-NTTN-3′ sequence.

In one aspect of the method, the deletion starts within about 5 nucleotides to about 15 nucleotides downstream of the 5′-NTTN-3′ sequence.

In one aspect of the method, the deletion starts within about 5 nucleotides to about 10 nucleotides downstream of the 5′-NTTN-3′ sequence.

In one aspect of the method, the deletion starts within about 10 nucleotides to about 15 nucleotides downstream of the 5′-NTTN-3′ sequence.

In one aspect of the method, the deletion ends within about 20 nucleotides to about 30 nucleotides of the 5′-NTTN-3′ sequence.

In one aspect of the method, the deletion ends within about 20 nucleotides to about 25 nucleotides of the 5′-NTTN-3′ sequence.

In one aspect of the method, the deletion ends within about 25 nucleotides to about 30 nucleotides of the 5′-NTTN-3′ sequence.

In one aspect of the method, the deletion ends within about 20 nucleotides to about 30 nucleotides downstream of the 5′-NTTN-3′ sequence.

In one aspect of the method, the deletion ends within about 20 nucleotides to about 25 nucleotides downstream of the 5′-NTTN-3′ sequence.

In one aspect of the method, the deletion ends within about 25 nucleotides to about 30 nucleotides downstream of the 5′-NTTN-3′ sequence.

In one aspect of the method, the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

In one aspect of the method, the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

In one aspect of the method, the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

In one aspect of the method, the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

In one aspect of the method, the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

In one aspect of the method, the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

In one aspect of the method, the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

In one aspect of the method, the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

In one aspect of the method, the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

In one aspect of the method, the 5′-NTTN-3′ sequence is 5′-CTTT-3′, 5′-CTTC-3′, 5′-GTTT-3′, 5′-GTTC-3′, 5′-TTTC-3′, 5′-GTTA-3′, or 5′-GTTG-3′.

In one aspect of the method, the deletion overlaps with a mutation in the B2M sequence.

In one aspect of the method, the deletion overlaps with an insertion in the B2M sequence.

In one aspect of the method, the deletion removes a repeat expansion of the B2M sequence or a portion thereof.

In one aspect of the method, the deletion disrupts one or both alleles of the B2M sequence.

In one aspect of the composition, RNA guide, nucleic acid, vector, cell, kit, or method described herein, the RNA guide does not consist of the sequence of:

(SEQ ID NO: 778)
AGAAAUCCGUCUUUCAUUGACGGAAUGUCGGAUGGAUGAAACC;
(SEQ ID NO: 779)
AGAAAUCCGUCUUUCAUUGACGGCUAUCUCUUGUACUACACUG;
(SEQ ID NO: 780)
AGAAAUCCGUCUUUCAUUGACGGACACGGCAGGCAUACUCAUC;
or
(SEQ ID NO: 781)
AGAAAUCCGUCUUUCAUUGACGGGUCACAGCCCAAGAUAGUUA.

In one aspect of the composition, RNA guide, nucleic acid, vector, cell, kit, or method described herein, the RNA guide comprises the sequence of any one of SEQ ID NOs: 1222-1230.

### Definitions

The present invention will be described with respect to particular, but the invention is not limited thereto but only by the claims. Terms as set forth hereinafter are generally to be understood in their common sense unless indicated otherwise.

As used herein, the term “activity” refers to a biological activity. In some embodiments, activity includes enzymatic activity, e.g., catalytic ability of an effector. For example, activity can include nuclease activity.

As used herein the term “B2M” refers to “02 microglobulin” or “beta-2 microglobulin.” B2M is a component of major histocompatibility complex (MHC) class I molecules, which are found on the surfaces of all nucleated vertebrate cells and function to display peptide fragments of proteins within the cells to cytotoxic T cells. SEQ ID NO: 773 as set forth herein provides an example of a B2M gene sequence. It is understood that spacer sequences described herein can target SEQ ID NO: 773 or the reverse complement thereof, depending upon whether they are indicated as “+” or “−” as set forth in Table 5. The target sequences listed in Table 5 and Table 6 are on the non-target strand of the B2M gene.

As used herein, the term “Cas12i polypeptide” (also referred to herein as Cas12i) refers to a polypeptide that binds to a target sequence on a target nucleic acid specified by an RNA guide, wherein the polypeptide has at least some amino acid sequence homology to a wild-type Cas12i polypeptide. In some embodiments, the Cas12i polypeptide comprises at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 1-5 and 11-18 of U.S. Pat. No. 10,808,245, which is incorporated by reference herein in its entirety. In some embodiments, a Cas12i polypeptide comprises at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NO: 3 (Cas12i1), SEQ ID NO: 5 (Cas12i2), SEQ ID NO: 14 (Cas12i3), or SEQ ID NO: 16 (Cas12i4) of U.S. Pat. No. 10,808,245, corresponding to SEQ ID NOs: 817, 772, 818, and 814 of the present application. In some embodiments, a Cas12i polypeptide of the disclosure is a Cas12i1 polypeptide or Cas12i2 polypeptide as described in PCT/US2021/025257. In some embodiments, the Cas12i polypeptide cleaves a target nucleic acid (e.g., as a nick or a double strand break).

As used herein, the term “complex” refers to a grouping of two or more molecules. In some embodiments, the complex comprises a polypeptide and a nucleic acid molecule interacting with (e.g., binding to, coming into contact with, adhering to) one another. As used herein, the term “complex” can refer to a grouping of an RNA guide and a polypeptide (e.g., a Cas12i polypeptide). As used herein, the term “complex” can refer to a grouping of an RNA guide, a polypeptide, and a target sequence. As used herein, the term “complex” can refer to a grouping of a B2M-targeting RNA guide and a Cas12i polypeptide.

As used herein, the term “protospacer adjacent motif” or “PAM” refers to a DNA sequence adjacent to a target sequence (e.g., a B2M target sequence) to which a complex comprising an RNA guide (e.g., a B2M-targeting RNA guide) and a Cas12i polypeptide binds. In the case of a double-stranded target, the RNA guide binds to a first strand of the target (e.g., the target strand or the spacer-complementary strand), and a PAM sequence as described herein is present in the second, complementary strand (e.g., the non-target strand or the non-spacer-complementary strand). As used herein, the term “adjacent” includes instances in which the RNA guide of a complex comprising an RNA guide and a Cas12i polypeptide specifically binds, interacts, or associates with a target sequence that is immediately adjacent to a PAM. In such instances, there are no nucleotides between the target sequence and the PAM. The term “adjacent” also includes instances in which there are a small number (e.g., 1, 2, 3, 4, or 5) of nucleotides between the target sequence, to which the RNA guide binds, and the PAM. In some embodiments, the PAM sequence as described herein is present in the non-target strand (e.g., the non-spacer-complementary strand). In such a case, the term “adjacent” includes a PAM sequence as described herein as being immediately adjacent to (or within a small number, e.g., 1, 2, 3, 4, or 5 nucleotides of) a sequence in the non-target strand.

As used herein, the term “RNA guide” refers to any RNA molecule that facilitates the targeting of a polypeptide (e.g., a Cas12i polypeptide) described herein to a target sequence (e.g., a sequence of a B2M gene). An RNA guide may be designed to include sequences that are complementary to a specific nucleic acid sequence (e.g., a B2M nucleic acid sequence). An RNA guide may comprise a DNA targeting sequence (i.e., a spacer sequence) and a direct repeat (DR) sequence. The term “crRNA” is also used herein to refer to an RNA guide.

In some embodiments, a spacer sequence is complementary to a target sequence. As used herein, the term “complementary” refers to the ability of nucleobases of a first nucleic acid molecule, such as an RNA guide, to base pair with nucleobases of a second nucleic acid molecule, such as a target sequence. Two complementary nucleic acid molecules are able to non-covalently bind under appropriate temperature and solution ionic strength conditions. In some embodiments, a first nucleic acid molecule (e.g., a spacer sequence of an RNA guide) comprises 100% complementarity to a second nucleic acid (e.g., a target sequence). In some embodiments, a first nucleic acid molecule (e.g., a spacer sequence of an RNA guide) is complementary to a second nucleic acid molecule (e.g., a target sequence) if the first nucleic acid molecule comprises at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second nucleic acid. As used herein, the term “substantially complementary” refers to a polynucleotide (e.g., a spacer sequence of an RNA guide) that has a certain level of complementarity to a target sequence. In some embodiments, the level of complementarity is such that the polynucleotide can hybridize to the target sequence with sufficient affinity to permit an effector polypeptide (e.g., Cas12i) that is complexed with the polynucleotide to act (e.g., cleave) on the target sequence. In some embodiments, a spacer sequence that is substantially complementary to a target sequence has less than 100% complementarity to the target sequence. In some embodiments, a spacer sequence that is substantially complementary to a target sequence has at least about 80%, 81%, 82%, 83%, 84%, 85%, 86%, 87%, 88%, 89%, 90%, 91%, 92%, 93%, 94%, 95%, 96%, 97%, 98%, or 99% complementarity to the target sequence. In some embodiments, an RNA guide with a spacer sequence that is substantially complementary to a target sequence has 100% complementarity to the target sequence.

As used herein, the terms “target” and “target sequence” refer to a nucleic acid sequence to which an RNA guide specifically binds. In some embodiments, the DNA targeting sequence (e.g., spacer) of an RNA guide binds to a target sequence. In the case of a double-stranded target, the RNA guide binds to a first strand of the target (i.e., the target strand or the spacer-complementary strand), and a PAM sequence as described herein is present in the second, complementary strand (i.e., the non-target strand or the non-spacer-complementary strand). In some embodiments, the target strand (i.e., the spacer-complementary strand) comprises a 5′-NAAN-3′ sequence. In some embodiments, the target sequence is a sequence within a B2M gene sequence, including, but not limited, to the sequence set forth in SEQ ID NO: 773 or the reverse complement thereof.

As used herein, the terms “upstream” and “downstream” refer to relative positions within a single nucleic acid (e.g., DNA) sequence in a nucleic acid molecule. “Upstream” and “downstream” relate to the 5′ to 3′ direction, respectively, in which RNA transcription occurs. A first sequence is upstream of a second sequence when the 3′ end of the first sequence occurs before the 5′ end of the second sequence. A first sequence is downstream of a second sequence when the 5′ end of the first sequence occurs after the 3′ end of the second sequence. In some embodiments, the 5′-NTTN-3′ sequence is upstream of an indel described herein, and a Cas12i-induced indel is downstream of the 5′-NTTN-3′ sequence.

### BRIEF DESCRIPTION OF THE DRAWINGS

FIG. 1 shows indel activity by variant Cas12i2 of SEQ ID NO: 782 and several individual RNA guides targeting B2M at various concentrations in HEK293T cells.

FIG. 2 shows indel activity by variant Cas12i2 of SEQ ID NO: 783 and several individual RNA guides targeting B2M at various concentrations in primary T cells. Error bars represent standard deviation of the mean of four technical replicates from one representative donor.

FIG. 3 shows B2M expression reduction by variant Cas12i2 of SEQ ID NO: 783 and several individual RNA guides targeting B2M at various concentrations in primary T cells. Error bars represent standard deviation of the mean of four technical replicates from one representative donor.

FIG. 4 shows viability of cells (via DAPI staining) seven days following introduction of variant Cas12i2 ribonucleoproteins (RNPs) targeting B2M at various concentrations in primary T cells. Error bars represent standard deviation of the mean of four technical replicates from one representative donor.

### DETAILED DESCRIPTION

The present disclosure relates to an RNA guide capable of binding to B2M and methods of use thereof. In some aspects, a composition comprising an RNA guide having one or more characteristics is described herein. In some aspects, a method of producing the RNA guide is described. In some aspects, a method of delivering a composition comprising the RNA guide is described.

### Composition

In some aspects, the invention described herein comprises compositions comprising an RNA guide targeting B2M. In some embodiments, the RNA guide is comprised of a direct repeat component and a spacer component. In some embodiments, the RNA guide binds a Cas12i polypeptide. In some embodiments, the spacer component is substantially complementary to a B2M target sequence, wherein the B2M target sequence is adjacent to a 5′-NTTN-3′ PAM sequence as described herein. In the case of a double-stranded target, the RNA guide binds to a first strand of the target (i.e., the target strand or the spacer-complementary strand) and a PAM sequence as described herein is present in the second, complementary strand (i.e., the non-target strand or the non-spacer-complementary strand).

In some embodiments, the invention described herein comprises compositions comprising a complex, wherein the complex comprises an RNA guide targeting B2M. In some embodiments, the invention comprises a complex comprising an RNA guide and a Cas12i polypeptide. In some embodiments, the RNA guide and the Cas12i polypeptide bind to each other in a molar ratio of about 1:1. In some embodiments, a complex comprising an RNA guide and a Cas12i polypeptide binds to a B2M target sequence. In some embodiments, a complex comprising an RNA guide targeting B2M and a Cas12i polypeptide binds to a B2M target sequence at a molar ratio of about 1:1. In some embodiments, the complex comprises enzymatic activity, such as nuclease activity, that can cleave the B2M target sequence. The RNA guide, the Cas12i polypeptide, and the B2M target sequence, either alone or together, do not naturally occur.

Use of the compositions disclosed herein has advantages over those of other known nuclease systems. Cas12i polypeptides are smaller than other nucleases. For example, Cas12i2 is 1,054 amino acids in length, whereas S. pyogenes Cas9 (SpCas9) is 1,368 amino acids in length, S. thermophilus Cas9 (StCas9) is 1,128 amino acids in length, FnCpf1 is 1,300 amino acids in length, AsCpf1 is 1,307 amino acids in length, and LbCpf1 is 1,246 amino acids in length. Cas12i RNA guides, which do not require a trans-activating CRISPR RNA (tracrRNA), are also smaller than Cas9 RNA guides. The smaller Cas12i polypeptide and RNA guide sizes are beneficial for delivery. Compositions comprising a Cas12i polypeptide also demonstrate decreased off-target activity compared to compositions comprising an SpCas9 polypeptide. See PCT/US2021/025257, which is incorporated by reference in its entirety. Furthermore, indels induced by compositions comprising a Cas12i polypeptide differ from indels induced by compositions comprising an SpCas9 polypeptide. For example, SpCas9 polypeptides primarily induce insertions and deletions of 1 nucleotide in length. However, Cas12i polypeptides induce larger deletions, which can be beneficial in disrupting a larger portion of a gene such as B2M.

### RNA Guide

In some embodiments, the composition described herein comprises an RNA guide targeting a B2M gene or a portion of B2M gene. In some embodiments, the composition described herein comprises two or more (e.g., 2, 3, 4, 5, 6, 7, 8, 9, or more) RNA guides targeting B2M.

The RNA guide may direct the Cas12i polypeptide as described herein to a B2M target sequence. Two or more RNA guides may target two or more separate Cas12i polypeptides (e.g., Cas12i polypeptides having the same or different sequence) as described herein to two or more (e.g., 2, 3, 4, 5, 6, 7, 8, 9, or more) B2M target sequences.

Those skilled in the art reading the below examples of particular kinds of RNA guides will understand that, in some embodiments, an RNA guide is B2M target-specific. That is, in some embodiments, an RNA guide binds specifically to one or more B2M target sequences (e.g., within a cell) and not to non-targeted sequences (e.g., non-specific DNA or random sequences within the same cell).

In some embodiments, the RNA guide comprises a spacer sequence followed by a direct repeat sequence, referring to the sequences in the 5′ to 3′ direction. In some embodiments, the RNA guide comprises a first direct repeat sequence followed by a spacer sequence and a second direct repeat sequence, referring to the sequences in the 5′ to 3′ direction. In some embodiments, the first and second direct repeats of such an RNA guide are identical. In some embodiments, the first and second direct repeats of such an RNA guide are different.

In some embodiments, the spacer sequence and the direct repeat sequence(s) of the RNA guide are present within the same RNA molecule. In some embodiments, the spacer and direct repeat sequences are linked directly to one another. In some embodiments, a short linker is present between the spacer and direct repeat sequences, e.g., an RNA linker of 1, 2, or 3 nucleotides in length. In some embodiments, the spacer sequence and the direct repeat sequence(s) of the RNA guide are present in separate molecules, which are joined to one another by base pairing interactions.

Additional information regarding exemplary direct repeat and spacer components of RNA guides is provided as follows.

Direct Repeat

In some embodiments, the RNA guide comprises a direct repeat sequence. In some embodiments, the direct repeat sequence of the RNA guide has a length of between 12-100, 13-75, 14-50, or 15-40 nucleotides (e.g., 15, 16, 17, 18, 19, 20, 21, 22, 23, 24, 25, 26, 27, 28, 29, 30, 31, 32, 33, 34, 35, 36, 37, 38, 39, or 40 nucleotides).

In some embodiments, the direct repeat sequence is or comprises a sequence of Table 1 or a portion of a sequence of Table 1. The direct repeat sequence can comprise nucleotide 1 through nucleotide 36 of any one of SEQ ID NOs: 1, 2, 3, 4, 5, 6, 7, or 8. The direct repeat sequence can comprise nucleotide 2 through nucleotide 36 of any one of SEQ ID NOs: 1, 2, 3, 4, 5, 6, 7, or 8. The direct repeat sequence can comprise nucleotide 3 through nucleotide 36 of any one of SEQ ID NOs: 1, 2, 3, 4, 5, 6, 7, or 8. The direct repeat sequence can comprise nucleotide 4 through nucleotide 36 of any one of SEQ ID NOs: 1, 2, 3, 4, 5, 6, 7, or 8. The direct repeat sequence can comprise nucleotide 5 through nucleotide 36 of any one of SEQ ID NOs: 1, 2, 3, 4, 5, 6, 7, or 8. The direct repeat sequence can comprise nucleotide 6 through nucleotide 36 of any one of SEQ ID NOs: 1, 2, 3, 4, 5, 6, 7, or 8. The direct repeat sequence can comprise nucleotide 7 through nucleotide 36 of any one of SEQ ID NOs: 1, 2, 3, 4, 5, 6, 7, or 8. The direct repeat sequence can comprise nucleotide 8 through nucleotide 36 of any one of SEQ ID NOs: 1, 2, 3, 4, 5, 6, 7, or 8. The direct repeat sequence can comprise nucleotide 9 through nucleotide 36 of any one of SEQ ID NOs: 1, 2, 3, 4, 5, 6, 7, or 8. The direct repeat sequence can comprise nucleotide 10 through nucleotide 36 of any one of SEQ ID NOs: 1, 2, 3, 4, 5, 6, 7, or 8. The direct repeat sequence can comprise nucleotide 11 through nucleotide 36 of any one of SEQ ID NOs: 1, 2, 3, 4, 5, 6, 7, or 8. The direct repeat sequence can comprise nucleotide 12 through nucleotide 36 of any one of SEQ ID NOs: 1, 2, 3, 4, 5, 6, 7, or 8. The direct repeat sequence can comprise nucleotide 13 through nucleotide 36 of any one of SEQ ID NOs: 1, 2, 3, 4, 5, 6, 7, or 8. The direct repeat sequence can comprise nucleotide 14 through nucleotide 36 of any one of SEQ ID NOs: 1, 2, 3, 4, 5, 6, 7, or 8. The direct repeat sequence can comprise nucleotide 1 through nucleotide 34 of SEQ ID NO: 9. The direct repeat sequence can comprise nucleotide 2 through nucleotide 34 of SEQ ID NO: 9. The direct repeat sequence can comprise nucleotide 3 through nucleotide 34 of SEQ ID NO: 9. The direct repeat sequence can comprise nucleotide 4 through nucleotide 34 of SEQ ID NO: 9. The direct repeat sequence can comprise nucleotide 5 through nucleotide 34 of SEQ ID NO: 9. The direct repeat sequence can comprise nucleotide 6 through nucleotide 34 of SEQ ID NO: 9. The direct repeat sequence can comprise nucleotide 7 through nucleotide 34 of SEQ ID NO: 9. The direct repeat sequence can comprise nucleotide 8 through nucleotide 34 of SEQ ID NO: 9. The direct repeat sequence can comprise nucleotide 9 through nucleotide 34 of SEQ ID NO: 9. The direct repeat sequence can comprise nucleotide 10 through nucleotide 34 of SEQ ID NO: 9. The direct repeat sequence can comprise nucleotide 11 through nucleotide 34 of SEQ ID NO: 9. The direct repeat sequence can comprise nucleotide 12 through nucleotide 34 of SEQ ID NO: 9. In some embodiments, the direct repeat sequence is set forth in SEQ ID NO: 10. In some embodiments, the direct repeat sequence comprises a portion of the sequence set forth in SEQ ID NO: 10.

In some embodiments, the direct repeat sequence has or comprises a sequence comprising at least 90% identity (e.g., at least 90%, 91%, 92%, 93%, 94%, 95%, 96%, 97%, 98% or 99% identity) to a sequence of Table 1 or a portion of a sequence of Table 1. The direct repeat sequence can have or comprise a sequence having at least 90% identity to a sequence comprising nucleotide 1 through nucleotide 36 of any one of SEQ ID NOs: 1, 2, 3, 4, 5, 6, 7, or 8. The direct repeat sequence can have or comprise a sequence having at least 90% identity to a sequence comprising 2 through nucleotide 36 of any one of SEQ ID NOs: 1, 2, 3, 4, 5, 6, 7, or 8. The direct repeat sequence can have or comprise a sequence having at least 90% identity to a sequence comprising 3 through nucleotide 36 of any one of SEQ ID NOs: 1, 2, 3, 4, 5, 6, 7, or 8. The direct repeat sequence can have or comprise a sequence having at least 90% identity to a sequence comprising 4 through nucleotide 36 of any one of SEQ ID NOs: 1, 2, 3, 4, 5, 6, 7, or 8. The direct repeat sequence can have or comprise a sequence having at least 90% identity to a sequence comprising 5 through nucleotide 36 of any one of SEQ ID NOs: 1, 2, 3, 4, 5, 6, 7, or 8. The direct repeat sequence can have or comprise a sequence having at least 90% identity to a sequence comprising 6 through nucleotide 36 of any one of SEQ ID NOs: 1, 2, 3, 4, 5, 6, 7, or 8. The direct repeat sequence can have or comprise a sequence having at least 90% identity to a sequence comprising 7 through nucleotide 36 of any one of SEQ ID NOs: 1, 2, 3, 4, 5, 6, 7, or 8. The direct repeat sequence can have or comprise a sequence having at least 90% identity to a sequence comprising 8 through nucleotide 36 of any one of SEQ ID NOs: 1, 2, 3, 4, 5, 6, 7, or 8. The direct repeat sequence can have or comprise a sequence having at least 90% identity to a sequence comprising 9 through nucleotide 36 of any one of SEQ ID NOs: 1, 2, 3, 4, 5, 6, 7, or 8. The direct repeat sequence can have or comprise a sequence having at least 90% identity to a sequence comprising 10 through nucleotide 36 of any one of SEQ ID NOs: 1, 2, 3, 4, 5, 6, 7, or 8. The direct repeat sequence can have or comprise a sequence having at least 90% identity to a sequence comprising 11 through nucleotide 36 of any one of SEQ ID NOs: 1, 2, 3, 4, 5, 6, 7, or 8. The direct repeat sequence can have or comprise a sequence having at least 90% identity to a sequence comprising 12 through nucleotide 36 of any one of SEQ ID NOs: 1, 2, 3, 4, 5, 6, 7, or 8. The direct repeat sequence can have or comprise a sequence having at least 90% identity to a sequence comprising 13 through nucleotide 36 of any one of SEQ ID NOs: 1, 2, 3, 4, 5, 6, 7, or 8. The direct repeat sequence can have or comprise a sequence having at least 90% identity to a sequence comprising 14 through nucleotide 36 of any one of SEQ ID NOs: 1, 2, 3, 4, 5, 6, 7, or 8. The direct repeat sequence can have or comprise a sequence having at least 90% identity to a sequence comprising 1 through nucleotide 34 of SEQ ID NO: 9. The direct repeat sequence can have or comprise a sequence having at least 90% identity to a sequence comprising 2 through nucleotide 34 of SEQ ID NO: 9. The direct repeat sequence can have or comprise a sequence having at least 90% identity to a sequence comprising 3 through nucleotide 34 of SEQ ID NO: 9. The direct repeat sequence can have or comprise a sequence having at least 90% identity to a sequence comprising 4 through nucleotide 34 of SEQ ID NO: 9. The direct repeat sequence can have or comprise a sequence having at least 90% identity to a sequence comprising 5 through nucleotide 34 of SEQ ID NO: 9. The direct repeat sequence can have or comprise a sequence having at least 90% identity to a sequence comprising 6 through nucleotide 34 of SEQ ID NO: 9. The direct repeat sequence can have or comprise a sequence having at least 90% identity to a sequence comprising 7 through nucleotide 34 of SEQ ID NO: 9. The direct repeat sequence can have or comprise a sequence having at least 90% identity to a sequence comprising 8 through nucleotide 34 of SEQ ID NO: 9. The direct repeat sequence can have or comprise a sequence having at least 90% identity to a sequence comprising 9 through nucleotide 34 of SEQ ID NO: 9. The direct repeat sequence can have or comprise a sequence having at least 90% identity to a sequence comprising 10 through nucleotide 34 of SEQ ID NO: 9. The direct repeat sequence can have or comprise a sequence having at least 90% identity to a sequence comprising 11 through nucleotide 34 of SEQ ID NO: 9. The direct repeat sequence can have or comprise a sequence having at least 90% identity to a sequence comprising 12 through nucleotide 34 of SEQ ID NO: 9. In some embodiments, the direct repeat sequence has at least 90% identity (e.g., at least 90%, 91%, 92%, 93%, 94%, 95%, 96%, 97%, 98% or 99% identity) to SEQ ID NO: 10. In some embodiments, the direct repeat sequence has at least 90% identity to a portion of the sequence set forth in SEQ ID NO: 10.

In some embodiments, compositions comprising a Cas12i2 polypeptide and an RNA guide comprising the direct repeat of SEQ ID NO: 10 and a spacer length of 20 nucleotides are capable of introducing indels into a B2M target sequence. See, e.g., Example 1, where indels were measured at eleven B2M target sequences following transient transfection of an RNA guide and Cas12i2 polypeptide of SEQ ID NO: 782, and Example 2, wherein indels were measured at four B2M target sequences following delivery of an RNA guide and Cas12i2 polypeptide of SEQ ID NO: 783 by RNP.

In some embodiments, the direct repeat sequence is or comprises a sequence that is at least 90% identical to the reverse complement of any one of SEQ ID NOs: 1-10. In some embodiments, the direct repeat sequence is or comprises the reverse complement of any one of SEQ ID NOs: 1-10.

TABLE 1
Cas12i2 direct repeat sequences.

| Sequence | |
|---|---|
| identifier | Direct Repeat Sequence |
| SEQ ID NO: 1 | GUUGCAAAACCCAAGAAAUCCGUCUUUCAUUGACGG |
| SEQ ID NO: 2 | AAUAGCGGCCCUAAGAAAUCCGUCUUUCAUUGACGG |
| SEQ ID NO: 3 | AUUGGAACUGGCGAGAAAUCCGUCUUUCAUUGACGG |
| SEQ ID NO: 4 | CCAGCAACACCUAAGAAAUCCGUCUUUCAUUGACGG |
| SEQ ID NO: 5 | CGGCGCUCGAAUAGGAAAUCCGUCUUUCAUUGACGG |
| SEQ ID NO: 6 | GUGGCAACACCUAAGAAAUCCGUCUUUCAUUGACGG |
| SEQ ID NO: 7 | GUUGCAACACCUAAGAAAUCCGUCUUUCAUUGACGG |
| SEQ ID NO: 8 | GUUGCAAUGCCUAAGAAAUCCGUCUUUCAUUGACGG |
| SEQ ID NO: 9 | GCAACACCUAAGAAAUCCGUCUUUCAUUGACGGG |
| SEQ ID NO: 10 | AGAAAUCCGUCUUUCAUUGACGG |

In some embodiments, the direct repeat sequence is a sequence of Table 2 or a portion of a sequence of Table 2. The direct repeat sequence can comprise nucleotide 1 through nucleotide 36 of any one of SEQ ID NOs: 788, 789, 790, 791, 792, 793, 794, 795, 796, 797, 798, 799, 800, 801, 802, 803, 804, or 805. The direct repeat sequence can comprise nucleotide 2 through nucleotide 36 of any one of SEQ ID NOs: 788, 789, 790, 791, 792, 793, 794, 795, 796, 797, 798, 799, 800, 801, 802, 803, 804, or 805. The direct repeat sequence can comprise nucleotide 3 through nucleotide 36 of any one of SEQ ID NOs: 788, 789, 790, 791, 792, 793, 794, 795, 796, 797, 798, 799, 800, 801, 802, 803, 804, or 805. The direct repeat sequence can comprise nucleotide 4 through nucleotide 36 of any one of SEQ ID NOs: 788, 789, 790, 791, 792, 793, 794, 795, 796, 797, 798, 799, 800, 801, 802, 803, 804, or 805. The direct repeat sequence can comprise nucleotide 5 through nucleotide 36 of any one of SEQ ID NOs: 788, 789, 790, 791, 792, 793, 794, 795, 796, 797, 798, 799, 800, 801, 802, 803, 804, or 805. The direct repeat sequence can comprise nucleotide 6 through nucleotide 36 of any one of SEQ ID NOs: 788, 789, 790, 791, 792, 793, 794, 795, 796, 797, 798, 799, 800, 801, 802, 803, 804, or 805. The direct repeat sequence can comprise nucleotide 7 through nucleotide 36 of any one of SEQ ID NOs: 788, 789, 790, 791, 792, 793, 794, 795, 796, 797, 798, 799, 800, 801, 802, 803, 804, or 805. The direct repeat sequence can comprise nucleotide 8 through nucleotide 36 of any one of SEQ ID NOs: 788, 789, 790, 791, 792, 793, 794, 795, 796, 797, 798, 799, 800, 801, 802, 803, 804, or 805. The direct repeat sequence can comprise nucleotide 9 through nucleotide 36 of any one of SEQ ID NOs: 788, 789, 790, 791, 792, 793, 794, 795, 796, 797, 798, 799, 800, 801, 802, 803, 804, or 805. The direct repeat sequence can comprise nucleotide 10 through nucleotide 36 of any one of SEQ ID NOs: 788, 789, 790, 791, 792, 793, 794, 795, 796, 797, 798, 799, 800, 801, 802, 803, 804, or 805. The direct repeat sequence can comprise nucleotide 11 through nucleotide 36 of any one of SEQ ID NOs: 788, 789, 790, 791, 792, 793, 794, 795, 796, 797, 798, 799, 800, 801, 802, 803, 804, or 805. The direct repeat sequence can comprise nucleotide 12 through nucleotide 36 of any one of SEQ ID NOs: 788, 789, 790, 791, 792, 793, 794, 795, 796, 797, 798, 799, 800, 801, 802, 803, 804, or 805. The direct repeat sequence can comprise nucleotide 13 through nucleotide 36 of any one of SEQ ID NOs: 788, 789, 790, 791, 792, 793, 794, 795, 796, 797, 798, 799, 800, 801, 802, 803, 804, or 805. The direct repeat sequence can comprise nucleotide 14 through nucleotide 36 of any one of SEQ ID NOs: 788, 789, 790, 791, 792, 793, 794, 795, 796, 797, 798, 799, 800, 801, 802, 803, 804, or 805.

In some embodiments, the direct repeat sequence has at least 95% identity (e.g., at least 95%, 96%, 97%, 98% or 99% identity) to a sequence of Table 2 or a portion of a sequence of Table 2. The direct repeat sequence can have at least 95% identity to a sequence comprising nucleotide 1 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 95% identity to a sequence comprising 2 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 95% identity to a sequence comprising 3 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 95% identity to a sequence comprising 4 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 95% identity to a sequence comprising 5 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 95% identity to a sequence comprising 6 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 95% identity to a sequence comprising 7 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 95% identity to a sequence comprising 8 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 95% identity to a sequence comprising 9 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 95% identity to a sequence comprising 10 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 95% identity to a sequence comprising 11 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 95% identity to a sequence comprising 12 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 95% identity to a sequence comprising 13 through nucleotide 36 of any one of SEQ ID NOs: 788, 789, 790, 791, 792, 793, 794, 795, 796, 797, 798, 799, 800, 801, 802, 803, 804, or 805.

In some embodiments, the direct repeat sequence has at least 90% identity (e.g., at least 90%, 91%, 92%, 93%, 94%, 95%, 96%, 97%, 98% or 99% identity) to a sequence of Table 2 or a portion of a sequence of Table 2. The direct repeat sequence can have at least 90% identity to a sequence comprising nucleotide 1 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 90% identity to a sequence comprising 2 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 90% identity to a sequence comprising 3 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 90% identity to a sequence comprising 4 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 90% identity to a sequence comprising 5 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 90% identity to a sequence comprising 6 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 90% identity to a sequence comprising 7 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 90% identity to a sequence comprising 8 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 90% identity to a sequence comprising 9 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 90% identity to a sequence comprising 10 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 90% identity to a sequence comprising 11 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 90% identity to a sequence comprising 12 through nucleotide 36 of any one of SEQ ID NOs: 788, 789, 790, 791, 792, 793, 794, 795, 796, 797, 798, 799, 800, 801, 802, 803, 804, or 805. The direct repeat sequence can have at least 90% identity to a sequence comprising 13 through nucleotide 36 of any one of SEQ ID NOs: 788, 789, 790, 791, 792, 793, 794, 795, 796, 797, 798, 799, 800, 801, 802, 803, 804, or 805.

In some embodiments, the direct repeat sequence is at least 90% identical to the reverse complement of any one of SEQ ID NOs: 788, 789, 790, 791, 792, 793, 794, 795, 796, 797, 798, 799, 800, 801, 802, 803, 804, or 805. In some embodiments, the direct repeat sequence is at least 95% identical to the reverse complement of any one of SEQ ID NOs: 788, 789, 790, 791, 792, 793, 794, 795, 796, 797, 798, 799, 800, 801, 802, 803, 804, or 805. In some embodiments, the direct repeat sequence is the reverse complement of any one of SEQ ID NOs: 788, 789, 790, 791, 792, 793, 794, 795, 796, 797, 798, 799, 800, 801, 802, 803, 804, or 805.

In some embodiments, the direct repeat sequence is at least 90% identical to SEQ ID NO: 806 or a portion of SEQ ID NO: 806. In some embodiments, the direct repeat sequence is at least 95% identical to SEQ ID NO: 806 or a portion of SEQ ID NO: 806. In some embodiments, the direct repeat sequence is 100% identical to SEQ ID NO: 806 or a portion of SEQ ID NO: 806.

TABLE 2
Cas12i4 direct repeat sequences.

| Sequence | |
|---|---|
| identifier | Direct Repeat Sequence |
| SEQ ID NO: | UCUCAACGAUAGUCAGACAUGUGUCCUCAGUGACAC |
| 788 | |
| SEQ ID NO: | UUUUAACAACACUCAGGCAUGUGUCCACAGUGACAC |
| 789 | |
| SEQ ID NO: | UUGAACGGAUACUCAGACAUGUGUUUCCAGUGACAC |
| 790 | |
| SEQ ID NO: | UGCCCUCAAUAGUCAGAUGUGUGUCCACAGUGACAC |
| 791 | |
| SEQ ID NO: | UCUCAAUGAUACUUAGAUACGUGUCCUCAGUGACAC |
| 792 | |
| SEQ ID NO: | UCUCAAUGAUACUCAGACAUGUGUCCCCAGUGACAC |
| 793 | |
| SEQ ID NO: | UCUCAAUGAUACUAAGACAUGUGUCCUCAGUGACAC |
| 794 | |
| SEQ ID NO: | UCUCAACUAUACUCAGACAUGUGUCCUCAGUGACAC |
| 795 | |
| SEQ ID NO: | UCUCAACGAUACUCAGACAUGUGUCCUCAGUGACAC |
| 796 | |
| SEQ ID NO: | UCUCAACGAUACUAAGAUAUGUGUCCUCAGCGACAC |
| 797 | |
| SEQ ID NO: | UCUCAACGAUACUAAGAUAUGUGUCCCCAGUGACAC |
| 798 | |
| SEQ ID NO: | UCUCAACGAUACUAAGAUAUGUGUCCACAGUGACAC |
| 799 | |
| SEQ ID NO: | UCUCAACAAUACUCAGACAUGUGUCCCCAGUGACAC |
| 800 | |
| SEQ ID NO: | UCUCAACAAUACUAAGGCAUGUGUCCCCAGUGACCC |
| 801 | |
| SEQ ID NO: | UCUCAAAGAUACUCAGACACGUGUCCCCAGUGACAC |
| 802 | |
| SEQ ID NO: | UCUCAAAAAUACUCAGACAUGUGUCCUCAGUGACAC |
| 803 | |
| SEQ ID NO: | GCGAAACAACAGUCAGACAUGUGUCCCCAGUGACAC |
| 804 | |
| SEQ ID NO: | CCUCAACGAUAUUAAGACAUGUGUCCGCAGUGACAC |
| 805 | |
| SEQ ID NO: | AGACAUGUGUCCUCAGUGACAC |
| 806 | |

In some embodiments, the direct repeat sequence is a sequence of Table 3 or a portion of a sequence of Table 3. In some embodiments, the direct repeat sequence has at least 95% identity (e.g., at least 95%, 96%, 97%, 98% or 99% identity) to a sequence of Table 3 or a portion of a sequence of Table 3. In some embodiments, the direct repeat sequence has at least 90% identity (e.g., at least 90%, 91%, 92%, 93%, 94%, 95%, 96%, 97%, 98% or 99% identity) to a sequence of Table 3 or a portion of a sequence of Table 3. In some embodiments, the direct repeat sequence is at least 90% identical to the reverse complement of any one of SEQ ID NOs: 807-809. In some embodiments, the direct repeat sequence is at least 95% identical to the reverse complement of any one of SEQ ID NOs: 807-809. In some embodiments, the direct repeat sequence is the reverse complement of any one of SEQ ID NOs: 807-809.

TABLE 3
Cas12i1 direct repeat sequences.

| Sequence | |
|---|---|
| identifier | Direct Repeat Sequence |
| SEQ ID NO: 807 | GUUGGAAUGACUAAUUUUUGUGCC |
| | CACCGUUGGCAC |
| SEQ ID NO: 808 | AAUUUUUGUGCCCAUCGUUGGCAC |
| SEQ ID NO: 809 | AUUUUUGUGCCCAUCGUUGGCAC |

In some embodiments, the direct repeat sequence is a sequence of Table 4 or a portion of a sequence of Table 4. In some embodiments, the direct repeat sequence has at least 95% identity (e.g., at least 95%, 96%, 97%, 98% or 99% identity) to a sequence of Table 4 or a portion of a sequence of Table 4. In some embodiments, the direct repeat sequence has at least 90% identity (e.g., at least 90%, 91%, 92%, 93%, 94%, 95%, 96%, 97%, 98% or 99% identity) to a sequence of Table 4 or a portion of a sequence of Table 4. In some embodiments, the direct repeat sequence is at least 90% identical to the reverse complement of any one of SEQ ID NOs: 810-812. In some embodiments, the direct repeat sequence is at least 95% identical to the reverse complement of any one of SEQ ID NOs: 810-812. In some embodiments, the direct repeat sequence is the reverse complement of any one of SEQ ID NOs: 810-812.

TABLE 4
Cas12i3 direct repeat sequences.

| Sequence | |
|---|---|
| identifier | Direct Repeat Sequence |
| SEQ ID NO: 810 | CUAGCAAUGACCUAAUAGUGUG |
| | UCCUUAGUUGACAU |
| SEQ ID NO: 811 | CCUACAAUACCUAAGAAAUCCG |
| | UCCUAAGUUGACGG |
| SEQ ID NO: 812 | AUAGUGUGUCCUUAGUUGACAU |

In some embodiments, a direct repeat sequence described herein comprises a uracil (U). In some embodiments, a direct repeat sequence described herein comprises a thymine (T). In some embodiments, a direct repeat sequence according to Tables 1-4 comprises a sequence comprising a thymine in one or more places indicated as uracil in Tables 1-4.

Spacer

In some embodiments, the RNA guide comprises a DNA targeting or spacer sequence. In some embodiments, the spacer sequence of the RNA guide has a length of between 12-100, 13-75, 14-50, or 15-30 nucleotides (e.g., 15, 16, 17, 18, 19, 20, 21, 22, 23, 24, 25, 26, 27, 28, 29, or 30 nucleotides) and is complementary a specific target sequence. In some embodiments, the spacer sequence is designed to be complementary to a specific DNA strand, e.g., of a genomic locus.

In some embodiments, the RNA guide spacer sequence is substantially identical to a complementary strand of a target sequence. In some embodiments, the RNA guide comprises a sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a complementary strand of a reference nucleic acid sequence, e.g., target sequence. The percent identity between two such nucleic acids can be determined manually by inspection of the two optimally aligned nucleic acid sequences or by using software programs or algorithms (e.g., BLAST, ALIGN, CLUSTAL) using standard parameters.

In some embodiments, the RNA guide comprises a spacer sequence that has a length of between 12-100, 13-75, 14-50, or 15-30 nucleotides (e.g., 15, 16, 17, 18, 19, 20, 21, 22, 23, 24, 25, 26, 27, 28, 29, or 30 nucleotides) and at least 80%, at least 90%, at least 95%, at least 96%, at least 97%, at least 98%, at least 99% complementary to a target sequence. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence. In some embodiments, the RNA guide comprises a sequence, e.g., RNA sequence, that is a length of up to 50 and at least 80%, at least 90%, at least 95%, at least 96%, at least 97%, at least 98%, at least 99% complementary to a target sequence. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence.

In some embodiments, the spacer sequence is or comprises a sequence of Table 5 or a portion of a sequence of Table 5. The target sequences listed in Table 5 and Table 6 are on the non-target strand of the B2M sequence. It should be understood that an indication of SEQ ID NOs: 391-770 or 1019-1218 should be considered as equivalent to a listing of SEQ ID NOs: 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, and 770, or 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, and 1218.

The spacer sequence can comprise nucleotide 1 through nucleotide 16 of any one of SEQ ID NOs: 391-770 or 1019-1218. The spacer sequence can comprise nucleotide 1 through nucleotide 17 of any one of SEQ ID NOs: 391-770 or 1019-1218. The spacer sequence can comprise nucleotide 1 through nucleotide 18 of any one of SEQ ID NOs: 391-770 or 1019-1218. The spacer sequence can comprise nucleotide 1 through nucleotide 19 of any one of SEQ ID NOs: 391-770 or 1019-1218. The spacer sequence can comprise nucleotide 1 through nucleotide 20 of any one of SEQ ID NOs: 391-770 or 1019-1218. The spacer sequence can comprise nucleotide 1 through nucleotide 21 of any one of SEQ ID NOs: 391-770. The spacer sequence can comprise nucleotide 1 through nucleotide 22 of any one of SEQ ID NOs: 391-552 and 554-770. The spacer sequence can comprise nucleotide 1 through nucleotide 23 of any one of SEQ ID NOs: 391-552 and 555-770. The spacer sequence can comprise nucleotide 1 through nucleotide 24 of any one of SEQ ID NOs: 391-552 and 556-770. The spacer sequence can comprise nucleotide 1 through nucleotide 25 of any one of SEQ ID NOs: 391-552 and 556-770. The spacer sequence can comprise nucleotide 1 through nucleotide 26 of any one of SEQ ID NOs: 391-552 and 556-770. The spacer sequence can comprise nucleotide 1 through nucleotide 27 of any one of SEQ ID NOs: 391-552 and 556-770. The spacer sequence can comprise nucleotide 1 through nucleotide 28 of any one of SEQ ID NOs: 391-552 and 556-770. The spacer sequence can comprise nucleotide 1 through nucleotide 29 of any one of SEQ ID NOs: 391-552 and 556-770. The spacer sequence can comprise nucleotide 1 through nucleotide 30 of any one of SEQ ID NOs: 391-540, 542-552, and 556-770.

In some embodiments, the spacer sequence has or comprises a sequence having at least 90% identity (e.g., at least 90%, 91%, 92%, 93%, 94%, 95%, 96%, 97%, 98% or 99% identity) to a sequence of Table 5 or a portion of a sequence of Table 5. The spacer sequence can have or comprise a sequence having at least 90% identity to a sequence comprising nucleotide 1 through nucleotide 16 of any one of SEQ ID NOs: 391-770 or 1019-1218. The spacer sequence can have or comprise a sequence having at least 90% identity to a sequence comprising nucleotide 1 through nucleotide 17 of any one of SEQ ID NOs: 391-770 or 1019-1218. The spacer sequence can have or comprise a sequence having at least 90% identity to a sequence comprising nucleotide 1 through nucleotide 18 of any one of SEQ ID NOs: 391-770 or 1019-1218. The spacer sequence can have or comprise a sequence having at least 90% identity to a sequence comprising nucleotide 1 through nucleotide 19 of any one of SEQ ID NOs: 391-770 or 1019-1218. The spacer sequence can have or comprise a sequence having at least 90% identity to a sequence comprising nucleotide 1 through nucleotide 20 of any one of SEQ ID NOs: 391-770 or 1019-1218. The spacer sequence can have or comprise a sequence having at least 90% identity to a sequence comprising nucleotide 1 through nucleotide 21 of any one of SEQ ID NOs: 391-770. The spacer sequence can have or comprise a sequence having at least 90% identity to a sequence comprising nucleotide 1 through nucleotide 22 of any one of SEQ ID NOs: 391-552 and 554-770. The spacer sequence can have or comprise a sequence having at least 90% identity to a sequence comprising nucleotide 1 through nucleotide 23 of any one of SEQ ID NOs: 391-552 and 555-770. The spacer sequence can have or comprise a sequence having at least 90% identity to a sequence comprising nucleotide 1 through nucleotide 24 of any one of SEQ ID NOs: 391-552 and 556-770. The spacer sequence can have or comprise a sequence having at least 90% identity to a sequence comprising nucleotide 1 through nucleotide 25 of any one of SEQ ID NOs: 391-552 and 556-770. The spacer sequence can have or comprise a sequence having at least 90% identity to a sequence comprising nucleotide 1 through nucleotide 26 of any one of SEQ ID NOs: 391-552 and 556-770. The spacer sequence can have or comprise a sequence having at least 90% identity to a sequence comprising nucleotide 1 through nucleotide 27 of any one of SEQ ID NOs: 391-552 and 556-770. The spacer sequence can have or comprise a sequence having at least 90% identity to a sequence comprising nucleotide 1 through nucleotide 28 of any one of SEQ ID NOs: 391-552 and 556-770. The spacer sequence can have or comprise a sequence having at least 90% identity to a sequence comprising nucleotide 1 through nucleotide 29 of any one of SEQ ID NOs: 391-552 and 556-770. The spacer sequence can have or comprise a sequence having at least 90% identity to a sequence comprising nucleotide 1 through nucleotide 30 of any one of SEQ ID NOs: 391-540, 542-552, and 556-770.

TABLE 5
Target and spacer sequences

| B2M exon | strand | PAM | target sequence | spacer sequence |
|---|---|---|---|---|
| B2M_exon_1 | + | TTTA | ATATAAGTGGAGGCGTCGCGCTG | AUAUAAGUGGAGGCGUCGCGCUGGCGGG |
| | | | GCGGGCA (SEQ ID NO: 11) | CA (SEQ ID NO: 391) |
| B2M_exon_1 | − | CTTA | TATTAAACGCGTGCCCAGCCAAT | UAUUAAACGCGUGCCCAGCCAAUCAGGA |
| | | | CAGGACA (SEQ ID NO: 12) | CA (SEQ ID NO: 392) |
| B2M_exon_1 | − | CTTC | AGGAATGCCCGCCAGCGCGACGC | AGGAAUGCCCGCCAGCGCGACGCCUCCA |
| | | | CTCCACT (SEQ ID NO: 13) | CU (SEQ ID NO: 393) |
| B2M_exon_1 | + | TTTC | TGGCCTGGAGGCTATCCAGCGTG | UGGCCUGGAGGCUAUCCAGCGUGAGUCU |
| | | | AGTCTCT (SEQ ID NO: 14) | CU (SEQ ID NO: 394) |
| B2M_exon_1 | + | CTTT | CTGGCCTGGAGGCTATCCAGCGT | CUGGCCUGGAGGCUAUCCAGCGUGAGUC |
| | | | GAGTCTC (SEQ ID NO: 15) | UC (SEQ ID NO: 395) |
| B2M_exon_1 | + | CTTA | GCTGTGCTCGCGCTACTCTCTCT | GCUGUGCUCGCGCUACUCUCUCUUUCUG |
| | | | TTCTGGC (SEQ ID NO: 16) | GC (SEQ ID NO: 396) |
| B2M_exon_1 | + | ATTC | GGGCCGAGATGTCTCGCTCCGTG | GGGCCGAGAUGUCUCGCUCCGUGGCCUU |
| | | | GCCTTAG (SEQ ID NO: 17) | AG (SEQ ID NO: 397) |
| B2M_exon_1 | + | ATTC | CTGAAGCTGACAGCATTCGGGCC | CUGAAGCUGACAGCAUUCGGGCCGAGAU |
| | | | GAGATGT (SEQ ID NO: 18) | GU (SEQ ID NO: 398) |
| B2M_exon_1 | + | GTTT | AATATAAGTGGAGGCGTCGCGCT | AAUAUAAGUGGAGGCGUCGCGCUGGCGG |
| | | | GGCGGGC (SEQ ID NO: 19) | GC (SEQ ID NO: 399) |
| B2M_exon_1 | + | ATTG | GCTGGGCACGCGTTTAATATAAG | GCUGGGCACGCGUUUAAUAUAAGUGGAG |
| | | | TGGAGGC (SEQ ID NO: 20) | GC (SEQ ID NO: 400) |
| B2M_exon_2 | + | TTTG | TCACAGCCCAAGATAGTTAAGTG | UCACAGCCCAAGAUAGUUAAGUGGGGUA |
| | | | GGGTAAG (SEQ ID NO: 21) | AG (SEQ ID NO: 401) |
| B2M_exon_2 | + | GTTA | AGTGGGGTAAGTCTTACATTCTT | AGUGGGGUAAGUCUUACAUUCUUUUGUA |
| | | | TTGTAAG (SEQ ID NO: 22) | AG (SEQ ID NO: 402) |
| B2M_exon_2 | − | TTTC | CATTCTCTGCTGGATGACGTGAG | CAUUCUCUGCUGGAUGACGUGAGUAAAC |
| | | | TAAACCT (SEQ ID NO: 23) | CU (SEQ ID NO: 403) |
| B2M_exon_2 | + | ATTC | TTTTGTAAGCTGCTGAAAGTTGT | UUUUGUAAGCUGCUGAAAGUUGUGUAUG |
| | | | GTATGAG (SEQ ID NO: 24) | AG (SEQ ID NO: 404) |
| B2M_exon_2 | + | CTTT | GTCACAGCCCAAGATAGTTAAGT | GUCACAGCCCAAGAUAGUUAAGUGGGGU |
| | | | GGGGTAA (SEQ ID NO: 25) | AA (SEQ ID NO: 405) |
| B2M_exon_2 | + | CTTA | CATTCTTTTGTAAGCTGCTGAAA | CAUUCUUUUGUAAGCUGCUGAAAGUUGU |
| | | | GTTGTGT (SEQ ID NO: 26) | GU (SEQ ID NO: 406) |
| B2M_exon_2 | + | ATTC | ACCCCCACTGAAAAAGATGAGTA | ACCCCCACUGAAAAAGAUGAGUAUGCCU |
| | | | TGCCTGC (SEQ ID NO: 27) | GC (SEQ ID NO: 407) |
| B2M_exon_2 | + | CTTT | CAGCAAGGACTGGTCTTTCTATC | CAGCAAGGACUGGUCUUUCUAUCUCUUG |
| | | | TCTTGTA (SEQ ID NO: 28) | UA (SEQ ID NO: 408) |
| B2M_exon_2 | + | TTTC | TATCTCTTGTACTACACTGAATT | UAUCUCUUGUACUACACUGAAUUCACCC |
| | | | CACCCCC (SEQ ID NO: 29) | CC (SEQ ID NO: 409) |
| B2M_exon_2 | + | CTTT | CTATCTCTTGTACTACACTGAAT | CUAUCUCUUGUACUACACUGAAUUCACC |
| | | | TCACCCC (SEQ ID NO: 30) | CC (SEQ ID NO: 410) |
| B2M_exon_2 | + | TTTC | AGCAAGGACTGGTCTTTCTATCT | AGCAAGGACUGGUCUUUCUAUCUCUUGU |
| | | | CTTGTAC (SEQ ID NO: 31) | AC (SEQ ID NO: 411) |
| B2M_exon_2 | + | CTTT | TGTAAGCTGCTGAAAGTTGTGTA | UGUAAGCUGCUGAAAGUUGUGUAUGAGU |
| | | | TGAGTAG (SEQ ID NO: 32) | AG (SEQ ID NO: 412) |
| B2M_exon_2 | + | CTTG | TCTTTCAGCAAGGACTGGTCTTT | UCUUUCAGCAAGGACUGGUCUUUCUAUC |
| | | | CTATCTC (SEQ ID NO: 33) | UC (SEQ ID NO: 413) |
| B2M_exon_2 | + | CTTG | TACTACACTGAATTCACCCCCAC | UACUACACUGAAUUCACCCCCACUGAAA |
| | | | TGAAAAA (SEQ ID NO: 34) | AA (SEQ ID NO: 414) |
| B2M_exon_2 | + | TTTT | GTAAGCTGCTGAAAGTTGTGTAT | GUAAGCUGCUGAAAGUUGUGUAUGAGUA |
| | | | GAGTAGT (SEQ ID NO: 35) | GU (SEQ ID NO: 415) |
| B2M_exon_2 | − | CTTA | CCCCACTTAACTATCTTGGGCTG | CCCCACUUAACUAUCUUGGGCUGUGACA |
| | | | TGACAAA (SEQ ID NO: 36) | AA (SEQ ID NO: 416) |
| B2M_exon_2 | − | CTTT | CAGCAGCTTACAAAAGAATGTAA | CAGCAGCUUACAAAAGAAUGUAAGACUU |
| | | | GACTTAC (SEQ ID NO: 37) | AC (SEQ ID NO: 417) |
| B2M_exon_2 | − | TTTC | AGCAGCTTACAAAAGAATGTAAG | AGCAGCUUACAAAAGAAUGUAAGACUUA |
| | | | ACTTACC (SEQ ID NO: 38) | CC (SEQ ID NO: 418) |
| B2M_exon_2 | − | CTTA | CAAAAGAATGTAAGACTTACCCC | CAAAAGAAUGUAAGACUUACCCCACUUA |
| | | | ACTTAAC (SEQ ID NO: 39) | AC (SEQ ID NO: 419) |
| B2M_exon_2 | + | ATTC | AGACTTGTCTTTCAGCAAGGACT | AGACUUGUCUUUCAGCAAGGACUGGUCU |
| | | | GGTCTTT (SEQ ID NO: 40) | UU (SEQ ID NO: 420) |
| B2M_exon_2 | − | CTTA | ACTATCTTGGGCTGTGACAAAGT | ACUAUCUUGGGCUGUGACAAAGUCACAU |
| | | | CACATGG (SEQ ID NO: 41) | GG (SEQ ID NO: 421) |
| B2M_exon_2 | − | CTTG | GGCTGTGACAAAGTCACATGGTT | GGCUGUGACAAAGUCACAUGGUUCACAC |
| | | | CACACGG (SEQ ID NO: 42) | GG (SEQ ID NO: 422) |
| B2M_exon_2 | − | GTTC | ACACGGCAGGCATACTCATCTTT | ACACGGCAGGCAUACUCAUCUUUUUCAG |
| | | | TTCAGTG (SEQ ID NO: 43) | UG (SEQ ID NO: 423) |
| B2M_exon_2 | − | CTTT | TTCAGTGGGGGTGAATTCAGTGT | UUCAGUGGGGGUGAAUUCAGUGUAGUAC |
| | | | AGTACAA (SEQ ID NO: 44) | AA (SEQ ID NO: 424) |
| B2M_exon_2 | − | TTTT | TCAGTGGGGGTGAATTCAGTGTA | UCAGUGGGGGUGAAUUCAGUGUAGUACA |
| | | | GTACAAG (SEQ ID NO: 45) | AG (SEQ ID NO: 425) |
| B2M_exon_2 | − | TTTT | CAGTGGGGGTGAATTCAGTGTAG | CAGUGGGGGUGAAUUCAGUGUAGUACAA |
| | | | TACAAGA (SEQ ID NO: 46) | GA (SEQ ID NO: 426) |
| B2M_exon_2 | − | TTTC | AGTGGGGGTGAATTCAGTGTAGT | AGUGGGGGUGAAUUCAGUGUAGUACAAG |
| | | | ACAAGAG (SEQ ID NO: 47) | AG (SEQ ID NO: 427) |
| B2M_exon_2 | − | ATTC | AGTGTAGTACAAGAGATAGAAAG | AGUGUAGUACAAGAGAUAGAAAGACCAG |
| | | | ACCAGTC (SEQ ID NO: 48) | UC (SEQ ID NO: 428) |
| B2M_exon_2 | − | CTTG | CTGAAAGACAAGTCTGAATGCTC | CUGAAAGACAAGUCUGAAUGCUCCACUU |
| | | | CACTTTT (SEQ ID NO: 49) | UU (SEQ ID NO: 429) |
| B2M_exon_2 | + | TTTG | TAAGCTGCTGAAAGTTGTGTATG | UAAGCUGCUGAAAGUUGUGUAUGAGUAG |
| | | | AGTAGTC (SEQ ID NO: 50) | UC (SEQ ID NO: 430) |
| B2M_exon_2 | + | ATTG | AAAAAGTGGAGCATTCAGACTTG | AAAAAGUGGAGCAUUCAGACUUGUCUUU |
| | | | TCTTTCA (SEQ ID NO: 51) | CA (SEQ ID NO: 431) |
| B2M_exon_2 | + | GTTT | CATCCATCCGACATTGAAGTTGA | CAUCCAUCCGACAUUGAAGUUGACUUAC |
| | | | CTTACTG (SEQ ID NO: 52) | UG (SEQ ID NO: 432) |
| B2M_exon_2 | + | GTTG | ACTTACTGAAGAATGGAGAGAGA | ACUUACUGAAGAAUGGAGAGAGAAUUGA |
| | | | ATTGAAA (SEQ ID NO: 53) | AA (SEQ ID NO: 433) |
| B2M_exon_2 | − | ATTA | ATATTGCCAGGGTATTTCACTTG | AUAUUGCCAGGGUAUUUCACUUGGGGCU |
| | | | GGGCTAA (SEQ ID NO: 54) | AA (SEQ ID NO: 434) |
| B2M_exon_2 | − | TTTG | GAGTACCTGAGGAATATCGGGAA | GAGUACCUGAGGAAUAUCGGGAAAAGAC |
| | | | AAGACAC (SEQ ID NO: 55) | AC (SEQ ID NO: 435) |
| B2M_exon_2 | − | CTTT | GGAGTACCTGAGGAATATCGGGA | GGAGUACCUGAGGAAUAUCGGGAAAAGA |
| | | | AAAGACA (SEQ ID NO: 56) | CA (SEQ ID NO: 436) |
| B2M_exon_2 | − | ATTC | TCTGCTGGATGACGTGAGTAAAC | UCUGCUGGAUGACGUGAGUAAACCUGAA |
| | | | CTGAATC (SEQ ID NO: 57) | UC (SEQ ID NO: 437) |
| B2M_exon_2 | − | CTTT | CCATTCTCTGCTGGATGACGTGA | CCAUUCUCUGCUGGAUGACGUGAGUAAA |
| | | | GTAAACC (SEQ ID NO: 58) | CC (SEQ ID NO: 438) |
| B2M_exon_2 | − | TTTG | ACTTTCCATTCTCTGCTGGATGA | ACUUUCCAUUCUCUGCUGGAUGACGUGA |
| | | | CGTGAGT (SEQ ID NO: 59) | GU (SEQ ID NO: 439) |
| B2M_exon_2 | − | ATTT | GACTTTCCATTCTCTGCTGGATG | GACUUUCCAUUCUCUGCUGGAUGACGUG |
| | | | ACGTGAG (SEQ ID NO: 60) | AG (SEQ ID NO: 440) |
| B2M_exon_2 | − | ATTC | AGGAAATTTGACTTTCCATTCTC | AGGAAAUUUGACUUUCCAUUCUCUGCUG |
| | | | TGCTGGA (SEQ ID NO: 61) | GA (SEQ ID NO: 441) |
| B2M_exon_2 | − | CTTC | AATGTCGGATGGATGAAACCCAG | AAUGUCGGAUGGAUGAAACCCAGACACA |
| | | | ACACATA (SEQ ID NO: 62) | UA (SEQ ID NO: 442) |
| B2M_exon_2 | − | CTTC | AGTAAGTCAACTTCAATGTCGGA | AGUAAGUCAACUUCAAUGUCGGAUGGAU |
| | | | TGGATGA (SEQ ID NO: 63) | GA (SEQ ID NO: 443) |
| B2M_exon_2 | − | ATTC | TTCAGTAAGTCAACTTCAATGTC | UUCAGUAAGUCAACUUCAAUGUCGGAUG |
| | | | GGATGGA (SEQ ID NO: 64) | GA (SEQ ID NO: 444) |
| B2M_exon_2 | − | ATTC | TCTCTCCATTCTTCAGTAAGTCA | UCUCUCCAUUCUUCAGUAAGUCAACUUC |
| | | | ACTTCAA (SEQ ID NO: 65) | AA (SEQ ID NO: 445) |
| B2M_exon_2 | − | TTTC | AATTCTCTCTCCATTCTTCAGTA | AAUUCUCUCUCCAUUCUUCAGUAAGUCA |
| | | | AGTCAAC (SEQ ID NO: 66) | AC (SEQ ID NO: 446) |
| B2M_exon_2 | + | CTTA | CTGAAGAATGGAGAGAGAATTGA | CUGAAGAAUGGAGAGAGAAUUGAAAAAG |
| | | | AAAAGTG (SEQ ID NO: 67) | UG (SEQ ID NO: 447) |
| B2M_exon_2 | − | TTTT | CAATTCTCTCTCCATTCTTCAGT | CAAUUCUCUCUCCAUUCUUCAGUAAGUC |
| | | | AAGTCAA (SEQ ID NO: 68) | AA (SEQ ID NO: 448) |
| B2M_exon_2 | + | CTTT | TCCCGATATTCCTCAGGTACTCC | UCCCGAUAUUCCUCAGGUACUCCAAAGA |
| | | | AAAGATT (SEQ ID NO: 69) | UU (SEQ ID NO: 449) |
| B2M_exon_2 | + | TTTT | CCCGATATTCCTCAGGTACTCCA | CCCGAUAUUCCUCAGGUACUCCAAAGAU |
| | | | AAGATTC (SEQ ID NO: 70) | UC (SEQ ID NO: 450) |
| B2M_exon_2 | + | TTTC | CCGATATTCCTCAGGTACTCCAA | CCGAUAUUCCUCAGGUACUCCAAAGAUU |
| | | | AGATTCA (SEQ ID NO: 71) | CA (SEQ ID NO: 451) |
| B2M_exon_2 | + | ATTC | CTCAGGTACTCCAAAGATTCAGG | CUCAGGUACUCCAAAGAUUCAGGUUUAC |
| | | | TTTACTC (SEQ ID NO: 72) | UC (SEQ ID NO: 452) |
| B2M_exon_2 | + | ATTC | AGGTTTACTCACGTCATCCAGCA | AGGUUUACUCACGUCAUCCAGCAGAGAA |
| | | | GAGAATG (SEQ ID NO: 73) | UG (SEQ ID NO: 453) |
| B2M_exon_2 | + | GTTT | ACTCACGTCATCCAGCAGAGAAT | ACUCACGUCAUCCAGCAGAGAAUGGAAA |
| | | | GGAAAGT (SEQ ID NO: 74) | GU (SEQ ID NO: 454) |
| B2M_exon_2 | + | TTTA | CTCACGTCATCCAGCAGAGAATG | CUCACGUCAUCCAGCAGAGAAUGGAAAG |
| | | | GAAAGTC (SEQ ID NO: 75) | UC (SEQ ID NO: 455) |
| B2M_exon_2 | + | ATTT | CCTGAATTGCTATGTGTCTGGGT | CCUGAAUUGCUAUGUGUCUGGGUUUCAU |
| | | | TTCATCC (SEQ ID NO: 76) | CC (SEQ ID NO: 456) |
| B2M_exon_2 | + | TTTC | CTGAATTGCTATGTGTCTGGGTT | CUGAAUUGCUAUGUGUCUGGGUUUCAUC |
| | | | TCATCCA (SEQ ID NO: 77) | CA (SEQ ID NO: 457) |
| B2M_exon_2 | + | ATTG | CTATGTGTCTGGGTTTCATCCAT | CUAUGUGUCUGGGUUUCAUCCAUCCGAC |
| | | | CCGACAT (SEQ ID NO: 78) | AU (SEQ ID NO: 458) |
| B2M_exon_2 | − | CTTT | TTCAATTCTCTCTCCATTCTTCA | UUCAAUUCUCUCUCCAUUCUUCAGUAAG |
| | | | GTAAGTC (SEQ ID NO: 79) | UC (SEQ ID NO: 459) |
| B2M_exon_2 | + | TTTC | ATCCATCCGACATTGAAGTTGAC | AUCCAUCCGACAUUGAAGUUGACUUACU |
| | | | TTACTGA (SEQ ID NO: 80) | GA (SEQ ID NO: 460) |
| B2M_exon_2 | + | ATTG | AAGTTGACTTACTGAAGAATGGA | AAGUUGACUUACUGAAGAAUGGAGAGAG |
| | | | GAGAGAA (SEQ ID NO: 81) | AA (SEQ ID NO: 461) |
| B2M_exon_2 | + | ATTA | ATGTGTCTTTTCCCGATATTCCT | AUGUGUCUUUUCCCGAUAUUCCUCAGGU |
| | | | CAGGTAC (SEQ ID NO: 82) | AC (SEQ ID NO: 462) |
| B2M_exon_2 | − | TTTT | TCAATTCTCTCTCCATTCTTCAG | UCAAUUCUCUCUCCAUUCUUCAGUAAGU |
| | | | TAAGTCA (SEQ ID NO: 83) | CA (SEQ ID NO: 463) |
| B2M_exon_3 | + | CTTA | ATGTCTTCCTTTTTTTTCTCCAC | AUGUCUUCCUUUUUUUUCUCCACUGUCU |
| | | | TGTCTTT (SEQ ID NO: 84) | UU (SEQ ID NO: 464) |
| B2M_exon_3 | − | CTTA | CCTCCATGATGCTGCTTACATGT | CCUCCAUGAUGCUGCUUACAUGUCUCGA |
| | | | CTCGATC (SEQ ID NO: 85) | UC (SEQ ID NO: 465) |
| B2M_exon_3 | − | CTTA | CATGTCTCGATCTATGAAAAAGA | CAUGUCUCGAUCUAUGAAAAAGACAGUG |
| | | | CAGTGGA (SEQ ID NO: 86) | GA (SEQ ID NO: 466) |
| B2M_exon_3 | + | CTTC | CTTTTTTTTCTCCACTGTCTTTT | CUUUUUUUUCUCCACUGUCUUUUUCAUA |
| | | | TCATAGA (SEQ ID NO: 87) | GA (SEQ ID NO: 467) |
| B2M_exon_3 | + | CTTT | TTTTTCTCCACTGTCTTTTTCAT | UUUUUCUCCACUGUCUUUUUCAUAGAUC |
| | | | AGATCGA (SEQ ID NO: 88) | GA (SEQ ID NO: 468) |
| B2M_exon_3 | + | TTTT | TTTTCTCCACTGTCTTTTTCATA | UUUUCUCCACUGUCUUUUUCAUAGAUCG |
| | | | GATCGAG (SEQ ID NO: 89) | AG (SEQ ID NO: 469) |
| B2M_exon_3 | + | TTTT | TTTCTCCACTGTCTTTTTCATAG | UUUCUCCACUGUCUUUUUCAUAGAUCGA |
| | | | ATCGAGA (SEQ ID NO: 90) | GA (SEQ ID NO: 470) |
| B2M_exon_3 | + | TTTT | TCTCCACTGTCTTTTTCATAGAT | UCUCCACUGUCUUUUUCAUAGAUCGAGA |
| | | | CGAGACA (SEQ ID NO: 91) | CA (SEQ ID NO: 471) |
| B2M_exon_3 | + | TTTT | CTCCACTGTCTTTTTCATAGATC | CUCCACUGUCUUUUUCAUAGAUCGAGAC |
| | | | GAGACAT (SEQ ID NO: 92) | AU (SEQ ID NO: 472) |
| B2M_exon_3 | + | TTTC | TCCACTGTCTTTTTCATAGATCG | UCCACUGUCUUUUUCAUAGAUCGAGACA |
| | | | AGACATG (SEQ ID NO: 93) | UG (SEQ ID NO: 473) |
| B2M_exon_3 | + | CTTT | TTCATAGATCGAGACATGTAAGC | UUCAUAGAUCGAGACAUGUAAGCAGCAU |
| | | | AGCATCA (SEQ ID NO: 94) | CA (SEQ ID NO: 474) |
| B2M_exon_3 | + | TTTT | TCATAGATCGAGACATGTAAGCA | UCAUAGAUCGAGACAUGUAAGCAGCAUC |
| | | | GCATCAT (SEQ ID NO: 95) | AU (SEQ ID NO: 475) |
| B2M_exon_3 | + | TTTT | CATAGATCGAGACATGTAAGCAG | CAUAGAUCGAGACAUGUAAGCAGCAUCA |
| | | | CATCATG (SEQ ID NO: 96) | UG (SEQ ID NO: 476) |
| B2M_exon_3 | + | TTTT | TTCTCCACTGTCTTTTTCATAGA | UUCUCCACUGUCUUUUUCAUAGAUCGAG |
| | | | TCGAGAC (SEQ ID NO: 97) | AC (SEQ ID NO: 477) |
| B2M_exon_3 | + | GTTT | TTGACCTTGAGAAAATGTTTTTG | UUGACCUUGAGAAAAUGUUUUUGUUUCA |
| | | | TTTCACT (SEQ ID NO: 98) | CU (SEQ ID NO: 478) |
| B2M_exon_3 | + | TTTC | ATAGATCGAGACATGTAAGCAGC | AUAGAUCGAGACAUGUAAGCAGCAUCAU |
| | | | ATCATGG (SEQ ID NO: 99) | GG (SEQ ID NO: 479) |
| B2M_exon_3 | − | TTTT | CTCAAGGTCAAAAACTTACCTCC | CUCAAGGUCAAAAACUUACCUCCAUGAU |
| | | | ATGATGC (SEQ ID NO: | GC (SEQ ID NO: 480) |
| | | | 100) | |
| B2M_exon_3 | − | ATTT | TCTCAAGGTCAAAAACTTACCTC | UCUCAAGGUCAAAAACUUACCUCCAUGA |
| | | | CATGATG (SEQ ID NO: | UG (SEQ ID NO: 481) |
| | | | 101) | |
| B2M_exon_3 | − | TTTC | TCAAGGTCAAAAACTTACCTCCA | UCAAGGUCAAAAACUUACCUCCAUGAUG |
| | | | TGATGCT (SEQ ID NO: | CU (SEQ ID NO: 482) |
| | | | 102) | |
| B2M_exon_3 | + | GTTT | TTGTTTCACTGTCCTGAGGACTA | UUGUUUCACUGUCCUGAGGACUAUUUAU |
| | | | TTTATAG (SEQ ID NO: | AG (SEQ ID NO: 483) |
| | | | 103) | |
| B2M_exon_3 | + | TTTT | TGTTTCACTGTCCTGAGGACTAT | UGUUUCACUGUCCUGAGGACUAUUUAUA |
| | | | TTATAGA (SEQ ID NO: | GA (SEQ ID NO: 484) |
| | | | 104) | |
| B2M_exon_3 | + | CTTG | AGAAAATGTTTTTGTTTCACTGT | AGAAAAUGUUUUUGUUUCACUGUCCUGA |
| | | | CCTGAGG (SEQ ID NO: | GG (SEQ ID NO: 485) |
| | | | 105) | |
| B2M_exon_3 | + | TTTG | ACCTTGAGAAAATGTTTTTGTTT | ACCUUGAGAAAAUGUUUUUGUUUCACUG |
| | | | CACTGTC (SEQ ID NO: | UC (SEQ ID NO: 486) |
| | | | 106) | |
| B2M_exon_3 | + | TTTT | GACCTTGAGAAAATGTTTTTGTT | GACCUUGAGAAAAUGUUUUUGUUUCACU |
| | | | TCACTGT (SEQ ID NO: | GU (SEQ ID NO: 487) |
| | | | 107) | |
| B2M_exon_3 | + | TTTT | TGACCTTGAGAAAATGTTTTTGT | UGACCUUGAGAAAAUGUUUUUGUUUCAC |
| | | | TTCACTG (SEQ ID NO: | UG (SEQ ID NO: 488) |
| | | | 108) | |
| B2M_exon_4 | − | ATTG | TATTAGGTATTACAAGTAATCTA | UAUUAGGUAUUACAAGUAAUCUAGAAAU |
| | | | GAAATGA (SEQ ID NO: | GA (SEQ ID NO: 489) |
| | | | 109) | |
| B2M_exon_4 | − | ATTT | ACATTGTATTAGGTATTACAAGT | ACAUUGUAUUAGGUAUUACAAGUAAUCU |
| | | | AATCTAG (SEQ ID NO: | AG (SEQ ID NO: 490) |
| | | | 110) | |
| B2M_exon_4 | − | TTTG | CATAGCATTTACATTGTATTAGG | CAUAGCAUUUACAUUGUAUUAGGUAUUA |
| | | | TATTACA (SEQ ID NO: | CA (SEQ ID NO: 491) |
| | | | 111) | |
| B2M_exon_4 | − | ATTT | GCATAGCATTTACATTGTATTAG | GCAUAGCAUUUACAUUGUAUUAGGUAUU |
| | | | GTATTAC (SEQ ID NO: | AC (SEQ ID NO: 492) |
| | | | 112) | |
| B2M_exon_4 | − | TTTA | CATTGTATTAGGTATTACAAGTA | CAUUGUAUUAGGUAUUACAAGUAAUCUA |
| | | | ATCTAGA (SEQ ID NO: | GA (SEQ ID NO: 493) |
| | | | 113) | |
| B2M_exon_4 | − | CTTA | AACAATAACAACTATTTGCATAG | AACAAUAACAACUAUUUGCAUAGCAUUU |
| | | | CATTTAC (SEQ ID NO: | AC (SEQ ID NO: 494) |
| | | | 114) | |
| B2M_exon_4 | − | TTTT | CTTGTCATTATTCCTTAAACAAT | CUUGUCAUUAUUCCUUAAACAAUAACAA |
| | | | AACAACT (SEQ ID NO: | CU (SEQ ID NO: 495) |
| | | | 115) | |
| B2M_exon_4 | − | ATTA | TTCCTTAAACAATAACAACTATT | UUCCUUAAACAAUAACAACUAUUUGCAU |
| | | | TGCATAG (SEQ ID NO: | AG (SEQ ID NO: 496) |
| | | | 116) | |
| B2M_exon_4 | − | CTTG | TCATTATTCCTTAAACAATAACA | UCAUUAUUCCUUAAACAAUAACAACUAU |
| | | | ACTATTT (SEQ ID NO: | UU (SEQ ID NO: 497) |
| | | | 117) | |
| B2M_exon_4 | − | TTTC | TTGTCATTATTCCTTAAACAATA | UUGUCAUUAUUCCUUAAACAAUAACAAC |
| | | | ACAACTA (SEQ ID NO: | UA (SEQ ID NO: 498) |
| | | | 118) | |
| B2M_exon_4 | − | TTTT | TCTTGTCATTATTCCTTAAACAA | UCUUGUCAUUAUUCCUUAAACAAUAACA |
| | | | TAACAAC (SEQ ID NO: | AC (SEQ ID NO: 499) |
| | | | 119) | |
| B2M_exon_4 | − | ATTA | GGTATTACAAGTAATCTAGAAAT | GGUAUUACAAGUAAUCUAGAAAUGAUUU |
| | | | GATTTAA (SEQ ID NO: | AA (SEQ ID NO: 500) |
| | | | 120) | |
| B2M_exon_4 | − | TTTT | TTCTTGTCATTATTCCTTAAACA | UUCUUGUCAUUAUUCCUUAAACAAUAAC |
| | | | ATAACAA (SEQ ID NO: | AA (SEQ ID NO: 501) |
| | | | 121) | |
| B2M_exon_4 | − | ATTC | CTTAAACAATAACAACTATTTGC | CUUAAACAAUAACAACUAUUUGCAUAGC |
| | | | ATAGCAT (SEQ ID NO: | AU (SEQ ID NO: 502) |
| | | | 122) | |
| B2M_exon_4 | − | ATTA | CAAGTAATCTAGAAATGATTTAA | CAAGUAAUCUAGAAAUGAUUUAAAGUAU |
| | | | AGTATAC (SEQ ID NO: | AC (SEQ ID NO: 503) |
| | | | 123) | |
| B2M_exon_4 | − | ATTC | CTTGCTAAAATATTAAATCCTTC | CUUGCUAAAAUAUUAAAUCCUUCAGAUA |
| | | | AGATACT (SEQ ID NO: | CU (SEQ ID NO: 504) |
| | | | 124) | |
| B2M_exon_4 | − | TTTA | AAGTATACAGGAGGATGTGGATA | AAGUAUACAGGAGGAUGUGGAUAGGUUA |
| | | | GGTTATA (SEQ ID NO: | UA (SEQ ID NO: 505) |
| | | | 125) | |
| B2M_exon_4 | − | TTTT | GCTCCCTCTTAGAGTCTGCATAC | GCUCCCUCUUAGAGUCUGCAUACUCCUC |
| | | | TCCTCAT (SEQ ID NO: | AU (SEQ ID NO: 506) |
| | | | 126) | |
| B2M_exon_4 | − | CTTT | TGCTCCCTCTTAGAGTCTGCATA | UGCUCCCUCUUAGAGUCUGCAUACUCCU |
| | | | CTCCTCA (SEQ ID NO: | CA (SEQ ID NO: 507) |
| | | | 127) | |
| B2M_exon_4 | − | CTTC | AGATACTTTTGCTCCCTCTTAGA | AGAUACUUUUGCUCCCUCUUAGAGUCUG |
| | | | GTCTGCA (SEQ ID NO: | CA (SEQ ID NO: 508) |
| | | | 128) | |
| B2M_exon_4 | − | ATTA | AATCCTTCAGATACTTTTGCTCC | AAUCCUUCAGAUACUUUUGCUCCCUCUU |
| | | | CTCTTAG (SEQ ID NO: | AG (SEQ ID NO: 509) |
| | | | 129) | |
| B2M_exon_4 | − | CTTG | CTAAAATATTAAATCCTTCAGAT | CUAAAAUAUUAAAUCCUUCAGAUACUUU |
| | | | ACTTTTG (SEQ ID NO: | UG (SEQ ID NO: 510) |
| | | | 130) | |
| B2M_exon_4 | − | TTTT | TTTCTTGTCATTATTCCTTAAAC | UUUCUUGUCAUUAUUCCUUAAACAAUAA |
| | | | AATAACA (SEQ ID NO: | CA (SEQ ID NO: 511) |
| | | | 131) | |
| B2M_exon_4 | − | ATTT | AAAGTATACAGGAGGATGTGGAT | AAAGUAUACAGGAGGAUGUGGAUAGGUU |
| | | | AGGTTAT (SEQ ID NO: | AU (SEQ ID NO: 512) |
| | | | 132) | |
| B2M_exon_4 | − | ATTG | TATATCTATTCCTTGCTAAAATA | UAUAUCUAUUCCUUGCUAAAAUAUUAAA |
| | | | TTAAATC (SEQ ID NO: | UC (SEQ ID NO: 513) |
| | | | 133) | |
| B2M_exon_4 | − | ATTT | GGTGTCCAAGAAGGGGTCCTGAA | GGUGUCCAAGAAGGGGUCCUGAAACCAA |
| | | | ACCAATC (SEQ ID NO: | UC (SEQ ID NO: 514) |
| | | | 134) | |
| B2M_exon_4 | − | CTTA | AATATCCATAGATTTGGTGTCCA | AAUAUCCAUAGAUUUGGUGUCCAAGAAG |
| | | | AGAAGGG (SEQ ID NO: | GG (SEQ ID NO: 515) |
| | | | 135) | |
| B2M_exon_4 | − | TTTA | TAGAAGGGACTTAAATATCCATA | UAGAAGGGACUUAAAUAUCCAUAGAUUU |
| | | | GATTTGG (SEQ ID NO: | GG (SEQ ID NO: 516) |
| | | | 136) | |
| B2M_exon_4 | − | TTTT | ATAGAAGGGACTTAAATATCCAT | AUAGAAGGGACUUAAAUAUCCAUAGAUU |
| | | | AGATTTG (SEQ ID NO: | UG (SEQ ID NO: 517) |
| | | | 137) | |
| B2M_exon_4 | − | ATTT | TATAGAAGGGACTTAAATATCCA | UAUAGAAGGGACUUAAAUAUCCAUAGAU |
| | | | TAGATTT (SEQ ID NO: | UU (SEQ ID NO: 518) |
| | | | 138) | |
| B2M_exon_4 | − | GTTA | TATGCAAATACTATACCATTTTA | UAUGCAAAUACUAUACCAUUUUAUAGAA |
| | | | TAGAAGG (SEQ ID NO: | GG (SEQ ID NO: 519) |
| | | | 139) | |
| B2M_exon_4 | − | TTTG | GTGTCCAAGAAGGGGTCCTGAAA | GUGUCCAAGAAGGGGUCCUGAAACCAAU |
| | | | CCAATCC (SEQ ID NO: | CC (SEQ ID NO: 520) |
| | | | 140) | |
| B2M_exon_4 | − | TTTT | TTTTCTTGTCATTATTCCTTAAA | UUUUCUUGUCAUUAUUCCUUAAACAAUA |
| | | | CAATAAC (SEQ ID NO: | AC (SEQ ID NO: 521) |
| | | | 141) | |
| B2M_exon_4 | − | TTTT | AAATCTGTTATTTGTCATTCAAA | AAAUCUGUUAUUUGUCAUUCAAAGUACA |
| | | | GTACAGC (SEQ ID NO: | GC (SEQ ID NO: 522) |
| | | | 142) | |
| B2M_exon_4 | − | TTTA | CTGAGCATGTACAGACTTTTTTT | CUGAGCAUGUACAGACUUUUUUUUCUUG |
| | | | TCTTGTC (SEQ ID NO: | UC (SEQ ID NO: 523) |
| | | | 143) | |
| B2M_exon_4 | + | TTTG | CTGAATCCACAGATGTGGAGCCC | CUGAAUCCACAGAUGUGGAGCCCCUGGA |
| | | | CTGGATA (SEQ ID NO: | UA (SEQ ID NO: 524) |
| | | | 144) | |
| B2M_exon_4 | + | GTTT | GCTGAATCCACAGATGTGGAGCC | GCUGAAUCCACAGAUGUGGAGCCCCUGG |
| | | | CCTGGAT (SEQ ID NO: | AU (SEQ ID NO: 525) |
| | | | 145) | |
| B2M_exon_4 | + | TTTG | ATCCATGGTTTGCTGAATCCACA | AUCCAUGGUUUGCUGAAUCCACAGAUGU |
| | | | GATGTGG (SEQ ID NO: | GG (SEQ ID NO: 526) |
| | | | 146) | |
| B2M_exon_4 | + | TTTT | GATCCATGGTTTGCTGAATCCAC | GAUCCAUGGUUUGCUGAAUCCACAGAUG |
| | | | AGATGTG (SEQ ID NO: | UG (SEQ ID NO: 527) |
| | | | 147) | |
| B2M_exon_4 | + | TTTT | TGATCCATGGTTTGCTGAATCCA | UGAUCCAUGGUUUGCUGAAUCCACAGAU |
| | | | CAGATGT (SEQ ID NO: | GU (SEQ ID NO: 528) |
| | | | 148) | |
| B2M_exon_4 | + | GTTT | TTGATCCATGGTTTGCTGAATCC | UUGAUCCAUGGUUUGCUGAAUCCACAGA |
| | | | ACAGATG (SEQ ID NO: | UG (SEQ ID NO: 529) |
| | | | 149) | |
| B2M_exon_4 | + | CTTT | GAATGACAAATAACAGATTTAAA | GAAUGACAAAUAACAGAUUUAAAAUUUU |
| | | | ATTTTCA (SEQ ID NO: | CA (SEQ ID NO: 530) |
| | | | 150) | |
| B2M_exon_4 | + | TTTC | CCCAGTGTTTTTGATCCATGGTT | CCCAGUGUUUUUGAUCCAUGGUUUGCUG |
| | | | TGCTGAA (SEQ ID NO: | AA (SEQ ID NO: 531) |
| | | | 151) | |
| B2M_exon_4 | + | TTTT | TCCCCAGTGTTTTTGATCCATGG | UCCCCAGUGUUUUUGAUCCAUGGUUUGC |
| | | | TTTGCTG (SEQ ID NO: | UG (SEQ ID NO: 532) |
| | | | 152) | |
| B2M_exon_4 | + | TTTT | TTCCCCAGTGTTTTTGATCCATG | UUCCCCAGUGUUUUUGAUCCAUGGUUUG |
| | | | GTTTGCT (SEQ ID NO: | CU (SEQ ID NO: 533) |
| | | | 153) | |
| B2M_exon_4 | + | TTTT | TTTCCCCAGTGTTTTTGATCCAT | UUUCCCCAGUGUUUUUGAUCCAUGGUUU |
| | | | GGTTTGC (SEQ ID NO: | GC (SEQ ID NO: 534) |
| | | | 154) | |
| B2M_exon_4 | + | CTTT | TTTTCCCCAGTGTTTTTGATCCA | UUUUCCCCAGUGUUUUUGAUCCAUGGUU |
| | | | TGGTTTG (SEQ ID NO: | UG (SEQ ID NO: 535) |
| | | | 155) | |
| B2M_exon_4 | + | TTTA | AGGAATAATGACAAGAAAAAAAA | AGGAAUAAUGACAAGAAAAAAAAGUCUG |
| | | | GTCTGTA (SEQ ID NO: | UA (SEQ ID NO: 536) |
| | | | 156) | |
| B2M_exon_4 | − | TTTG | CTCCCTCTTAGAGTCTGCATACT | CUCCCUCUUAGAGUCUGCAUACUCCUCA |
| | | | CCTCATG (SEQ ID NO: | UG (SEQ ID NO: 537) |
| | | | 157) | |
| B2M_exon_4 | + | TTTT | CCCCAGTGTTTTTGATCCATGGT | CCCCAGUGUUUUUGAUCCAUGGUUUGCU |
| | | | TTGCTGA (SEQ ID NO: | GA (SEQ ID NO: 538) |
| | | | 158) | |
| B2M_exon_4 | − | CTTT | TTTTTCTTGTCATTATTCCTTAA | UUUUUCUUGUCAUUAUUCCUUAAACAAU |
| | | | ACAATAA (SEQ ID NO: | AA (SEQ ID NO: 539) |
| | | | 159) | |
| B2M_exon_4 | + | TTTG | AATGACAAATAACAGATTTAAAA | AAUGACAAAUAACAGAUUUAAAAUUUUC |
| | | | TTTTCAA (SEQ ID NO: | AA (SEQ ID NO: 540) |
| | | | 160) | |
| B2M_exon_4 | + | TTTA | AAATTTTCAAGGCATAGTTTTAT | AAAUUUUCAAGGCAUAGUUUUAUACCUG |
| | | | ACCTGA (SEQ ID NO: 161) | A (SEQ ID NO: 541) |
| B2M_exon_4 | − | CTTT | ACTGAGCATGTACAGACTTTTTT | ACUGAGCAUGUACAGACUUUUUUUUCUU |
| | | | TTCTTGT (SEQ ID NO: | GU (SEQ ID NO: 542) |
| | | | 162) | |
| B2M_exon_4 | − | GTTG | TGTCTTTACTGAGCATGTACAGA | UGUCUUUACUGAGCAUGUACAGACUUUU |
| | | | CTTTTTT (SEQ ID NO: | UU (SEQ ID NO: 543) |
| | | | 163) | |
| B2M_exon_4 | − | ATTC | AGCAAACCATGGATCAAAAACAC | AGCAAACCAUGGAUCAAAAACACUGGGG |
| | | | TGGGGAA (SEQ ID NO: | AA (SEQ ID NO: 544) |
| | | | 164) | |
| B2M_exon_4 | − | CTTC | CGTATCCAGGGGCTCCACATCTG | CGUAUCCAGGGGCUCCACAUCUGUGGAU |
| | | | TGGATTC (SEQ ID NO: | UC (SEQ ID NO: 545) |
| | | | 165) | |
| B2M_exon_4 | − | ATTC | AAAGTACAGCGGGCCTTCCGTAT | AAAGUACAGCGGGCCUUCCGUAUCCAGG |
| | | | CCAGGGG (SEQ ID NO: | GG (SEQ ID NO: 546) |
| | | | 166) | |
| B2M_exon_4 | − | TTTG | TCATTCAAAGTACAGCGGGCCTT | UCAUUCAAAGUACAGCGGGCCUUCCGUA |
| | | | CCGTATC (SEQ ID NO: | UC (SEQ ID NO: 547) |
| | | | 167) | |
| B2M_exon_4 | + | ATTT | AAAATTTTCAAGGCATAGTTTTA | AAAAUUUUCAAGGCAUAGUUUUAUACCU |
| | | | TACCTGA (SEQ ID NO: | GA (SEQ ID NO: 548) |
| | | | 168) | |
| B2M_exon_4 | − | ATTT | GTCATTCAAAGTACAGCGGGCCT | GUCAUUCAAAGUACAGCGGGCCUUCCGU |
| | | | TCCGTAT (SEQ ID NO: | AU (SEQ ID NO: 549) |
| | | | 169) | |
| B2M_exon_4 | − | TTTA | AATCTGTTATTTGTCATTCAAAG | AAUCUGUUAUUUGUCAUUCAAAGUACAG |
| | | | TACAGCG (SEQ ID NO: | CG (SEQ ID NO: 550) |
| | | | 170) | |
| B2M_exon_4 | − | ATTT | TAAATCTGTTATTTGTCATTCAA | UAAAUCUGUUAUUUGUCAUUCAAAGUAC |
| | | | AGTACAG (SEQ ID NO: | AG (SEQ ID NO: 551) |
| | | | 171) | |
| B2M_exon_4 | − | CTTG | AAAATTTTAAATCTGTTATTTGT | AAAAUUUUAAAUCUGUUAUUUGUCAUUC |
| | | | CATTCAA (SEQ ID NO: | AA (SEQ ID NO: 552) |
| | | | 172) | |
| B2M_exon_4 | + | TTTC | AAGGCATAGTTTTATACCTGA | AAGGCAUAGUUUUAUACCUGA (SEQ |
| | | | (SEQ ID NO: 173) | ID NO: 553) |
| B2M_exon_4 | + | TTTT | CAAGGCATAGTTTTATACCTGA | CAAGGCAUAGUUUUAUACCUGA (SEQ |
| | | | (SEQ ID NO: 174) | ID NO: 554) |
| B2M_exon_4 | + | ATTT | TCAAGGCATAGTTTTATACCTGA | UCAAGGCAUAGUUUUAUACCUGA (SEQ |
| | | | (SEQ ID NO: 175) | ID NO: 555) |
| B2M_exon_4 | − | GTTA | TTTGTCATTCAAAGTACAGCGGG | UUUGUCAUUCAAAGUACAGCGGGCCUUC |
| | | | CCTTCCG (SEQ ID NO: | CG (SEQ ID NO: 556) |
| | | | 176) | |
| B2M_exon_4 | − | CTTA | GAGTCTGCATACTCCTCATGACC | GAGUCUGCAUACUCCUCAUGACCUGGCC |
| | | | TGGCCCG (SEQ ID NO: | CG (SEQ ID NO: 557) |
| | | | 177) | |
| B2M_exon_4 | − | CTTA | ACTATCTTAACAAGCTTTGAGTG | ACUAUCUUAACAAGCUUUGAGUGCAAGA |
| | | | CAAGAGA (SEQ ID NO: | GA (SEQ ID NO: 558) |
| | | | 178) | |
| B2M_exon_4 | − | TTTA | ACTTCTTTGAGCATCAGATTCCT | ACUUCUUUGAGCAUCAGAUUCCUAAUCU |
| | | | AATCIGG (SEQ ID NO: | GG (SEQ ID NO: 559) |
| | | | 179) | |
| B2M_exon_4 | − | ATTA | TATTTCTAAATTTTCCCCCAAAT | UAUUUCUAAAUUUUCCCCCAAAUUCUAA |
| | | | TCTAAGC (SEQ ID NO: | GC (SEQ ID NO: 560) |
| | | | 180) | |
| B2M_exon_4 | − | ATTT | CTAAATTTTCCCCCAAATTCTAA | CUAAAUUUUCCCCCAAAUUCUAAGCAGA |
| | | | GCAGAGT (SEQ ID NO: | GU (SEQ ID NO: 561) |
| | | | 181) | |
| B2M_exon_4 | − | TTTC | TAAATTTTCCCCCAAATTCTAAG | UAAAUUUUCCCCCAAAUUCUAAGCAGAG |
| | | | CAGAGTA (SEQ ID NO: | UA (SEQ ID NO: 562) |
| | | | 182) | |
| B2M_exon_4 | − | ATTT | TCCCCCAAATTCTAAGCAGAGTA | UCCCCCAAAUUCUAAGCAGAGUAUGUAA |
| | | | TGTAAAT (SEQ ID NO: | AU (SEQ ID NO: 563) |
| | | | 183) | |
| B2M_exon_4 | − | TTTT | CCCCCAAATTCTAAGCAGAGTAT | CCCCCAAAUUCUAAGCAGAGUAUGUAAA |
| | | | GTAAATT (SEQ ID NO: | UU (SEQ ID NO: 564) |
| | | | 184) | |
| B2M_exon_4 | − | TTTC | CCCCAAATTCTAAGCAGAGTATG | CCCCAAAUUCUAAGCAGAGUAUGUAAAU |
| | | | TAAATTG (SEQ ID NO: | UG (SEQ ID NO: 565) |
| | | | 185) | |
| B2M_exon_4 | − | ATTC | TAAGCAGAGTATGTAAATTGGAA | UAAGCAGAGUAUGUAAAUUGGAAGUUAA |
| | | | GITAACT (SEQ ID NO: | CU (SEQ ID NO: 566) |
| | | | 186) | |
| B2M_exon_4 | − | ATTG | GAAGTTAACTTATGCACGCTTAA | GAAGUUAACUUAUGCACGCUUAACUAUC |
| | | | CTATCTT (SEQ ID NO: | UU (SEQ ID NO: 567) |
| | | | 187) | |
| B2M_exon_4 | − | GTTA | ACTTATGCACGCTTAACTATCTT | ACUUAUGCACGCUUAACUAUCUUAACAA |
| | | | AACAAGC (SEQ ID NO: | GC (SEQ ID NO: 568) |
| | | | 188) | |
| B2M_exon_4 | − | CTTA | TGCACGCTTAACTATCTTAACAA | UGCACGCUUAACUAUCUUAACAAGCUUU |
| | | | GCTTTGA (SEQ ID NO: | GA (SEQ ID NO: 569) |
| | | | 189) | |
| B2M_exon_4 | + | GTTT | AAGGAATAATGACAAGAAAAAAA | AAGGAAUAAUGACAAGAAAAAAAAGUCU |
| | | | AGTCTGT (SEQ ID NO: | GU (SEQ ID NO: 570) |
| | | | 190) | |
| B2M_exon_4 | − | CTTA | ACAAGCTTTGAGTGCAAGAGATT | ACAAGCUUUGAGUGCAAGAGAUUGAAGA |
| | | | GAAGAGT (SEQ ID NO: | GU (SEQ ID NO: 571) |
| | | | 191) | |
| B2M_exon_4 | − | CTTT | GAGTGCAAGAGATTGAAGAGTTC | GAGUGCAAGAGAUUGAAGAGUUCAAAUC |
| | | | AAATCTG (SEQ ID NO: | UG (SEQ ID NO: 572) |
| | | | 192) | |
| B2M_exon_4 | − | TTTG | AGTGCAAGAGATTGAAGAGTTCA | AGUGCAAGAGAUUGAAGAGUUCAAAUCU |
| | | | AATCTGA (SEQ ID NO: | GA (SEQ ID NO: 573) |
| | | | 193) | |
| B2M_exon_4 | − | ATTG | AAGAGTTCAAATCTGACCAAGAT | AAGAGUUCAAAUCUGACCAAGAUGUUGA |
| | | | GTTGATG (SEQ ID NO: | UG (SEQ ID NO: 574) |
| | | | 194) | |
| B2M_exon_4 | − | GTTC | AAATCTGACCAAGATGTTGATGT | AAAUCUGACCAAGAUGUUGAUGUUGGAU |
| | | | TGGATAA (SEQ ID NO: | AA (SEQ ID NO: 575) |
| | | | 195) | |
| B2M_exon_4 | − | GTTG | ATGTTGGATAAGAGAATTCTCTG | AUGUUGGAUAAGAGAAUUCUCUGCUCCC |
| | | | CTCCCCA (SEQ ID NO: | CA (SEQ ID NO: 576) |
| | | | 196) | |
| B2M_exon_4 | − | ATTC | ATCCAATCCAAATGCGGCATCTT | AUCCAAUCCAAAUGCGGCAUCUUCAAAC |
| | | | CAAACCT (SEQ ID NO: | CU (SEQ ID NO: 577) |
| | | | 197) | |
| B2M_exon_4 | − | TTTG | GAATTCATCCAATCCAAATGCGG | GAAUUCAUCCAAUCCAAAUGCGGCAUCU |
| | | | CATCTTC (SEQ ID NO: | UC (SEQ ID NO: 578) |
| | | | 198) | |
| B2M_exon_4 | − | ATTT | GGAATTCATCCAATCCAAATGCG | GGAAUUCAUCCAAUCCAAAUGCGGCAUC |
| | | | GCATCTT (SEQ ID NO: | UU (SEQ ID NO: 579) |
| | | | 199) | |
| B2M_exon_4 | − | ATTA | AAAAGCAAGCAAGCAGAATTTGG | AAAAGCAAGCAAGCAGAAUUUGGAAUUC |
| | | | AATTCAT (SEQ ID NO: | AU (SEQ ID NO: 580) |
| | | | 200) | |
| B2M_exon_4 | − | TTTG | TGCATAAAGTGTAAGTGTATAAG | UGCAUAAAGUGUAAGUGUAUAAGCAUAU |
| | | | CATATCA (SEQ ID NO: | CA (SEQ ID NO: 581) |
| | | | 201) | |
| B2M_exon_4 | − | TTTT | GTGCATAAAGTGTAAGTGTATAA | GUGCAUAAAGUGUAAGUGUAUAAGCAUA |
| | | | GCATATC (SEQ ID NO: | UC (SEQ ID NO: 582) |
| | | | 202) | |
| B2M_exon_4 | − | TTTC | CAATAATCCTGTCAATTATATTT | CAAUAAUCCUGUCAAUUAUAUUUCUAAA |
| | | | CTAAATT (SEQ ID NO: | UU (SEQ ID NO: 583) |
| | | | 203) | |
| B2M_exon_4 | − | ATTT | TGTGCATAAAGTGTAAGTGTATA | UGUGCAUAAAGUGUAAGUGUAUAAGCAU |
| | | | AGCATAT (SEQ ID NO: | AU (SEQ ID NO: 584) |
| | | | 204) | |
| B2M_exon_4 | − | ATTA | TTATAACCCTACATTTTGTGCAT | UUAUAACCCUACAUUUUGUGCAUAAAGU |
| | | | AAAGTGT (SEQ ID NO: | GU (SEQ ID NO: 585) |
| | | | 205) | |
| B2M_exon_4 | − | GTTA | ACATTATTATAACCCTACATTTT | ACAUUAUUAUAACCCUACAUUUUGUGCA |
| | | | GTGCATA (SEQ ID NO: | UA (SEQ ID NO: 586) |
| | | | 206) | |
| B2M_exon_4 | − | ATTA | TAAAGAAGATCATGTCCATGTTA | UAAAGAAGAUCAUGUCCAUGUUAACAUU |
| | | | ACATTAT (SEQ ID NO: | AU (SEQ ID NO: 587) |
| | | | 207) | |
| B2M_exon_4 | − | GTTG | CCAGCCCTCCTAGAGCTACCTGT | CCAGCCCUCCUAGAGCUACCUGUGGAGC |
| | | | GGAGCAA (SEQ ID NO: | AA (SEQ ID NO: 588) |
| | | | 208) | |
| B2M_exon_4 | − | ATTC | TCTGCTCCCCACCTCTAAGTTGC | UCUGCUCCCCACCUCUAAGUUGCCAGCC |
| | | | CAGCCCT (SEQ ID NO: | CU (SEQ ID NO: 589) |
| | | | 209) | |
| B2M_exon_4 | − | GTTG | GATAAGAGAATTCTCTGCTCCCC | GAUAAGAGAAUUCUCUGCUCCCCACCUC |
| | | | ACCTCTA (SEQ ID NO: | UA (SEQ ID NO: 590) |
| | | | 210) | |
| B2M_exon_4 | − | ATTA | TAACCCTACATTTTGTGCATAAA | UAACCCUACAUUUUGUGCAUAAAGUGUA |
| | | | GTGTAAG (SEQ ID NO: | AG (SEQ ID NO: 591) |
| | | | 211) | |
| B2M_exon_4 | − | ATTT | CCAATAATCCTGTCAATTATATT | CCAAUAAUCCUGUCAAUUAUAUUUCUAA |
| | | | TCTAAAT (SEQ ID NO: | AU (SEQ ID NO: 592) |
| | | | 212) | |
| B2M_exon_4 | − | ATTA | TAACAAATTTCCAATAATCCTGT | UAACAAAUUUCCAAUAAUCCUGUCAAUU |
| | | | CAATTAT (SEQ ID NO: | AU (SEQ ID NO: 593) |
| | | | 213) | |
| B2M_exon_4 | − | ATTC | ATTATAACAAATTTCCAATAATC | AUUAUAACAAAUUUCCAAUAAUCCUGUC |
| | | | CTGTCAA (SEQ ID NO: | AA (SEQ ID NO: 594) |
| | | | 214) | |
| B2M_exon_4 | − | GTTT | CCCTGTTTGAAAATAAAGGGGTA | CCCUGUUUGAAAAUAAAGGGGUAAUAGU |
| | | | ATAGTGG (SEQ ID NO: | GG (SEQ ID NO: 595) |
| | | | 215) | |
| B2M_exon_4 | − | CTTG | AAGACTGTTTCCCTGTTTGAAAA | AAGACUGUUUCCCUGUUUGAAAAUAAAG |
| | | | TAAAGGG (SEQ ID NO: | GG (SEQ ID NO: 596) |
| | | | 216) | |
| B2M_exon_4 | − | TTTA | CCAAGTGGAACTTGAAGACTGTT | CCAAGUGGAACUUGAAGACUGUUUCCCU |
| | | | TCCCTGT (SEQ ID NO: | GU (SEQ ID NO: 597) |
| | | | 217) | |
| B2M_exon_4 | − | TTTT | ACCAAGTGGAACTTGAAGACTGT | ACCAAGUGGAACUUGAAGACUGUUUCCC |
| | | | TTCCCTG (SEQ ID NO: | UG (SEQ ID NO: 598) |
| | | | 218) | |
| B2M_exon_4 | − | TTTT | TACCAAGTGGAACTTGAAGACTG | UACCAAGUGGAACUUGAAGACUGUUUCC |
| | | | TTTCCCT (SEQ ID NO: | CU (SEQ ID NO: 599) |
| | | | 219) | |
| B2M_exon_4 | − | TTTT | TTACCAAGTGGAACTTGAAGACT | UUACCAAGUGGAACUUGAAGACUGUUUC |
| | | | GTTTCCC (SEQ ID NO: | CC (SEQ ID NO: 600) |
| | | | 220) | |
| B2M_exon_4 | − | TTTC | CCTGTTTGAAAATAAAGGGGTAA | CCUGUUUGAAAAUAAAGGGGUAAUAGUG |
| | | | TAGTGGG (SEQ ID NO: | GG (SEQ ID NO: 601) |
| | | | 221) | |
| B2M_exon_4 | − | ATTT | TTTACCAAGTGGAACTTGAAGAC | UUUACCAAGUGGAACUUGAAGACUGUUU |
| | | | TGTTTCC (SEQ ID NO: | CC (SEQ ID NO: 602) |
| | | | 222) | |
| B2M_exon_4 | − | TTTA | CACTGTGAGCCAAACTCTATATA | CACUGUGAGCCAAACUCUAUAUACAAGG |
| | | | CAAGGGG (SEQ ID NO: | GG (SEQ ID NO: 603) |
| | | | 223) | |
| B2M_exon_4 | − | CTTT | ACACTGTGAGCCAAACTCTATAT | ACACUGUGAGCCAAACUCUAUAUACAAG |
| | | | ACAAGGG (SEQ ID NO: | GG (SEQ ID NO: 604) |
| | | | 224) | |
| B2M_exon_4 | − | ATTC | CTAATCTGGAAAATGTGAATCAC | CUAAUCUGGAAAAUGUGAAUCACUGAGG |
| | | | TGAGGCC (SEQ ID NO: | CC (SEQ ID NO: 605) |
| | | | 225) | |
| B2M_exon_4 | − | TTTG | AGCATCAGATTCCTAATCTGGAA | AGCAUCAGAUUCCUAAUCUGGAAAAUGU |
| | | | AATGTGA (SEQ ID NO: | GA (SEQ ID NO: 606) |
| | | | 226) | |
| B2M_exon_4 | − | CTTT | GAGCATCAGATTCCTAATCTGGA | GAGCAUCAGAUUCCUAAUCUGGAAAAUG |
| | | | AAATGTG (SEQ ID NO: | UG (SEQ ID NO: 607) |
| | | | 227) | |
| B2M_exon_4 | − | CTTC | TTTGAGCATCAGATTCCTAATCT | UUUGAGCAUCAGAUUCCUAAUCUGGAAA |
| | | | GGAAAAT (SEQ ID NO: | AU (SEQ ID NO: 608) |
| | | | 228) | |
| B2M_exon_4 | − | GTTC | ACATTTTTTACCAAGTGGAACTT | ACAUUUUUUACCAAGUGGAACUUGAAGA |
| | | | GAAGACT (SEQ ID NO: | CU (SEQ ID NO: 609) |
| | | | 229) | |
| B2M_exon_4 | − | ATTT | AACTTCTTTGAGCATCAGATTCC | AACUUCUUUGAGCAUCAGAUUCCUAAUC |
| | | | TAATCTG (SEQ ID NO: | UG (SEQ ID NO: 610) |
| | | | 230) | |
| B2M_exon_4 | − | GTTT | GAAAATAAAGGGGTAATAGTGGG | GAAAAUAAAGGGGUAAUAGUGGGAGUGA |
| | | | AGTGAGA (SEQ ID NO: | GA (SEQ ID NO: 611) |
| | | | 231) | |
| B2M_exon_4 | − | GTTT | TATGATTTATTTAACTTGTGGAA | UAUGAUUUAUUUAACUUGUGGAACAAAA |
| | | | CAAAAAT (SEQ ID NO: | AU (SEQ ID NO: 612) |
| | | | 232) | |
| B2M_exon_4 | − | TTTC | ATTCATTATAACAAATTTCCAAT | AUUCAUUAUAACAAAUUUCCAAUAAUCC |
| | | | AATCCTG (SEQ ID NO: | UG (SEQ ID NO: 613) |
| | | | 233) | |
| B2M_exon_4 | − | GTTT | CATTCATTATAACAAATTTCCAA | CAUUCAUUAUAACAAAUUUCCAAUAAUC |
| | | | TAATCCT (SEQ ID NO: | CU (SEQ ID NO: 614) |
| | | | 234) | |
| B2M_exon_4 | − | CTTA | TATGACAAAATGTTTCATTCATT | UAUGACAAAAUGUUUCAUUCAUUAUAAC |
| | | | ATAACAA (SEQ ID NO: | AA (SEQ ID NO: 615) |
| | | | 235) | |
| B2M_exon_4 | − | TTTA | TCAAATGTATAAGAAGTAAATAT | UCAAAUGUAUAAGAAGUAAAUAUGAAUC |
| | | | GAATCTT (SEQ ID NO: | UU (SEQ ID NO: 616) |
| | | | 236) | |
| B2M_exon_4 | − | CTTT | ATCAAATGTATAAGAAGTAAATA | AUCAAAUGUAUAAGAAGUAAAUAUGAAU |
| | | | TGAATCT (SEQ ID NO: | CU (SEQ ID NO: 617) |
| | | | 237) | |
| B2M_exon_4 | − | CTTA | CTTTATCAAATGTATAAGAAGTA | CUUUAUCAAAUGUAUAAGAAGUAAAUAU |
| | | | AATATGA (SEQ ID NO: | GA (SEQ ID NO: 618) |
| | | | 238) | |
| B2M_exon_4 | − | TTTG | AAAATAAAGGGGTAATAGTGGGA | AAAAUAAAGGGGUAAUAGUGGGAGUGAG |
| | | | GTGAGAT (SEQ ID NO: | AU (SEQ ID NO: 619) |
| | | | 239) | |
| B2M_exon_4 | − | ATTA | ACCACAACCATGCCTTACTTTAT | ACCACAACCAUGCCUUACUUUAUCAAAU |
| | | | CAAATGT (SEQ ID NO: | GU (SEQ ID NO: 620) |
| | | | 240) | |
| B2M_exon_4 | − | TTTA | ACTTGTGGAACAAAAATAAACCA | ACUUGUGGAACAAAAAUAAACCAGAUUA |
| | | | GATTAAC (SEQ ID NO: | AC (SEQ ID NO: 621) |
| | | | 241) | |
| B2M_exon_4 | − | ATTT | AACTTGTGGAACAAAAATAAACC | AACUUGUGGAACAAAAAUAAACCAGAUU |
| | | | AGATTAA (SEQ ID NO: | AA (SEQ ID NO: 622) |
| | | | 242) | |
| B2M_exon_4 | − | TTTA | TTTAACTTGTGGAACAAAAATAA | UUUAACUUGUGGAACAAAAAAAACCAG |
| | | | ACCAGAT (SEQ ID NO: | AU (SEQ ID NO: 623) |
| | | | 243) | |
| B2M_exon_4 | − | ATTT | ATTTAACTTGTGGAACAAAAATA | AUUUAACUUGUGGAACAAAAAUAAACCA |
| | | | AACCAGA (SEQ ID NO: | GA (SEQ ID NO: 624) |
| | | | 244) | |
| B2M_exon_4 | − | TTTA | TGATTTATTTAACTTGTGGAACA | UGAUUUAUUUAACUUGUGGAACAAAAAU |
| | | | AAAATAA (SEQ ID NO: | AA (SEQ ID NO: 625) |
| | | | 245) | |
| B2M_exon_4 | − | TTTT | ATGATTTATTTAACTTGTGGAAC | AUGAUUUAUUUAACUUGUGGAACAAAAA |
| | | | AAAAATA (SEQ ID NO: | UA (SEQ ID NO: 626) |
| | | | 246) | |
| B2M_exon_4 | − | CTTG | TGGAACAAAAATAAACCAGATTA | UGGAACAAAAAUAAACCAGAUUAACCAC |
| | | | ACCACAA (SEQ ID NO: | AA (SEQ ID NO: 627) |
| | | | 247) | |
| B2M_exon_4 | + | ATTG | TTTAAGGAATAATGACAAGAAAA | UUUAAGGAAUAAUGACAAGAAAAAAAAG |
| | | | AAAAGTC (SEQ ID NO: | UC (SEQ ID NO: 628) |
| | | | 248) | |
| B2M_exon_4 | + | CTTT | ATTTTCAAACAGGGAAACAGTCT | AUUUUCAAACAGGGAAACAGUCUUCAAG |
| | | | TCAAGTT (SEQ ID NO: | UU (SEQ ID NO: 629) |
| | | | 249) | |
| B2M_exon_4 | + | GTTG | TTATTGTTTAAGGAATAATGACA | UUAUUGUUUAAGGAAUAAUGACAAGAAA |
| | | | AGAAAAA (SEQ ID NO: | AA (SEQ ID NO: 630) |
| | | | 250) | |
| B2M_exon_4 | + | CTTT | GAGTGCTGTCTCCATGTTTGATG | GAGUGCUGUCUCCAUGUUUGAUGUAUCU |
| | | | TATCTGA (SEQ ID NO: | GA (SEQ ID NO: 631) |
| | | | 251) | |
| B2M_exon_4 | + | TTTG | AGTGCTGTCTCCATGTTTGATGT | AGUGCUGUCUCCAUGUUUGAUGUAUCUG |
| | | | ATCTGAG (SEQ ID NO: | AG (SEQ ID NO: 632) |
| | | | 252) | |
| B2M_exon_4 | + | GTTT | GATGTATCTGAGCAGGTTGCTCC | GAUGUAUCUGAGCAGGUUGCUCCACAGG |
| | | | ACAGGTA (SEQ ID NO: | UA (SEQ ID NO: 633) |
| | | | 253) | |
| B2M_exon_4 | + | TTTG | ATGTATCTGAGCAGGTTGCTCCA | AUGUAUCUGAGCAGGUUGCUCCACAGGU |
| | | | CAGGTAG (SEQ ID NO: | AG (SEQ ID NO: 634) |
| | | | 254) | |
| B2M_exon_4 | + | GTTG | CTCCACAGGTAGCTCTAGGAGGG | CUCCACAGGUAGCUCUAGGAGGGCUGGC |
| | | | CTGGCAA (SEQ ID NO: | AA (SEQ ID NO: 635) |
| | | | 255) | |
| B2M_exon_4 | + | CTTA | GAGGTGGGGAGCAGAGAATTCTC | GAGGUGGGGAGCAGAGAAUUCUCUUAUC |
| | | | TTATCCA (SEQ ID NO: | CA (SEQ ID NO: 636) |
| | | | 256) | |
| B2M_exon_4 | + | ATTC | TCTTATCCAACATCAACATCTTG | UCUUAUCCAACAUCAACAUCUUGGUCAG |
| | | | GTCAGAT (SEQ ID NO: | AU (SEQ ID NO: 637) |
| | | | 257) | |
| B2M_exon_4 | + | CTTA | TCCAACATCAACATCTTGGTCAG | UCCAACAUCAACAUCUUGGUCAGAUUUG |
| | | | ATTTGAA (SEQ ID NO: | AA (SEQ ID NO: 638) |
| | | | 258) | |
| B2M_exon_4 | + | CTTG | GTCAGATTTGAACTCTTCAATCT | GUCAGAUUUGAACUCUUCAAUCUCUUGC |
| | | | CTTGCAC (SEQ ID NO: | AC (SEQ ID NO: 639) |
| | | | 259) | |
| B2M_exon_4 | + | ATTT | GAACTCTTCAATCTCTTGCACTC | GAACUCUUCAAUCUCUUGCACUCAAAGC |
| | | | AAAGCTT (SEQ ID NO: | UU (SEQ ID NO: 640) |
| | | | 260) | |
| B2M_exon_4 | + | TTTG | AACTCTTCAATCTCTTGCACTCA | AACUCUUCAAUCUCUUGCACUCAAAGCU |
| | | | AAGCTTG (SEQ ID NO: | UG (SEQ ID NO: 641) |
| | | | 261) | |
| B2M_exon_4 | + | CTTC | AATCTCTTGCACTCAAAGCTTGT | AAUCUCUUGCACUCAAAGCUUGUUAAGA |
| | | | TAAGATA (SEQ ID NO: | UA (SEQ ID NO: 642) |
| | | | 262) | |
| B2M_exon_4 | + | CTTG | CACTCAAAGCTTGTTAAGATAGT | CACUCAAAGCUUGUUAAGAUAGUUAAGC |
| | | | TAAGCGT (SEQ ID NO: | GU (SEQ ID NO: 643) |
| | | | 263) | |
| B2M_exon_4 | + | CTTG | TTAAGATAGTTAAGCGTGCATAA | UUAAGAUAGUUAAGCGUGCAUAAGUUAA |
| | | | GTTAACT (SEQ ID NO: | CU (SEQ ID NO: 644) |
| | | | 264) | |
| B2M_exon_4 | + | GTTA | AGATAGTTAAGCGTGCATAAGTT | AGAUAGUUAAGCGUGCAUAAGUUAACUU |
| | | | AACTTCC (SEQ ID NO: | CC (SEQ ID NO: 645) |
| | | | 265) | |
| B2M_exon_4 | + | GTTA | AGCGTGCATAAGTTAACTTCCAA | AGCGUGCAUAAGUUAACUUCCAAUUUAC |
| | | | TTTACAT (SEQ ID NO: | AU (SEQ ID NO: 646) |
| | | | 266) | |
| B2M_exon_4 | + | GTTA | ACTTCCAATTTACATACTCTGCT | ACUUCCAAUUUACAUACUCUGCUUAGAA |
| | | | TAGAATT (SEQ ID NO: | UU (SEQ ID NO: 647) |
| | | | 267) | |
| B2M_exon_4 | + | GTTA | TAATGAATGAAACATTTTGTCAT | UAAUGAAUGAAACAUUUUGUCAUAUAAG |
| | | | ATAAGAT (SEQ ID NO: | AU (SEQ ID NO: 648) |
| | | | 268) | |
| B2M_exon_4 | + | TTTG | TTATAATGAATGAAACATTTTGT | UUAUAAUGAAUGAAACAUUUUGUCAUAU |
| | | | CATATAA (SEQ ID NO: | AA (SEQ ID NO: 649) |
| | | | 269) | |
| B2M_exon_4 | + | ATTT | GTTATAATGAATGAAACATTTTG | GUUAUAAUGAAUGAAACAUUUUGUCAUA |
| | | | TCATATA (SEQ ID NO: | UA (SEQ ID NO: 650) |
| | | | 270) | |
| B2M_exon_4 | + | ATTG | GAAATTTGTTATAATGAATGAAA | GAAAUUUGUUAUAAUGAAUGAAACAUUU |
| | | | CATTTTG (SEQ ID NO: | UG (SEQ ID NO: 651) |
| | | | 271) | |
| B2M_exon_4 | + | ATTA | TTGGAAATTTGTTATAATGAATG | UUGGAAAUUUGUUAUAAUGAAUGAAACA |
| | | | AAACATT (SEQ ID NO: | UU (SEQ ID NO: 652) |
| | | | 272) | |
| B2M_exon_4 | + | ATTG | ACAGGATTATTGGAAATTTGTTA | ACAGGAUUAUUGGAAAUUUGUUAUAAUG |
| | | | TAATGAA (SEQ ID NO: | AA (SEQ ID NO: 653) |
| | | | 273) | |
| B2M_exon_4 | + | ATTC | TACTTTGAGTGCTGTCTCCATGT | UACUUUGAGUGCUGUCUCCAUGUUUGAU |
| | | | TTGATGT (SEQ ID NO: | GU (SEQ ID NO: 654) |
| | | | 274) | |
| B2M_exon_4 | + | TTTA | GAAATATAATTGACAGGATTATT | GAAAUAUAAUUGACAGGAUUAUUGGAAA |
| | | | GGAAATT (SEQ ID NO: | UU (SEQ ID NO: 655) |
| | | | 275) | |
| B2M_exon_4 | + | TTTG | GGGGAAAATTTAGAAATATAATT | GGGGAAAAUUUAGAAAUAUAAUUGACAG |
| | | | GACAGGA (SEQ ID NO: | GA (SEQ ID NO: 656) |
| | | | 276) | |
| B2M_exon_4 | + | ATTT | GGGGGAAAATTTAGAAATATAAT | GGGGGAAAAUUUAGAAAUAUAAUUGACA |
| | | | TGACAGG (SEQ ID NO: | GG (SEQ ID NO: 657) |
| | | | 277) | |
| B2M_exon_4 | + | CTTA | GAATTTGGGGGAAAATTTAGAAA | GAAUUUGGGGGAAAAUUUAGAAAUAUAA |
| | | | TATAATT (SEQ ID NO: | UU (SEQ ID NO: 658) |
| | | | 278) | |
| B2M_exon_4 | + | TTTA | CATACTCTGCTTAGAATTTGGGG | CAUACUCUGCUUAGAAUUUGGGGGAAAA |
| | | | GAAAATT (SEQ ID NO: | UU (SEQ ID NO: 659) |
| | | | 279) | |
| B2M_exon_4 | + | ATTT | ACATACTCTGCTTAGAATTTGGG | ACAUACUCUGCUUAGAAUUUGGGGGAAA |
| | | | GGAAAAT (SEQ ID NO: | AU (SEQ ID NO: 660) |
| | | | 280) | |
| B2M_exon_4 | + | CTTC | CAATTTACATACTCTGCTTAGAA | CAAUUUACAUACUCUGCUUAGAAUUUGG |
| | | | TTTGGGG (SEQ ID NO: | GG (SEQ ID NO: 661) |
| | | | 281) | |
| B2M_exon_4 | + | ATTT | AGAAATATAATTGACAGGATTAT | AGAAAUAUAAUUGACAGGAUUAUUGGAA |
| | | | TGGAAAT (SEQ ID NO: | AU (SEQ ID NO: 662) |
| | | | 282) | |
| B2M_exon_4 | + | TTTA | TAATTCTACTTTGAGTGCTGTCT | UAAUUCUACUUUGAGUGCUGUCUCCAUG |
| | | | CCATGTT (SEQ ID NO: | UU (SEQ ID NO: 663) |
| | | | 283) | |
| B2M_exon_4 | + | CTTT | ATAATTCTACTTTGAGTGCTGTC | AUAAUUCUACUUUGAGUGCUGUCUCCAU |
| | | | TCCATGT (SEQ ID NO: | GU (SEQ ID NO: 664) |
| | | | 284) | |
| B2M_exon_4 | + | CTTC | TTTATAATTCTACTTTGAGTGCT | UUUAUAAUUCUACUUUGAGUGCUGUCUC |
| | | | GTCTCCA (SEQ ID NO: | CA (SEQ ID NO: 665) |
| | | | 285) | |
| B2M_exon_4 | + | TTTG | AAGATGCCGCATTTGGATTGGAT | AAGAUGCCGCAUUUGGAUUGGAUGAAUU |
| | | | GAATTCC (SEQ ID NO: | CC (SEQ ID NO: 666) |
| | | | 286) | |
| B2M_exon_4 | + | GTTT | GAAGATGCCGCATTTGGATTGGA | GAAGAUGCCGCAUUUGGAUUGGAUGAAU |
| | | | TGAATTC (SEQ ID NO: | UC (SEQ ID NO: 667) |
| | | | 287) | |
| B2M_exon_4 | + | TTTC | AGGTTTGAAGATGCCGCATTTGG | AGGUUUGAAGAUGCCGCAUUUGGAUUGG |
| | | | ATTGGAT (SEQ ID NO: | AU (SEQ ID NO: 668) |
| | | | 288) | |
| B2M_exon_4 | + | TTTT | CAGGTTTGAAGATGCCGCATTTG | CAGGUUUGAAGAUGCCGCAUUUGGAUUG |
| | | | GATTGGA (SEQ ID NO: | GA (SEQ ID NO: 669) |
| | | | 289) | |
| B2M_exon_4 | + | CTTT | TCAGGTTTGAAGATGCCGCATTT | UCAGGUUUGAAGAUGCCGCAUUUGGAUU |
| | | | GGATTGG (SEQ ID NO: | GG (SEQ ID NO: 670) |
| | | | 290) | |
| B2M_exon_4 | + | TTTC | TTTTCAGGTTTGAAGATGCCGCA | UUUUCAGGUUUGAAGAUGCCGCAUUUGG |
| | | | TTTGGAT (SEQ ID NO: | AU (SEQ ID NO: 671) |
| | | | 291) | |
| B2M_exon_4 | + | ATTT | GGATTGGATGAATTCCAAATTCT | GGAUUGGAUGAAUUCCAAAUUCUGCUUG |
| | | | GCTTGCT (SEQ ID NO: | CU (SEQ ID NO: 672) |
| | | | 292) | |
| B2M_exon_4 | + | TTTT | CTTTTCAGGTTTGAAGATGCCGC | CUUUUCAGGUUUGAAGAUGCCGCAUUUG |
| | | | ATTTGGA (SEQ ID NO: | GA (SEQ ID NO: 673) |
| | | | 293) | |
| B2M_exon_4 | + | TTTC | TTTTCTTTTCAGGTTTGAAGATG | UUUUCUUUUCAGGUUUGAAGAUGCCGCA |
| | | | CCGCATT (SEQ ID NO: | UU (SEQ ID NO: 674) |
| | | | 294) | |
| B2M_exon_4 | + | TTTT | CTTTTCTTTTCAGGTTTGAAGAT | CUUUUCUUUUCAGGUUUGAAGAUGCCGC |
| | | | GCCGCAT (SEQ ID NO: | AU (SEQ ID NO: 675) |
| | | | 295) | |
| B2M_exon_4 | + | TTTT | TCTTTTCTTTTCAGGTTTGAAGA | UCUUUUCUUUUCAGGUUUGAAGAUGCCG |
| | | | TGCCGCA (SEQ ID NO: | CA (SEQ ID NO: 676) |
| | | | 296) | |
| B2M_exon_4 | + | CTTT | TTCTTTTCTTTTCAGGTTTGAAG | UUCUUUUCUUUUCAGGUUUGAAGAUGCC |
| | | | ATGCCGC (SEQ ID NO: | GC (SEQ ID NO: 677) |
| | | | 297) | |
| B2M_exon_4 | + | ATTG | CTAACCTTTTTCTTTTCTTTTCA | CUAACCUUUUUCUUUUCUUUUCAGGUUU |
| | | | GGTTTGA (SEQ ID NO: | GA (SEQ ID NO: 678) |
| | | | 298) | |
| B2M_exon_4 | + | ATTC | ATTGCTAACCTTTTTCTTTTCTT | AUUGCUAACCUUUUUCUUUUUUUUCAG |
| | | | TTCAGGT (SEQ ID NO: | GU (SEQ ID NO: 679) |
| | | | 299) | |
| B2M_exon_4 | + | CTTT | TCTTTTCAGGTTTGAAGATGCCG | UCUUUUCAGGUUUGAAGAUGCCGCAUUU |
| | | | CATTTGG (SEQ ID NO: | GG (SEQ ID NO: 680) |
| | | | 300) | |
| B2M_exon_4 | + | ATTT | TGTCATATAAGATTCATATTTAC | UGUCAUAUAAGAUUCAUAUUUACUUCUU |
| | | | TTCTTAT (SEQ ID NO: | AU (SEQ ID NO: 681) |
| | | | 301) | |
| B2M_exon_4 | + | TTTG | GATTGGATGAATTCCAAATTCTG | GAUUGGAUGAAUUCCAAAUUCUGCUUGC |
| | | | CTTGCTT (SEQ ID NO: | UU (SEQ ID NO: 682) |
| | | | 302) | |
| B2M_exon_4 | + | ATTC | CAAATTCTGCTTGCTTGCTTTTT | CAAAUUCUGCUUGCUUGCUUUUUAAUAU |
| | | | AATATTG (SEQ ID NO: | UG (SEQ ID NO: 683) |
| | | | 303) | |
| B2M_exon_4 | + | GTTA | ACATGGACATGATCTTCTTTATA | ACAUGGACAUGAUCUUCUUUAUAAUUCU |
| | | | ATTCTAC (SEQ ID NO: | AC (SEQ ID NO: 684) |
| | | | 304) | |
| B2M_exon_4 | + | GTTA | TAATAATGTTAACATGGACATGA | UAAUAAUGUUAACAUGGACAUGAUCUUC |
| | | | TCTTCTT (SEQ ID NO: | UU (SEQ ID NO: 685) |
| | | | 305) | |
| B2M_exon_4 | + | TTTA | TGCACAAAATGTAGGGTTATAAT | UGCACAAAAUGUAGGGUUAUAAUAAUGU |
| | | | AATGTTA (SEQ ID NO: | UA (SEQ ID NO: 686) |
| | | | 306) | |
| B2M_exon_4 | + | CTTT | ATGCACAAAATGTAGGGTTATAA | AUGCACAAAAUGUAGGGUUAUAAUAAUG |
| | | | TAATGTT (SEQ ID NO: | UU (SEQ ID NO: 687) |
| | | | 307) | |
| B2M_exon_4 | + | CTTA | CACTTTATGCACAAAATGTAGGG | CACUUUAUGCACAAAAUGUAGGGUUAUA |
| | | | TTATAAT (SEQ ID NO: | AU (SEQ ID NO: 688) |
| | | | 308) | |
| B2M_exon_4 | + | CTTA | TACACTTACACTTTATGCACAAA | UACACUUACACUUUAUGCACAAAAUGUA |
| | | | ATGTAGG (SEQ ID NO: | GG (SEQ ID NO: 689) |
| | | | 309) | |
| B2M_exon_4 | + | ATTG | GATGAATTCCAAATTCTGCTTGC | GAUGAAUUCCAAAUUCUGCUUGCUUGCU |
| | | | TTGCTTT (SEQ ID NO: | UU (SEQ ID NO: 690) |
| | | | 310) | |
| B2M_exon_4 | + | ATTG | ATATGCTTATACACTTACACTTT | AUAUGCUUAUACACUUACACUUUAUGCA |
| | | | ATGCACA (SEQ ID NO: | CA (SEQ ID NO: 691) |
| | | | 311) | |
| B2M_exon_4 | + | TTTT | AATATTGATATGCTTATACACTT | AAUAUUGAUAUGCUUAUACACUUACACU |
| | | | ACACTTT (SEQ ID NO: | UU (SEQ ID NO: 692) |
| | | | 312) | |
| B2M_exon_4 | + | TTTT | TAATATTGATATGCTTATACACT | UAAUAUUGAUAUGCUUAUACACUUACAC |
| | | | TACACTT (SEQ ID NO: | UU (SEQ ID NO: 693) |
| | | | 313) | |
| B2M_exon_4 | + | CTTT | TTAATATTGATATGCTTATACAC | UUAAUAUUGAUAUGCUUAUACACUUACA |
| | | | TTACACT (SEQ ID NO: | CU (SEQ ID NO: 694) |
| | | | 314) | |
| B2M_exon_4 | + | CTTG | CTTTTTAATATTGATATGCTTAT | CUUUUUAAUAUUGAUAUGCUUAUACACU |
| | | | ACACTTA (SEQ ID NO: | UA (SEQ ID NO: 695) |
| | | | 315) | |
| B2M_exon_4 | + | CTTG | CTTGCTTTTTAATATTGATATGC | CUUGCUUUUUAAUAUUGAUAUGCUUAUA |
| | | | TTATACA (SEQ ID NO: | CA (SEQ ID NO: 696) |
| | | | 316) | |
| B2M_exon_4 | + | ATTC | TGCTTGCTTGCTTTTTAATATTG | UGCUUGCUUGCUUUUUAAUAUUGAUAUG |
| | | | ATATGCT (SEQ ID NO: | CU (SEQ ID NO: 697) |
| | | | 317) | |
| B2M_exon_4 | + | TTTA | ATATTGATATGCTTATACACTTA | AUAUUGAUAUGCUUAUACACUUACACUU |
| | | | CACTTTA (SEQ ID NO: | UA (SEQ ID NO: 698) |
| | | | 318) | |
| B2M_exon_4 | + | GTTA | TTGTTTAAGGAATAATGACAAGA | UUGUUUAAGGAAUAAUGACAAGAAAAAA |
| | | | AAAAAAA (SEQ ID NO: | AA (SEQ ID NO: 699) |
| | | | 319) | |
| B2M_exon_4 | + | TTTT | GTCATATAAGATTCATATTTACT | GUCAUAUAAGAUUCAUAUUUACUUCUUA |
| | | | TCTTATA (SEQ ID NO: | UA (SEQ ID NO: 700) |
| | | | 320) | |
| B2M_exon_4 | + | ATTC | ATATTTACTTCTTATACATTTGA | AUAUUUACUUCUUAUACAUUUGAUAAAG |
| | | | TAAAGTA (SEQ ID NO: | UA (SEQ ID NO: 701) |
| | | | 321) | |
| B2M_exon_4 | + | GTTA | AATGGCATAGTTGGGGTGACACA | AAUGGCAUAGUUGGGGUGACACAGCUGU |
| | | | GCTGTCT (SEQ ID NO: | CU (SEQ ID NO: 702) |
| | | | 322) | |
| B2M_exon_4 | + | GTTG | GGGTGACACAGCTGTCTAGTGGG | GGGUGACACAGCUGUCUAGUGGGAGGCC |
| | | | AGGCCAG (SEQ ID NO: | AG (SEQ ID NO: 703) |
| | | | 323) | |
| B2M_exon_4 | + | CTTC | TATATTTTAGCCAGCGTTCTTTC | UAUAUUUUAGCCAGCGUUCUUUCCUGCG |
| | | | CTGCGGG (SEQ ID NO: | GG (SEQ ID NO: 704) |
| | | | 324) | |
| B2M_exon_4 | + | ATTT | TAGCCAGCGTTCTTTCCTGCGGG | UAGCCAGCGUUCUUUCCUGCGGGCCAGG |
| | | | CCAGGTC (SEQ ID NO: | UC (SEQ ID NO: 705) |
| | | | 325) | |
| B2M_exon_4 | + | TTTT | AGCCAGCGTTCTTTCCTGCGGGC | AGCCAGCGUUCUUUCCUGCGGGCCAGGU |
| | | | CAGGTCA (SEQ ID NO: | CA (SEQ ID NO: 706) |
| | | | 326) | |
| B2M_exon_4 | + | TTTA | GCCAGCGTTCTTTCCTGCGGGCC | GCCAGCGUUCUUUCCUGCGGGCCAGGUC |
| | | | AGGTCAT (SEQ ID NO: | AU (SEQ ID NO: 707) |
| | | | 327) | |
| B2M_exon_4 | + | GTTC | TTTCCTGCGGGCCAGGTCATGAG | UUUCCUGCGGGCCAGGUCAUGAGGAGUA |
| | | | GAGTATG (SEQ ID NO: | UG (SEQ ID NO: 708) |
| | | | 328) | |
| B2M_exon_4 | + | CTTT | CCTGCGGGCCAGGTCATGAGGAG | CCUGCGGGCCAGGUCAUGAGGAGUAUGC |
| | | | TATGCAG (SEQ ID NO: | AG (SEQ ID NO: 709) |
| | | | 329) | |
| B2M_exon_4 | + | TTTC | CTGCGGGCCAGGTCATGAGGAGT | CUGCGGGCCAGGUCAUGAGGAGUAUGCA |
| | | | ATGCAGA (SEQ ID NO: | GA (SEQ ID NO: 710) |
| | | | 330) | |
| B2M_exon_4 | + | ATTT | AATATTTTAGCAAGGAATAGATA | AAUAUUUUAGCAAGGAAUAGAUAUACAA |
| | | | TACAATC (SEQ ID NO: | UC (SEQ ID NO: 711) |
| | | | 331) | |
| B2M_exon_4 | + | TTTA | ATATTTTAGCAAGGAATAGATAT | AUAUUUUAGCAAGGAAUAGAUAUACAAU |
| | | | ACAATCA (SEQ ID NO: | CA (SEQ ID NO: 712) |
| | | | 332) | |
| B2M_exon_4 | + | ATTT | TAGCAAGGAATAGATATACAATC | UAGCAAGGAAUAGAUAUACAAUCAUCCC |
| | | | ATCCCTT (SEQ ID NO: | UU (SEQ ID NO: 713) |
| | | | 333) | |
| B2M_exon_4 | + | TTTT | AGCAAGGAATAGATATACAATCA | AGCAAGGAAUAGAUAUACAAUCAUCCCU |
| | | | TCCCTTG (SEQ ID NO: | UG (SEQ ID NO: 714) |
| | | | 334) | |
| B2M_exon_4 | + | TTTA | GCAAGGAATAGATATACAATCAT | GCAAGGAAUAGAUAUACAAUCAUCCCUU |
| | | | CCCTTGG (SEQ ID NO: | GG (SEQ ID NO: 715) |
| | | | 335) | |
| B2M_exon_4 | + | CTTG | GTCTCCCTGGGGGATTGGTTTCA | GUCUCCCUGGGGGAUUGGUUUCAGGACC |
| | | | GGACCCC (SEQ ID NO: | CC (SEQ ID NO: 716) |
| | | | 336) | |
| B2M_exon_4 | + | ATTG | GTTTCAGGACCCCTTCTTGGACA | GUUUCAGGACCCCUUCUUGGACACCAAA |
| | | | CCAAATC (SEQ ID NO: | UC (SEQ ID NO: 717) |
| | | | 337) | |
| B2M_exon_4 | + | GTTT | CAGGACCCCTTCTTGGACACCAA | CAGGACCCCUUCUUGGACACCAAAUCUA |
| | | | ATCTATG (SEQ ID NO: | UG (SEQ ID NO: 718) |
| | | | 338) | |
| B2M_exon_4 | + | CTTG | TAATACCTAATACAATGTAAATG | UAAUACCUAAUACAAUGUAAAUGCUAUG |
| | | | CTATGCA (SEQ ID NO: | CA (SEQ ID NO: 719) |
| | | | 339) | |
| B2M_exon_4 | + | ATTA | CTTGTAATACCTAATACAATGTA | CUUGUAAUACCUAAUACAAUGUAAAUGC |
| | | | AATGCTA (SEQ ID NO: | UA (SEQ ID NO: 720) |
| | | | 340) | |
| B2M_exon_4 | + | TTTC | TAGATTACTTGTAATACCTAATA | UAGAUUACUUGUAAUACCUAAUACAAUG |
| | | | CAATGTA (SEQ ID NO: | UA (SEQ ID NO: 721) |
| | | | 341) | |
| B2M_exon_4 | + | ATTT | CTAGATTACTTGTAATACCTAAT | CUAGAUUACUUGUAAUACCUAAUACAAU |
| | | | ACAATGT (SEQ ID NO: | GU (SEQ ID NO: 722) |
| | | | 342) | |
| B2M_exon_4 | + | TTTA | AATCATTTCTAGATTACTTGTAA | AAUCAUUUCUAGAUUACUUGUAAUACCU |
| | | | TACCTAA (SEQ ID NO: | AA (SEQ ID NO: 723) |
| | | | 343) | |
| B2M_exon_4 | + | CTTT | AAATCATTTCTAGATTACTTGTA | AAAUCAUUUCUAGAUUACUUGUAAUACC |
| | | | ATACCTA (SEQ ID NO: | UA (SEQ ID NO: 724) |
| | | | 344) | |
| B2M_exon_4 | + | ATTA | GGAATCTGATGCTCAAAGAAGTT | GGAAUCUGAUGCUCAAAGAAGUUAAAUG |
| | | | AAATGGC (SEQ ID NO: | GC (SEQ ID NO: 725) |
| | | | 345) | |
| B2M_exon_4 | + | TTTG | CATATAACCTATCCACATCCTCC | CAUAUAACCUAUCCACAUCCUCCUGUAU |
| | | | TGTATAC (SEQ ID NO: | AC (SEQ ID NO: 726) |
| | | | 346) | |
| B2M_exon_4 | + | CTTC | TATAAAATGGTATAGTATTTGCA | UAUAAAAUGGUAUAGUAUUUGCAUAUAA |
| | | | TATAACC (SEQ ID NO: | CC (SEQ ID NO: 727) |
| | | | 347) | |
| B2M_exon_4 | + | TTTA | AGTCCCTTCTATAAAATGGTATA | AGUCCCUUCUAUAAAAUGGUAUAGUAUU |
| | | | GTATTTG (SEQ ID NO: | UG (SEQ ID NO: 728) |
| | | | 348) | |
| B2M_exon_4 | + | ATTT | AAGTCCCTTCTATAAAATGGTAT | AAGUCCCUUCUAUAAAAUGGUAUAGUAU |
| | | | AGTATTT (SEQ ID NO: | UU (SEQ ID NO: 729) |
| | | | 349) | |
| B2M_exon_4 | + | CTTG | GACACCAAATCTATGGATATTTA | GACACCAAAUCUAUGGAUAUUUAAGUCC |
| | | | AGTCCCT (SEQ ID NO: | CU (SEQ ID NO: 730) |
| | | | 350) | |
| B2M_exon_4 | + | CTTC | TTGGACACCAAATCTATGGATAT | UUGGACACCAAAUCUAUGGAUAUUUAAG |
| | | | TTAAGTC (SEQ ID NO: | UC (SEQ ID NO: 731) |
| | | | 351) | |
| B2M_exon_4 | + | TTTC | AGGACCCCTTCTTGGACACCAAA | AGGACCCCUUCUUGGACACCAAAUCUAU |
| | | | TCTATGG (SEQ ID NO: | GG (SEQ ID NO: 732) |
| | | | 352) | |
| B2M_exon_4 | + | ATTT | GCATATAACCTATCCACATCCTC | GCAUAUAACCUAUCCACAUCCUCCUGUA |
| | | | CTGTATA (SEQ ID NO: | UA (SEQ ID NO: 733) |
| | | | 353) | |
| B2M_exon_4 | + | TTTC | CAGATTAGGAATCTGATGCTCAA | CAGAUUAGGAAUCUGAUGCUCAAAGAAG |
| | | | AGAAGTT (SEQ ID NO: | UU (SEQ ID NO: 734) |
| | | | 354) | |
| B2M_exon_4 | + | TTTT | CCAGATTAGGAATCTGATGCTCA | CCAGAUUAGGAAUCUGAUGCUCAAAGAA |
| | | | AAGAAGT (SEQ ID NO: | GU (SEQ ID NO: 735) |
| | | | 355) | |
| B2M_exon_4 | + | ATTT | TCCAGATTAGGAATCTGATGCTC | UCCAGAUUAGGAAUCUGAUGCUCAAAGA |
| | | | AAAGAAG (SEQ ID NO: | AG (SEQ ID NO: 736) |
| | | | 356) | |
| B2M_exon_4 | + | TTTG | TTCCACAAGTTAAATAAATCATA | UUCCACAAGUUAAAUAAAUCAUAAAACU |
| | | | AAACTTG (SEQ ID NO: | UG (SEQ ID NO: 737) |
| | | | 357) | |
| B2M_exon_4 | + | TTTT | GTTCCACAAGTTAAATAAATCAT | GUUCCACAAGUUAAAUAAAUCAUAAAAC |
| | | | AAAACTT (SEQ ID NO: | UU (SEQ ID NO: 738) |
| | | | 358) | |
| B2M_exon_4 | + | TTTT | TGTTCCACAAGTTAAATAAATCA | UGUUCCACAAGUUAAAUAAAUCAUAAAA |
| | | | TAAAACT (SEQ ID NO: | CU (SEQ ID NO: 739) |
| | | | 359) | |
| B2M_exon_4 | + | ATTT | TTGTTCCACAAGTTAAATAAATC | UUGUUCCACAAGUUAAAUAAAUCAUAAA |
| | | | ATAAAAC (SEQ ID NO: | AC (SEQ ID NO: 740) |
| | | | 360) | |
| B2M_exon_4 | + | TTTA | TTTTTGTTCCACAAGTTAAATAA | UUUUUGUUCCACAAGUUAAAUAAAUCAU |
| | | | ATCATAA (SEQ ID NO: | AA (SEQ ID NO: 741) |
| | | | 361) | |
| B2M_exon_4 | + | GTTT | ATTTTTGTTCCACAAGTTAAATA | AUUUUUGUUCCACAAGUUAAAUAAAUCA |
| | | | AATCATA (SEQ ID NO: | UA (SEQ ID NO: 742) |
| | | | 362) | |
| B2M_exon_4 | + | GTTC | CACAAGTTAAATAAATCATAAAA | CACAAGUUAAAUAAAUCAUAAAACUUGA |
| | | | CTTGATG (SEQ ID NO: | UG (SEQ ID NO: 743) |
| | | | 363) | |
| B2M_exon_4 | + | GTTA | ATCTGGTTTATTTTTGTTCCACA | AUCUGGUUUAUUUUUGUUCCACAAGUUA |
| | | | AGTTAAA (SEQ ID NO: | AA (SEQ ID NO: 744) |
| | | | 364) | |
| B2M_exon_4 | + | TTTG | ATAAAGTAAGGCATGGTTGTGGT | AUAAAGUAAGGCAUGGUUGUGGUUAAUC |
| | | | TAATCTG (SEQ ID NO: | UG (SEQ ID NO: 745) |
| | | | 365) | |
| B2M_exon_4 | + | ATTT | GATAAAGTAAGGCATGGTTGTGG | GAUAAAGUAAGGCAUGGUUGUGGUUAAU |
| | | | TTAATCT (SEQ ID NO: | CU (SEQ ID NO: 746) |
| | | | 366) | |
| B2M_exon_4 | + | CTTA | TACATTTGATAAAGTAAGGCATG | UACAUUUGAUAAAGUAAGGCAUGGUUGU |
| | | | GTTGTGG (SEQ ID NO: | GG (SEQ ID NO: 747) |
| | | | 367) | |
| B2M_exon_4 | + | CTTC | TTATACATTTGATAAAGTAAGGC | UUAUACAUUUGAUAAAGUAAGGCAUGGU |
| | | | ATGGTTG (SEQ ID NO: | UG (SEQ ID NO: 748) |
| | | | 368) | |
| B2M_exon_4 | + | TTTA | CTTCTTATACATTTGATAAAGTA | CUUCUUAUACAUUUGAUAAAGUAAGGCA |
| | | | AGGCATG (SEQ ID NO: | UG (SEQ ID NO: 749) |
| | | | 369) | |
| B2M_exon_4 | + | ATTT | ACTTCTTATACATTTGATAAAGT | ACUUCUUAUACAUUUGAUAAAGUAAGGC |
| | | | AAGGCAT (SEQ ID NO: | AU (SEQ ID NO: 750) |
| | | | 370) | |
| B2M_exon_4 | + | GTTG | TGGTTAATCTGGTTTATTTTTGT | UGGUUAAUCUGGUUUAUUUUUGUUCCAC |
| | | | TCCACAA (SEQ ID NO: | AA (SEQ ID NO: 751) |
| | | | 371) | |
| B2M_exon_4 | + | TTTG | TCATATAAGATTCATATTTACTT | UCAUAUAAGAUUCAUAUUUACUUCUUAU |
| | | | CTTATAC (SEQ ID NO: | AC (SEQ ID NO: 752) |
| | | | 372) | |
| B2M_exon_4 | + | GTTA | AATAAATCATAAAACTTGATGTG | AAUAAAUCAUAAAACUUGAUGUGUUAUC |
| | | | TTATCTC (SEQ ID NO: | UC (SEQ ID NO: 753) |
| | | | 373) | |
| B2M_exon_4 | + | GTTA | TCTCTTATATCTCACTCCCACTA | UCUCUUAUAUCUCACUCCCACUAUUACC |
| | | | TTACCCC (SEQ ID NO: | CC (SEQ ID NO: 754) |
| | | | 374) | |
| B2M_exon_4 | + | ATTC | ACATTTTCCAGATTAGGAATCTG | ACAUUUUCCAGAUUAGGAAUCUGAUGCU |
| | | | ATGCTCA (SEQ ID NO: | CA (SEQ ID NO: 755) |
| | | | 375) | |
| B2M_exon_4 | + | TTTG | GCTCACAGTGTAAAGGGCCTCAG | GCUCACAGUGUAAAGGGCCUCAGUGAUU |
| | | | TGATTCA (SEQ ID NO: | CA (SEQ ID NO: 756) |
| | | | 376) | |
| B2M_exon_4 | + | GTTT | GGCTCACAGTGTAAAGGGCCTCA | GGCUCACAGUGUAAAGGGCCUCAGUGAU |
| | | | GTGATTC (SEQ ID NO: | UC (SEQ ID NO: 757) |
| | | | 377) | |
| B2M_exon_4 | + | CTTG | TATATAGAGTTTGGCTCACAGTG | UAUAUAGAGUUUGGCUCACAGUGUAAAG |
| | | | TAAAGGG (SEQ ID NO: | GG (SEQ ID NO: 758) |
| | | | 378) | |
| B2M_exon_4 | + | CTTG | GTAAAAAATGTGAACCCCTTGTA | GUAAAAAAUGUGAACCCCUUGUAUAUAG |
| | | | TATAGAG (SEQ ID NO: | AG (SEQ ID NO: 759) |
| | | | 379) | |
| B2M_exon_4 | + | GTTC | CACTTGGTAAAAAATGTGAACCC | CACUUGGUAAAAAAUGUGAACCCCUUGU |
| | | | CTTGTAT (SEQ ID NO: | AU (SEQ ID NO: 760) |
| | | | 380) | |
| B2M_exon_4 | + | CTTG | ATGTGTTATCTCTTATATCTCAC | AUGUGUUAUCUCUUAUAUCUCACUCCCA |
| | | | TCCCACT (SEQ ID NO: | CU (SEQ ID NO: 761) |
| | | | 381) | |
| B2M_exon_4 | + | CTTC | AAGTTCCACTTGGTAAAAAATGT | AAGUUCCACUUGGUAAAAAAUGUGAACC |
| | | | GAACCCC (SEQ ID NO: | CC (SEQ ID NO: 762) |
| | | | 382) | |
| B2M_exon_4 | + | TTTT | CAAACAGGGAAACAGTCTTCAAG | CAAACAGGGAAACAGUCUUCAAGUUCCA |
| | | | TTCCACT (SEQ ID NO: | CU (SEQ ID NO: 763) |
| | | | 383) | |
| B2M_exon_4 | + | ATTT | TCAAACAGGGAAACAGTCTTCAA | UCAAACAGGGAAACAGUCUUCAAGUUCC |
| | | | GTTCCAC (SEQ ID NO: | AC (SEQ ID NO: 764) |
| | | | 384) | |
| B2M_exon_4 | + | TTTA | TTTTCAAACAGGGAAACAGTCTT | UUUUCAAACAGGGAAACAGUCUUCAAGU |
| | | | CAAGTTC (SEQ ID NO: | UC (SEQ ID NO: 765) |
| | | | 385) | |
| B2M_exon_4 | − | CTTC | AAACCTGAAAAGAAAAGAAAAAG | AAACCUGAAAAGAAAAGAAAAAGGUUAG |
| | | | GTTAGCA (SEQ ID NO: | CA (SEQ ID NO: 766) |
| | | | 386) | |
| B2M_exon_4 | + | ATTA | CCCCTTTATTTTCAAACAGGGAA | CCCCUUUAUUUUCAAACAGGGAAACAGU |
| | | | ACAGTCT (SEQ ID NO: | CU (SEQ ID NO: 767) |
| | | | 387) | |
| B2M_exon_4 | + | CTTA | TATCTCACTCCCACTATTACCCC | UAUCUCACUCCCACUAUUACCCCUUUAU |
| | | | TTTATTT (SEQ ID NO: | UU (SEQ ID NO: 768) |
| | | | 388) | |
| B2M_exon_4 | + | TTTC | AAACAGGGAAACAGTCTTCAAGT | AAACAGGGAAACAGUCUUCAAGUUCCAC |
| | | | TCCACTT (SEQ ID NO: | UU (SEQ ID NO: 769) |
| | | | 389) | |
| B2M_exon_4 | − | GTTA | GCAATGAATTTATTTTATTTGGA | GCAAUGAAUUUAUUUUAUUUGGAUUGCA |
| | | | TTGCAGA (SEQ ID NO: | GA (SEQ ID NO: 770) |
| | | | 390) | |

| | | | seq | | seq | |
|---|---|---|---|---|---|---|
| | | | id | | id | |
| annotation | strand | pam | no | target_seq | no | spacer |
| intron | + | ATT | 819 | CTGAAGCTGACAGCATTCGG | 1019 | CUGAAGCUGACAGCAUUCGG |
| | | C | | | | |
| intron | + | CTT | 820 | CTGGCCTGGAGGCTATCCAG | 1020 | CUGGCCUGGAGGCUAUCCAG |
| | | T | | | | |
| intron | + | TTT | 821 | TGGCCTGGAGGCTATCCAGC | 1021 | UGGCCUGGAGGCUAUCCAGC |
| | | C | | | | |
| intron | + | CTT | 822 | CTCTCCCGCTCTGCACCCTC | 1022 | CUCUCCCGCUCUGCACCCUC |
| | | C | | | | |
| intron | + | CTT | 823 | CCTTCTCCAAGTTCTCCTTG | 1023 | CCUUCUCCAAGUUCUCCUUG |
| | | C | | | | |
| intron | + | CTT | 824 | TCCAAGTTCTCCTTGGTGGC | 1024 | UCCAAGUUCUCCUUGGUGGC |
| | | C | | | | |
| intron | + | GTT | 825 | TCCTTGGTGGCCCGCCGTGG | 1025 | UCCUUGGUGGCCCGCCGUGG |
| | | C | | | | |
| intron | + | CTT | 826 | GTGGCCCGCCGTGGGGCTAG | 1026 | GUGGCCCGCCGUGGGGCUAG |
| | | G | | | | |
| intron | + | CTT | 827 | CCCCTTTCGGCGGGGAGCAG | 1027 | CCCCUUUCGGCGGGGAGCAG |
| | | G | | | | |
| intron | + | CTT | 828 | CGGCGGGGAGCAGGGGAGAC | 1028 | CGGCGGGGAGCAGGGGAGAC |
| | | T | | | | |
| intron | + | TTT | 829 | GGCGGGGAGCAGGGGAGACC | 1029 | GGCGGGGAGCAGGGGAGACC |
| | | C | | | | |
| intron | + | CTT | 830 | GGCCTACGGCGACGGGAGGG | 1030 | GGCCUACGGCGACGGGAGGG |
| | | T | | | | |
| intron | + | TTT | 831 | GCCTACGGCGACGGGAGGGT | 1031 | GCCUACGGCGACGGGAGGGU |
| | | G | | | | |
| intron | + | GTT | 832 | AGGGCGTCGATAAGCGTCAG | 1032 | AGGGCGUCGAUAAGCGUCAG |
| | | T | | | | |
| intron | + | TTT | 833 | GGGCGTCGATAAGCGTCAGA | 1033 | GGGCGUCGAUAAGCGUCAGA |
| | | A | | | | |
| intron | + | GTT | 834 | GGGGAGGGTTTCTCTTCCGC | 1034 | GGGGAGGGUUUCUCUUCCGC |
| | | G | | | | |
| intron | + | GTT | 835 | CTCTTCCGCTCTTTCGCGGG | 1035 | CUCUUCCGCUCUUUCGCGGG |
| | | T | | | | |
| intron | + | TTT | 836 | TCTTCCGCTCTTTCGCGGGG | 1036 | UCUUCCGCUCUUUCGCGGGG |
| | | C | | | | |
| intron | + | CTT | 837 | CGCTCTTTCGCGGGGCCTCT | 1037 | CGCUCUUUCGCGGGGCCUCU |
| | | C | | | | |
| intron | + | CTT | 838 | CGCGGGGCCTCTGGCTCCCC | 1038 | CGCGGGGCCUCUGGCUCCCC |
| | | T | | | | |
| intron | + | TTT | 839 | GCGGGGCCTCTGGCTCCCCC | 1039 | GCGGGGCCUCUGGCUCCCCC |
| | | C | | | | |
| intron | + | GTT | 840 | GTGAACGCGTGGAGGGGCGC | 1040 | GUGAACGCGUGGAGGGGCGC |
| | | T | | | | |
| intron | + | TTT | 841 | TGAACGCGTGGAGGGGCGCT | 1041 | UGAACGCGUGGAGGGGCGCU |
| | | G | | | | |
| intron | + | CTT | 842 | GGGTCTGGGGGAGGCGTCGC | 1042 | GGGUCUGGGGGAGGCGUCGC |
| | | G | | | | |
| intron | − | CTT | 843 | CCCGGGCGACGCCTCCCCCA | 1043 | CCCGGGCGACGCCUCCCCCA |
| | | A | | | | |
| intron | − | GTT | 844 | ACAAACCTCAGCGCCGCGCC | 1044 | ACAAACCUCAGCGCCGCGCC |
| | | C | | | | |
| intron | − | CTT | 845 | GGGACGAGCCTACCCGTCCC | 1045 | GGGACGAGCCUACCCGUCCC |
| | | T | | | | |
| intron | − | TTT | 846 | GGACGAGCCTACCCGTCCCC | 1046 | GGACGAGCCUACCCGUCCCC |
| | | G | | | | |
| intron | − | CTT | 847 | TCGACGCCCTAAACTTTGTC | 1047 | UCGACGCCCUAAACUUUGUC |
| | | A | | | | |
| intron | − | CTT | 848 | GTCCCGACCCTCCCGTCGCC | 1048 | GUCCCGACCCUCCCGUCGCC |
| | | T | | | | |
| intron | − | TTT | 849 | TCCCGACCCTCCCGTCGCCG | 1049 | UCCCGACCCUCCCGUCGCCG |
| | | G | | | | |
| intron | − | CTT | 850 | CCCACTCCCAGGCCACCCCG | 1050 | CCCACUCCCAGGCCACCCCG |
| | | C | | | | |
| intron | − | CTT | 851 | CCCGAGATCCAGCCCTGGAC | 1051 | CCCGAGAUCCAGCCCUGGAC |
| | | C | | | | |
| intron | − | CTT | 852 | GAGAAGGGAAGTCACGGAGC | 1052 | GAGAAGGGAAGUCACGGAGC |
| | | G | | | | |
| intron | − | CTT | 853 | AGGAATGCCCGCCAGCGCGA | 1053 | AGGAAUGCCCGCCAGCGCGA |
| | | C | | | | |
| intron | + | ATT | 854 | TGAGGGAAAGATACCAAGTC | 1054 | UGAGGGAAAGAUACCAAGUC |
| | | A | | | | |
| intron | + | GTT | 855 | ATTCTTCAAAATGGAGGTGG | 1055 | AUUCUUCAAAAUGGAGGUGG |
| | | T | | | | |
| intron | + | TTT | 856 | TTCTTCAAAATGGAGGTGGC | 1056 | UUCUUCAAAAUGGAGGUGGC |
| | | A | | | | |
| intron | + | ATT | 857 | TTCAAAATGGAGGTGGCTTG | 1057 | UUCAAAAUGGAGGUGGCUUG |
| | | C | | | | |
| intron | + | CTT | 858 | AAAATGGAGGTGGCTTGTTG | 1058 | AAAAUGGAGGUGGCUUGUUG |
| | | C | | | | |
| intron | + | CTT | 859 | TTGGGAAGGTGGAAGCTCAT | 1059 | UUGGGAAGGUGGAAGCUCAU |
| | | G | | | | |
| intron | + | GTT | 860 | GGAAGGTGGAAGCTCATTTG | 1060 | GGAAGGUGGAAGCUCAUUUG |
| | | G | | | | |
| intron | + | ATT | 861 | GGCCAGAGTGGAAATGGAAT | 1061 | GGCCAGAGUGGAAAUGGAAU |
| | | T | | | | |
| intron | + | TTT | 862 | GCCAGAGTGGAAATGGAATT | 1062 | GCCAGAGUGGAAAUGGAAUU |
| | | G | | | | |
| intron | + | ATT | 863 | GGAGAAATCGATGACCAAAT | 1063 | GGAGAAAUCGAUGACCAAAU |
| | | G | | | | |
| intron | + | CTT | 864 | GTGCCTGATATAGCTTGACA | 1064 | GUGCCUGAUAUAGCUUGACA |
| | | G | | | | |
| intron | + | CTT | 865 | ACACCAAGTTAGCCCCAAGT | 1065 | ACACCAAGUUAGCCCCAAGU |
| | | G | | | | |
| intron | + | GTT | 866 | GCCCCAAGTGAAATACCCTG | 1066 | GCCCCAAGUGAAAUACCCUG |
| | | A | | | | |
| intron | + | ATT | 867 | ATGTGTCTTTTCCCGATATT | 1067 | AUGUGUCUUUUCCCGAUAUU |
| | | A | | | | |
| intron | + | GTT | 868 | AGTGGGGTAAGTCTTACATT | 1068 | AGUGGGGUAAGUCUUACAUU |
| | | A | | | | |
| intron | + | CTT | 869 | CATTCTTTTGTAAGCTGCTG | 1069 | CAUUCUUUUGUAAGCUGCUG |
| | | A | | | | |
| intron | + | ATT | 870 | TTTTGTAAGCTGCTGAAAGT | 1070 | UUUUGUAAGCUGCUGAAAGU |
| | | C | | | | |
| intron | + | CTT | 871 | TGTAAGCTGCTGAAAGTTGT | 1071 | UGUAAGCUGCUGAAAGUUGU |
| | | T | | | | |
| intron | + | TTT | 872 | GTAAGCTGCTGAAAGTTGTG | 1072 | GUAAGCUGCUGAAAGUUGUG |
| | | T | | | | |
| intron | + | TTT | 873 | TAAGCTGCTGAAAGTTGTGT | 1073 | UAAGCUGCUGAAAGUUGUGU |
| | | G | | | | |
| intron | + | GTT | 874 | TGTATGAGTAGTCATATCAT | 1074 | UGUAUGAGUAGUCAUAUCAU |
| | | G | | | | |
| intron | + | CTT | 875 | GATATAAAAAAGGTCTATGG | 1075 | GAUAUAAAAAAGGUCUAUGG |
| | | T | | | | |
| intron | + | TTT | 876 | ATATAAAAAAGGTCTATGGC | 1076 | AUAUAAAAAAGGUCUAUGGC |
| | | G | | | | |
| intron | + | ATT | 877 | GGATTGTCAGGGAATGTTCT | 1077 | GGAUUGUCAGGGAAUGUUCU |
| | | G | | | | |
| intron | + | ATT | 878 | TCAGGGAATGTTCTTAAAGA | 1078 | UCAGGGAAUGUUCUUAAAGA |
| | | G | | | | |
| intron | + | GTT | 879 | TTAAAGATCAGATTAGTGGC | 1079 | UUAAAGAUCAGAUUAGUGGC |
| | | C | | | | |
| intron | + | CTT | 880 | AAGATCAGATTAGTGGCACC | 1080 | AAGAUCAGAUUAGUGGCACC |
| | | A | | | | |
| intron | + | ATT | 881 | GTGGCACCTGCTGAGATACT | 1081 | GUGGCACCUGCUGAGAUACU |
| | | A | | | | |
| intron | + | GTT | 882 | CTGAACCAGTAGTTTCCCTG | 1082 | CUGAACCAGUAGUUUCCCUG |
| | | T | | | | |
| intron | + | TTT | 883 | TGAACCAGTAGTTTCCCTGC | 1083 | UGAACCAGUAGUUUCCCUGC |
| | | C | | | | |
| intron | + | GTT | 884 | CCCTGCAGTTGAGCAGGGAG | 1084 | CCCUGCAGUUGAGCAGGGAG |
| | | T | | | | |
| intron | + | TTT | 885 | CCTGCAGTTGAGCAGGGAGC | 1085 | CCUGCAGUUGAGCAGGGAGC |
| | | C | | | | |
| intron | + | GTT | 886 | AGCAGGGAGCAGCAGCAGCA | 1086 | AGCAGGGAGCAGCAGCAGCA |
| | | G | | | | |
| intron | + | CTT | 887 | CACAAATACATATACACTCT | 1087 | CACAAAUACAUAUACACUCU |
| | | G | | | | |
| intron | + | CTT | 888 | ACACTTCTTACCTACTGGCT | 1088 | ACACUUCUUACCUACUGGCU |
| | | A | | | | |
| intron | + | CTT | 889 | TTACCTACTGGCTTCCTCTA | 1089 | UUACCUACUGGCUUCCUCUA |
| | | C | | | | |
| intron | + | CTT | 890 | CCTACTGGCTTCCTCTAGCT | 1090 | CCUACUGGCUUCCUCUAGCU |
| | | A | | | | |
| intron | + | CTT | 891 | CTCTAGCTTTTGTGGCAGCT | 1091 | CUCUAGCUUUUGUGGCAGCU |
| | | C | | | | |
| intron | + | CTT | 892 | TGTGGCAGCTTCAGGTATAT | 1092 | UGUGGCAGCUUCAGGUAUAU |
| | | T | | | | |
| intron | + | TTT | 893 | GTGGCAGCTTCAGGTATATT | 1093 | GUGGCAGCUUCAGGUAUAUU |
| | | T | | | | |
| intron | + | TTT | 894 | TGGCAGCTTCAGGTATATTT | 1094 | UGGCAGCUUCAGGUAUAUUU |
| | | G | | | | |
| intron | + | CTT | 895 | AGGTATATTTAGCACTGAAC | 1095 | AGGUAUAUUUAGCACUGAAC |
| | | C | | | | |
| intron | + | ATT | 896 | AGCACTGAACGAACATCTCA | 1096 | AGCACUGAACGAACAUCUCA |
| | | T | | | | |
| intron | + | TTT | 897 | GCACTGAACGAACATCTCAA | 1097 | GCACUGAACGAACAUCUCAA |
| | | A | | | | |
| intron | + | CTT | 898 | GTTTGTAAGTCCTGCTGTCC | 1098 | GUUUGUAAGUCCUGCUGUCC |
| | | T | | | | |
| intron | + | TTT | 899 | TTTGTAAGTCCTGCTGTCCT | 1099 | UUUGUAAGUCCUGCUGUCCU |
| | | G | | | | |
| intron | + | GTT | 900 | GTAAGTCCTGCTGTCCTAGC | 1100 | GUAAGUCCUGCUGUCCUAGC |
| | | T | | | | |
| intron | + | TTT | 901 | TAAGTCCTGCTGTCCTAGCA | 1101 | UAAGUCCUGCUGUCCUAGCA |
| | | G | | | | |
| intron | + | CTT | 902 | TCCAGTACTTTCTGGCTGGA | 1102 | UCCAGUACUUUCUGGCUGGA |
| | | C | | | | |
| intron | + | CTT | 903 | CTGGCTGGATTGGTATCTGA | 1103 | CUGGCUGGAUUGGUAUCUGA |
| | | T | | | | |
| intron | + | TTT | 904 | TGGCTGGATTGGTATCTGAG | 1104 | UGGCUGGAUUGGUAUCUGAG |
| | | C | | | | |
| intron | + | ATT | 905 | GTATCTGAGGCTAGTAGGAA | 1105 | GUAUCUGAGGCUAGUAGGAA |
| | | G | | | | |
| intron | + | CTT | 906 | TTCCTGCTGGGTAGCTCTAA | 1106 | UUCCUGCUGGGUAGCUCUAA |
| | | G | | | | |
| intron | + | GTT | 907 | CTGCTGGGTAGCTCTAAACA | 1107 | CUGCUGGGUAGCUCUAAACA |
| | | C | | | | |
| intron | + | ATT | 908 | ATGGGTAGGAACAGCAGCCT | 1108 | AUGGGUAGGAACAGCAGCCU |
| | | C | | | | |
| intron | + | ATT | 909 | TGCCAGCCTTATTTCTAACC | 1109 | UGCCAGCCUUAUUUCUAACC |
| | | C | | | | |
| intron | + | CTT | 910 | TTTCTAACCATTTTAGACAT | 1110 | UUUCUAACCAUUUUAGACAU |
| | | A | | | | |
| intron | + | ATT | 911 | CTAACCATTTTAGACATTTG | 1111 | CUAACCAUUUUAGACAUUUG |
| | | T | | | | |
| intron | + | TTT | 912 | TAACCATTTTAGACATTTGT | 1112 | UAACCAUUUUAGACAUUUGU |
| | | C | | | | |
| intron | + | ATT | 913 | TAGACATTTGTTAGTACATG | 1113 | UAGACAUUUGUUAGUACAUG |
| | | T | | | | |
| intron | + | TTT | 914 | AGACATTTGTTAGTACATGG | 1114 | AGACAUUUGUUAGUACAUGG |
| | | T | | | | |
| intron | + | TTT | 915 | GACATTTGTTAGTACATGGT | 1115 | GACAUUUGUUAGUACAUGGU |
| | | A | | | | |
| intron | + | ATT | 916 | GTTAGTACATGGTATTTTAA | 1116 | GUUAGUACAUGGUAUUUUAA |
| | | T | | | | |
| intron | + | TTT | 917 | TTAGTACATGGTATTTTAAA | 1117 | UUAGUACAUGGUAUUUUAAA |
| | | G | | | | |
| intron | + | GTT | 918 | GTACATGGTATTTTAAAAGT | 1118 | GUACAUGGUAUUUUAAAAGU |
| | | A | | | | |
| intron | + | ATT | 919 | TAAAAGTAAAACTTAATGTC | 1119 | UAAAAGUAAAACUUAAUGUC |
| | | T | | | | |
| intron | + | TTT | 920 | AAAAGTAAAACTTAATGTCT | 1120 | AAAAGUAAAACUUAAUGUCU |
| | | T | | | | |
| intron | + | TTT | 921 | AAAGTAAAACTTAATGTCTT | 1121 | AAAGUAAAACUUAAUGUCUU |
| | | A | | | | |
| intron | + | CTT | 922 | ATGTCTTCCTTTTTTTTCTC | 1122 | AUGUCUUCCUUUUUUUUCUC |
| | | A | | | | |
| intron | + | CTT | 923 | CTTTTTTTTCTCCACTGTCT | 1123 | CUUUUUUUUCUCCACUGUCU |
| | | C | | | | |
| intron | + | CTT | 924 | TTTTTCTCCACTGTCTTTTT | 1124 | UUUUUCUCCACUGUCUUUUU |
| | | T | | | | |
| intron | + | TTT | 925 | TTTTCTCCACTGTCTTTTTC | 1125 | UUUUCUCCACUGUCUUUUUC |
| | | T | | | | |
| intron | + | CTT | 926 | TTCATAGATCGAGACATGTA | 1126 | UUCAUAGAUCGAGACAUGUA |
| | | T | | | | |
| intron | + | TTT | 927 | TCATAGATCGAGACATGTAA | 1127 | UCAUAGAUCGAGACAUGUAA |
| | | T | | | | |
| intron | + | TTT | 928 | CATAGATCGAGACATGTAAG | 1128 | CAUAGAUCGAGACAUGUAAG |
| | | T | | | | |
| intron | + | TTT | 929 | ATAGATCGAGACATGTAAGC | 1129 | AUAGAUCGAGACAUGUAAGC |
| | | C | | | | |
| intron | + | GTT | 930 | TTGACCTTGAGAAAATGTTT | 1130 | UUGACCUUGAGAAAAUGUUU |
| | | T | | | | |
| intron | + | TTT | 931 | TGACCTTGAGAAAATGTTTT | 1131 | UGACCUUGAGAAAAUGUUUU |
| | | T | | | | |
| intron | + | TTT | 932 | GACCTTGAGAAAATGTTTTT | 1132 | GACCUUGAGAAAAUGUUUUU |
| | | T | | | | |
| intron | + | TTT | 933 | ACCTTGAGAAAATGTTTTTG | 1133 | ACCUUGAGAAAAUGUUUUUG |
| | | G | | | | |
| intron | + | CTT | 934 | AGAAAATGTTTTTGTTTCAC | 1134 | AGAAAAUGUUUUUGUUUCAC |
| | | G | | | | |
| intron | + | GTT | 935 | TTGTTTCACTGTCCTGAGGA | 1135 | UUGUUUCACUGUCCUGAGGA |
| | | T | | | | |
| intron | + | TTT | 936 | TGTTTCACTGTCCTGAGGAC | 1136 | UGUUUCACUGUCCUGAGGAC |
| | | T | | | | |
| intron | + | TTT | 937 | GTTTCACTGTCCTGAGGACT | 1137 | GUUUCACUGUCCUGAGGACU |
| | | T | | | | |
| intron | + | TTT | 938 | TTTCACTGTCCTGAGGACTA | 1138 | UUUCACUGUCCUGAGGACUA |
| | | G | | | | |
| intron | + | GTT | 939 | CACTGTCCTGAGGACTATTT | 1139 | CACUGUCCUGAGGACUAUUU |
| | | T | | | | |
| intron | + | TTT | 940 | ACTGTCCTGAGGACTATTTA | 1140 | ACUGUCCUGAGGACUAUUUA |
| | | C | | | | |
| intron | + | ATT | 941 | ATAGACAGCTCTAACATGAT | 1141 | AUAGACAGCUCUAACAUGAU |
| | | T | | | | |
| intron | + | TTT | 942 | TAGACAGCTCTAACATGATA | 1142 | UAGACAGCUCUAACAUGAUA |
| | | A | | | | |
| intron | − | GTT | 943 | TCATGTTAGAGCTGTCTATA | 1143 | UCAUGUUAGAGCUGUCUAUA |
| | | A | | | | |
| intron | − | GTT | 944 | GAGCTGTCTATAAATAGTCC | 1144 | GAGCUGUCUAUAAAUAGUCC |
| | | A | | | | |
| intron | − | ATT | 945 | TCTCAAGGTCAAAAACTTAC | 1145 | UCUCAAGGUCAAAAACUUAC |
| | | T | | | | |
| intron | − | TTT | 946 | CTCAAGGTCAAAAACTTACC | 1146 | CUCAAGGUCAAAAACUUACC |
| | | T | | | | |
| intron | − | TTT | 947 | TCAAGGTCAAAAACTTACCT | 1147 | UCAAGGUCAAAAACUUACCU |
| | | C | | | | |
| intron | − | CTT | 948 | CATGTCTCGATCTATGAAAA | 1148 | CAUGUCUCGAUCUAUGAAAA |
| | | A | | | | |
| intron | − | ATT | 949 | AGTTTTACTTTTAAAATACC | 1149 | AGUUUUACUUUUAAAAUACC |
| | | A | | | | |
| intron | − | GTT | 950 | TACTTTTAAAATACCATGTA | 1150 | UACUUUUAAAAUACCAUGUA |
| | | T | | | | |
| intron | − | TTT | 951 | ACTTTTAAAATACCATGTAC | 1151 | ACUUUUAAAAUACCAUGUAC |
| | | T | | | | |
| intron | − | TTT | 952 | CTTTTAAAATACCATGTACT | 1152 | CUUUUAAAAUACCAUGUACU |
| | | A | | | | |
| intron | − | CTT | 953 | TAAAATACCATGTACTAACA | 1153 | UAAAAUACCAUGUACUAACA |
| | | T | | | | |
| intron | − | TTT | 954 | AAAATACCATGTACTAACAA | 1154 | AAAAUACCAUGUACUAACAA |
| | | T | | | | |
| intron | − | TTT | 955 | AAATACCATGTACTAACAAA | 1155 | AAAUACCAUGUACUAACAAA |
| | | A | | | | |
| intron | − | GTT | 956 | GAAATAAGGCTGGCAGAATA | 1156 | GAAAUAAGGCUGGCAGAAUA |
| | | A | | | | |
| intron | − | GTT | 957 | CTACCCATGAATACATTGTT | 1157 | CUACCCAUGAAUACAUUGUU |
| | | C | | | | |
| intron | − | ATT | 958 | TTTAGAGCTACCCAGCAGGA | 1158 | UUUAGAGCUACCCAGCAGGA |
| | | G | | | | |
| intron | − | GTT | 959 | AGAGCTACCCAGCAGGAACA | 1159 | AGAGCUACCCAGCAGGAACA |
| | | T | | | | |
| intron | − | TTT | 960 | GAGCTACCCAGCAGGAACAA | 1160 | GAGCUACCCAGCAGGAACAA |
| | | A | | | | |
| intron | − | CTT | 961 | CTACTAGCCTCAGATACCAA | 1161 | CUACUAGCCUCAGAUACCAA |
| | | C | | | | |
| intron | − | ATT | 962 | TAGGATGCTAGGACAGCAGG | 1162 | UAGGAUGCUAGGACAGCAGG |
| | | A | | | | |
| intron | − | CTT | 963 | CAAACAAAGGCCTATACCTT | 1163 | CAAACAAAGGCCUAUACCUU |
| | | A | | | | |
| intron | − | CTT | 964 | TTGAGATGTTCGTTCAGTGC | 1164 | UUGAGAUGUUCGUUCAGUGC |
| | | C | | | | |
| intron | − | CTT | 965 | AGATGTTCGTTCAGTGCTAA | 1165 | AGAUGUUCGUUCAGUGCUAA |
| | | G | | | | |
| intron | − | GTT | 966 | GTTCAGTGCTAAATATACCT | 1166 | GUUCAGUGCUAAAUAUACCU |
| | | C | | | | |
| intron | − | GTT | 967 | AGTGCTAAATATACCTGAAG | 1167 | AGUGCUAAAUAUACCUGAAG |
| | | C | | | | |
| intron | − | GTT | 968 | AGAGTGTATATGTATTTGTG | 1168 | AGAGUGUAUAUGUAUUUGUG |
| | | A | | | | |
| intron | − | ATT | 969 | GTGCAAGTGCTGCTGCTGCT | 1169 | GUGCAAGUGCUGCUGCUGCU |
| | | T | | | | |
| intron | − | TTT | 970 | TGCAAGTGCTGCTGCTGCTC | 1170 | UGCAAGUGCUGCUGCUGCUC |
| | | G | | | | |
| intron | − | GTT | 971 | AGAAACCATGCTGTGCATCA | 1171 | AGAAACCAUGCUGUGCAUCA |
| | | C | | | | |
| intron | − | CTT | 972 | AAGAACATTCCCTGACAATC | 1172 | AAGAACAUUCCCUGACAAUC |
| | | T | | | | |
| intron | − | TTT | 973 | AGAACATTCCCTGACAATCC | 1173 | AGAACAUUCCCUGACAAUCC |
| | | A | | | | |
| intron | − | ATT | 974 | CCTGACAATCCCAATATGCA | 1174 | CCUGACAAUCCCAAUAUGCA |
| | | C | | | | |
| intron | − | ATT | 975 | TTTATATCAGATGGGATGGG | 1175 | UUUAUAUCAGAUGGGAUGGG |
| | | G | | | | |
| intron | − | GTT | 976 | ATATCAGATGGGATGGGACT | 1176 | AUAUCAGAUGGGAUGGGACU |
| | | T | | | | |
| intron | − | TTT | 977 | TATCAGATGGGATGGGACTC | 1177 | UAUCAGAUGGGAUGGGACUC |
| | | A | | | | |
| intron | − | ATT | 978 | AGGGTAGTATGGCCATAGAC | 1178 | AGGGUAGUAUGGCCAUAGAC |
| | | C | | | | |
| intron | − | CTT | 979 | TTTATATCAAAGCAGCTTTA | 1179 | UUUAUAUCAAAGCAGCUUUA |
| | | T | | | | |
| intron | − | TTT | 980 | TTATATCAAAGCAGCTTTAT | 1180 | UUAUAUCAAAGCAGCUUUAU |
| | | T | | | | |
| intron | − | TTT | 981 | TATATCAAAGCAGCTTTATG | 1181 | UAUAUCAAAGCAGCUUUAUG |
| | | T | | | | |
| intron | − | TTT | 982 | ATATCAAAGCAGCTTTATGA | 1182 | AUAUCAAAGCAGCUUUAUGA |
| | | T | | | | |
| intron | − | TTT | 983 | TATCAAAGCAGCTTTATGAT | 1183 | UAUCAAAGCAGCUUUAUGAU |
| | | A | | | | |
| intron | − | CTT | 984 | ATGATATGACTACTCATACA | 1184 | AUGAUAUGACUACUCAUACA |
| | | T | | | | |
| intron | − | TTT | 985 | TGATATGACTACTCATACAC | 1185 | UGAUAUGACUACUCAUACAC |
| | | A | | | | |
| intron | − | CTT | 986 | CAGCAGCTTACAAAAGAATG | 1186 | CAGCAGCUUACAAAAGAAUG |
| | | T | | | | |
| intron | − | TTT | 987 | AGCAGCTTACAAAAGAATGT | 1187 | AGCAGCUUACAAAAGAAUGU |
| | | C | | | | |
| intron | − | CTT | 988 | GGAGTACCTGAGGAATATCG | 1188 | GGAGUACCUGAGGAAUAUCG |
| | | T | | | | |
| intron | − | TTT | 989 | GAGTACCTGAGGAATATCGG | 1189 | GAGUACCUGAGGAAUAUCGG |
| | | G | | | | |
| intron | − | ATT | 990 | ATATTGCCAGGGTATTTCAC | 1190 | AUAUUGCCAGGGUAUUUCAC |
| | | A | | | | |
| intron | − | ATT | 991 | CCAGGGTATTTCACTTGGGG | 1191 | CCAGGGUAUUUCACUUGGGG |
| | | G | | | | |
| intron | − | ATT | 992 | CACTTGGGGCTAACTTGGTG | 1192 | CACUUGGGGCUAACUUGGUG |
| | | T | | | | |
| intron | − | TTT | 993 | ACTTGGGGCTAACTTGGTGT | 1193 | ACUUGGGGCUAACUUGGUGU |
| | | C | | | | |
| intron | − | CTT | 994 | GGGCTAACTTGGTGTCAAGC | 1194 | GGGCUAACUUGGUGUCAAGC |
| | | G | | | | |
| intron | − | CTT | 995 | GTGTCAAGCTATATCAGGCA | 1195 | GUGUCAAGCUAUAUCAGGCA |
| | | G | | | | |
| intron | − | GTT | 996 | ACATTTGGTCATCGATTTCT | 1196 | ACAUUUGGUCAUCGAUUUCU |
| | | T | | | | |
| intron | − | TTT | 997 | CATTTGGTCATCGATTTCTC | 1197 | CAUUUGGUCAUCGAUUUCUC |
| | | A | | | | |
| intron | − | ATT | 998 | GGTCATCGATTTCTCCCAAT | 1198 | GGUCAUCGAUUUCUCCCAAU |
| | | T | | | | |
| intron | − | TTT | 999 | GTCATCGATTTCTCCCAATT | 1199 | GUCAUCGAUUUCUCCCAAUU |
| | | G | | | | |
| intron | − | ATT | 1000 | CTCCCAATTCCATTTCCACT | 1200 | CUCCCAAUUCCAUUUCCACU |
| | | T | | | | |
| intron | − | TTT | 1001 | TCCCAATTCCATTTCCACTC | 1201 | UCCCAAUUCCAUUUCCACUC |
| | | C | | | | |
| intron | − | ATT | 1002 | CATTTCCACTCTGGCCAAAT | 1202 | CAUUUCCACUCUGGCCAAAU |
| | | C | | | | |
| intron | − | ATT | 1003 | CCACTCTGGCCAAATGAGCT | 1203 | CCACUCUGGCCAAAUGAGCU |
| | | T | | | | |
| intron | − | TTT | 1004 | CACTCTGGCCAAATGAGCTT | 1204 | CACUCUGGCCAAAUGAGCUU |
| | | C | | | | |
| intron | − | CTT | 1005 | CACCTTCCCAACAAGCCACC | 1205 | CACCUUCCCAACAAGCCACC |
| | | C | | | | |
| intron | − | CTT | 1006 | CCAACAAGCCACCTCCATTT | 1206 | CCAACAAGCCACCUCCAUUU |
| | | C | | | | |
| intron | − | ATT | 1007 | TGAAGAATAAACCGTGACTT | 1207 | UGAAGAAUAAACCGUGACUU |
| | | T | | | | |
| intron | − | TTT | 1008 | GAAGAATAAACCGTGACTTG | 1208 | GAAGAAUAAACCGUGACUUG |
| | | T | | | | |
| intron | − | TTT | 1009 | AAGAATAAACCGTGACTTGG | 1209 | AAGAAUAAACCGUGACUUGG |
| | | G | | | | |
| intron | − | CTT | 1010 | GTATCTTTCCCTCATAATTC | 1210 | GUAUCUUUCCCUCAUAAUUC |
| | | G | | | | |
| intron | − | CTT | 1011 | CCCTCATAATTCCTCTATAC | 1211 | CCCUCAUAAUUCCUCUAUAC |
| | | T | | | | |
| intron | − | TTT | 1012 | CCTCATAATTCCTCTATACA | 1212 | CCUCAUAAUUCCUCUAUACA |
| | | C | | | | |
| intron | − | ATT | 1013 | CTCTATACATGCCTTTTTTG | 1213 | CUCUAUACAUGCCUUUUUUG |
| | | C | | | | |
| intron | − | CTT | 1014 | TTTGTTTTTTTTCTAGCAGA | 1214 | UUUGUUUUUUUUCUAGCAGA |
| | | T | | | | |
| intron | − | TTT | 1015 | TTGTTTTTTTTCTAGCAGAT | 1215 | UUGUUUUUUUUCUAGCAGAU |
| | | T | | | | |
| intron | − | TTT | 1016 | TGTTTTTTTTCTAGCAGATT | 1216 | UGUUUUUUUUCUAGCAGAUU |
| | | T | | | | |
| intron | − | TTT | 1017 | GTTTTTTTTCTAGCAGATTT | 1217 | GUUUUUUUUCUAGCAGAUUU |
| | | T | | | | |
| intron | − | TTT | 1018 | TTTTTTTTCTAGCAGATTTC | 1218 | UUUUUUUUCUAGCAGAUUUC |
| | | G | | | | |

The invention includes all combinations of the direct repeats and spacers listed above, consistent with the disclosure herein. In embodiments, the RNA guide does not consist of the sequence of
AGAAAUCCGUCUUUCAUUGACGGAAUGUCGGAUGGAUGAAACC (SEQ ID NO: 778);
AGAAAUCCGUCUUUCAUUGACGGCUAUCUCUUGUACUACACUG (SEQ ID NO: 779);
AGAAAUCCGUCUUUCAUUGACGGACACGGCAGGCAUACUCAUC (SEQ ID NO: 780); or
AGAAAUCCGUCUUUCAUUGACGGGUCACAGCCCAAGAUAGUUA (SEQ ID NO: 781).

In some embodiments, a spacer sequence described herein comprises a uracil (U). In some embodiments, a spacer sequence described herein comprises a thymine (T). In some embodiments, a spacer sequence according to Table 5 comprises a sequence comprising a thymine in one or more places indicated as uracil in Table 5.

Modifications

The RNA guide may include one or more covalent modifications with respect to a reference sequence, in particular the parent polyribonucleotide, which are included within the scope of this invention.

Exemplary modifications can include any modification to the sugar, the nucleobase, the internucleoside linkage (e.g. to a linking phosphate/to a phosphodiester linkage/to the phosphodiester backbone), and any combination thereof. Some of the exemplary modifications provided herein are described in detail below.

The RNA guide may include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O-(1-thiophosphate)-cytidine (a-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotide (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

### Cas12i Polypeptide

In some embodiments, the composition of the present invention includes a Cas12i polypeptide as described in PCT/US2019/022375.

In some embodiments, the composition of the present invention includes a Cas12i2 polypeptide described herein (e.g., a polypeptide comprising SEQ ID NO: 772 and/or encoded by SEQ ID NO: 771). In some embodiments, the Cas12i2 polypeptide comprises at least one RuvC domain.

A nucleic acid sequence encoding the Cas12i2 polypeptide described herein may be substantially identical to a reference nucleic acid sequence, e.g., SEQ ID NO: 771. In some embodiments, the Cas12i2 polypeptide is encoded by a nucleic acid comprising a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence, e.g., SEQ ID NO: 771. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions of temperature and ionic strength (e.g., within a range of medium to high stringency). See, e.g., Tijssen, “Hybridization with Nucleic Acid Probes. Part I. Theory and Nucleic Acid Preparation” (Laboratory Techniques in Biochemistry and Molecular Biology, Vol 24).

In some embodiments, the Cas12i2 polypeptide is encoded by a nucleic acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity, but not 100% sequence identity, to a reference nucleic acid sequence, e.g., SEQ ID NO: 771.

In some embodiments, the Cas12i2 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 772.

In some embodiments, the present invention describes a Cas12i2 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 772. Homology or identity can be determined by amino acid sequence alignment, e.g., using a program such as BLAST, ALIGN, or CLUSTAL, as described herein.

Also provided is a Cas12i2 polypeptide of the present invention having enzymatic activity, e.g., nuclease or endonuclease activity, and comprising an amino acid sequence which differs from the amino acid sequences of SEQ ID NO: 772 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the Cas12i2 polypeptide comprises a polypeptide having a sequence of SEQ ID NO: 782, SEQ ID NO: 783, SEQ ID NO: 784, SEQ ID NO: 785, or SEQ ID NO: 786.

In some embodiments, the Cas12i2 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 782, SEQ ID NO: 783, SEQ ID NO: 784, SEQ ID NO: 785, or SEQ ID NO: 786. In some embodiments, a Cas12i2 polypeptide having at least 50%, 60%, 65%, 70%, 75%, 80%, 85%, 90%, 91%, 92%, 93%, 94%, 95%, 96%, 97%, 98%, 99%, or 100% identity to SEQ ID NO: 782, SEQ ID NO: 783, SEQ ID NO: 784, SEQ ID NO: 785, or SEQ ID NO: 786 maintains the amino acid changes (or at least 1, 2, 3 etc. of these changes) that differentiate the polypeptide from its respective parent/reference sequence.

In some embodiments, the present invention describes a Cas12i2 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 782, SEQ ID NO: 783, SEQ ID NO: 784, SEQ ID NO: 785, or SEQ ID NO: 786. Homology or identity can be determined by amino acid sequence alignment, e.g., using a program such as BLAST, ALIGN, or CLUSTAL, as described herein.

Also provided is a Cas12i2 polypeptide of the present invention having enzymatic activity, e.g., nuclease or endonuclease activity, and comprising an amino acid sequence which differs from the amino acid sequences of SEQ ID NO: 782, SEQ ID NO: 783, SEQ ID NO: 784, SEQ ID NO: 785, or SEQ ID NO: 786 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present invention includes a Cas12i4 polypeptide described herein (e.g., a polypeptide comprising SEQ ID NO: 814 and/or encoded by SEQ ID NO: 787). In some embodiments, the Cas12i4 polypeptide comprises at least one RuvC domain.

A nucleic acid sequence encoding the Cas12i4 polypeptide described herein may be substantially identical to a reference nucleic acid sequence, e.g., SEQ ID NO: 787. In some embodiments, the Cas12i4 polypeptide is encoded by a nucleic acid comprising a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence, e.g., SEQ ID NO: 787. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions of temperature and ionic strength (e.g., within a range of medium to high stringency).

In some embodiments, the Cas12i4 polypeptide is encoded by a nucleic acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity, but not 100% sequence identity, to a reference nucleic acid sequence, e.g., SEQ ID NO: 787.

In some embodiments, the Cas12i4 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 814.

In some embodiments, the present invention describes a Cas12i4 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 814. Homology or identity can be determined by amino acid sequence alignment, e.g., using a program such as BLAST, ALIGN, or CLUSTAL, as described herein.

Also provided is a Cas12i4 polypeptide of the present invention having enzymatic activity, e.g., nuclease or endonuclease activity, and comprising an amino acid sequence which differs from the amino acid sequences of SEQ ID NO: 814 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the Cas12i4 polypeptide comprises a polypeptide having a sequence of SEQ ID NO: 815 or SEQ ID NO: 816.

In some embodiments, the Cas12i4 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 815 or SEQ ID NO: 816. In some embodiments, a Cas12i4 polypeptide having at least 50%, 60%, 65%, 70%, 75%, 80%, 85%, 90%, 91%, 92%, 93%, 94%, 95%, 96%, 97%, 98%, 99%, or 100% identity to SEQ ID NO: 815 or SEQ ID NO: 816 maintains the amino acid changes (or at least 1, 2, 3 etc. of these changes) that differentiate it from its respective parent/reference sequence.

In some embodiments, the present invention describes a Cas12i4 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 815 or SEQ ID NO: 816. Homology or identity can be determined by amino acid sequence alignment, e.g., using a program such as BLAST, ALIGN, or CLUSTAL, as described herein.

Also provided is a Cas12i4 polypeptide of the present invention having enzymatic activity, e.g., nuclease or endonuclease activity, and comprising an amino acid sequence which differs from the amino acid sequences of SEQ ID NO: 815 or SEQ ID NO: 816 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present invention includes a Cas12i1 polypeptide described herein (e.g., a polypeptide comprising SEQ ID NO: 817). In some embodiments, the Cas12i4 polypeptide comprises at least one RuvC domain.

In some embodiments, the Cas12i1 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 817.

In some embodiments, the present invention describes a Cas12i1 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 817. Homology or identity can be determined by amino acid sequence alignment, e.g., using a program such as BLAST, ALIGN, or CLUSTAL, as described herein.

Also provided is a Cas12i1 polypeptide of the present invention having enzymatic activity, e.g., nuclease or endonuclease activity, and comprising an amino acid sequence which differs from the amino acid sequences of SEQ ID NO: 817 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

In some embodiments, the composition of the present invention includes a Cas12i3 polypeptide described herein (e.g., a polypeptide comprising SEQ ID NO: 818). In some embodiments, the Cas12i4 polypeptide comprises at least one RuvC domain.

In some embodiments, the Cas12i3 polypeptide of the present invention comprises a polypeptide sequence having at least 50%, 60%, 65%, 70%, 75%, 80%, 85%, 90%, 91%, 92%, 93%, 94%, 95%, 96%, 97%, 98%, 99%, or 100% identity to SEQ ID NO: 818.

In some embodiments, the present invention describes a Cas12i3 polypeptide having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 818. Homology or identity can be determined by amino acid sequence alignment, e.g., using a program such as BLAST, ALIGN, or CLUSTAL, as described herein.

Also provided is a Cas12i3 polypeptide of the present invention having enzymatic activity, e.g., nuclease or endonuclease activity, and comprising an amino acid sequence which differs from the amino acid sequences of SEQ ID NO: 818 by 50, 40, 35, 30, 25, 20, 19, 18, 17, 16, 15, 14, 13, 12, 11, 10, 9, 8, 7, 6, 5, 4, 3, 2, 1, or 0 amino acid residue(s), when aligned using any of the previously described alignment methods.

Although the changes described herein may be one or more amino acid changes, changes to the Cas12i polypeptide may also be of a substantive nature, such as fusion of polypeptides as amino- and/or carboxyl-terminal extensions. For example, the Cas12i polypeptide may contain additional peptides, e.g., one or more peptides. Examples of additional peptides may include epitope peptides for labelling, such as a polyhistidine tag (His-tag), Myc, and FLAG. In some embodiments, the Cas12i polypeptide described herein can be fused to a detectable moiety such as a fluorescent protein (e.g., green fluorescent protein (GFP) or yellow fluorescent protein (YFP)).

In some embodiments, the Cas12i polypeptide comprises at least one (e.g., two, three, four, five, six, or more) nuclear localization signal (NLS). In some embodiments, the Cas12i polypeptide comprises at least one (e.g., two, three, four, five, six, or more) nuclear export signal (NES). In some embodiments, the Cas12i polypeptide comprises at least one (e.g., two, three, four, five, six, or more) NLS and at least one (e.g., two, three, four, five, six, or more) NES.

In some embodiments, the Cas12i polypeptide described herein can be self-inactivating. See, Epstein et al., “Engineering a Self-Inactivating CRISPR System for AAV Vectors,” Mol. Ther., 24 (2016): S50, which is incorporated by reference in its entirety.

In some embodiments, the nucleotide sequence encoding the Cas12i polypeptide described herein can be codon-optimized for use in a particular host cell or organism. For example, the nucleic acid can be codon-optimized for any non-human eukaryote including mice, rats, rabbits, dogs, livestock, or non-human primates. Codon usage tables are readily available, for example, at the “Codon Usage Database” available at www.kazusa.orjp/codon/and these tables can be adapted in a number of ways. See Nakamura et al. Nucl. Acids Res. 28:292 (2000), which is incorporated herein by reference in its entirety. Computer algorithms for codon optimizing a particular sequence for expression in a particular host cell are also available, such as Gene Forge (Aptagen; Jacobus, PA).

### Target Sequence

In some embodiments, the target sequence is within a B2M gene or a locus of a B2M gene. In some embodiments, the B2M gene is a mammalian gene. In some embodiments, the B2M gene is a human gene. For example, in some embodiments, the target sequence is within the sequence of SEQ ID NO: 773 or the reverse complement thereof. In some embodiments, the target sequence is within an exon of the B2M gene set forth in SEQ ID NO: 773 (or the reverse complement thereof), e.g., within a sequence of SEQ ID NO: 774, 775, 776, or 777 (or a reverse complement thereof). Target sequences within an exon of the B2M gene of SEQ ID NO: 773 (and the reverse complement thereof) are set forth in Table 5. In some embodiments, the target sequence is within an intron of the B2M gene set forth in SEQ ID NO: 773 (or the reverse complement thereof), e.g., within a sequence of SEQ ID NO: 1219, 1220, or 1221 (or a reverse complement thereof). Target sequences within an intron of the B2M gene of SEQ ID NO: 773 (or the reverse complement thereof) are set forth in Table 5. In some embodiments, the target sequence is within a variant (e.g., a polymorphic variant) of the B2M gene sequence set forth in SEQ ID NO: 773 or the reverse complement thereof. In some embodiments, the B2M gene sequence is a homolog of the sequence set forth in SEQ ID NO: 773 or the reverse complement thereof. For examples, in some embodiments, the B2M gene sequence is a non-human B2M sequence.

In some embodiments, the target sequence is adjacent to a 5′-NTTN-3′ PAM sequence, wherein N is any nucleotide. The 5′-NTTN-3′ sequence may be immediately adjacent to the target sequence or, for example, within a small number (e.g., 1, 2, 3, 4, or 5) of nucleotides of the target sequence. In some embodiments the 5′-NTTN-3′ sequence is 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′, wherein Y is C or T, B is any nucleotide except for A, D is any nucleotide except for C, and R is A or G. In some embodiments, the 5′-NTTN-3′ sequence is 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

In some embodiments, the target sequence is single-stranded (e.g., single-stranded DNA). In some embodiments, the target sequence is double-stranded (e.g., double-stranded DNA). In some embodiments, the target sequence comprises both single-stranded and double-stranded regions. In some embodiments, the target sequence is linear. In some embodiments, the target sequence is circular. In some embodiments, the target sequence comprises one or more modified nucleotides, such as methylated nucleotides, damaged nucleotides, or nucleotides analogs. In some embodiments, the target sequence is not modified. In some embodiments, the RNA guide binds to a first strand of a double-stranded target sequence (e.g., the target strand or the spacer-complementary strand), and the 5′-NTTN-3′ PAM sequence is present in the second, complementary strand (e.g., the non-target strand or the non-spacer-complementary strand). In some embodiments, the RNA guide binds adjacent to a 5′-NAAN-3′ sequence on the target strand (e.g., the spacer-complementary strand).

In some embodiments, the target sequence is present in a cell. In some embodiments, the target sequence is present in the nucleus of the cell. In some embodiments, the target sequence is endogenous to the cell. In some embodiments, the target sequence is a genomic DNA. In some embodiments, the target sequence is a chromosomal DNA. In some embodiments, the target sequence is a protein-coding gene or a functional region thereof, such as a coding region, or a regulatory element, such as a promoter, enhancer, a 5′ or 3′ untranslated region, etc. In some embodiments, the target sequence is a plasmid.

In some embodiments, the target sequence is present in a readily accessible region of the target sequence. In some embodiments, the target sequence is in an exon of a target gene. In some embodiments, the target sequence is across an exon-intron junction of a target gene. In some embodiments, the target sequence is present in a non-coding region, such as a regulatory region of a gene. In some embodiments, wherein the target sequence is exogenous to a cell, the target sequence comprises a sequence that is not found in the genome of the cell.

In some embodiments, the target sequence is exogenous to a cell. In some embodiments, the target sequence is a horizontally transferred plasmid. In some embodiments, the target sequence is integrated in the genome of the cell. In some embodiments, the target sequence is not integrated in the genome of the cell. In some embodiments, the target sequence is a plasmid in the cell. In some embodiments, the target sequence is present in an extrachromosomal array.

In some embodiments, the target sequence is an isolated nucleic acid, such as an isolated DNA or an isolated RNA. In some embodiments, the target sequence is present in a cell-free environment. In some embodiments, the target sequence is an isolated vector, such as a plasmid. In some embodiments, the target sequence is an ultrapure plasmid.

The target sequence is a locus of the B2M gene that hybridizes to the RNA guide. In some embodiments, a cell has only one copy of the target sequence. In some embodiments, a cell has more than one copy, such as at least about any one of 2, 3, 4, 5, 10, 100, or more copies of the target sequence.

In some embodiments, a B2M target sequence is selected to be edited by a Cas12i polypeptide and an RNA guide using one or more of the following criteria. First, in some embodiments, a target sequence near the 5′ end of the B2M coding sequence is selected. For example, in some embodiments, an RNA guide is designed to target a sequence in exon 1 (SEQ ID NO: 774) or exon 2 (SEQ ID NO: 775). Second, in some embodiments, a target sequence adjacent to a 5′-CTTY-3′ PAM sequence is selected. For example, in some embodiments, an RNA guide is designed to target a sequence adjacent to a 5′-CTTT-3′ or 5′-CTTC-3′ sequence. Third, in some embodiments, a target sequence having low sequence similarity to other genomic sequences is selected. For example, for each target sequence, potential non-target sites can be identified by searching for other genomic sequences adjacent to a PAM sequence and calculating the Levenshtein distance between the target sequence and the PAM-adjacent sequences. The Levenshtein distance (e.g., edit distance) corresponds to the minimum number of edits (e.g., insertions, deletions, or substitutions) required to change one sequence into another (e.g., to change the sequence of a potential non-target locus into the sequence of the on-target locus). Following this analysis, RNA guides are designed for target sequences that do not have potential off-target sequences with a Levenshtein distance of 0 or 1.

### Production

The present invention includes methods for production of the RNA guide, methods for production of the polypeptide, and methods for complexing the RNA guide and Cas12i polypeptide.

### RNA Guide

In some embodiments, the RNA guide is made by in vitro transcription of a DNA template. Thus, for example, in some embodiments, the RNA guide is generated by in vitro transcription of a DNA template encoding the RNA guide using an upstream promoter sequence (e.g., a T7 polymerase promoter sequence). In some embodiments, the DNA template encodes multiple RNA guides or the in vitro transcription reaction includes multiple different DNA templates, each encoding a different RNA guide. In some embodiments, the RNA guide is made using chemical synthetic methods. In some embodiments, the RNA guide is made by expressing the RNA guide sequence in cells transfected with a plasmid including sequences that encode the RNA guide. In some embodiments, the plasmid encodes multiple different RNA guides. In some embodiments, multiple different plasmids, each encoding a different RNA guide, are transfected into the cells. In some embodiments, the RNA guide is expressed from a plasmid that encodes the RNA guide and also encodes a Cas12i polypeptide. In some embodiments, the RNA guide is expressed from a plasmid that expresses the RNA guide but not a Cas12i polypeptide. In some embodiments, the RNA guide is purchased from a commercial vendor. In some embodiments, the RNA guide is synthesized using one or more modified nucleotide, e.g., as described above.

### Cas12i Polypeptide

In some embodiments, the Cas12i polypeptide of the present invention can be prepared by (a) culturing bacteria which produce the Cas12i polypeptide of the present invention, isolating the Cas12i polypeptide, optionally, purifying the Cas12i polypeptide, and complexing the Cas12i polypeptide with an RNA guide. The Cas12i polypeptide can be also prepared by (b) a known genetic engineering technique, specifically, by isolating a gene encoding the Cas12i polypeptide of the present invention from bacteria, constructing a recombinant expression vector, and then transferring the vector into an appropriate host cell that expresses the RNA guide for expression of a recombinant protein that complexes with the RNA guide in the host cell. Alternatively, the Cas12i polypeptide can be prepared by (c) an in vitro coupled transcription-translation system and then complexing with an RNA guide.

In some embodiments, a host cell is used to express the Cas12i polypeptide. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of the Cas12i polypeptide. After expression of the Cas12i polypeptide, the host cells can be collected and Cas12i polypeptide purified from the cultures etc. according to conventional methods (for example, filtration, centrifugation, cell disruption, gel filtration chromatography, ion exchange chromatography, etc.).

In some embodiments, the methods for Cas12i polypeptide expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of the Cas12i polypeptide. In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids or more of the Cas12i polypeptide.

A variety of methods can be used to determine the level of production of a Cas12i polypeptide in a host cell. Such methods include, but are not limited to, for example, methods that utilize either polyclonal or monoclonal antibodies specific for the Cas12i polypeptide or a labeling tag as described elsewhere herein. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of the Cas12i polypeptide in a cell, comprising providing a polyribonucleotide encoding the Cas12i polypeptide to a host cell wherein the polyribonucleotide encodes the Cas12i polypeptide, expressing the Cas12i polypeptide in the cell, and obtaining the Cas12i polypeptide from the cell.

### Complexing

In some embodiments, an RNA guide targeting B2M is complexed with a Cas12i polypeptide to form a ribonucleoprotein. In some embodiments, complexation of the RNA guide and Cas12i polypeptide occurs at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., or 55° C. In some embodiments, the RNA guide does not dissociate from the Cas12i polypeptide at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours.

In some embodiments, the RNA guide and Cas12i polypeptide are complexed in a complexation buffer. In some embodiments, the Cas12i polypeptide is stored in a buffer that is replaced with a complexation buffer to form a complex with the RNA guide. In some embodiments, the Cas12i polypeptide is stored in a complexation buffer.

In some embodiments, the complexation buffer has a pH in a range of about 7.3 to 8.6. In one embodiment, the pH of the complexation buffer is about 7.3. In one embodiment, the pH of the complexation buffer is about 7.4. In one embodiment, the pH of the complexation buffer is about 7.5. In one embodiment, the pH of the complexation buffer is about 7.6. In one embodiment, the pH of the complexation buffer is about 7.7. In one embodiment, the pH of the complexation buffer is about 7.8. In one embodiment, the pH of the complexation buffer is about 7.9. In one embodiment, the pH of the complexation buffer is about 8.0. In one embodiment, the pH of the complexation buffer is about 8.1. In one embodiment, the pH of the complexation buffer is about 8.2. In one embodiment, the pH of the complexation buffer is about 8.3. In one embodiment, the pH of the complexation buffer is about 8.4. In one embodiment, the pH of the complexation buffer is about 8.5. In one embodiment, the pH of the complexation buffer is about 8.6.

In some embodiments, the Cas12i polypeptide can be overexpressed and complexed with the RNA guide in a host cell prior to purification as described herein. In some embodiments, mRNA or DNA encoding the Cas12i polypeptide is introduced into a cell so that the Cas12i polypeptide is expressed in the cell. In some embodiments, the RNA guide is also introduced into the cell, whether simultaneously, separately, or sequentially from a single mRNA or DNA construct, such that the ribonucleoprotein complex is formed in the cell.

### Delivery

Compositions or complexes described herein may be formulated, for example, including a carrier, such as a carrier and/or a polymeric carrier, e.g., a liposome, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding the Cas12i polypeptide, RNA guide, donor DNA, etc.), one or more transcripts thereof, and/or a pre-formed RNA guide/Cas12i polypeptide complex to a cell, where a ternary complex is formed. Exemplary intracellular delivery methods, include, but are not limited to: viruses or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnectofection or magnet assisted transfection, particle bombardment; and hybrid methods, such as nucleofection. In some embodiments, the present application further provides cells produced by such methods, and organisms (such as animals, plants, or fungi) comprising or produced from such cells.

In some embodiments, the Cas12i component and the RNA guide component are delivered together. For example, in some embodiments, the Cas12i component and the RNA guide component are packaged together in a single AAV particle. In another example, in some embodiments, the Cas12i component and the RNA guide component are delivered together via lipid nanoparticles (LNPs). In some embodiments, the Cas12i component and the RNA guide component are delivered separately. For example, in some embodiments, the Cas12i component and the RNA guide are packaged into separate AAV particles. In another example, in some embodiments, the Cas12i component is delivered by a first delivery mechanism and the RNA guide is delivered by a second delivery mechanism.

### Cells

Compositions or complexes described herein can be delivered to a variety of cells. In some embodiments, the cell is an isolated cell. In some embodiments, the cell is in cell culture or a co-culture of two or more cell types. In some embodiments, the cell is ex vivo. In some embodiments, the cell is obtained from a living organism and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the cell is an archaeal cell or derived from an archaeal cell.

In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T, MF7, K562, HeLa, CHO, and transgenic varieties thereof. Cell lines are available from a variety of sources known to those with skill in the art (see, e.g., the American Type Culture Collection (ATCC) (Manassas, Va.)). In some embodiments, the cell is an immortal or immortalized cell.

In some embodiments, the cell is a primary cell. In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a differentiated cell. For example, in some embodiments, the differentiated cell is a muscle cell (e.g., a myocyte), a fat cell (e.g., an adipocyte), a bone cell (e.g., an osteoblast, osteocyte, osteoclast), a blood cell (e.g., a monocyte, a lymphocyte, a neutrophil, an eosinophil, a basophil, a macrophage, a erythrocyte, or a platelet), a nerve cell (e.g., a neuron), an epithelial cell, an immune cell (e.g., a lymphocyte, a neutrophil, a monocyte, or a macrophage), a liver cell (e.g., a hepatocyte), a fibroblast, or a sex cell. In some embodiments, the cell is a terminally differentiated cell. For example, in some embodiments, the terminally differentiated cell is a neuronal cell, an adipocyte, a cardiomyocyte, a skeletal muscle cell, an epidermal cell, or a gut cell. In some embodiments, the cell is an immune cell. In some embodiments, the immune cell is a T cell. In some embodiments, the immune cell is a B cell. In some embodiments, the immune cell is a Natural Killer (NK) cell. In some embodiments, the immune cell is a Tumor Infiltrating Lymphocyte (TIL). In some embodiments, the cell is a mammalian cell, e.g., a human cell or a murine cell. In some embodiments, the murine cell is derived from a wild-type mouse, an immunosuppressed mouse, or a disease-specific mouse model. In some embodiments, the cell is a cell within a living tissue, organ, or organism.

### Methods

The disclosure also provides methods of modifying a target sequence within the B2M gene. In some embodiments, the methods comprise introducing a B2M-targeting RNA guide and a Cas12i polypeptide into a cell. The B2M-targeting RNA guide and Cas12i polypeptide can be introduced as a ribonucleoprotein complex into a cell. The B2M-targeting RNA guide and Cas12i polypeptide can be introduced on a nucleic acid vector. The Cas12i polypeptide can be introduced as an mRNA. The RNA guide can be introduced directly into the cell.

In some embodiments, the sequence of the B2M gene is set forth in SEQ ID NO: 773 (or the reverse complement thereof). In some embodiments, the target sequence is in an exon of a B2M gene, such as an exon having a sequence set forth in any one of SEQ ID NO: 774, SEQ ID NO: 775, SEQ ID NO: 776, or SEQ ID NO: 777 (or the reverse complement thereof). In some embodiments, the target sequence is in an intron of a B2M gene (e.g., an intron of the sequence set forth in SEQ ID NO: 773, or the reverse complement thereof), such as an intron having a sequence set forth in any one of SEQ ID NO: 1219, SEQ ID NO: 1220, or SEQ ID NO: 1221 (or the reverse complement thereof). In other embodiments, the sequence of the B2M gene is a variant of the sequence set forth in SEQ ID NO: 773 (or the reverse complement thereof) or a homolog of the sequence set forth in SEQ ID NO: 773 (or the reverse complement thereof). For example, in some embodiments, the target sequence is polymorphic variant of the B2M sequence set forth in SEQ ID NO: 773 (or the reverse complement thereof) or a non-human form of the B2M gene.

In some embodiments, an RNA guide as disclosed herein is designed to be complementary to a target sequence that is adjacent to a 5′-NTTN-3′ PAM sequence. The 5′-NTTN-3′ sequence may be immediately adjacent to the target sequence or, for example, within a small number (e.g., 1, 2, 3, 4, or 5) of nucleotides of the target sequence. In some embodiments the 5′-NTTN-3′ sequence is 5′-NTTY-3′, 5′-NTTC-3′, 5′-NTTT-3′, 5′-NTTA-3′, 5′-NTTB-3′, 5′-NTTG-3′, 5′-CTTY-3′, 5‘-DTTR’3′, 5′-CTTR-3′, 5′-DTTT-3′, 5′-ATTN-3′, or 5′-GTTN-3′, wherein Y is C or T, B is any nucleotide except for A, D is any nucleotide except for C, and R is A or G. In some embodiments, the 5′-NTTN-3′ sequence is 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′. In some embodiments, the RNA guide is designed to bind to a first strand of a double-stranded target sequence (e.g., the target strand or the spacer-complementary strand), and the 5′-NTTN-3′ PAM sequence is present in the second, complementary strand (e.g., the non-target strand or the non-spacer-complementary strand).

In some embodiments, the RNA guide binds adjacent to a 5′-NAAN-3′ sequence on the target strand (e.g., the spacer-complementary strand).

In some embodiments, the Cas12i polypeptide has enzymatic activity (e.g., nuclease activity). In some embodiments, the Cas12i polypeptide induces one or more DNA double-stranded breaks in the cell. In some embodiments, the Cas12i polypeptide induces one or more DNA single-stranded breaks in the cell.

In some embodiments, the Cas12i polypeptide induces one or more DNA nicks in the cell. In some embodiments, DNA breaks and/or nicks result in formation of one or more indels (e.g., one or more deletions).

In some embodiments, an RNA guide disclosed herein forms a complex with the Cas12i polypeptide and directs the Cas12i polypeptide to a target sequence adjacent to a 5′-NTTN-3′ sequence. In some embodiments, the complex induces a deletion (e.g., a nucleotide deletion or DNA deletion) adjacent to the 5′-NTTN-3′ sequence. In some embodiments, the complex induces a deletion adjacent to a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the complex induces a deletion adjacent to a T/C-rich sequence.

In some embodiments, the deletion is downstream of a 5′-NTTN-3′ sequence. In some embodiments, the deletion is downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion is downstream of a T/C-rich sequence.

In some embodiments, the deletion alters expression of the B2M gene. In some embodiments, the deletion alters function of the B2M gene. In some embodiments, the deletion inactivates the B2M gene. In some embodiments, the deletion is a frameshifting deletion. In some embodiments, the deletion is a non-frameshifting deletion. In some embodiments, the deletion leads to cell toxicity or cell death (e.g., apoptosis).

In some embodiments, the deletion starts within about 5 to about 15 nucleotides (e.g., about 3, 4, 5, 6, 7, 8, 9, 10, 11, 12, 13, 14, 15, 16, or 17 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence.

In some embodiments, the deletion starts within about 5 to about 10 nucleotides (e.g., about 3, 4, 5, 6, 7, 8, 9, 10, 11, or 12 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence.

In some embodiments, the deletion starts within about 10 to about 15 nucleotides (e.g., about 8, 9, 10, 11, 12, 13, 14, 15, 16, or 17 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence.

In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a T/C-rich sequence.

In some embodiments, the deletion ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion ends within about 20 to about 25 nucleotides (e.g., about 17, 18, 19, 20, 21, 22, 23, 24, 25, 26, 27, or 28 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 20 to about 25 nucleotides (e.g., about 17, 18, 19, 20, 21, 22, 23, 24, 25, 26, 27, or 28 nucleotides) downstream of a T/C-rich sequence.

In some embodiments, the deletion ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion ends within about 25 to about 30 nucleotides (e.g., about 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion ends within about 25 to about 30 nucleotides (e.g., about 22, 23, 24, 25, 26, 27, 28, 29, 30, 31, 32, or 33 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion ends within about 25 to about 30 nucleotides (e.g., about 22, 23, 24, 25, 26, 27, 28, 29, 30, 31, 32, or 33 nucleotides) downstream of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides and ends within about 20 to about 25 nucleotides (e.g., about 17, 18, 19, 20, 21, 22, 23, 24, 25, 26, 27, or 28 nucleotides) (e.g., about 3, 4, 5, 6, 7, 8, 9, 10, 11, or 12 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 20 to about 25 nucleotides (e.g., about 17, 18, 19, 20, 21, 22, 23, 24, 25, 26, 27, or 28 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence and ends within about 25 to about 30 nucleotides (e.g., about 22, 23, 24, 25, 26, 27, 28, 29, 30, 31, 32, or 33 nucleotides) downstream of the 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion is up to about 50 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides). In some embodiments, the deletion is up to about 40 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides). In some embodiments, the deletion is between about 4 nucleotides and about 40 nucleotides in length (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides). In some embodiments, the deletion is between about 4 nucleotides and about 25 nucleotides in length (e.g., about 3, 4, 5, 6, 7, 8, 9, 10, 11, 12, 13, 14, 15, 16, 17, 18, 19, 20, 21, 22, 23, 24, 25, 26, 27, or 28 nucleotides). In some embodiments, the deletion is between about 10 nucleotides and about 25 nucleotides in length (e.g., about 7, 8, 9, 10, 11, 12, 13, 14, 15, 16, 17, 18, 19, 20, 21, 22, 23, 24, 25, 26, 27, or 28 nucleotides). In some embodiments, the deletion is between about 10 nucleotides and about 15 nucleotides in length (e.g., about 7, 8, 9, 10, 11, 12, 13, 14, 15, 16, or 17 nucleotides).

In some embodiments, the methods described herein are used to engineer a cell comprising a deletion as described herein in a B2M gene.

Compositions, vectors, nucleic acids, RNA guides and cells disclosed herein may be used in therapy. Compositions, vectors, nucleic acids, RNA guides and cells disclosed herein may be used in methods of treating a disease or condition in a subject. Any suitable delivery or administration method known in the art may be used to deliver compositions, vectors, nucleic acids, RNA guides and cells disclosed herein. Such methods may involve contacting a target sequence with a composition, vector, nucleic acid, or RNA guide disclosed herein. Such methods may involve a method of editing a B2M sequence as disclosed herein. In some embodiments, a cell engineered using an RNA guide disclosed herein is used for ex vivo gene therapy. In some embodiments, a cell engineered using an RNA guide disclosed herein is used for CAR T-cell therapy.

### Kits

The invention also provides kits or systems that can be used, for example, to carry out a method described herein. In some embodiments, the kits or systems include an RNA guide and a Cas12i polypeptide. In some embodiments, the kits or systems include a polynucleotide that encodes such a Cas12i polypeptide, and optionally the polynucleotide is comprised within a vector, e.g., as described herein. In some embodiments, the kits or systems include a polynucleotide that encodes an RNA guide disclosed herein. The Cas12i polypeptide and the RNA guide (e.g., as a ribonucleoprotein) can be packaged within the same or other vessel within a kit or system or can be packaged in separate vials or other vessels, the contents of which can be mixed prior to use. The kits or systems can additionally include, optionally, a buffer and/or instructions for use of the RNA guide and Cas12i polypeptide.

All references and publications cited herein are hereby incorporated by reference.

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1—Editing of B2M in a Mammalian Cell Via Transfection

This Example describes indel assessment on multiple B2M targets using Cas12i2 and RNA guide compositions introduced into mammalian cells by transient transfection.

Variant Cas12i2 of SEQ ID NO: 782 was cloned with a CMV promoter in a pcda3.1 backbone (Invitrogen). The plasmids were then maxi-prepped and diluted to 1 μg/L. For RNA guide preparation, a dsDNA fragment encoding an RNA guide was derived by ultramers containing the target sequence scaffold, and the U6 promoter. Ultramers were resuspended in 10 mM Tris·HCl at a pH of 7.5 to a final stock concentration of 100 μM. Working stocks were subsequently diluted to 10 μM, again using 10 mM Tris·HCl to serve as the template for the PCR reaction. The amplification of the RNA guide was done in 50 μL reactions with the following components: 0.02 μl of aforementioned template, 2.5 μl forward primer, 2.5 μl reverse primer, 25 μL NEB HiFi Polymerase, and 20 μl water. Cycling conditions were: 1×(30 s at 98° C.), 30×(10 s at 98° C., 15 s at 67° C.), 1× (2 min at 72° C.). PCR products were cleaned up with a 1.8× SPRI treatment and normalized to 25 ng/L. The prepared RNA guide sequences and their corresponding target sequences are shown in Table 6.

TABLE 6
RNA guide and Target Sequences for Transient Transfection.

| Target | RNA Guide | Target Sequence |
|---|---|---|
| B2M_exon1_target1 | AGAAAUCCGUCUUUCAUUGACGGAGGAAUGC | AGGAATGCCCGCCAGCGCGA |
| | CCGCCAGCGCGA(SEQ ID NO: 1222) | (SEQ ID NO: 1231) |
| B2M_exon1_target2 | AGAAAUCCGUCUUUCAUUGACGGCUGGCCUG | CTGGCCTGGAGGCTATCCAG |
| | GAGGCUAUCCAG (SEQ ID NO: 1223) | (SEQ ID NO: 1232) |
| B2M_exon2_target1 | AGAAAUCCGUCUUUCAUUGACGGUCCCGAUA | TCCCGATATTCCTCAGGTAC |
| | UUCCUCAGGUAC (SEQ ID NO: 1224) | (SEQ ID NO: 1233) |
| B2M_exon2_target2 | AGAAAUCCGUCUUUCAUUGACGGGG | GGAGTACCTGAGGAATATCG |
| | AGUACCUGAGGAAUAUCG (SEQ ID NO: 1225) | (SEQ ID NO: 1234) |
| B2M_exon2_target3 | AGAAAUCCGUCUUUCAUUGACGGCC | CCATTCTCTGCTGGATGACG |
| | AUUCUCUGCUGGAUGACG (SEQ ID NO: 1226) | (SEQ ID NO: 1235) |
| B2M_exon2_target4 | AGAAAUCCGUCUUUCAUUGACGGAA | AATGTCGGATGGATGAAACC |
| | UGUCGGAUGGAUGAAACC (SEQ ID NO: 778) | (SEQ ID NO: 1236) |
| B2M_exon2_target5 | AGAAAUCCGUCUUUCAUUGACGGAG | AGTAAGTCAACTTCAATGTC |
| | UAAGUCAACUUCAAUGUC (SEQ ID NO: 1227) | (SEQ ID NO: 1237) |
| B2M_exon2_target6 | AGAAAUCCGUCUUUCAUUGACGGUU | TTCAATTCTCTCTCCATTCT |
| | CAAUUCUCUCUCCAUUCU (SEQ ID NO: 1228) | (SEQ ID NO: 1238) |
| B2M_exon2_target7 | AGAAAUCCGUCUUUCAUUGACGGCA | CAGCAAGGACTGGTCTTTCT |
| | GCAAGGACUGGUCUUUCU (SEQ ID NO: 1229) | (SEQ ID NO: 1239) |
| B2M_exon2_target8 | AGAAAUCCGUCUUUCAUUGACGGCU | CTATCTCTTGTACTACACTG |
| | AUCUCUUGUACUACACUG (SEQ ID NO: 779) | (SEQ ID NO: 1240) |
| B2M_exon2_target9 | AGAAAUCCGUCUUUCAUUGACGGUU | TTCAGTGGGGGTGAATTCAG |
| | CAGUGGGGGUGAAUUCAG (SEQ ID NO: 1230) | (SEQ ID NO: 1241) |

Approximately 16 hours prior to transfection, 100 μl of 25,000 HEK293T cells in DMEM/10% FBS+Pen/Strep were plated into each well of a 96-well plate. On the day of transfection, the cells were 70-90% confluent. For each well to be transfected, a mixture of 0.5 μl of Lipofectamine 2000 and 9.5 μl of Opti-MEM was prepared and then incubated at room temperature for 5-20 minutes (Solution 1). After incubation, the lipofectamine:OptiMEM mixture was added to a separate mixture containing 182 ng of effector plasmid and 14 ng of RNA guide and water up to 10 μL (Solution 2). The solution 1 and solution 2 mixtures were mixed by pipetting up and down and then incubated at room temperature for 25 minutes. Following incubation, 20 μL of the Solution 1 and Solution 2 mixture were added dropwise to each well of a 96 well plate containing the cells. 72 hours post transfection, cells are trypsinized by adding 10 μL of TrypLE to the center of each well and incubated for approximately 5 minutes. 100 μL of D10 media was then added to each well and mixed to resuspend cells. The cells were then spun down at 500 g for 10 minutes, and the supernatant was discarded. QuickExtract buffer was added to ⅕ the amount of the original cell suspension volume. Cells were incubated at 65° C. for 15 minutes, 68° C. for 15 minutes, and 98° C. for 10 minutes.

Samples for Next Generation Sequencing were prepared by two rounds of PCR. The first round (PCR1) was used to amplify specific genomic regions depending on the target. PCR1 products were purified by column purification. Round 2 PCR (PCR2) was done to add Illumina adapters and indexes. Reactions were then pooled, loaded onto a 2% E-gel EX for 10 minutes and gel extracted. Sequencing runs were done with a 150 cycle NextSeq v2.5 mid or high output kit.

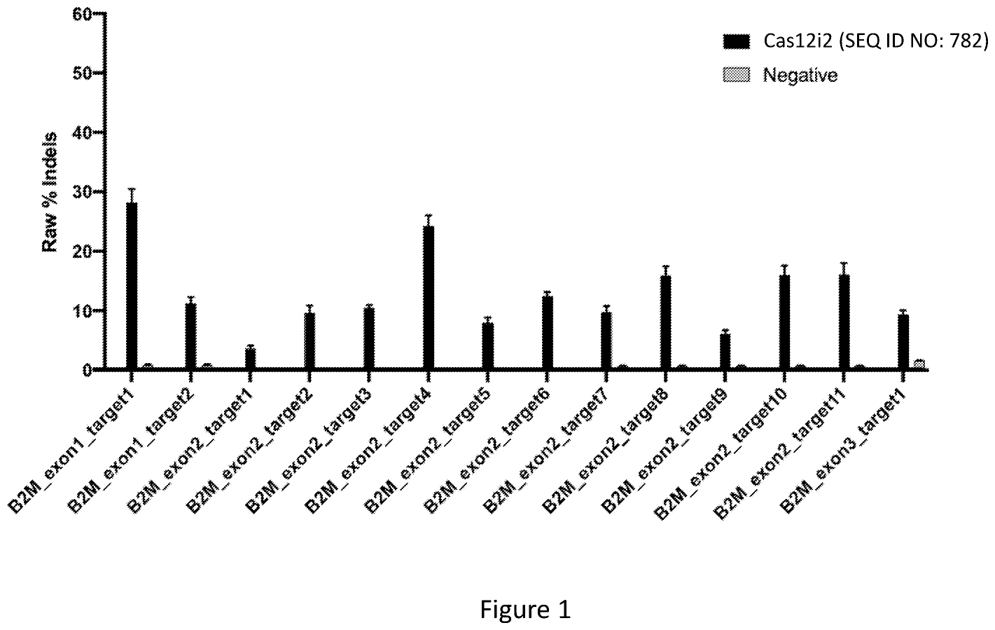

As shown in FIG. 1, each of the eleven tested RNA guides induced indels in B2M target sequences. Therefore, RNA guides and the variant Cas12i2 of SEQ ID NO: 782 were able to target B2M targets in exon 1 and exon 2 in mammalian cells.

### Example 2—Editing of B2M in a Mammalian Cell by RNP Electroporation

This Example describes ribonucleoprotein (RNP) transfection followed by FACS staining and indel assessment on multiple B2M target sequences using a Cas12i polypeptide in mammalian cells.

CD3+ T cells from three individual donors were revived and counted using an automated cell counter. A sample from each donor was collected and stained for CD3ε and DAPI for flow cytometry analysis of surface expression and viability, respectively. Cell density was adjusted to 1e6 cells/mL and cells were stimulated for 3 days with a cocktail of anti-CD3:CD28 antibodies.

Variant Cas12i2 RNP complexation reactions were made by mixing purified variant Cas12i2 (400 μM; SEQ ID NO: 783) with RNA guide (1 mM in 250 mM NaCl; see sequences in Table 7) at a 1:1 (effector:RNA guide) volume ratio (2.5:1 RNA guide:effector molar ratio). SpCas9 RNP complexation reactions were made by mixing purified SpCas9 (Aldevron; 62 μM) with sgRNA (1 mM in water; see sequences in Table 7) at a 6.45:1 (effector:sgRNA) volume ratio (2.5:1 sgRNA:effector molar ratio). For “effector only” controls, variant Cas12i2 or SpCas9 were mixed with Protein Storage Buffer (25 mM Tris, pH 7.5, 250 mM NaCl, 1 mM TCEP, 50% glycerol) at the same volume ratio as the RNA guide or sgRNA, respectively. Additional controls were included: SpCas9 (Aldevron) with either Lethal #1 (transfection control guide), pooled CD3, or ROSA26 sgRNAs and SpCas9 (Horizon) with either Lethal #1, pooled CD3, or ROSA26 sgRNAs. Complexations were incubated at 37° C. for 30-60 min. Following incubation, RNPs were diluted to 20 μM, 50 μM, 100 μM, or 160 μM effector concentration for variant Cas12i2 and 20 μM or 50 μM for SpCas9.

TABLE 7
RNA guide sequences for RNP transfection.

| | | | | Target | |
|---|---|---|---|---|---|
| Guide Name | Gene | Effector | PAM | Strand | RNA guide |
| Cas12i2_B2M | B2M | Cas12i2 | CTTC | TS | AGAAAUCCGUCUUUCAUUGACGGAAUGUCGGAU |
| exon2_target4 | | | | | GGAUGAAACC (SEQ ID NO: 778) |
| Cas12i2_B2M | B2M | Cas12i2 | CTTT | BS | AGAAAUCCGUCUUUCAUUGACGGCUAUCUCUUG |
| exon2_target8 | | | | | UACUACACUG (SEQ ID NO: 779) |
| Cas12i2_B2M | B2M | Cas12i2 | GTTC | TS | AGAAAUCCGUCUUUCAUUGACGGACACGGCAGG |
| exon2_target10 | | | | | CAUACUCAUC (SEQ ID NO: 780) |
| Cas12i2_B2M | B2M | Cas12i2 | CTTT | BS | AGAAAUCCGUCUUUCAUUGACGGGUCACAGCCC |
| exon2_target11 | | | | | AAGAUAGUUA (SEQ ID NO: 781) |
| SpCas9_B2M | B2M | SpCas9 | TGG | BS | mG*mG*mC*CGAGAUGUCUCGCUCCGGUUUUAG |
| exon1_target1 | | | | | AGCUAGAAAUAGCAAGUUAAAAUAAGGCUAGUC |
| | | | | | CGUUAUCAACUUGAAAAAGUGGCACCGAGUCGG |
| | | | | | UGCmU*mU*mU*U (SEQ ID NO: 813) |

Diluted complexed reactions were dispensed at 2 μL per well into a 384-well electroporation plate. Cell suspensions were collected and counted using an automated cell counter. Cell density was adjusted to 1.1e7 cells/mL in P3 buffer and was dispensed at 2e5 cells/reaction (18 μL). Final concentration of variant Cas12i2 RNPs was 2 μM, 5 μM, 10 μM, or 16 μM. Final concentration of SpCas9 RNPs was 2 or 5 μM. The following controls were set up: unelectroporated cells only, cells in P3 primary cell buffer (Lonza #VXP-3032) only, cells in Protein Storage Buffer only. The plate was electroporated using an electroporation device (program EO-115-AA, Lonza HT), excluding the unelectroporated conditions. Each well was split into four 96-well editing plates (containing 200 μL total volume) using robotics (StarLab Hamilton). Editing plates were incubated for 7 days at 37° C. with 100 μL media replacement at day 4.

After 7 days, plates were spun down and the supernatant was removed. Pellets were resuspended in 200 μL of PBS. 100 μL of sample was collected and stained with either the antibody panel (anti-B2M) or anti-CD3E antibody (lethal #1, pooled CD3E, ROSA26, Protein Storage Buffer and unelectroporated for Cas9 controls). All cells were stained with DAPI to assess viability. Remaining cell suspension was transferred to a 96-well PCR plate and pelleted at 500×g for 5 min. Supernatants were removed and pellets were frozen at −80° C.

For gDNA extraction, pellets were thawed to room temperature and resuspended in appropriate volume of DNA extraction buffer (QuickExtract) to give final concentration of 1000 cells/μL. Samples were then cycled in PCR machine at 65° C. for 15 min, 68° C. for 15 min, 98° C. for 10 min. Samples were then frozen at −20° C.

Samples for Next Generation Sequencing (NGS) were prepared by rounds of PCR. The first round (PCR I) was used to amplify the genomic regions flanking the target site and add NGS adapters. The second round (PCR II) was used to add NGS indexes. Reactions were then pooled, purified by column purification, and quantified on a fluorometer (Qubit). Sequencing runs were done using a 150 cycle NGS instrument (NextSeq v2.5) mid or high output kit and run on an NGS instrument (NextSeq 550).

For NGS analysis, the indel mapping function used a sample's fastq file, the amplicon reference sequence, and the forward primer sequence. For each read, a kmer-scanning algorithm was used to calculate the edit operations (match, mismatch, insertion, deletion) between the read and the reference sequence. In order to remove small amounts of primer dimer present in some samples, the first 30nt of each read was required to match the reference and reads where over half of the mapping nucleotides were mismatches were filtered out as well. Up to 50,000 reads passing those filters were used for analysis, and reads were counted as an indel read if they contained an insertion or deletion. The indel % was calculated as the number of indel-containing reads divided by the number of reads analyzed (reads passing filters up to 50,000). The QC standard for the minimum number of reads passing filters was 10,000.

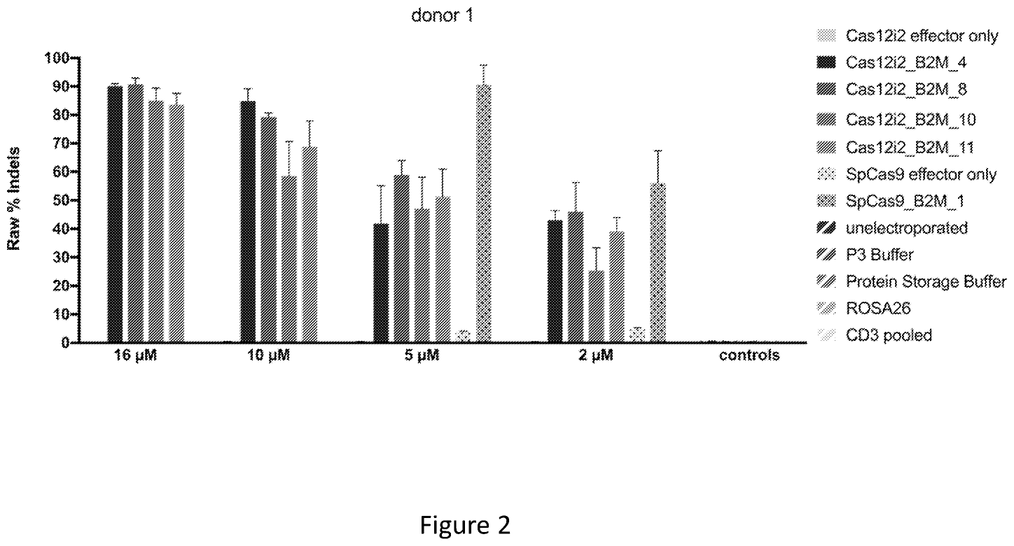

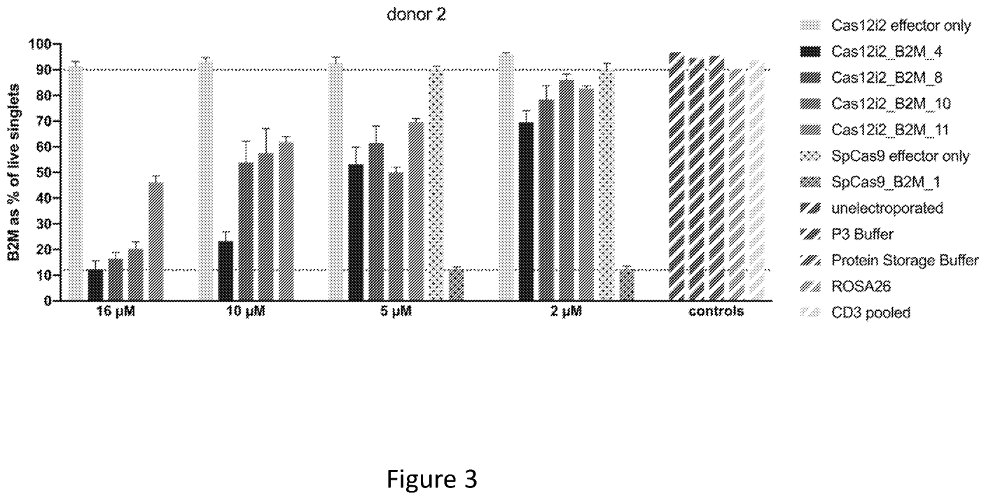

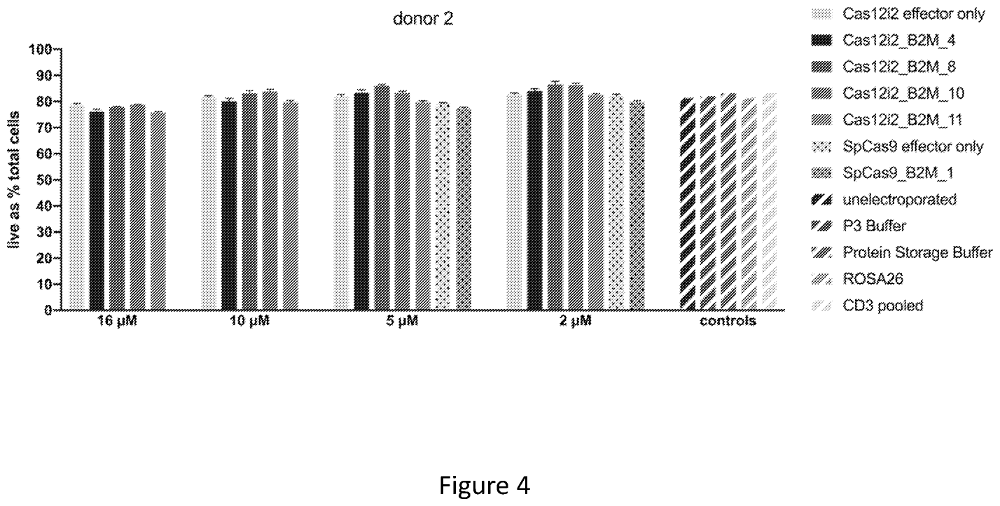

These results demonstrated robust indel activity by variant Cas12i2 RNP targeting multiple B2M targets in primary T cells (FIG. 2), with activity peaking at 16 μM. Flow cytometry staining showed significant reduction of B2M protein expression in T cells following variant Cas12i2 RNP (FIG. 3). Cell viability remained high for all conditions seven days post electroporation of the Cas12i2 RNPs targeting B2M (FIG. 4).

This Example thus shows how to measure viability of cells, e.g., T cells, electroporated with the RNA guide/Cas12i polypeptide complexes described herein, expression of B2M in the cells, and activity on B2M target sequences (indel %) in the cells.

This Example further shows that RNA guides and the variant Cas12i2 of SEQ ID NO: 783 were able to target B2M targets in exon 2 in mammalian cells.

| | |
|---|---|
| Nucleotide | 60 atgagcagcg cgatcaaaag ctacaagagc gttctgcgtc cgaacgagcg taagaaccaa |
| sequence | 120 ctgctgaaaa gcaccattca gtgcctggaa gacggtagcg cgttcttttt caagatgctg |
| encoding | 180 caaggcctgt ttggtggcat caccccggag attgttcgtt tcagcaccga acaggagaaa |
| Cas12i2- | 240 cagcaacagg atatcgcgct gtggtgcgcg gttaactggt tccgtccggt gagccaagac |
| SEQ ID NO: 771 | 300 agcctgaccc acaccattgc gagcgataac ctggtggaga agtttgagga atactatggt |
| | 360 ggcaccgcga gcgacgcgat caaacagtac ttcagcgcga gcattggcga aagctactat |
| | 420 tggaacgact gccgtcaaca gtactatgat ctgtgccgtg agctgggtgt tgaggtgagc |
| | 480 gacctgaccc atgatctgga gatcctgtgc cgtgaaaagt gcctggcggt tgcgaccgag |
| | 540 agcaaccaga acaacagcat cattagcgtt ctgtttggca ccggcgaaaa agaggaccgt |
| | 600 agcgtgaaac tgcgtatcac caagaaaatt ctggaggcga tcagcaacct gaaagaaatc |
| | 660 ccgaagaacg ttgcgccgat tcaagagatc attctgaacg tggcgaaagc gaccaaggaa |
| | 720 accttccgtc aggtgtatgc gggtaacctg ggtgcgccga gcaccctgga gaaatttatc |
| | 780 gcgaaggacg gccaaaaaga gttcgatctg aagaaactgc agaccgacct gaagaaaggt |
| | 840 attcgtggta aaagcaagga gcgtgattgg tgctgccagg aagagctgcg tagctacgtg |
| | 900 gagcaaaaca ccatccagta tgacctgtgg gcgtggggcg aaatgttcaa caaagcgcac |
| | 960 accgcgctga aaatcaagag cacccgtaac tacaactttg cgaagcaacg tctggaacag |
| | 1020 ttcaaagaga ttcagagcct gaacaacctg ctggttgtga agaagctgaa cgactttttc |
| | 1080 gatagcgaat ttttcagcgg cgaggaaacc tacaccatct gcgttcacca tctgggtggc |
| | 1140 aaggacctga gcaaactgta taaggcgtgg gaggatgatc cggcggaccc ggaaaacgcg |
| | 1200 attgtggttc tgtgcgacga tctgaaaaac aactttaaga aagagccgat ccgtaacatt |
| | 1260 ctgcgttaca tcttcaccat tcgtcaagaa tgcagcgcgc aggacatcct ggcggcggcg |
| | 1320 aagtacaacc aacagctgga tcgttataaa agccaaaagg cgaacccgag cgttctgggt |
| | 1380 aaccagggct ttacctggac caacgcggtg atcctgccgg agaaggcgca gcgtaacgac |
| | 1440 cgtccgaaca gcctggatct gcgtatttgg ctgtacctga aactgcgtca cccggacggt |
| | 1500 cgttggaaga aacaccatat cccgttctac gatacccgtt tcttccaaga aatttatgcg |
| | 1560 gcgggcaaca gcccggttga cacctgccag tttcgtaccc cgcgtttcgg ttatcacctg |
| | 1620 ccgaaactga ccgatcagac cgcgatccgt gttaacaaga aacatgtgaa agcggcgaag |
| | 1680 ccgaaactga ccgatcagac cgcgatccgt gttaacaaga aacatgtgaa agcggcgaag |
| | 1740 atcaccgaaa ttagcgcgac catcaacagc aaaggtcaag tgcgtattcc ggttaagttt |
| | 1800 gacgtgggtc gtcaaaaagg caccctgcag atcggtgacc gtttctgcgg ctacgatcaa |
| | 1860 aaccagaccg cgagccacgc gtatagcctg tgggaagtgg ttaaagaggg tcaataccat |
| | 1920 aaagagctgg gctgctttgt tcgtttcatc agcagcggtg acatcgtgag cattaccgag |
| | 1980 aaccgtggca accaatttga tcagctgagc tatgaaggtc tggcgtaccc gcaatatgcg |
| | 2040 gactggcgta agaaagcgag caagttcgtg agcctgtggc agatcaccaa gaaaaacaag |
| | 2100 aaaaaggaaa tcgtgaccgt tgaagcgaaa gagaagtttg acgcgatctg caagtaccag |
| | 2160 ccgcgtctgt ataaattcaa caaggagtac gcgtatctgc tgcgtgatat tgttcgtggc |
| | 2220 aaaagcctgg tggaactgca acagattcgt caagagatct ttcgtttcat tgaacaggac |
| | 2280 tgcggtgtta cccgtctggg cagcctgagc ctgagcaccc tggaaaccgt gaaagcggtt |
| | 2340 aagggtatca tttacagcta ttttagcacc gcgctgaacg cgagcaagaa caacccgatc |
| | 2400 agcgacgaac agcgtaaaga gtttgatccg gaactgttcg cgctgctgga aaagctggag |
| | 2460 ctgattcgta cccgtaaaaa gaaacaaaaa gtggaacgta tcgcgaacag cctgattcag |
| | 2520 acctgcctgg agaacaacat caagttcatt cgtggtgaag gcgacctgag caccaccaac |
| | 2580 aacgcgacca agaaaaaggc gaacagccgt agcatggatt ggttggcgcg tggtgttttt |
| | 2640 aacaaaatcc gtcaactggc gccgatgcac aacattaccc tgttcggttg cggcagcctg |
| | 2700 tacaccagcc accaggaccc gctggtgcat cgtaacccgg ataaagcgat gaagtgccgt |
| | 2760 tgggcggcga tcccggttaa ggacattggc gattgggtgc tgcgtaagct gagccaaaac |
| | 2820 ctgcgtgcga aaaacatcgg caccggcgag tactatcacc aaggtgttaa agagttcctg |
| | 2880 agccattatg aactgcagga cctggaggaa gagctgctga agtggcgtag cgatcgtaaa |
| | 2940 agcaacattc cgtgctgggt gctgcagaac cgtctggcgg agaagctggg caacaaagaa |
| | 3000 gcggtggttt acatcccggt tcgtggtggc cgtatttatt ttgcgaccca caaggtggcg |
| | 3060 accggtgcgg tgagcatcgt tttcgaccaa aaacaagtgt gggtttgcaa cgcggatcat |
| | 3120 gttgcggcgg cgaacatcgc gctgaccgtg aagggtattg gcgaacaaag cagcgacgaa |
| | 3162 gagaacccgg atggtagccg tatcaaactg cagctgacca gc |
| Cas12i2 | MSSAIKSYKSVLRPNERKNQLLKSTIQCLEDGSAFFFKMLQGLEGGITPEIVRESTEQEK |
| amino acid | QQQDIALWCAVNWFRPVSQDSLTHTIASDNLVEKFEEYYGGTASDAIKQYFSASIGESYY |
| sequence- | WNDCRQQYYDLCRELGVEVSDLTHDLEILCREKCLAVATESNQNNSIISVLFGTGEKEDR |
| SEQ ID NO: 772 | SVKLRITKKILEAISNLKEIPKNVAPIQEIILNVAKATKETFRQVYAGNLGAPSTLEKFI |
| | AKDGQKEFDLKKLQTDLKKVIRGKSKERDWCCQEELRSYVEQNTIQYDLWAWGEMENKAH |
| | TALKIKSTRNYNFAKQRLEQFKEIQSLNNLLVVKKLNDFFDSEFFSGEETYTICVHHLGG |
| | KDLSKLYKAWEDDPADPENAIVVLCDDLKNNFKKEPIRNILRYIFTIRQECSAQDILAAA |
| | KYNQQLDRYKSQKANPSVLGNQGFTWTNAVILPEKAQRNDRPNSLDLRIWLYLKLRHPDG |
| | RWKKHHIPFYDTRFFQEIYAAGNSPVDTCQFRTPRFGYHLPKLTDQTAIRVNKKHVKAAK |
| | TEARIRLAIQQGTLPVSNLKITEISATINSKGQVRIPVKFDVGRQKGTLQIGDRFCGYDQ |
| | NQTASHAYSLWEVVKEGQYHKELGCFVRFISSGDIVSITENRGNQFDQLSYEGLAYPQYA |
| | DWRKKASKFVSLWQITKKNKKKEIVTVEAKEKFDAICKYQPRLYKENKEYAYLLRDIVRG |
| | KSLVELQQIRQEIFRFIEQDCGVTRLGSLSLSTLETVKAVKGIIYSYFSTALNASKNNPI |
| | SDEQRKEFDPELFALLEKLELIRTRKKKQKVERIANSLIQTCLENNIKFIRGEGDLSTIN |
| | NATKKKANSRSMDWLARGVENKIRQLAPMHNITLFGCGSLYTSHQDPLVHRNPDKAMKCR |
| | WAAIPVKDIGDWVLRKLSQNLRAKNIGTGEYYHQGVKEFLSHYELQDLEEELLKWRSDRK |
| | SNIPCWVLQNRLAEKLGNKEAVVYIPVRGGRIYFATHKVATGAVSIVFDQKQVWVCNADH |
| | VAAANIALTVKGIGEQSSDEENPDGSRIKLQLTS |
| B2M- | ACTTAGCATCTCTGGGGCCAGTCTGCAAAGCGAGGGGGCAGCCTTAATGTGCCTCCAGCCTGAAGT |
| SEQ ID NO: 773 | CCTAGAATGAGCGCCCGGTGTCCCAAGCTGGGGCGCGCACCCCAGATCGGAGGGCGCCGATGTACA |
| | GACAGCAAACTCACCCAGTCTAGTGCATGCCTTCTTAAACATCACGAGACTCTAAGAAAAGGAAAC |
| | TGAAAACGGGAAAGTCCCTCTCTCTAACCTGGCACTGCGTCGCTGGCTTGGAGACAGGTGACGGTC |
| | CCTGCGGGCCTTGTCCTGATTGGCTGGGCACGCGTTTAATATAAGTGGAGGCGTCGCGCTGGCGGG |
| | CATTCCTGAAGCTGACAGCATTCGGGCCGAGATGTCTCGCTCCGTGGCCTTAGCTGTGCTCGCGCT |
| | ACTCTCTCTTTCTGGCCTGGAGGCTATCCAGCGTGAGTCTCTCCTACCCTCCCGCTCTGGTCCTTC |
| | CTCTCCCGCTCTGCACCCTCTGTGGCCCTCGCTGTGCTCTCTCGCTCCGTGACTTCCCTTCTCCAA |
| | GTTCTCCTTGGTGGCCCGCCGTGGGGCTAGTCCAGGGCTGGATCTCGGGGAAGCGGCGGGGTGGCC |
| | TGGGAGTGGGGAAGGGGGTGCGCACCCGGGACGCGCGCTACTTGCCCCTTTCGGCGGGGAGCAGGG |
| | GAGACCTTTGGCCTACGGCGACGGGAGGGTCGGGACAAAGTTTAGGGCGTCGATAAGCGTCAGAGC |
| | GCCGAGGTTGGGGGAGGGTTTCTCTTCCGCTCTTTCGCGGGGCCTCTGGCTCCCCCAGCGCAGCTG |
| | GAGTGGGGGACGGGTAGGCTCGTCCCAAAGGCGCGGCGCTGAGGTTTGTGAACGCGTGGAGGGGCG |
| | CTTGGGGTCTGGGGGAGGCGTCGCCCGGGTAAGCCTGTCTGCTGCGGCTCTGCTTCCCTTAGACTG |
| | GAGAGCTGTGGACTTCGTCTAGGCGCCCGCTAAGTTCGCATGTCCTAGCACCTCTGGGTCTATGTG |
| | GGGCCACACCGTGGGGAGGAAACAGCACGCGACGTTTGTAGAATGCTTGGCTGTGATACAAAGCGG |
| | TTTCGAATAATTAACTTATTTGTTCCCATCACATGTCACTTTTAAAAAATTATAAGAACTACCCGT |
| | TATTGACATCTTTCTGTGTGCCAAGGACTTTATGTGCTTTGCGTCATTTAATTTTGAAAACAGTTA |
| | TCTTCCGCCATAGATAACTACTATGGTTATCTTCTGCCTCTCACAGATGAAGAAACTAAGGCACCG |
| | AGATTTTAAGAAACTTAATTACACAGGGGATAAATGGCAGCAATCGAGATTGAAGTCAAGCCTAAC |
| | CAGGGCTTTTGCGGGAGCGCATGCCTTTTGGCTGTAATTCGTGCATTTTTTTTTAAGAAAAACGCC |
| | TGCCTTCTGCGTGAGATTCTCCAGAGCAAACTGGGCGGCATGGGCCCTGTGGTCTTTTCGTACAGA |
| | GGGCTTCCTCTTTGGCTCTTTGCCTGGTTGTTTCCAAGATGTACTGTGCCTCTTACTTTCGGTTTT |
| | GAAAACATGAGGGGGTTGGGCGTGGTAGCTTACGCCTGTAATCCCAGCACTTAGGGAGGCCGAGGC |
| | GGGAGGATGGCTTGAGGTCCGTAGTTGAGACCAGCCTGGCCAACATGGTGAAGCCTGGTCTCTACA |
| | AAAAATAATAACAAAAATTAGCCGGGTGTGGTGGCTCGTGCCTGTGGTCCCAGCTGCTCCGGTGGC |
| | TGAGGCGGGAGGATCTCTTGAGCTTAGGCTTTTGAGCTATCATGGCGCCAGTGCACTCCAGCGTGG |
| | GCAACAGAGCGAGACCCTGTCTCTCAAAAAAGAAAAAAAAAAAAAAAGAAAGAGAAAAGAAAAGAA |
| | AGAAAGAAGTGAAGGTTTGTCAGTCAGGGGAGCTGTAAAACCATTAATAAAGATAATCCAAGATGG |
| | TTACCAAGACTGTTGAGGACGCCAGAGATCTTGAGCACTTTCTAAGTACCTGGCAATACACTAAGC |
| | GCGCTCACCTTTTCCTCTGGCAAAACATGATCGAAAGCAGAATGTTTTGATCATGAGAAAATTGCA |
| | TTTAATTTGAATACAATTTATTTACAACATAAAGGATAATGTATATATCACCACCATTACTGGTAT |
| | TTGCTGGTTATGTTAGATGTCATTTTAAAAAATAACAATCTGATATTTAAAAAAAAATCTTATTTT |
| | GAAAATTTCCAAAGTAATACATGCCATGCATAGACCATTTCTGGAAGATACCACAAGAAACATGTA |
| | ATGATGATTGCCTCTGAAGGTCTATTTTCCTCCTCTGACCTGTGTGTGGGTTTTGTTTTTGTTTTA |
| | CTGTGGGCATAAATTAATTTTTCAGTTAAGTTTTGGAAGCTTAAATAACTCTCCAAAAGTCATAAA |
| | GCCAGTAACTGGTTGAGCCCAAATTCAAACCCAGCCTGTCTGATACTTGTCCTCTTCTTAGAAAAG |
| | ATTACAGTGATGCTCTCACAAAATCTTGCCGCCTTCCCTCAAACAGAGAGTTCCAGGCAGGATGAA |
| | TCTGTGCTCTGATCCCTGAGGCATTTAATATGTTCTTATTATTAGAAGCTCAGATGCAAAGAGCTC |
| | TCTTAGCTTTTAATGTTATGAAAAAAATCAGGTCTTCATTAGATTCCCCAATCCACCTCTTGATGG |
| | GGCTAGTAGCCTTTCCTTAATGATAGGGTGTTTCTAGAGAGATATATCTGGTCAAGGTGGCCTGGT |
| | ACTCCTCCTTCTCCCCACAGCCTCCCAGACAAGGAGGAGTAGCTGCCTTTTAGTGATCATGTACCC |
| | TGAATATAAGTGTATTTAAAAGAATTTTATACACATATATTTAGTGTCAATCTGTATATTTAGTAG |
| | CACTAACACTTCTCTTCATTTTCAATGAAAAATATAGAGTTTATAATATTTTCTTCCCACTTCCCC |
| | ATGGATGGTCTAGTCATGCCTCTCATTTTGGAAAGTACTGTTTCTGAAACATTAGGCAATATATTC |
| | CCAACCTGGCTAGTTTACAGCAATCACCTGTGGATGCTAATTAAAACGCAAATCCCACTGTCACAT |
| | GCATTACTCCATTTGATCATAATGGAAAGTATGTTCTGTCCCATTTGCCATAGTCCTCACCTATCC |
| | CTGTTGTATTTTATCGGGTCCAACTCAACCATTTAAGGTATTTGCCAGCTCTTGTATGCATTTAGG |
| | TTTTGTTTCTTTGTTTTTTAGCTCATGAAATTAGGTACAAAGTCAGAGAGGGGTCTGGCATATAAA |
| | ACCTCAGCAGAAATAAAGAGGTTTTGTTGTTTGGTAAGAACATACCTTGGGTTGGTTGGGCACGGT |
| | GGCTCGTGCCTGTAATCCCAACACTTTGGGAGGCCAAGGCAGGCTGATCACTTGAAGTTGGGAGTT |
| | CAAGACCAGCCTGGCCAACATGGTGAAATCCCGTCTCTACTGAAAATACAAAAATTAACCAGGCAT |
| | GGTGGTGTGTGCCTGTAGTCCCAGGAATCACTTGAACCCAGGAGGCGGAGGTTGCAGTGAGCTGAG |
| | ATCTCACCACTGCACACTGCACTCCAGCCTGGGCAATGGAATGAGATTCCATCCCAAAAAATAAAA |
| | AAATAAAAAAATAAAGAACATACCTTGGGTTGATCCACTTAGGAACCTCAGATAATAACATCTGCC |
| | ACGTATAGAGCAATTGCTATGTCCCAGGCACTCTACTAGACACTTCATACAGTTTAGAAAATCAGA |
| | TGGGTGTAGATCAAGGCAGGAGCAGGAACCAAAAAGAAAGGCATAAACATAAGAAAAAAAATGGAA |
| | GGGGTGGAAACAGAGTACAATAACATGAGTAATTTGATGGGGGCTATTATGAACTGAGAAATGAAC |
| | TTTGAAAAGTATCTTGGGGCCAAATCATGTAGACTCTTGAGTGATGTGTTAAGGAATGCTATGAGT |
| | GCTGAGAGGGCATCAGAAGTCCTTGAGAGCCTCCAGAGAAAGGCTCTTAAAAATGCAGCGCAATCT |
| | CCAGTGACAGAAGATACTGCTAGAAATCTGCTAGAAAAAAAACAAAAAAGGCATGTATAGAGGAAT |
| | TATGAGGGAAAGATACCAAGTCACGGTTTATTCTTCAAAATGGAGGTGGCTTGTTGGGAAGGTGGA |
| | AGCTCATTTGGCCAGAGTGGAAATGGAATTGGGAGAAATCGATGACCAAATGTAAACACTTGGTGC |
| | CTGATATAGCTTGACACCAAGTTAGCCCCAAGTGAAATACCCTGGCAATATTAATGTGTCTTTTCC |
| | CGATATTCCTCAGGTACTCCAAAGATTCAGGTTTACTCACGTCATCCAGCAGAGAATGGAAAGTCA |
| | AATTTCCTGAATTGCTATGTGTCTGGGTTTCATCCATCCGACATTGAAGTTGACTTACTGAAGAAT |
| | GGAGAGAGAATTGAAAAAGTGGAGCATTCAGACTTGTCTTTCAGCAAGGACTGGTCTTTCTATCTC |
| | TTGTACTACACTGAATTCACCCCCACTGAAAAAGATGAGTATGCCTGCCGTGTGAACCATGTGACT |
| | TTGTCACAGCCCAAGATAGTTAAGTGGGGTAAGTCTTACATTCTTTTGTAAGCTGCTGAAAGTTGT |
| | GTATGAGTAGTCATATCATAAAGCTGCTTTGATATAAAAAAGGTCTATGGCCATACTACCCTGAAT |
| | GAGTCCCATCCCATCTGATATAAACAATCTGCATATTGGGATTGTCAGGGAATGTTCTTAAAGATC |
| | AGATTAGTGGCACCTGCTGAGATACTGATGCACAGCATGGTTTCTGAACCAGTAGTTTCCCTGCAG |
| | TTGAGCAGGGAGCAGCAGCAGCACTTGCACAAATACATATACACTCTTAACACTTCTTACCTACTG |
| | GCTTCCTCTAGCTTTTGTGGCAGCTTCAGGTATATTTAGCACTGAACGAACATCTCAAGAAGGTAT |
| | AGGCCTTTGTTTGTAAGTCCTGCTGTCCTAGCATCCTATAATCCTGGACTTCTCCAGTACTTTCTG |
| | GCTGGATTGGTATCTGAGGCTAGTAGGAAGGGCTTGTTCCTGCTGGGTAGCTCTAAACAATGTATT |
| | CATGGGTAGGAACAGCAGCCTATTCTGCCAGCCTTATTTCTAACCATTTTAGACATTTGTTAGTAC |
| | ATGGTATTTTAAAAGTAAAACTTAATGTCTTCCTTTTTTTTCTCCACTGTCTTTTTCATAGATCGA |
| | GACATGTAAGCAGCATCATGGAGGTAAGTTTTTGACCTTGAGAAAATGTTTTTGTTTCACTGTCCT |
| | GAGGACTATTTATAGACAGCTCTAACATGATAACCCTCACTATGTGGAGAACATTGACAGAGTAAC |
| | ATTTTAGCAGGGAAAGAAGAATCCTACAGGGTCATGTTCCCTTCTCCTGTGGAGTGGCATGAAGAA |
| | GGTGTATGGCCCCAGGTATGGCCATATTACTGACCCTCTACAGAGAGGGCAAAGGAACTGCCAGTA |
| | TGGTATTGCAGGATAAAGGCAGGTGGTTACCCACATTACCTGCAAGGCTTTGATCTTTCTTCTGCC |
| | ATTTCCACATTGGACATCTCTGCTGAGGAGAGAAAATGAACCACTCTTTTCCTTTGTATAATGTTG |
| | TTTTATTCTTCAGACAGAAGAGAGGAGTTATACAGCTCTGCAGACATCCCATTCCTGTATGGGGAC |
| | TGTGTTTGCCTCTTAGAGGTTCCCAGGCCACTAGAGGAGATAAAGGGAAACAGATTGTTATAACTT |
| | GATATAATGATACTATAATAGATGTAACTACAAGGAGCTCCAGAAGCAAGAGAGAGGGAGGAACTT |
| | GGACTTCTCTGCATCTTTAGTTGGAGTCCAAAGGCTTTTCAATGAAATTCTACTGCCCAGGGTACA |
| | TTGATGCTGAAACCCCATTCAAATCTCCTGTTATATTCTAGAACAGGGAATTGATTTGGGAGAGCA |
| | TCAGGAAGGTGGATGATCTGCCCAGTCACACTGTTAGTAAATTGTAGAGCCAGGACCTGAACTCTA |
| | ATATAGTCATGTGTTACTTAATGACGGGGACATGTTCTGAGAAATGCTTACACAAACCTAGGTGTT |
| | GTAGCCTACTACACGCATAGGCTACATGGTATAGCCTATTGCTCCTAGACTACAAACCTGTACAGC |
| | CTGTTACTGTACTGAATACTGTGGGCAGTTGTAACACAATGGTAAGTATTTGTGTATCTAAACATA |
| | GAAGTTGCAGTAAAAATATGCTATTTTAATCTTATGAGACCACTGTCATATATACAGTCCATCATT |
| | GACCAAAACATCATATCAGCATTTTTTCTTCTAAGATTTTGGGAGCACCAAAGGGATACACTAACA |
| | GGATATACTCTTTATAATGGGTTTGGAGAACTGTCTGCAGCTACTTCTTTTAAAAAGGTGATCTAC |
| | ACAGTAGAAATTAGACAAGTTTGGTAATGAGATCTGCAATCCAAATAAAATAAATTCATTGCTAAC |
| | CTTTTTCTTTTCTTTTCAGGTTTGAAGATGCCGCATTTGGATTGGATGAATTCCAAATTCTGCTTG |
| | CTTGCTTTTTAATATTGATATGCTTATACACTTACACTTTATGCACAAAATGTAGGGTTATAATAA |
| | TGTTAACATGGACATGATCTTCTTTATAATTCTACTTTGAGTGCTGTCTCCATGTTTGATGTATCT |
| | GAGCAGGTTGCTCCACAGGTAGCTCTAGGAGGGCTGGCAACTTAGAGGTGGGGAGCAGAGAATTCT |
| | CTTATCCAACATCAACATCTTGGTCAGATTTGAACTCTTCAATCTCTTGCACTCAAAGCTTGTTAA |
| | GATAGTTAAGCGTGCATAAGTTAACTTCCAATTTACATACTCTGCTTAGAATTTGGGGGAAAATTT |
| | AGAAATATAATTGACAGGATTATTGGAAATTTGTTATAATGAATGAAACATTTTGTCATATAAGAT |
| | TCATATTTACTTCTTATACATTTGATAAAGTAAGGCATGGTTGTGGTTAATCTGGTTTATTTTTGT |
| | TCCACAAGTTAAATAAATCATAAAACTTGATGTGTTATCTCTTATATCTCACTCCCACTATTACCC |
| | CTTTATTTTCAAACAGGGAAACAGTCTTCAAGTTCCACTTGGTAAAAAATGTGAACCCCTTGTATA |
| | TAGAGTTTGGCTCACAGTGTAAAGGGCCTCAGTGATTCACATTTTCCAGATTAGGAATCTGATGCT |
| | CAAAGAAGTTAAATGGCATAGTTGGGGTGACACAGCTGTCTAGTGGGAGGCCAGCCTTCTATATTT |
| | TAGCCAGCGTTCTTTCCTGCGGGCCAGGTCATGAGGAGTATGCAGACTCTAAGAGGGAGCAAAAGT |
| | ATCTGAAGGATTTAATATTTTAGCAAGGAATAGATATACAATCATCCCTTGGTCTCCCTGGGGGAT |
| | TGGTTTCAGGACCCCTTCTTGGACACCAAATCTATGGATATTTAAGTCCCTTCTATAAAATGGTAT |
| | AGTATTTGCATATAACCTATCCACATCCTCCTGTATACTTTAAATCATTTCTAGATTACTTGTAAT |
| | ACCTAATACAATGTAAATGCTATGCAAATAGTTGTTATTGTTTAAGGAATAATGACAAGAAAAAAA |
| | AGTCTGTACATGCTCAGTAAAGACACAACCATCCCTTTTTTTCCCCAGTGTTTTTGATCCATGGTT |
| | TGCTGAATCCACAGATGTGGAGCCCCTGGATACGGAAGGCCCGCTGTACTTTGAATGACAAATAAC |
| | AGATTTAAAATTTTCAAGGCATAGTTTTATACCTGA |
| B2M-Exon 1- | GATTGGCTGGGCACGCGTTTAATATAAGTGGAGGCGTCGCGCTGGCGGGCATTCCTGAAGCTGACA |
| SEQ ID NO: 774 | GCATTCGGGCCGAGATGTCTCGCTCCGTGGCCTTAGCTGTGCTCGCGCTACTCTCTCTTTCTGGCC |
| | TGGAGGCTATCCAGCGTGAGTCTCTCCTACCCTCCCGCTCTGGTCCTTCCTCTCCCGCTCTGCAC |
| B2M-Exon 2- | AAGTGAAATACCCTGGCAATATTAATGTGTCTTTTCCCGATATTCCTCAGGTACTCCAAAGATTCA |
| SEQ ID NO: 775 | GGTTTACTCACGTCATCCAGCAGAGAATGGAAAGTCAAATTTCCTGAATTGCTATGTGTCTGGGTT |
| | TCATCCATCCGACATTGAAGTTGACTTACTGAAGAATGGAGAGAGAATTGAAAAAGTGGAGCATTC |
| | AGACTTGTCTTTCAGCAAGGACTGGTCTTTCTATCTCTTGTACTACACTGAATTCACCCCCACTGA |
| | AAAAGATGAGTATGCCTGCCGTGTGAACCATGTGACTTTGTCACAGCCCAAGATAGTTAAGTGGGG |
| | TAAGTCTTACATTCTTTTGTAAGCTGCTGAAAGTTGTGTATGAGTAGTC |
| B2M-Exon 3- | AAAGTAAAACTTAATGTCTTCCTTTTTTTTCTCCACTGTCTTTTTCATAGATCGAGACATGTAAGC |
| SEQ ID NO: 776 | AGCATCATGGAGGTAAGTTTTTGACCTTGAGAAAATGTTTTTGTTTCACTGTCCTGAGGACT |
| B2M-Exon 4- | GCAATCCAAATAAAATAAATTCATTGCTAACCTTTTTCTTTTCTTTTCAGGTTTGAAGATGCCGCA |
| SEQ ID NO: 777 | TTTGGATTGGATGAATTCCAAATTCTGCTTGCTTGCTTTTTAATATTGATATGCTTATACACTTAC |
| | ACTTTATGCACAAAATGTAGGGTTATAATAATGTTAACATGGACATGATCTTCTTTATAATTCTAC |
| | TTTGAGTGCTGTCTCCATGTTTGATGTATCTGAGCAGGTTGCTCCACAGGTAGCTCTAGGAGGGCT |
| | GGCAACTTAGAGGTGGGGAGCAGAGAATTCTCTTATCCAACATCAACATCTTGGTCAGATTTGAAC |
| | TCTTCAATCTCTTGCACTCAAAGCTTGTTAAGATAGTTAAGCGTGCATAAGTTAACTTCCAATTTA |
| | CATACTCTGCTTAGAATTTGGGGGAAAATTTAGAAATATAATTGACAGGATTATTGGAAATTTGTT |
| | ATAATGAATGAAACATTTTGTCATATAAGATTCATATTTACTTCTTATACATTTGATAAAGTAAGG |
| | CATGGTTGTGGTTAATCTGGTTTATTTTTGTTCCACAAGTTAAATAAATCATAAAACTTGATGTGT |
| | TATCTCTTATATCTCACTCCCACTATTACCCCTTTATTTTCAAACAGGGAAACAGTCTTCAAGTTC |
| | CACTTGGTAAAAAATGTGAACCCCTTGTATATAGAGTTTGGCTCACAGTGTAAAGGGCCTCAGTGA |
| | TTCACATTTTCCAGATTAGGAATCTGATGCTCAAAGAAGTTAAATGGCATAGTTGGGGTGACACAG |
| | CTGTCTAGTGGGAGGCCAGCCTTCTATATTTTAGCCAGCGTTCTTTCCTGCGGGCCAGGTCATGAG |
| | GAGTATGCAGACTCTAAGAGGGAGCAAAAGTATCTGAAGGATTTAATATTTTAGCAAGGAATAGAT |
| | ATACAATCATCCCTTGGTCTCCCTGGGGGATTGGTTTCAGGACCCCTTCTTGGACACCAAATCTAT |
| | GGATATTTAAGTCCCTTCTATAAAATGGTATAGTATTTGCATATAACCTATCCACATCCTCCTGTA |
| | TACTTTAAATCATTTCTAGATTACTTGTAATACCTAATACAATGTAAATGCTATGCAAATAGTTGT |
| | TATTGTTTAAGGAATAATGACAAGAAAAAAAAGTCTGTACATGCTCAGTAAAGACACAACCATCCC |
| | TTTTTTTCCCCAGTGTTTTTGATCCATGGTTTGCTGAATCCACAGATGTGGAGCCCCTGGATACGG |
| | AAGGCCCGCTGTACTTTGAATGACAAATAACAGATTTAAAATTTTCAAGGCATAGTTTTATACCTG |
| | A |
| SEQ ID NO: 782 | MSSAIKSYKS VLRPNERKNQ LLKSTIQCLE DGSAFFFKML QGLEGGITPE IVRESTEQEK |
| (Variant | QQQDIALWCA VNWFRPVSQD SLTHTIASDN LVEKFEEYYG GTASDAIKQY FSASIGESYY |
| Cas12i2 of | WNDCRQQYYD LCRELGVEVS DLTHDLEILC REKCLAVATE SNQNNSIISV LFGTGEKEDR |
| SEQ ID NO: 3 | SVKLRITKKI LEAISNLKEI PKNVAPIQEI ILNVAKATKE TFRQVYAGNL GAPSTLEKFI |
| of PCT/US2021/ | AKDGQKEFDL KKLQTDLKKV IRGKSKERDW CCQEELRSYV EQNTIQYDLW AWGEMENKAH |
| 025257) | TALKIKSTRN YNFAKQRLEQ FKEIQSLNNL LVVKKLNDFF DSEFFSGEET YTICVHHLGG |
| | KDLSKLYKAW EDDPADPENA IVVLCDDLKN NFKKEPIRNI LRYIFTIRQE CSAQDILAAA |
| | KYNQQLDRYK SQKANPSVLG NQGETWTNAV ILPEKAQRND RPNSLDLRIW LYLKLRHPDG |
| | RWKKHHIPFY DTRFFQEIYA AGNSPVDTCQ FRTPRFGYHL PKLTDQTAIR VNKKHVKAAK |
| | TEARIRLAIQ QGTLPVSNLK ITEISATINS KGQVRIPVKF RVGRQKGTLQ IGDRFCGYDQ |
| | NQTASHAYSL WEVVKEGQYH KELGCFVRFI SSGDIVSITE NRGNQFDQLS YEGLAYPQYA |
| | DWRKKASKFV SLWQITKKNK KKEIVTVEAK EKFDAICKYQ PRLYKENKEY AYLLRDIVRG |
| | KSLVELQQIR QEIFRFIEQD CGVTRLGSLS LSTLETVKAV KGIIYSYFST ALNASKNNPI |
| | SDEQRKEFDP ELFALLEKLE LIRTRKKKQK VERIANSLIQ TCLENNIKFI RGEGDLSTIN |
| | NATKKKANSR SMDWLARGVF NKIRQLAPMH NITLFGCGSL YTSHQDPLVH RNPDKAMKCR |
| | WAAIPVKDIG RWVLRKLSQN LRAKNRGTGE YYHQGVKEFL SHYELQDLEE ELLKWRSDRK |
| | SNIPCWVLQN RLAEKLGNKE AVVYIPVRGG RIYFATHKVA TGAVSIVEDQ KQVWVCNADH |
| | VAAANIALTG KGIGEQSSDE ENPDGSRIKL QLTS |
| SEQ ID NO: 783 | MSSAIKSYKS VLRPNERKNQ LLKSTIQCLE DGSAFFFKML QGLFGGITPE IVRFSTEQEK |
| (Variant | QQQDIALWCA VNWFRPVSQD SLTHTIASDN LVEKFEEYYG GTASDAIKQY FSASIGESYY |
| Cas12i2 of | WNDCRQQYYD LCRELGVEVS DLTHDLEILC REKCLAVATE SNQNNSIISV LFGTGEKEDR |
| SEQ ID NO: 4 | SVKLRITKKI LEAISNLKEI PKNVAPIQEI ILNVAKATKE TFRQVYAGNL GAPSTLEKFI |
| of PCT/US2021/ | AKDGQKEFDL KKLQTDLKKV IRGKSKERDW CCQEELRSYV EQNTIQYDLW AWGEMENKAH |
| 025257) | TALKIKSTRN YNFAKQRLEQ FKEIQSLNNL LVVKKLNDFF DSEFFSGEET YTICVHHLGG |
| | KDLSKLYKAW EDDPADPENA IVVLCDDLKN NFKKEPIRNI LRYIFTIRQE CSAQDILAAA |
| | KYNQQLDRYK SQKANPSVLG NQGFTWTNAV ILPEKAQRND RPNSLDLRIW LYLKLRHPDG |
| | RWKKHHIPFY DTRFFQEIYA AGNSPVDTCQ FRTPRFGYHL PKLTDQTAIR VNKKHVKAAK |
| | TEARIRLAIQ QGTLPVSNLK ITEISATINS KGQVRIPVKF RVGRQKGTLQ IGDRFCGYDQ |
| | NQTASHAYSL WEVVKEGQYH KELGCFVRFI SSGDIVSITE NRGNQFDQLS YEGLAYPQYA |
| | DWRKKASKFV SLWQITKKNK KKEIVTVEAK EKFDAICKYQ PRLYKENKEY AYLLRDIVRG |
| | KSLVELQQIR QEIFRFIEQD CGVTRLGSLS LSTLETVKAV KGIIYSYFST ALNASKNNPI |
| | SDEQRKEFDP ELFALLEKLE LIRTRKKKQK VERIANSLIQ TCLENNIKFI RGEGDLSTIN |
| | NATKKKANSR SMDWLARGVF NKIRQLAPMH NITLFGCGSL YTSHQDPLVH RNPDKAMKCR |
| | WAAIPVKDIG DWVLRKLSQN LRAKNRGTGE YYHQGVKEFL SHYELQDLEE ELLKWRSDRK |
| | SNIPCWVLQN RLAEKLGNKE AVVYIPVRGG RIYFATHKVA TGAVSIVEDQ KQVWVCNADH |
| | VAAANIALTG KGIGEQSSDE ENPDGSRIKL QLTS |
| SEQ ID NO: 784 | MSSAIKSYKS VLRPNERKNQ LLKSTIQCLE DGSAFFFKML QGLEGGITPE IVRESTEQEK |
| (Variant | QQQDIALWCA VNWERPVSQD SLTHTIASDN LVEKFEEYYG GTASDAIKQY FSASIGESYY |
| Cas12i2 of | WNDCRQQYYD LCRELGVEVS DLTHDLEILC REKCLAVATE SNQNNSIISV LFGTGEKEDR |
| SEQ ID NO: 5 | SVKLRITKKI LEAISNLKEI PKNVAPIQEI ILNVAKATKE TFRQVYAGNL GAPSTLEKFI |
| of PCT/US2021/ | AKDGQKEFDL KKLQTDLKKV IRGKSKERDW CCQEELRSYV EQNTIQYDLW AWGEMENKAH |
| 025257) | TALKIKSTRN YNFAKQRLEQ FKEIQSLNNL LVVKKLNDFF DSEFFSGEET YTICVHHLGG |
| | KDLSKLYKAW EDDPADPENA IVVLCDDLKN NFKKEPIRNI LRYIFTIRQE CSAQDILAAA |
| | KYNQQLDRYK SQKANPSVLG NQGETWTNAV ILPEKAQRND RPNSLDLRIW LYLKLRHPDG |
| | RWKKHHIPFY DTRFFQEIYA AGNSPVDTCQ FRTPRFGYHL PKLTDQTAIR VNKKHVKAAK |
| | TEARIRLAIQ QGTLPVSNLK ITEISATINS KGQVRIPVKF RVGRQKGTLQ IGDRFCGYDQ |
| | NQTASHAYSL WEVVKEGQYH KELGCFVRFI SSGDIVSITE NRGNQFDQLS YEGLAYPQYA |
| | DWRKKASKFV SLWQITKKNK KKEIVTVEAK EKFDAICKYQ PRLYKENKEY AYLLRDIVRG |
| | KSLVELQQIR QEIFRFIEQD CGVTRLGSLS LSTLETVKAV KGIIYSYFST ALNASKNNPI |
| | SDEQRKEFDP ELFALLEKLE LIRTRKKKQK VERIANSLIQ TCLENNIKFI RGEGDLSTIN |
| | NATKKKANSR SMDWLARGVF NKIRQLAPMH NITLFGCGSL YTSHQDPLVH RNPDKAMKCR |
| | WAAIPVKDIG DWVLRKLSQN LRAKNRGTGE YYHQGVKEFL SHYELQDLEE ELLKWRSDRK |
| | SNIPCWVLQN RLAEKLGNKE AVVYIPVRGG RIYFATHKVA TGAVSIVEDQ KQVWVCNADH |
| | VAAANIALTG KGIGEQSSDE ENPDGGRIKL QLTS |
| SEQ ID NO: 785 | MSSAIKSYKS VLRPNERKNQ LLKSTIQCLE DGSAFFFKML QGLEGGITPE IVRESTEQEK |
| (Variant | QQQDIALWCA VNWFRPVSQD SLTHTIASDN LVEKFEEYYG GTASDAIKQY FSASIGESYY |
| Cas12i2 of | WNDCRQQYYD LCRELGVEVS DLTHDLEILC REKCLAVATE SNQNNSIISV LFGTGEKEDR |
| SEQ ID NO: 495 of | SVKLRITKKI LEAISNLKEI PKNVAPIQEI ILNVAKATKE TFRQVYAGNL GAPSTLEKFI |
| PCT/US2021/ | AKDGQKEFDL KKLQTDLKKV IRGKSKERDW CCQEELRSYV EQNTIQYDLW AWGEMENKAH |
| 025257) | TALKIKSTRN YNFAKQRLEQ FKEIQSLNNL LVVKKLNDFF DSEFFSGEET YTICVHHLGG |
| | KDLSKLYKAW EDDPADPENA IVVLCDDLKN NFKKEPIRNI LRYIFTIRQE CSAQDILAAA |
| | KYNQQLDRYK SQKANPSVLG NQGFTWTNAV ILPEKAQRND RPNSLDLRIW LYLKLRHPDG |
| | RWKKHHIPFY DTRFFQEIYA AGNSPVDTCQ FRTPRFGYHL PKLTDQTAIR VNKKHVKAAK |
| | TEARIRLAIQ QGTLPVSNLK ITEISATINS KGQVRIPVKF RVGRQKGTLQ IGDRFCGYDQ |
| | NQTASHAYSL WEVVKEGQYH KELRCRVRFI SSGDIVSITE NRGNQFDQLS YEGLAYPQYA |
| | DWRKKASKFV SLWQITKKNK KKEIVTVEAK EKFDAICKYQ PRLYKENKEY AYLLRDIVRG |
| | KSLVELQQIR QEIFRFIEQD CGVTRLGSLS LSTLETVKAV KGIIYSYEST ALNASKNNPI |
| | SDEQRKEFDP ELFALLEKLE LIRTRKKKQK VERIANSLIQ TCLENNIKFI RGEGDLSTTN |
| | NATKKKANSR SMDWLARGVF NKIRQLAPMH NITLFGCGSL YTSHQDPLVH RNPDKAMKCR |
| | WAAIPVKDIG DWVLRKLSQN LRAKNRGTGE YYHQGVKEFL SHYELQDLEE ELLKWRSDRK |
| | SNIPCWVLQN RLAEKLGNKE AVVYIPVRGG RIYFATHKVA TGAVSIVEDQ KQVWVCNADH |
| | VAAANIALTG KGIGRQSSDE ENPDGGRIKL QLTS |
| SEQ ID NO: 786 | MSSAIKSYKS VLRPNERKNQ LLKSTIQCLE DGSAFFFKML QGLFGGITPE IVRESTEQEK |
| (Variant | QQQDIALWCA VNWFRPVSQD SLTHTIASDN LVEKFEEYYG GTASDAIKQY FSASIGESYY |
| Cas12i2 of | WNDCRQQYYD LCRELGVEVS DLTHDLEILC REKCLAVATE SNQNNSIISV LFGTGEKEDR |
| SEQ ID NO: 496 of | SVKLRITKKI LEAISNLKEI PKNVAPIQEI ILNVAKATKE TFRQVYAGNL GAPSTLEKFI |
| PCT/US2021/ | AKDGQKEFDL KKLQTDLKKV IRGKSKERDW CCQEELRSYV EQNTIQYDLW AWGEMFNKAH |
| 025257) | TALKIKSTRN YNFAKQRLEQ FKEIQSLNNL LVVKKLNDFF DSEFFSGEET YTICVHHLGG |
| | KDLSKLYKAW EDDPADPENA IVVLCDDLKN NFKKEPIRNI LRYIFTIRQE CSAQDILAAA |
| | KYNQQLDRYK SQKANPSVLG NQGFTWTNAV ILPEKAQRND RPNSLDLRIW LYLKLRHPDG |
| | RWKKHHIPFY DTRFFQEIYA AGNSPVDTCQ FRTPRFGYHL PKLTDQTAIR VNKKHVKAAK |
| | TEARIRLAIQ QGTLPVSNLK ITEISATINS KGQVRIPVKF RVGRQKGTLQ IGDRFCGYDQ |
| | NQTASHAYSL WEVVKEGQYH KELRCRVRFI SSGDIVSITE NRGNQFDQLS YEGLAYPQYA |
| | DWRKKASKFV SLWQITKKNK KKEIVTVEAK EKFDAICKYQ PRLYKFNKEY AYLLRDIVRG |
| | KSLVELQQIR QEIFRFIEQD CGVTRLGSLS LSTLETVKAV KGIIYSYFST ALNASKNNPI |
| | SDEQRKEFDP ELFALLEKLE LIRTRKKKQK VERIANSLIQ TCLENNIKFI RGEGDLSTTN |
| | NATKKKANSR SMDWLARGVF NKIRQLATMH NITLFGCGSL YTSHQDPLVH RNPDKAMKCR |
| | WAAIPVKDIG DWVLRKLSQN LRAKNRGTGE YYHQGVKEFL SHYELQDLEE ELLKWRSDRK |
| | SNIPCWVLQN RLAEKLGNKE AVVYIPVRGG RIYFATHKVA TGAVSIVEDQ KQVWVCNADH |
| | VAAANIALTG KGIGRQSSDE ENPDGGRIKL QLTS |
| SEQ ID NO: 787 | ATGGCTTCCATCTCTAGGCCATACGGCACCAAGCTGCGACCGGACGCACGGAAGAAGGAGATGCTC |
| (Nucleotide | GATAAGTTCTTTAATACACTGACTAAGGGTCAGCGCGTGTTCGCAGACCTGGCCCTGTGCATCTAT |
| sequence | GGCTCCCTGACCCTGGAGATGGCCAAGTCTCTGGAGCCAGAAAGTGATTCAGAACTGGTGTGCGCT |
| encoding | ATTGGGTGGTTTCGGCTGGTGGACAAGACCATCTGGTCCAAGGATGGCATCAAGCAGGAGAATCTG |
| Cas12i4) | GTGAAACAGTACGAAGCCTATTCCGGAAAGGAGGCTTCTGAAGTGGTCAAAACATACCTGAACAGC |
| | CCCAGCTCCGACAAGTACGTGTGGATCGATTGCAGGCAGAAATTCCTGAGGTTTCAGCGCGAGCTC |
| | GGCACTCGCAACCTGTCCGAGGACTTCGAATGTATGCTCTTTGAACAGTACATTAGACTGACCAAG |
| | GGCGAGATCGAAGGGTATGCCGCTATTTCAAATATGTTCGGAAACGGCGAGAAGGAAGACCGGAGC |
| | AAGAAAAGAATGTACGCTACACGGATGAAAGATTGGCTGGAGGCAAACGAAAATATCACTTGGGAG |
| | CAGTATAGAGAGGCCCTGAAGAACCAGCTGAATGCTAAAAACCTGGAGCAGGTTGTGGCCAATTAC |
| | AAGGGGAACGCTGGCGGGGCAGACCCCTTCTTTAAGTATAGCTTCTCCAAAGAGGGAATGGTGAGC |
| | AAGAAAGAACATGCACAGCAGCTCGACAAGTTCAAAACCGTCCTGAAGAACAAAGCCCGGGACCTG |
| | AATTTTCCAAACAAGGAGAAGCTGAAGCAGTACCTGGAGGCCGAAATCGGCATTCCGGTCGACGCT |
| | AACGTGTACTCCCAGATGTTCTCTAACGGGGTGAGTGAGGTCCAGCCTAAGACCACACGGAATATG |
| | TCTTTTAGTAACGAGAAACTGGATCTGCTCACTGAACTGAAGGACCTGAACAAGGGCGATGGGTTC |
| | GAGTACGCCAGAGAAGTGCTGAACGGGTTCTTTGACTCCGAGCTCCACACTACCGAGGATAAGTTT |
| | AATATCACCTCTAGGTACCTGGGAGGCGACAAATCAAACCGCCTGAGCAAACTCTATAAGATCTGG |
| | AAGAAAGAGGGTGTGGACTGCGAGGAAGGCATTCAGCAGTTCTGTGAAGCCGTCAAAGATAAGATG |
| | GGCCAGATCCCCATTCGAAATGTGCTGAAGTACCTGTGGCAGTTCCGGGAGACAGTCAGTGCCGAG |
| | GATTTTGAAGCAGCCGCTAAGGCTAACCATCTGGAGGAAAAGATCAGCCGGGTGAAAGCCCACCCA |
| | ATCGTGATTAGCAATAGGTACTGGGCTTTTGGGACTTCCGCACTGGTGGGAAACATTATGCCCGCA |
| | GACAAGAGGCATCAGGGAGAGTATGCCGGTCAGAATTTCAAAATGTGGCTGGAGGCTGAACTGCAC |
| | TACGATGGCAAGAAAGCAAAGCACCATCTGCCTTTTTATAACGCCCGCTTCTTTGAGGAAGTGTAC |
| | TGCTATCACCCCTCTGTCGCCGAGATCACTCCTTTCAAAACCAAGCAGTTTGGCTGTGAAATCGGG |
| | AAGGACATTCCAGATTACGTGAGCGTCGCTCTGAAGGACAATCCGTATAAGAAAGCAACCAAACGA |
| | ATCCTGCGTGCAATCTACAATCCCGTCGCCAACACAACTGGCGTTGATAAGACCACAAACTGCAGC |
| | TTCATGATCAAACGCGAGAATGACGAATATAAGCTGGTCATCAACCGAAAAATTTCCGTGGATCGG |
| | CCTAAGAGAATCGAAGTGGGCAGGACAATTATGGGGTACGACCGCAATCAGACAGCTAGCGATACT |
| | TATTGGATTGGCCGGCTGGTGCCACCTGGAACCCGGGGCGCATACCGCATCGGAGAGTGGAGCGTC |
| | CAGTATATTAAGTCCGGGCCTGTCCTGTCTAGTACTCAGGGAGTTAACAATTCCACTACCGACCAG |
| | CTGGTGTACAACGGCATGCCATCAAGCTCCGAGCGGTTCAAGGCCTGGAAGAAAGCCAGAATGGCT |
| | TTTATCCGAAAACTCATTCGTCAGCTGAATGACGAGGGACTGGAATCTAAGGGTCAGGATTATATC |
| | CCCGAGAACCCTTCTAGTTTCGATGTGCGGGGCGAAACCCTGTACGTCTTTAACAGTAATTATCTG |
| | AAGGCCCTGGTGAGCAAACACAGAAAGGCCAAGAAACCTGTTGAGGGGATCCTGGACGAGATTGAA |
| | GCCTGGACATCTAAAGACAAGGATTCATGCAGCCTGATGCGGCTGAGCAGCCTGAGCGATGCTTCC |
| | ATGCAGGGAATCGCCAGCCTGAAGAGTCTGATTAACAGCTACTTCAACAAGAATGGCTGTAAAACC |
| | ATCGAGGACAAAGAAAAGTTTAATCCCGTGCTGTATGCCAAGCTGGTTGAGGTGGAACAGCGGAGA |
| | ACAAACAAGCGGTCTGAGAAAGTGGGAAGAATCGCAGGTAGTCTGGAGCAGCTGGCCCTGCTGAAC |
| | GGGGTTGAGGTGGTCATCGGCGAAGCTGACCTGGGGGAGGTCGAAAAAGGAAAGAGTAAGAAACAG |
| | AATTCACGGAACATGGATTGGTGCGCAAAGCAGGTGGCACAGCGGCTGGAGTACAAACTGGCCTTC |
| | CATGGAATCGGTTACTTTGGAGTGAACCCCATGTATACCAGCCACCAGGACCCTTTCGAACATAGG |
| | CGCGTGGCTGATCACATCGTCATGCGAGCACGTTTTGAGGAAGTCAACGTGGAGAACATTGCCGAA |
| | TGGCACGTGCGAAATTTCTCAAACTACCTGCGTGCAGACAGCGGCACTGGGCTGTACTATAAGCAG |
| | GCCACCATGGACTTCCTGAAACATTACGGTCTGGAGGAACACGCTGAGGGCCTGGAAAATAAGAAA |
| | ATCAAGTTCTATGACTTTAGAAAGATCCTGGAGGATAAAAACCTGACAAGCGTGATCATTCCAAAG |
| | AGGGGCGGGCGCATCTACATGGCCACCAACCCAGTGACATCCGACTCTACCCCGATTACATACGCC |
| | GGCAAGACTTATAATAGGTGTAACGCTGATGAGGTGGCAGCCGCTAATATCGTTATTTCTGTGCTG |
| | GCTCCCCGCAGTAAGAAAAACGAGGAACAGGACGATATCCCTCTGATTACCAAGAAAGCCGAGAGT |
| | AAGTCACCACCGAAAGACCGGAAGAGATCAAAAACAAGCCAGCTGCCTCAGAAA |
| SEQ ID NO: 814 | MASISRPYGTKLRPDARKKEMLDKFFNTLTKGQRVFADLALCIYGSLTLEMAKSLEPESDSELVCA |
| Cas1214 | IGWFRLVDKTIWSKDGIKQENLVKQYEAYSGKEASEVVKTYLNSPSSDKYVWIDCRQKFLRFQREL |
| amino acid | GTRNLSEDFECMLFEQYIRLIKGEIEGYAAISNMFGNGEKEDRSKKRMYATRMKDWLEANENITWE |
| sequence of | QYREALKNQLNAKNLEQVVANYKGNAGGADPFFKYSFSKEGMVSKKEHAQQLDKFKTVLKNKARDL |
| SEQ ID NO: 14 | NFPNKEKLKQYLEAEIGIPVDANVYSQMFSNGVSEVQPKTTRNMSFSNEKLDLLTELKDLNKGDGF |
| of U.S. Pat. No. | EYAREVLNGFFDSELHTTEDKFNITSRYLGGDKSNRLSKLYKIWKKEGVDCEEGIQQFCEAVKDKM |
| 10,808,245) | GQIPIRNVLKYLWQFRETVSAEDFEAAAKANHLEEKISRVKAHPIVISNRYWAFGTSALVGNIMPA |
| | DKRHQGEYAGQNFKMWLEAELHYDGKKAKHHLPFYNARFFEEVYCYHPSVAEITPFKTKQFGCEIG |
| | KDIPDYVSVALKDNPYKKATKRILRAIYNPVANTTGVDKTTNCSFMIKRENDEYKLVINRKISVDR |
| | PKRIEVGRTIMGYDRNQTASDTYWIGRLVPPGTRGAYRIGEWSVQYIKSGPVLSSTQGVNNSTTDQ |
| | LVYNGMPSSSERFKAWKKARMAFIRKLIRQLNDEGLESKGQDYIPENPSSFDVRGETLYVENSNYL |
| | KALVSKHRKAKKPVEGILDEIEAWTSKDKDSCSLMRLSSLSDASMQGIASLKSLINSYFNKNGCKT |
| | IEDKEKFNPVLYAKLVEVEQRRTNKRSEKVGRIAGSLEQLALLNGVEVVIGEADLGEVEKGKSKKQ |
| | NSRNMDWCAKQVAQRLEYKLAFHGIGYFGVNPMYTSHQDPFEHRRVADHIVMRARFEEVNVENIAE |
| | WHVRNFSNYLRADSGTGLYYKQATMDFLKHYGLEEHAEGLENKKIKFYDERKILEDKNLTSVIIPK |
| | RGGRIYMATNPVTSDSTPITYAGKTYNRCNADEVAAANIVISVLAPRSKKNEEQDDIPLITKKAES |
| | KSPPKDRKRSKTSQLPQK |
| SEQ ID NO: 815 | MASISRPYGT KLRPDARKKE MLDKFFNTLT KGQRVFADLA LCIYGSLTLE MAKSLEPESD |
| (Variant Cas12i4) | SELVCAIGWF RLVDKTIWSK DGIKQENLVK QYEAYSGKEA SEVVKTYLNS PSSDKYVWID |
| | CRQKFLRFQR ELGTRNLSED FECMLFEQYI RLTKGEIEGY AAISNMEGNG EKEDRSKKRM |
| | YATRMKDWLE ANENITWEQY REALKNQLNA KNLEQVVANY KGNAGGADPF FKYSFSKEGM |
| | VSKKEHAQQL DKFKTVLKNK ARDLNFPNKE KLKQYLEAEI GIPVDANVYS QMFSNGVSEV |
| | QPKTTRNMSF SNEKLDLLTE LKDLNKGDGF EYAREVLNGF FDSELHTTED KENITSRYLG |
| | GDKSNRLSKL YKIWKKEGVD CEEGIQQFCE AVKDKMGQIP IRNVLKYLWQ FRETVSAEDF |
| | EAAAKANHLE EKISRVKAHP IVISNRYWAF GTSALVGNIM PADKRHQGEY AGQNFKMWLE |
| | AELHYDGKKA KHHLPFYNAR FFEEVYCYHP SVAEITPFKT KQFGCEIGKD IPDYVSVALK |
| | DNPYKKATKR ILRAIYNPVA NTTGVDKTIN CSFMIKREND EYKLVINRKI SRDRPKRIEV |
| | GRTIMGYDRN QTASDTYWIG RLVPPGTRGA YRIGEWSVQY IKSGPVLSST QGVNNSTTDQ |
| | LVYNGMPSSS ERFKAWKKAR MAFIRKLIRQ LNDEGLESKG QDYIPENPSS FDVRGETLYV |
| | FNSNYLKALV SKHRKAKKPV EGILDEIEAW TSKDKDSCSL MRLSSLSDAS MQGIASLKSL |
| | INSYFNKNGC KTIEDKEKFN PVLYAKLVEV EQRRINKRSE KVGRIAGSLE QLALLNGVEV |
| | VIGEADLGEV EKGKSKKQNS RNMDWCAKQV AQRLEYKLAF HGIGYFGVNP MYTSHQDPFE |
| | HRRVADHIVM RARFEEVNVE NIAEWHVRNF SNYLRADSGT GLYYKQATMD FLKHYGLEEH |
| | AEGLENKKIK FYDERKILED KNLISVIIPK RGGRIYMATN PVTSDSTPIT YAGKTYNRCN |
| | ADEVAAANIV ISVLAPRSKK NREQDDIPLI TKKAESKSPP KDRKRSKTSQ LPQK |
| SEQ ID NO: 816 | MASISRPYGT KLRPDARKKE MLDKFFNTLT KGQRVFADLA LCIYGSLTLE MAKSLEPESD |
| (Variant Cas12i4) | SELVCAIGWF RLVDKTIWSK DGIKQENLVK QYEAYSGKEA SEVVKTYLNS PSSDKYVWID |
| | CRQKFLRFQR ELGTRNLSED FECMLFEQYI RLTKGEIEGY AAISNMEGNG EKEDRSKKRM |
| | YATRMKDWLE ANENITWEQY REALKNQLNA KNLEQVVANY KGNAGGADPF FKYSFSKEGM |
| | VSKKEHAQQL DKFKTVLKNK ARDLNFPNKE KLKQYLEAEI GIPVDANVYS QMFSNGVSEV |
| | QPKTTRNMSF SNEKLDLLTE LKDLNKGDGF EYAREVLNGF FDSELHTTED KENITSRYLG |
| | GDKSNRLSKL YKIWKKEGVD CEEGIQQFCE AVKDKMGQIP IRNVLKYLWQ FRETVSAEDF |
| | EAAAKANHLE EKISRVKAHP IVISNRYWAF GTSALVGNIM PADKRHQGEY AGQNFKMWLR |
| | AELHYDGKKA KHHLPFYNAR FFEEVYCYHP SVAEITPFKT KQFGCEIGKD IPDYVSVALK |
| | DNPYKKATKR ILRAIYNPVA NTTRVDKTTN CSFMIKREND EYKLVINRKI SRDRPKRIEV |
| | GRTIMGYDRN QTASDTYWIG RLVPPGTRGA YRIGEWSVQY IKSGPVLSST QGVNNSTTDQ |
| | LVYNGMPSSS ERFKAWKKAR MAFIRKLIRQ LNDEGLESKG QDYIPENPSS FDVRGETLYV |
| | FNSNYLKALV SKHRKAKKPV EGILDEIEAW TSKDKDSCSL MRLSSLSDAS MQGIASLKSL |
| | INSYFNKNGC KTIEDKEKFN PVLYAKLVEV EQRRINKRSE KVGRIAGSLE QLALLNGVEV |
| | VIGEADLGEV EKGKSKKQNS RNMDWCAKQV AQRLEYKLAF HGIGYFGVNP MYTSHQDPFE |
| | HRRVADHIVM RARFEEVNVE NIAEWHVRNF SNYLRADSGT GLYYKQATMD FLKHYGLEEH |
| | AEGLENKKIK FYDERKILED KNLISVIIPK RGGRIYMATN PVTSDSTPIT YAGKTYNRCN |
| | ADEVAAANIV ISVLAPRSKK NREQDDIPLI TKKAESKSPP KDRKRSKTSQ LPQK |
| SEQ ID NO: 817 | MSNKEKNASETRKAYTTKMIPRSHDRMKLLGNFMDYLMDGTPIFFELWNQFGGGIDRDIISGTANK |
| (Cas12i1 of | DKISDDLLLAVNWFKVMPINSKPQGVSPSNLANLFQQYSGSEPDIQAQEYFASNEDTEKHQWKDMR |
| SEQ ID NO: 3 | VEYERLLAELQLSRSDMHHDLKLMYKEKCIGLSLSTAHYITSVMFGTGAKNNRQTKHQFYSKVIQL |
| of U.S. Pat. No. | LEESTQINSVEQLASIILKAGDCDSYRKLRIRCSRKGATPSILKIVQDYELGTNHDDEVNVPSLIA |
| 10,808,245) | NLKEKLGRFEYECEWKCMEKIKAFLASKVGPYYLGSYSAMLENALSPIKGMTTKNCKFVLKQIDAK |
| | NDIKYENEPFGKIVEGFFDSPYFESDTNVKWVLHPHHIGESNIKTLWEDLNAIHSKYEEDIASLSE |
| | DKKEKRIKVYQGDVCQTINTYCEEVGKEAKTPLVQLLRYLYSRKDDIAVDKIIDGITFLSKKHKVE |
| | KQKINPVIQKYPSFNFGNNSKLLGKIISPKDKLKHNLKCNRNQVDNYIWIEIKVLNTKTMRWEKHH |
| | YALSSTRFLEEVYYPATSENPPDALAARFRTKINGYEGKPALSAEQIEQIRSAPVGLRKVKKRQMR |
| | LEAARQQNLLPRYTWGKDFNINICKRGNNFEVTLATKVKKKKEKNYKVVLGYDANIVRKNTYAAIE |
| | AHANGDGVIDYNDLPVKPIESGFVTVESQVRDKSYDQLSYNGVKLLYCKPHVESRRSFLEKYRNGT |
| | MKDNRGNNIQIDEMKDFEAIADDETSLYYFNMKYCKLLQSSIRNHSSQAKEYREEIFELLRDGKLS |
| | VLKLSSLSNLSFVMFKVAKSLIGTYFGHLLKKPKNSKSDVKAPPITDEDKQKADPEMFALRLALEE |
| | KRLNKVKSKKEVIANKIVAKALELRDKYGPVLIKGENISDTTKKGKKSSTNSFLMDWLARGVANKV |
| | KEMVMMHQGLEFVEVNPNFTSHQDPFVHKNPENTFRARYSRCTPSELTEKNRKEILSFLSDKPSKR |
| | PTNAYYNEGAMAFLATYGLKKNDVLGVSLEKFKQIMANILHQRSEDQLLFPSRGGMFYLATYKLDA |
| | DATSVNWNGKQFWVCNADLVAAYNVGLVDIQKDFKKK |
| SEQ ID NO: 818 | MSISNNNILPYNPKLLPDDRKHKMLVDTFNQLDLIRNNLHDMIIALYGALKYDNIKQFASKEKPHI |
| (Cas12i3 of | SADALCSINWFRLVKINERKPAIESNQIISKFIQYSGHTPDKYALSHITGNHEPSHKWIDCREYAI |
| SEQ ID NO: 14 | NYARIMHLSFSQFQDLATACLNCKILILNGTLTSSWAWGANSALFGGSDKENFSVKAKILNSFIEN |
| of U.S. Pat. No. | LKDEMNTTKFQVVEKVCQQIGSSDAADLFDLYRSTVKDGNRGPATGRNPKVMNLFSQDGEISSEQR |
| 10,808,245) | EDFIESFQKVMQEKNSKQIIPHLDKLKYHLVKQSGLYDIYSWAAAIKNANSTIVASNSSNLNTILN |
| | KTEKQQTFEELRKDEKIVACSKILLSVNDTLPEDLHYNPSTSNLGKNLDVFFDLLNENSVHTIENK |
| | EEKNKIVKECVNQYMEECKGLNKPPMPVLLTFISDYAHKHQAQDFLSAAKMNFIDLKIKSIKVVPT |
| | VHGSSPYTWISNLSKKNKDGKMIRTPNSSLIGWIIPPEEIHDQKFAGQNPIIWAVLRVYCNNKWEM |
| | HHFPFSDSRFFTEVYAYKPNLPYLPGGENRSKRFGYRHSTNLSNESRQILLDKSKYAKANKSVLRC |
| | MENMTHNVVFDPKTSLNIRIKTDKNNSPVLDDKGRITFVMQINHRILEKYNNTKIEIGDRILAYDQ |
| | NQSENHTYAILQRTEEGSHAHQFNGWYVRVLETGKVTSIVQGLSGPIDQLNYDGMPVTSHKENCWQ |
| | ADRSAFVSQFASLKISETETFDEAYQAINAQGAYTWNLFYLRILRKALRVCHMENINQFREEILAI |
| | SKNRLSPMSLGSLSQNSLKMIRAFKSIINCYMSRMSFVDELQKKEGDLELHTIMRLTDNKLNDKRV |
| | EKINRASSFLINKAHSMGCKMIVGESDLPVADSKTSKKQNVDRMDWCARALSHKVEYACKLMGLAY |
| | RGIPAYMSSHQDPLVHLVESKRSVLRPRFVVADKSDVKQHHLDNLRRMLNSKTKVGTAVYYREAVE |
| | LMCEELGIHKTDMAKGKVSLSDFVDKFIGEKAIFPQRGGRFYMSTKRLTTGAKLICYSGSDVWLSD |
| | ADEIAAINIGMFVVCDQTGAFKKKKKEKLDDEECDILPFRPM |
| B2M intron 1 | GTGAGTCTCTCCTACCCTCCCGCTCTGGTCCTTCCTCTCCCGCTCTGCACCCTCTGTGGCCCTCGC |
| (SEQ ID NO: 1219) | TGTGCTCTCTCGCTCCGTGACTTCCCTTCTCCAAGTTCTCCTTGGTGGCCCGCCGTGGGGCTAGTC |
| | CAGGGCTGGATCTCGGGGAAGCGGCGGGGTGGCCTGGGAGTGGGGAAGGGGGTGCGCACCCGGGAC |
| | GCGCGCTACTTGCCCCTTTCGGCGGGGAGCAGGGGAGACCTTTGGCCTACGGCGACGGGAGGGTCG |
| | GGACAAAGTTTAGGGCGTCGATAAGCGTCAGAGCGCCGAGGTTGGGGGAGGGTTTCTCTTCCGCTC |
| | TTTCGCGGGGCCTCTGGCTCCCCCAGCGCAGCTGGAGTGGGGGACGGGTAGGCTCGTCCCAAAGGC |
| | GCGGCGCTGAGGTTTGTGAACGCGTGGAGGGGCGCTTGGGGTCTGGGGGAGGCGTCGCCCGGGTAA |
| | GCCTGTCTGCTGCGGCTCTGCTTCCCTTAGACTGGAGAGCTGTGGACTTCGTCTAGGCGCCCGCTA |
| | AGTTCGCATGTCCTAGCACCTCTGGGTCTATGTGGGGCCACACCGTGGGGAGGAAACAGCACGCGA |
| | CGTTTGTAGAATGCTTGGCTGTGATACAAAGCGGTTTCGAATAATTAACTTATTTGTTCCCATCAC |
| | ATGTCACTTTTAAAAAATTATAAGAACTACCCGTTATTGACATCTTTCTGTGTGCCAAGGACTTTA |
| | TGTGCTTTGCGTCATTTAATTTTGAAAACAGTTATCTTCCGCCATAGATAACTACTATGGTTATCT |
| | TCTGCCTCTCACAGATGAAGAAACTAAGGCACCGAGATTTTAAGAAACTTAATTACACAGGGGATA |
| | AATGGCAGCAATCGAGATTGAAGTCAAGCCTAACCAGGGCTTTTGCGGGAGCGCATGCCTTTTGGC |
| | TGTAATTCGTGCATTTTTTTTTAAGAAAAACGCCTGCCTTCTGCGTGAGATTCTCCAGAGCAAACT |
| | GGGCGGCATGGGCCCTGTGGTCTTTTCGTACAGAGGGCTTCCTCTTTGGCTCTTTGCCTGGTTGTT |
| | TCCAAGATGTACTGTGCCTCTTACTTTCGGTTTTGAAAACATGAGGGGGTTGGGCGTGGTAGCTTA |
| | CGCCTGTAATCCCAGCACTTAGGGAGGCCGAGGCGGGAGGATGGCTTGAGGTCCGTAGTTGAGACC |
| | AGCCTGGCCAACATGGTGAAGCCTGGTCTCTACAAAAAATAATAACAAAAATTAGCCGGGTGTGGT |
| | GGCTCGTGCCTGTGGTCCCAGCTGCTCCGGTGGCTGAGGCGGGAGGATCTCTTGAGCTTAGGCTTT |
| | TGAGCTATCATGGCGCCAGTGCACTCCAGCGTGGGCAACAGAGCGAGACCCTGTCTCTCAAAAAAG |
| | AAAAAAAAAAAAAAAGAAAGAGAAAAGAAAAGAAAGAAAGAAGTGAAGGTTTGTCAGTCAGGGGAG |
| | CTGTAAAACCATTAATAAAGATAATCCAAGATGGTTACCAAGACTGTTGAGGACGCCAGAGATCTT |
| | GAGCACTTTCTAAGTACCTGGCAATACACTAAGCGCGCTCACCTTTTCCTCTGGCAAAACATGATC |
| | GAAAGCAGAATGTTTTGATCATGAGAAAATTGCATTTAATTTGAATACAATTTATTTACAACATAA |
| | AGGATAATGTATATATCACCACCATTACTGGTATTTGCTGGTTATGTTAGATGTCATTTTAAAAAA |
| | TAACAATCTGATATTTAAAAAAAAATCTTATTTTGAAAATTTCCAAAGTAATACATGCCATGCATA |
| | GACCATTTCTGGAAGATACCACAAGAAACATGTAATGATGATTGCCTCTGAAGGTCTATTTTCCTC |
| | CTCTGACCTGTGTGTGGGTTTTGTTTTTGTTTTACTGTGGGCATAAATTAATTTTTCAGTTAAGTT |
| | TTGGAAGCTTAAATAACTCTCCAAAAGTCATAAAGCCAGTAACTGGTTGAGCCCAAATTCAAACCC |
| | AGCCTGTCTGATACTTGTCCTCTTCTTAGAAAAGATTACAGTGATGCTCTCACAAAATCTTGCCGC |
| | CTTCCCTCAAACAGAGAGTTCCAGGCAGGATGAATCTGTGCTCTGATCCCTGAGGCATTTAATATG |
| | TTCTTATTATTAGAAGCTCAGATGCAAAGAGCTCTCTTAGCTTTTAATGTTATGAAAAAAATCAGG |
| | TCTTCATTAGATTCCCCAATCCACCTCTTGATGGGGCTAGTAGCCTTTCCTTAATGATAGGGTGTT |
| | TCTAGAGAGATATATCTGGTCAAGGTGGCCTGGTACTCCTCCTTCTCCCCACAGCCTCCCAGACAA |
| | GGAGGAGTAGCTGCCTTTTAGTGATCATGTACCCTGAATATAAGTGTATTTAAAAGAATTTTATAC |
| | ACATATATTTAGTGTCAATCTGTATATTTAGTAGCACTAACACTTCTCTTCATTTTCAATGAAAAA |
| | TATAGAGTTTATAATATTTTCTTCCCACTTCCCCATGGATGGTCTAGTCATGCCTCTCATTTTGGA |
| | AAGTACTGTTTCTGAAACATTAGGCAATATATTCCCAACCTGGCTAGTTTACAGCAATCACCTGTG |
| | GATGCTAATTAAAACGCAAATCCCACTGTCACATGCATTACTCCATTTGATCATAATGGAAAGTAT |
| | GTTCTGTCCCATTTGCCATAGTCCTCACCTATCCCTGTTGTATTTTATCGGGTCCAACTCAACCAT |
| | TTAAGGTATTTGCCAGCTCTTGTATGCATTTAGGTTTTGTTTCTTTGTTTTTTAGCTCATGAAATT |
| | AGGTACAAAGTCAGAGAGGGGTCTGGCATATAAAACCTCAGCAGAAATAAAGAGGTTTTGTTGTTT |
| | GGTAAGAACATACCTTGGGTTGGTTGGGCACGGTGGCTCGTGCCTGTAATCCCAACACTTTGGGAG |
| | GCCAAGGCAGGCTGATCACTTGAAGTTGGGAGTTCAAGACCAGCCTGGCCAACATGGTGAAATCCC |
| | GTCTCTACTGAAAATACAAAAATTAACCAGGCATGGTGGTGTGTGCCTGTAGTCCCAGGAATCACT |
| | TGAACCCAGGAGGCGGAGGTTGCAGTGAGCTGAGATCTCACCACTGCACACTGCACTCCAGCCTGG |
| | GCAATGGAATGAGATTCCATCCCAAAAAATAAAAAAATAAAAAAATAAAGAACATACCTTGGGTTG |
| | ATCCACTTAGGAACCTCAGATAATAACATCTGCCACGTATAGAGCAATTGCTATGTCCCAGGCACT |
| | CTACTAGACACTTCATACAGTTTAGAAAATCAGATGGGTGTAGATCAAGGCAGGAGCAGGAACCAA |
| | AAAGAAAGGCATAAACATAAGAAAAAAAATGGAAGGGGTGGAAACAGAGTACAATAACATGAGTAA |
| | TTTGATGGGGGCTATTATGAACTGAGAAATGAACTTTGAAAAGTATCTTGGGGCCAAATCATGTAG |
| | ACTCTTGAGTGATGTGTTAAGGAATGCTATGAGTGCTGAGAGGGCATCAGAAGTCCTTGAGAGCCT |
| | CCAGAGAAAGGCTCTTAAAAATGCAGCGCAATCTCCAGTGACAGAAGATACTGCTAGAAATCTGCT |
| | AGAAAAAAAACAAAAAAGGCATGTATAGAGGAATTATGAGGGAAAGATACCAAGTCACGGTTTATT |
| | CTTCAAAATGGAGGTGGCTTGTTGGGAAGGTGGAAGCTCATTTGGCCAGAGTGGAAATGGAATTGG |
| | GAGAAATCGATGACCAAATGTAAACACTTGGTGCCTGATATAGCTTGACACCAAGTTAGCCCCAAG |
| | TGAAATACCCTGGCAATATTAATGTGTCTTTTCCCGATATTCCTCAG |
| B2M intron 2 | GTAAGTCTTACATTCTTTTGTAAGCTGCTGAAAGTTGTGTATGAGTAGTCATATCATAAAGCTGCT |
| (SEQ ID NO: 1220) | TTGATATAAAAAAGGTCTATGGCCATACTACCCTGAATGAGTCCCATCCCATCTGATATAAACAAT |
| | CTGCATATTGGGATTGTCAGGGAATGTTCTTAAAGATCAGATTAGTGGCACCTGCTGAGATACTGA |
| | TGCACAGCATGGTTTCTGAACCAGTAGTTTCCCTGCAGTTGAGCAGGGAGCAGCAGCAGCACTTGC |
| | ACAAATACATATACACTCTTAACACTTCTTACCTACTGGCTTCCTCTAGCTTTTGTGGCAGCTTCA |
| | GGTATATTTAGCACTGAACGAACATCTCAAGAAGGTATAGGCCTTTGTTTGTAAGTCCTGCTGTCC |
| | TAGCATCCTATAATCCTGGACTTCTCCAGTACTTTCTGGCTGGATTGGTATCTGAGGCTAGTAGGA |
| | AGGGCTTGTTCCTGCTGGGTAGCTCTAAACAATGTATTCATGGGTAGGAACAGCAGCCTATTCTGC |
| | CAGCCTTATTTCTAACCATTTTAGACATTTGTTAGTACATGGTATTTTAAAAGTAAAACTTAATGT |
| | CTTCCTTTTTTTTCTCCACTGTCTTTTTCATAG |
| B2M intron 3 | GTAAGTTTTTGACCTTGAGAAAATGTTTTTGTTTCACTGTCCTGAGGACTATTTATAGACAGCTCT |
| (SEQ ID NO: 1221) | AACATGATAACCCTCACTATGTGGAGAACATTGACAGAGTAACATTTTAGCAGGGAAAGAAGAATC |
| | CTACAGGGTCATGTTCCCTTCTCCTGTGGAGTGGCATGAAGAAGGTGTATGGCCCCAGGTATGGCC |
| | ATATTACTGACCCTCTACAGAGAGGGCAAAGGAACTGCCAGTATGGTATTGCAGGATAAAGGCAGG |
| | TGGTTACCCACATTACCTGCAAGGCTTTGATCTTTCTTCTGCCATTTCCACATTGGACATCTCTGC |
| | TGAGGAGAGAAAATGAACCACTCTTTTCCTTTGTATAATGTTGTTTTATTCTTCAGACAGAAGAGA |
| | GGAGTTATACAGCTCTGCAGACATCCCATTCCTGTATGGGGACTGTGTTTGCCTCTTAGAGGTTCC |
| | CAGGCCACTAGAGGAGATAAAGGGAAACAGATTGTTATAACTTGATATAATGATACTATAATAGAT |
| | GTAACTACAAGGAGCTCCAGAAGCAAGAGAGAGGGAGGAACTTGGACTTCTCTGCATCTTTAGTTG |
| | GAGTCCAAAGGCTTTTCAATGAAATTCTACTGCCCAGGGTACATTGATGCTGAAACCCCATTCAAA |
| | TCTCCTGTTATATTCTAGAACAGGGAATTGATTTGGGAGAGCATCAGGAAGGTGGATGATCTGCCC |
| | AGTCACACTGTTAGTAAATTGTAGAGCCAGGACCTGAACTCTAATATAGTCATGTGTTACTTAATG |
| | ACGGGGACATGTTCTGAGAAATGCTTACACAAACCTAGGTGTTGTAGCCTACTACACGCATAGGCT |
| | ACATGGTATAGCCTATTGCTCCTAGACTACAAACCTGTACAGCCTGTTACTGTACTGAATACTGTG |
| | GGCAGTTGTAACACAATGGTAAGTATTTGTGTATCTAAACATAGAAGTTGCAGTAAAAATATGCTA |
| | TTTTAATCTTATGAGACCACTGTCATATATACAGTCCATCATTGACCAAAACATCATATCAGCATT |
| | TTTTCTTCTAAGATTTTGGGAGCACCAAAGGGATACACTAACAGGATATACTCTTTATAATGGGTT |
| | TGGAGAACTGTCTGCAGCTACTTCTTTTAAAAAGGTGATCTACACAGTAGAAATTAGACAAGTTTG |
| | GTAATGAGATCTGCAATCCAAATAAAATAAATTCATTGCTAACCTTTTTCTTTTCTTTTCAG |

What is claimed is:

1. A composition comprising an RNA guide, wherein the RNA guide comprises (i) a spacer sequence that is substantially complementary to a target sequence within a B2M gene and (ii) a direct repeat sequence; wherein the target sequence is adjacent to a protospacer adjacent motif (PAM) comprising the sequence 5′-NTTN-3′.

2. The composition of claim 1, wherein the target sequence is within exon 1, exon 2, exon 3, exon 4, intron 1, intron 2, or intron 3 of the B2M gene.

3. The composition of claim 1 or 2, wherein the B2M gene comprises the sequence of SEQ ID NO: 773, the reverse complement of SEQ ID NO: 773, a variant of SEQ ID NO: 773, or the reverse complement of a variant of SEQ ID NO: 773.

4. The composition of any one of claims 1 to 3, wherein the spacer sequence comprises:
a. nucleotide 1 through nucleotide 16 of a sequence that is at least 90% identical to a sequence of any one of SEQ ID NOs: 391-770 or 1019-1218;
b. nucleotide 1 through nucleotide 17 of a sequence that is at least 90% identical to a sequence of any one of SEQ ID NOs: 391-770 or 1019-1218;
c. nucleotide 1 through nucleotide 18 of a sequence that is at least 90% identical to a sequence of any one of SEQ ID NOs: 391-770 or 1019-1218;
d. nucleotide 1 through nucleotide 19 of a sequence that is at least 90% identical to a sequence of any one of SEQ ID NOs: 391-770 or 1019-1218;
e. nucleotide 1 through nucleotide 20 of a sequence that is at least 90% identical to a sequence of any one of SEQ ID NOs: 391-770 or 1019-1218;
f. nucleotide 1 through nucleotide 21 of a sequence that is at least 90% identical to a sequence of any one of SEQ ID NOs: 391-770;
g. nucleotide 1 through nucleotide 22 of a sequence that is at least 90% identical to a sequence of any one of SEQ ID NOs: 391-552 and 554-770;
h. nucleotide 1 through nucleotide 23 of a sequence that is at least 90% identical to a sequence of any one of SEQ ID NOs: 391-552 and 555-770;
i. nucleotide 1 through nucleotide 24 of a sequence that is at least 90% identical to a sequence of any one of SEQ ID NOs: 391-552 and 556-770;
j. nucleotide 1 through nucleotide 25 of a sequence that is at least 90% identical to a sequence of any one of SEQ ID NOs: 391-552 and 556-770;
k. nucleotide 1 through nucleotide 26 of a sequence that is at least 90% identical to a sequence of any one of SEQ ID NOs: 391-552 and 556-770;
l. nucleotide 1 through nucleotide 27 of a sequence that is at least 90% identical to a sequence of any one of SEQ ID NOs: 391-552 and 556-770;
m. nucleotide 1 through nucleotide 28 of a sequence that is at least 90% identical to a sequence of any one of SEQ ID NOs: 391-552 and 556-770;
n. nucleotide 1 through nucleotide 29 of a sequence that is at least 90% identical to a sequence of any one of SEQ ID NOs: 391-552 and 556-770; or
o. nucleotide 1 through nucleotide 30 of a sequence that is at least 90% identical to a sequence of any one of SEQ ID NOs: 391-540, 542-552, and 556-770.

5. The composition of any one of claims 1 to 4, wherein the spacer sequence comprises:
a. nucleotide 1 through nucleotide 16 of any one of SEQ ID NOs: 391-770 or 1019-1218;
b. nucleotide 1 through nucleotide 17 of any one of SEQ ID NOs: 391-770 or 1019-1218;
c. nucleotide 1 through nucleotide 18 of any one of SEQ ID NOs: 391-770 or 1019-1218;
d. nucleotide 1 through nucleotide 19 of any one of SEQ ID NOs: 391-770 or 1019-1218;
e. nucleotide 1 through nucleotide 20 of any one of SEQ ID NOs: 391-770 or 1019-1218;
f. nucleotide 1 through nucleotide 21 of any one of SEQ ID NOs: 391-770;
g. nucleotide 1 through nucleotide 22 of any one of SEQ ID NOs: 391-552 and 554-770;
h. nucleotide 1 through nucleotide 23 of any one of SEQ ID NOs: 391-552 and 555-770;
i. nucleotide 1 through nucleotide 24 of any one of SEQ ID NOs: 391-552 and 556-770;
j. nucleotide 1 through nucleotide 25 of any one of SEQ ID NOs: 391-552 and 556-770;
k. nucleotide 1 through nucleotide 26 of any one of SEQ ID NOs: 391-552 and 556-770;
l. nucleotide 1 through nucleotide 27 of any one of SEQ ID NOs: 391-552 and 556-770;
m. nucleotide 1 through nucleotide 28 of any one of SEQ ID NOs: 391-552 and 556-770;
n. nucleotide 1 through nucleotide 29 of any one of SEQ ID NOs: 391-552 and 556-770; or
o. nucleotide 1 through nucleotide 30 of any one of SEQ ID NOs: 391-540, 542-552, and 556-770.

6. The composition of any one of claims 1 to 5, wherein the direct repeat comprises:
a. nucleotide 1 through nucleotide 36 of a sequence that is at least 90% identical to a sequence of any one of SEQ ID NOs: 1-8;
b. nucleotide 2 through nucleotide 36 of a sequence that is at least 90% identical to a sequence of any one of SEQ ID NOs: 1-8;
c. nucleotide 3 through nucleotide 36 of a sequence that is at least 90% identical to a sequence of any one of SEQ ID NOs: 1-8;
d. nucleotide 4 through nucleotide 36 of a sequence that is at least 90% identical to a sequence of any one of SEQ ID NOs: 1-8;
e. nucleotide 5 through nucleotide 36 of a sequence that is at least 90% identical to a sequence of any one of SEQ ID NOs: 1-8;
f. nucleotide 6 through nucleotide 36 of a sequence that is at least 90% identical to a sequence of any one of SEQ ID NOs: 1-8;
g. nucleotide 7 through nucleotide 36 of a sequence that is at least 90% identical to a sequence of any one of SEQ ID NOs: 1-8;
h. nucleotide 8 through nucleotide 36 of a sequence that is at least 90% identical to a sequence of any one of SEQ ID NOs: 1-8;
i. nucleotide 9 through nucleotide 36 of a sequence that is at least 90% identical to a sequence of any one of SEQ ID NOs: 1-8;
j. nucleotide 10 through nucleotide 36 of a sequence that is at least 90% identical to a sequence of any one of SEQ ID NOs: 1-8;
k. nucleotide 11 through nucleotide 36 of a sequence that is at least 90% identical to a sequence of any one of SEQ ID NOs: 1-8;
l. nucleotide 12 through nucleotide 36 of a sequence that is at least 90% identical to a sequence of any one of SEQ ID NOs: 1-8;
m. nucleotide 13 through nucleotide 36 of a sequence that is at least 90% identical to a sequence of any one of SEQ ID NOs: 1-8;
n. nucleotide 14 through nucleotide 36 of a sequence that is at least 90% identical to a sequence of any one of SEQ ID NOs: 1-8;
o. nucleotide 1 through nucleotide 34 of a sequence that is at least 90% identical to a sequence of SEQ ID NO: 9;
p. nucleotide 2 through nucleotide 34 of a sequence that is at least 90% identical to a sequence of SEQ ID NO: 9;
q. nucleotide 3 through nucleotide 34 of a sequence that is at least 90% identical to a sequence of SEQ ID NO: 9;
r. nucleotide 4 through nucleotide 34 of a sequence that is at least 90% identical to a sequence of SEQ ID NO: 9;
s. nucleotide 5 through nucleotide 34 of a sequence that is at least 90% identical to a sequence of SEQ ID NO:9;
t. nucleotide 6 through nucleotide 34 of a sequence that is at least 90% identical to a sequence of SEQ ID NO: 9;
u. nucleotide 7 through nucleotide 34 of a sequence that is at least 90% identical to a sequence of SEQ ID NO: 9;
v. nucleotide 8 through nucleotide 34 of a sequence that is at least 90% identical to a sequence of SEQ ID NO: 9;
w. nucleotide 9 through nucleotide 34 of a sequence that is at least 90% identical to a sequence of SEQ ID NO: 9;
x. nucleotide 10 through nucleotide 34 of a sequence that is at least 90% identical to a sequence of SEQ ID NO: 9;
y. nucleotide 11 through nucleotide 34 of a sequence that is at least 90% identical to a sequence of SEQ ID NO: 9;
z. nucleotide 12 through nucleotide 34 of a sequence that is at least 90% identical to a sequence of SEQ ID NO: 9; or
aa. a sequence that is at least 90% identical to a sequence of SEQ ID NO: 10 or a portion thereof.

7. The composition of any one of claims 1 to 6, wherein the direct repeat comprises:
a. nucleotide 1 through nucleotide 36 of any one of SEQ ID NOs: 1-8;
b. nucleotide 2 through nucleotide 36 of any one of SEQ ID NOs: 1-8;
c. nucleotide 3 through nucleotide 36 of any one of SEQ ID NOs: 1-8;
d. nucleotide 4 through nucleotide 36 of any one of SEQ ID NOs: 1-8;
e. nucleotide 5 through nucleotide 36 of any one of SEQ ID NOs: 1-8;
f. nucleotide 6 through nucleotide 36 of any one of SEQ ID NOs: 1-8;
g. nucleotide 7 through nucleotide 36 of any one of SEQ ID NOs: 1-8;
h. nucleotide 8 through nucleotide 36 of any one of SEQ ID NOs: 1-8;
i. nucleotide 9 through nucleotide 36 of any one of SEQ ID NOs: 1-8;
j. nucleotide 10 through nucleotide 36 of any one of SEQ ID NOs: 1-8;
k. nucleotide 11 through nucleotide 36 of any one of SEQ ID NOs: 1-8;
l. nucleotide 12 through nucleotide 36 of any one of SEQ ID NOs: 1-8;
m. nucleotide 13 through nucleotide 36 of any one of SEQ ID NOs: 1-8;
n. nucleotide 14 through nucleotide 36 of any one of SEQ ID NOs: 1-8;
o. nucleotide 1 through nucleotide 34 of SEQ ID NO: 9;
p. nucleotide 2 through nucleotide 34 of SEQ ID NO: 9;
q. nucleotide 3 through nucleotide 34 of SEQ ID NO: 9;
r. nucleotide 4 through nucleotide 34 of SEQ ID NO: 9;
s. nucleotide 5 through nucleotide 34 of SEQ ID NO: 9;
t. nucleotide 6 through nucleotide 34 of SEQ ID NO: 9;
u. nucleotide 7 through nucleotide 34 of SEQ ID NO: 9;
v. nucleotide 8 through nucleotide 34 of SEQ ID NO: 9;
w. nucleotide 9 through nucleotide 34 of SEQ ID NO: 9;
x. nucleotide 10 through nucleotide 34 of SEQ ID NO: 9;
y. nucleotide 11 through nucleotide 34 of SEQ ID NO: 9;
z. nucleotide 12 through nucleotide 34 of SEQ ID NO: 9; or
aa. SEQ ID NO: 10 or a portion thereof.

8. The composition of any one of claims 1 to 5, wherein the direct repeat comprises:
a. nucleotide 1 through nucleotide 36 of a sequence that is at least 90% identical to a sequence of any one of SEQ ID NOs: 788-805;
b. nucleotide 2 through nucleotide 36 of a sequence that is at least 90% identical to a sequence of any one of SEQ ID NOs: 788-805;
c. nucleotide 3 through nucleotide 36 of a sequence that is at least 90% identical to a sequence of any one of SEQ ID NOs: 788-805;
d. nucleotide 4 through nucleotide 36 of a sequence that is at least 90% identical to a sequence of any one of SEQ ID NOs: 788-805;
e. nucleotide 5 through nucleotide 36 of a sequence that is at least 90% identical to a sequence of any one of SEQ ID NOs: 788-805;
f. nucleotide 6 through nucleotide 36 of a sequence that is at least 90% identical to a sequence of any one of SEQ ID NOs: 788-805;
g. nucleotide 7 through nucleotide 36 of a sequence that is at least 90% identical to a sequence of any one of SEQ ID NOs: 788-805;
h. nucleotide 8 through nucleotide 36 of a sequence that is at least 90% identical to a sequence of any one of SEQ ID NOs: 788-805;
i. nucleotide 9 through nucleotide 36 of a sequence that is at least 90% identical to a sequence of any one of SEQ ID NOs: 788-805;
j. nucleotide 10 through nucleotide 36 of a sequence that is at least 90% identical to a sequence of any one of SEQ ID NOs: 788-805;
k. nucleotide 11 through nucleotide 36 of a sequence that is at least 90% identical to a sequence of any one of SEQ ID NOs: 788-805;
l. nucleotide 12 through nucleotide 36 of a sequence that is at least 90% identical to a sequence of any one of SEQ ID NOs: 788-805;
m. nucleotide 13 through nucleotide 36 of a sequence that is at least 90% identical to a sequence of any one of SEQ ID NOs: 788-805;
n. nucleotide 14 through nucleotide 36 of a sequence that is at least 90% identical to a sequence of any one of SEQ ID NOs: 788-805; or
o. a sequence that is at least 90% identical to a sequence of SEQ ID NO: 806 or a portion thereof.

9. The composition of any one of claims 1 to 5 or 8, wherein the direct repeat comprises:
a. nucleotide 1 through nucleotide 36 of any one of SEQ ID NOs: 788-805;
b. nucleotide 2 through nucleotide 36 of any one of SEQ ID NOs: 788-805;
c. nucleotide 3 through nucleotide 36 of any one of SEQ ID NOs: 788-805;
d. nucleotide 4 through nucleotide 36 of any one of SEQ ID NOs: 788-805;
e. nucleotide 5 through nucleotide 36 of any one of SEQ ID NOs: 788-805;
f. nucleotide 6 through nucleotide 36 of any one of SEQ ID NOs: 788-805;
g. nucleotide 7 through nucleotide 36 of any one of SEQ ID NOs: 788-805;
h. nucleotide 8 through nucleotide 36 of any one of SEQ ID NOs: 788-805;
i. nucleotide 9 through nucleotide 36 of any one of SEQ ID NOs: 788-805;
j. nucleotide 10 through nucleotide 36 of any one of SEQ ID NOs: 788-805;
k. nucleotide 11 through nucleotide 36 of any one of SEQ ID NOs: 788-805;
l. nucleotide 12 through nucleotide 36 of any one of SEQ ID NOs: 788-805;
m. nucleotide 13 through nucleotide 36 of any one of SEQ ID NOs: 788-805;
n. nucleotide 14 through nucleotide 36 of any one of SEQ ID NOs: 788-805; or
o. SEQ ID NO: 806 or a portion thereof.

10. The composition of any one of claims 1 to 5, wherein the direct repeat comprises:
a. nucleotide 1 through nucleotide 36 of a sequence that is at least 90% identical to SEQ ID NO: 807;
b. nucleotide 2 through nucleotide 36 of a sequence that is at least 90% identical to SEQ ID NO: 807;
c. nucleotide 3 through nucleotide 36 of a sequence that is at least 90% identical to SEQ ID NO: 807;
d. nucleotide 4 through nucleotide 36 of a sequence that is at least 90% identical to SEQ ID NO: 807;
e. nucleotide 5 through nucleotide 36 of a sequence that is at least 90% identical to SEQ ID NO: 807;
f. nucleotide 6 through nucleotide 36 of a sequence that is at least 90% identical to SEQ ID NO: 807;
g. nucleotide 7 through nucleotide 36 of a sequence that is at least 90% identical to SEQ ID NO: 807;
h. nucleotide 8 through nucleotide 36 of a sequence that is at least 90% identical to SEQ ID NO: 807;
i. nucleotide 9 through nucleotide 36 of a sequence that is at least 90% identical to SEQ ID NO: 807;
j. nucleotide 10 through nucleotide 36 of a sequence that is at least 90% identical to SEQ ID NO: 807;
k. nucleotide 11 through nucleotide 36 of a sequence that is at least 90% identical to SEQ ID NO: 807;
l. nucleotide 12 through nucleotide 36 of a sequence that is at least 90% identical to SEQ ID NO: 807;
m. nucleotide 13 through nucleotide 36 of a sequence that is at least 90% identical to SEQ ID NO: 807;
n. nucleotide 14 through nucleotide 36 of a sequence that is at least 90% identical to SEQ ID NO: 807; or
o. a sequence that is at least 90% identical to a sequence of SEQ ID NO: 808 or SEQ ID NO: 809 or a portion thereof.

11. The composition of any one of claims 1 to 5 or 10, wherein the direct repeat comprises:
a. nucleotide 1 through nucleotide 36 of SEQ ID NO: 807;
b. nucleotide 2 through nucleotide 36 of SEQ ID NO: 807;
c. nucleotide 3 through nucleotide 36 of SEQ ID NO: 807;
d. nucleotide 4 through nucleotide 36 of SEQ ID NO: 807;
e. nucleotide 5 through nucleotide 36 of SEQ ID NO: 807;
f. nucleotide 6 through nucleotide 36 of SEQ ID NO: 807;
g. nucleotide 7 through nucleotide 36 of SEQ ID NO: 807;
h. nucleotide 8 through nucleotide 36 of SEQ ID NO: 807;
i. nucleotide 9 through nucleotide 36 of SEQ ID NO: 807;
j. nucleotide 10 through nucleotide 36 of SEQ ID NO: 807;
k. nucleotide 11 through nucleotide 36 of SEQ ID NO: 807;
l. nucleotide 12 through nucleotide 36 of SEQ ID NO: 807;
m. nucleotide 13 through nucleotide 36 of SEQ ID NO: 807;
n. nucleotide 14 through nucleotide 36 of SEQ ID NO: 807; or
o. SEQ ID NO: 808 or SEQ ID NO: 809 or a portion thereof.

12. The composition of any one of claims 1 to 5, wherein the direct repeat comprises:
a. nucleotide 1 through nucleotide 36 of a sequence that is at least 90% identical to a sequence of SEQ ID NO: 810 or SEQ ID NO: 811;
b. nucleotide 2 through nucleotide 36 of a sequence that is at least 90% identical to a sequence of SEQ ID NO: 810 or SEQ ID NO: 811;
c. nucleotide 3 through nucleotide 36 of a sequence that is at least 90% identical to a sequence of SEQ ID NO: 810 or SEQ ID NO: 811;
d. nucleotide 4 through nucleotide 36 of a sequence that is at least 90% identical to a sequence of SEQ ID NO: 810 or SEQ ID NO: 811;
e. nucleotide 5 through nucleotide 36 of a sequence that is at least 90% identical to a sequence of SEQ ID NO: 810 or SEQ ID NO: 811;
f. nucleotide 6 through nucleotide 36 of a sequence that is at least 90% identical to a sequence of SEQ ID NO: 810 or SEQ ID NO: 811;
g. nucleotide 7 through nucleotide 36 of a sequence that is at least 90% identical to a sequence of SEQ ID NO: 810 or SEQ ID NO: 811;
h. nucleotide 8 through nucleotide 36 of a sequence that is at least 90% identical to a sequence of SEQ ID NO: 810 or SEQ ID NO: 811;
i. nucleotide 9 through nucleotide 36 of a sequence that is at least 90% identical to a sequence of SEQ ID NO: 810 or SEQ ID NO: 811;
j. nucleotide 10 through nucleotide 36 of a sequence that is at least 90% identical to a sequence of SEQ ID NO: 810 or SEQ ID NO: 811;
k. nucleotide 11 through nucleotide 36 of a sequence that is at least 90% identical to a sequence of SEQ ID NO: 810 or SEQ ID NO: 811;
l. nucleotide 12 through nucleotide 36 of a sequence that is at least 90% identical to a sequence of SEQ ID NO: 810 or SEQ ID NO: 811;
m. nucleotide 13 through nucleotide 36 of a sequence that is at least 90% identical to a sequence of SEQ ID NO: 810 or SEQ ID NO: 811;
n. nucleotide 14 through nucleotide 36 of a sequence that is at least 90% identical to a sequence of SEQ ID NO: 810 or SEQ ID NO: 811;
o. nucleotide 15 through nucleotide 36 of a sequence that is at least 90% identical to a sequence of SEQ ID NO: 810 or SEQ ID NO: 811; or
p. a sequence that is at least 90% identical to a sequence of SEQ ID NO: 812 or a portion thereof.

13. The composition of any one of claims 1 to 5 or 12, wherein the direct repeat comprises:
a. nucleotide 1 through nucleotide 36 of SEQ ID NO: 810 or SEQ ID NO: 811;
b. nucleotide 2 through nucleotide 36 of SEQ ID NO: 810 or SEQ ID NO: 811;
c. nucleotide 3 through nucleotide 36 of SEQ ID NO: 810 or SEQ ID NO: 811;
d. nucleotide 4 through nucleotide 36 of SEQ ID NO: 810 or SEQ ID NO: 811;
e. nucleotide 5 through nucleotide 36 of SEQ ID NO: 810 or SEQ ID NO: 811;
f. nucleotide 6 through nucleotide 36 of SEQ ID NO: 810 or SEQ ID NO: 811;
g. nucleotide 7 through nucleotide 36 of SEQ ID NO: 810 or SEQ ID NO: 811;
h. nucleotide 8 through nucleotide 36 of SEQ ID NO: 810 or SEQ ID NO: 811;
i. nucleotide 9 through nucleotide 36 of SEQ ID NO: 810 or SEQ ID NO: 811;
j. nucleotide 10 through nucleotide 36 of SEQ ID NO: 810 or SEQ ID NO: 811;
k. nucleotide 11 through nucleotide 36 of SEQ ID NO: 810 or SEQ ID NO: 811;
l. nucleotide 12 through nucleotide 36 of SEQ ID NO: 810 or SEQ ID NO: 811;
m. nucleotide 13 through nucleotide 36 of SEQ ID NO: 810 or SEQ ID NO: 811;
n. nucleotide 14 through nucleotide 36 of SEQ ID NO: 810 or SEQ ID NO: 811;
o. nucleotide 15 through nucleotide 36 of SEQ ID NO: 810 or SEQ ID NO: 811; or
p. SEQ ID NO: 812 or a portion thereof.

14. The composition of any one of claims 1 to 13, wherein the spacer sequence is substantially complementary to the complement of a sequence of any one of SEQ ID NOs: 11-390 or 819-1018.

15. The composition of claim 1, wherein the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

16. The composition of claim 1 or 15, wherein the target sequence is immediately adjacent to the PAM sequence.

17. The composition of any one of claims 1 to 16, wherein the composition further comprises a Cas12i polypeptide.

18. The composition of claim 17, wherein the Cas12i polypeptide is:
a. a Cas12i2 polypeptide comprising a sequence that is at least 90% identical to the sequence of SEQ ID NO: 772, SEQ ID NO: 782, SEQ ID NO: 783, SEQ ID NO: 784, SEQ ID NO: 785, or SEQ ID NO: 786;
b. a Cas12i4 polypeptide comprising a sequence that is at least 90% identical to the sequence of SEQ ID NO: 814, SEQ ID NO: 815, or SEQ ID NO: 816;
c. a Cas12i1 polypeptide comprising a sequence that is at least 90% identical to the sequence of SEQ ID NO: 817; or
d. a Cas12i3 polypeptide comprising a sequence that is at least 90% identical to the sequence of SEQ ID NO: 818.

19. The composition of claim 18, wherein the Cas12i polypeptide is:
a. a Cas12i2 polypeptide comprising a sequence of SEQ ID NO: 772, SEQ ID NO: 782, SEQ ID NO: 783, SEQ ID NO: 784, SEQ ID NO: 785, or SEQ ID NO: 786;
b. a Cas12i4 polypeptide comprising a sequence of SEQ ID NO: 814, SEQ ID NO: 815, or SEQ ID NO: 816;
c. a Cas12i1 polypeptide comprising a sequence of SEQ ID NO: 817; or
d. a Cas12i3 polypeptide comprising a sequence of SEQ ID NO: 818.

20. The composition of any one of claims 17 to 19, wherein the RNA guide and the Cas12i polypeptide form a ribonucleoprotein complex.

21. The composition of claim 20, wherein the ribonucleoprotein complex binds a target nucleic acid.

22. The composition of claim 20 or 21, wherein the composition is present within a cell.

23. The composition of any one of claims 17 to 22, wherein the RNA guide and the Cas12i polypeptide are encoded in a vector, e.g., expression vector.

24. The composition of claim 23, wherein the RNA guide and the Cas12i polypeptide are encoded in a single vector or the RNA guide is encoded in a first vector and the Cas12i polypeptide is encoded in a second vector.

25. An RNA guide comprising (i) a spacer sequence that is substantially complementary to a target sequence within a B2M gene and (ii) a direct repeat sequence.

26. The RNA guide of claim 25, wherein the target sequence is within exon 1, exon 2, exon 3, exon 4, intron 1, intron 2, or intron 3 of the B2M gene.

27. The RNA guide of claim 25 or 26, wherein the B2M gene comprises the sequence of SEQ ID NO: 773, the reverse complement of SEQ ID NO: 773, a variant of SEQ ID NO: 773, or the reverse complement of a variant of SEQ ID NO: 773.

28. The RNA guide of any one of claims 25 to 27, wherein the spacer sequence comprises:
a. nucleotide 1 through nucleotide 16 of a sequence that is at least 90% identical to a sequence of any one of SEQ ID NOs: 391-770 or 1019-1218;
b. nucleotide 1 through nucleotide 17 of a sequence that is at least 90% identical to a sequence of any one of SEQ ID NOs: 391-770 or 1019-1218;
c. nucleotide 1 through nucleotide 18 of a sequence that is at least 90% identical to a sequence of any one of SEQ ID NOs: 391-770 or 1019-1218;
d. nucleotide 1 through nucleotide 19 of a sequence that is at least 90% identical to a sequence of any one of SEQ ID NOs: 391-770 or 1019-1218;
e. nucleotide 1 through nucleotide 20 of a sequence that is at least 90% identical to a sequence of any one of SEQ ID NOs: 391-770 or 1019-1218;
f. nucleotide 1 through nucleotide 21 of a sequence that is at least 90% identical to a sequence of any one of SEQ ID NOs: 391-770;
g. nucleotide 1 through nucleotide 22 of a sequence that is at least 90% identical to a sequence of any one of SEQ ID NOs: 391-552 and 554-770;
h. nucleotide 1 through nucleotide 23 of a sequence that is at least 90% identical to a sequence of any one of SEQ ID NOs: 391-552 and 555-770;
i. nucleotide 1 through nucleotide 24 of a sequence that is at least 90% identical to a sequence of any one of SEQ ID NOs: 391-552 and 556-770;
j. nucleotide 1 through nucleotide 25 of a sequence that is at least 90% identical to a sequence of any one of SEQ ID NOs: 391-552 and 556-770;
k. nucleotide 1 through nucleotide 26 of a sequence that is at least 90% identical to a sequence of any one of SEQ ID NOs: 391-552 and 556-770;
l. nucleotide 1 through nucleotide 27 of a sequence that is at least 90% identical to a sequence of any one of SEQ ID NOs: 391-552 and 556-770;
m. nucleotide 1 through nucleotide 28 of a sequence that is at least 90% identical to a sequence of any one of SEQ ID NOs: 391-552 and 556-770;
n. nucleotide 1 through nucleotide 29 of a sequence that is at least 90% identical to a sequence of any one of SEQ ID NOs: 391-552 and 556-770; or
o. nucleotide 1 through nucleotide 30 of a sequence that is at least 90% identical to a sequence of any one of SEQ ID NOs: 391-540, 542-552, and 556-770.

29. The RNA guide of any one of claims 25 to 28, wherein the spacer sequence comprises:
a. nucleotide 1 through nucleotide 16 of any one of SEQ ID NOs: 391-770 or 1019-1218;
b. nucleotide 1 through nucleotide 17 of any one of SEQ ID NOs: 391-770 or 1019-1218;
c. nucleotide 1 through nucleotide 18 of any one of SEQ ID NOs: 391-770 or 1019-1218;
d. nucleotide 1 through nucleotide 19 of any one of SEQ ID NOs: 391-770 or 1019-1218;
e. nucleotide 1 through nucleotide 20 of any one of SEQ ID NOs: 391-770 or 1019-1218;
f. nucleotide 1 through nucleotide 21 of any one of SEQ ID NOs: 391-770;
g. nucleotide 1 through nucleotide 22 of any one of SEQ ID NOs: 391-552 and 554-770;
h. nucleotide 1 through nucleotide 23 of any one of SEQ ID NOs: 391-552 and 555-770;
i. nucleotide 1 through nucleotide 24 of any one of SEQ ID NOs: 391-552 and 556-770;
j. nucleotide 1 through nucleotide 25 of any one of SEQ ID NOs: 391-552 and 556-770;
k. nucleotide 1 through nucleotide 26 of any one of SEQ ID NOs: 391-552 and 556-770;
l. nucleotide 1 through nucleotide 27 of any one of SEQ ID NOs: 391-552 and 556-770;
m. nucleotide 1 through nucleotide 28 of any one of SEQ ID NOs: 391-552 and 556-770;
n. nucleotide 1 through nucleotide 29 of any one of SEQ ID NOs: 391-552 and 556-770; or
o. nucleotide 1 through nucleotide 30 of any one of SEQ ID NOs: 391-540, 542-552, and 556-770.

30. The RNA guide of any one of claims 25 to 29, wherein the direct repeat comprises:
a. nucleotide 1 through nucleotide 36 of a sequence that is at least 90% identical to a sequence of any one of SEQ ID NOs: 1-8;
b. nucleotide 2 through nucleotide 36 of a sequence that is at least 90% identical to a sequence of any one of SEQ ID NOs: 1-8;
c. nucleotide 3 through nucleotide 36 of a sequence that is at least 90% identical to a sequence of any one of SEQ ID NOs: 1-8;
d. nucleotide 4 through nucleotide 36 of a sequence that is at least 90% identical to a sequence of any one of SEQ ID NOs: 1-8;
e. nucleotide 5 through nucleotide 36 of a sequence that is at least 90% identical to a sequence of any one of SEQ ID NOs: 1-8;
f. nucleotide 6 through nucleotide 36 of a sequence that is at least 90% identical to a sequence of any one of SEQ ID NOs: 1-8;
g. nucleotide 7 through nucleotide 36 of a sequence that is at least 90% identical to a sequence of any one of SEQ ID NOs: 1-8;
h. nucleotide 8 through nucleotide 36 of a sequence that is at least 90% identical to a sequence of any one of SEQ ID NOs: 1-8;
i. nucleotide 9 through nucleotide 36 of a sequence that is at least 90% identical to a sequence of any one of SEQ ID NOs: 1-8;
j. nucleotide 10 through nucleotide 36 of a sequence that is at least 90% identical to a sequence of any one of SEQ ID NOs: 1-8;
k. nucleotide 11 through nucleotide 36 of a sequence that is at least 90% identical to a sequence of any one of SEQ ID NOs: 1-8;
l. nucleotide 12 through nucleotide 36 of a sequence that is at least 90% identical to a sequence of any one of SEQ ID NOs: 1-8;
m. nucleotide 13 through nucleotide 36 of a sequence that is at least 90% identical to a sequence of any one of SEQ ID NOs: 1-8;
n. nucleotide 14 through nucleotide 36 of a sequence that is at least 90% identical to a sequence of any one of SEQ ID NOs: 1-8;
o. nucleotide 1 through nucleotide 34 of a sequence that is at least 90% identical to a sequence of SEQ ID NO: 9;
p. nucleotide 2 through nucleotide 34 of a sequence that is at least 90% identical to a sequence of SEQ ID NO: 9;
q. nucleotide 3 through nucleotide 34 of a sequence that is at least 90% identical to a sequence of SEQ ID NO: 9;
r. nucleotide 4 through nucleotide 34 of a sequence that is at least 90% identical to a sequence of SEQ ID NO: 9;
s. nucleotide 5 through nucleotide 34 of a sequence that is at least 90% identical to a sequence of SEQ ID NO: 9;
t. nucleotide 6 through nucleotide 34 of a sequence that is at least 90% identical to a sequence of SEQ ID NO: 9;
u. nucleotide 7 through nucleotide 34 of a sequence that is at least 90% identical to a sequence of SEQ ID NO:9;
v. nucleotide 8 through nucleotide 34 of a sequence that is at least 90% identical to a sequence of SEQ ID NO: 9;
w. nucleotide 9 through nucleotide 34 of a sequence that is at least 90% identical to a sequence of SEQ ID NO: 9;
x. nucleotide 10 through nucleotide 34 of a sequence that is at least 90% identical to a sequence of SEQ ID NO: 9;
y. nucleotide 11 through nucleotide 34 of a sequence that is at least 90% identical to a sequence of SEQ ID NO: 9;
z. nucleotide 12 through nucleotide 34 of a sequence that is at least 90% identical to a sequence of SEQ ID NO: 9; or
aa. a sequence that is at least 90% identical to a sequence of SEQ ID NO: 10 or a portion thereof.

31. The RNA guide of any one of claims 25 to 30, wherein the direct repeat comprises:
a. nucleotide 1 through nucleotide 36 of any one of SEQ ID NOs: 1-8;
b. nucleotide 2 through nucleotide 36 of any one of SEQ ID NOs: 1-8;
c. nucleotide 3 through nucleotide 36 of any one of SEQ ID NOs: 1-8;
d. nucleotide 4 through nucleotide 36 of any one of SEQ ID NOs: 1-8;
e. nucleotide 5 through nucleotide 36 of any one of SEQ ID NOs: 1-8;
f. nucleotide 6 through nucleotide 36 of any one of SEQ ID NOs: 1-8;
g. nucleotide 7 through nucleotide 36 of any one of SEQ ID NOs: 1-8;
h. nucleotide 8 through nucleotide 36 of any one of SEQ ID NOs: 1-8;
i. nucleotide 9 through nucleotide 36 of any one of SEQ ID NOs: 1-8;
j. nucleotide 10 through nucleotide 36 of any one of SEQ ID NOs: 1-8;
k. nucleotide 11 through nucleotide 36 of any one of SEQ ID NOs: 1-8;
l. nucleotide 12 through nucleotide 36 of any one of SEQ ID NOs: 1-8;
m. nucleotide 13 through nucleotide 36 of any one of SEQ ID NOs: 1-8;
n. nucleotide 14 through nucleotide 36 of any one of SEQ ID NOs: 1-8;
o. nucleotide 1 through nucleotide 34 of SEQ ID NO: 9;
p. nucleotide 2 through nucleotide 34 of SEQ ID NO: 9;
q. nucleotide 3 through nucleotide 34 of SEQ ID NO: 9;
r. nucleotide 4 through nucleotide 34 of SEQ ID NO: 9;
s. nucleotide 5 through nucleotide 34 of SEQ ID NO: 9;
t. nucleotide 6 through nucleotide 34 of SEQ ID NO: 9;
u. nucleotide 7 through nucleotide 34 of SEQ ID NO: 9;
v. nucleotide 8 through nucleotide 34 of SEQ ID NO: 9;
w. nucleotide 9 through nucleotide 34 of SEQ ID NO: 9;
x. nucleotide 10 through nucleotide 34 of SEQ ID NO: 9;
y. nucleotide 11 through nucleotide 34 of SEQ ID NO: 9;
z. nucleotide 12 through nucleotide 34 of SEQ ID NO: 9; or
aa. SEQ ID NO: 10 or a portion thereof.

32. The RNA guide of any one of claims 25 to 29, wherein the direct repeat comprises:
a. nucleotide 1 through nucleotide 36 of a sequence that is at least 90% identical to a sequence of any one of SEQ ID NOs: 788-805;
b. nucleotide 2 through nucleotide 36 of a sequence that is at least 90% identical to a sequence of any one of SEQ ID NOs: 788-805;
c. nucleotide 3 through nucleotide 36 of a sequence that is at least 90% identical to a sequence of any one of SEQ ID NOs: 788-805;
d. nucleotide 4 through nucleotide 36 of a sequence that is at least 90% identical to a sequence of any one of SEQ ID NOs: 788-805;
e. nucleotide 5 through nucleotide 36 of a sequence that is at least 90% identical to a sequence of any one of SEQ ID NOs: 788-805;
f. nucleotide 6 through nucleotide 36 of a sequence that is at least 90% identical to a sequence of any one of SEQ ID NOs: 788-805;
g. nucleotide 7 through nucleotide 36 of a sequence that is at least 90% identical to a sequence of any one of SEQ ID NOs: 788-805;
h. nucleotide 8 through nucleotide 36 of a sequence that is at least 90% identical to a sequence of any one of SEQ ID NOs: 788-805;
i. nucleotide 9 through nucleotide 36 of a sequence that is at least 90% identical to a sequence of any one of SEQ ID NOs: 788-805;
j. nucleotide 10 through nucleotide 36 of a sequence that is at least 90% identical to a sequence of any one of SEQ ID NOs: 788-805;
k. nucleotide 11 through nucleotide 36 of a sequence that is at least 90% identical to a sequence of any one of SEQ ID NOs: 788-805;
l. nucleotide 12 through nucleotide 36 of a sequence that is at least 90% identical to a sequence of any one of SEQ ID NOs: 788-805;
m. nucleotide 13 through nucleotide 36 of a sequence that is at least 90% identical to a sequence of any one of SEQ ID NOs: 788-805;
n. nucleotide 14 through nucleotide 36 of a sequence that is at least 90% identical to a sequence of any one of SEQ ID NOs: 788-805; or
o. a sequence that is at least 90% identical to a sequence of SEQ ID NO: 806 or a portion thereof.

33. The RNA guide of any one of claims 25 to 29 or 32, wherein the direct repeat comprises:
a. nucleotide 1 through nucleotide 36 of any one of SEQ ID NOs: 788-805;
b. nucleotide 2 through nucleotide 36 of any one of SEQ ID NOs: 788-805;
c. nucleotide 3 through nucleotide 36 of any one of SEQ ID NOs: 788-805;
d. nucleotide 4 through nucleotide 36 of any one of SEQ ID NOs: 788-805;
e. nucleotide 5 through nucleotide 36 of any one of SEQ ID NOs: 788-805;
f. nucleotide 6 through nucleotide 36 of any one of SEQ ID NOs: 788-805;
g. nucleotide 7 through nucleotide 36 of any one of SEQ ID NOs: 788-805;
h. nucleotide 8 through nucleotide 36 of any one of SEQ ID NOs: 788-805;
i. nucleotide 9 through nucleotide 36 of any one of SEQ ID NOs: 788-805;
j. nucleotide 10 through nucleotide 36 of any one of SEQ ID NOs: 788-805;
k. nucleotide 11 through nucleotide 36 of any one of SEQ ID NOs: 788-805;
l. nucleotide 12 through nucleotide 36 of any one of SEQ ID NOs: 788-805;
m. nucleotide 13 through nucleotide 36 of any one of SEQ ID NOs: 788-805;
n. nucleotide 14 through nucleotide 36 of any one of SEQ ID NOs: 788-805; or
o. SEQ ID NO: 806 or a portion thereof.

34. The RNA guide of any one of claims 25 to 29, wherein the direct repeat comprises:
a. nucleotide 1 through nucleotide 36 of a sequence that is at least 90% identical to SEQ ID NO: 807;
b. nucleotide 2 through nucleotide 36 of a sequence that is at least 90% identical to SEQ ID NO: 807;
c. nucleotide 3 through nucleotide 36 of a sequence that is at least 90% identical to SEQ ID NO: 807;
d. nucleotide 4 through nucleotide 36 of a sequence that is at least 90% identical to SEQ ID NO: 807;
e. nucleotide 5 through nucleotide 36 of a sequence that is at least 90% identical to SEQ ID NO: 807;
f. nucleotide 6 through nucleotide 36 of a sequence that is at least 90% identical to SEQ ID NO: 807;
g. nucleotide 7 through nucleotide 36 of a sequence that is at least 90% identical to SEQ ID NO: 807;
h. nucleotide 8 through nucleotide 36 of a sequence that is at least 90% identical to SEQ ID NO: 807;
i. nucleotide 9 through nucleotide 36 of a sequence that is at least 90% identical to SEQ ID NO: 807;
j. nucleotide 10 through nucleotide 36 of a sequence that is at least 90% identical to SEQ ID NO: 807;
k. nucleotide 11 through nucleotide 36 of a sequence that is at least 90% identical to SEQ ID NO: 807;
l. nucleotide 12 through nucleotide 36 of a sequence that is at least 90% identical to SEQ ID NO: 807;
m. nucleotide 13 through nucleotide 36 of a sequence that is at least 90% identical to SEQ ID NO: 807;
n. nucleotide 14 through nucleotide 36 of a sequence that is at least 90% identical to SEQ ID NO: 807; or
o. a sequence that is at least 90% identical to a sequence of SEQ ID NO: 808 or SEQ ID NO: 809 or a portion thereof.

35. The RNA guide of any one of claims 25 to 29 or 34, wherein the direct repeat comprises:
a. nucleotide 1 through nucleotide 36 of SEQ ID NO: 807;
b. nucleotide 2 through nucleotide 36 of SEQ ID NO: 807;
c. nucleotide 3 through nucleotide 36 of SEQ ID NO: 807;
d. nucleotide 4 through nucleotide 36 of SEQ ID NO: 807;
e. nucleotide 5 through nucleotide 36 of SEQ ID NO: 807;
f. nucleotide 6 through nucleotide 36 of SEQ ID NO: 807;
g. nucleotide 7 through nucleotide 36 of SEQ ID NO: 807;
h. nucleotide 8 through nucleotide 36 of SEQ ID NO: 807;
i. nucleotide 9 through nucleotide 36 of SEQ ID NO: 807;
j. nucleotide 10 through nucleotide 36 of SEQ ID NO: 807;
k. nucleotide 11 through nucleotide 36 of SEQ ID NO: 807;
l. nucleotide 12 through nucleotide 36 of SEQ ID NO: 807;
m. nucleotide 13 through nucleotide 36 of SEQ ID NO: 807;
n. nucleotide 14 through nucleotide 36 of SEQ ID NO: 807; or
o. SEQ ID NO: 808 or SEQ ID NO: 809 or a portion thereof.

36. The RNA guide of any one of claims 25 to 29, wherein the direct repeat comprises:
a. nucleotide 1 through nucleotide 36 of a sequence that is at least 90% identical to a sequence of SEQ ID NO: 810 or SEQ ID NO: 811;
b. nucleotide 2 through nucleotide 36 of a sequence that is at least 90% identical to a sequence of SEQ ID NO: 810 or SEQ ID NO: 811;
c. nucleotide 3 through nucleotide 36 of a sequence that is at least 90% identical to a sequence of SEQ ID NO: 810 or SEQ ID NO: 811;
d. nucleotide 4 through nucleotide 36 of a sequence that is at least 90% identical to a sequence of SEQ ID NO: 810 or SEQ ID NO: 811;
e. nucleotide 5 through nucleotide 36 of a sequence that is at least 90% identical to a sequence of SEQ ID NO: 810 or SEQ ID NO: 811;
f. nucleotide 6 through nucleotide 36 of a sequence that is at least 90% identical to a sequence of SEQ ID NO: 810 or SEQ ID NO: 811;
g. nucleotide 7 through nucleotide 36 of a sequence that is at least 90% identical to a sequence of SEQ ID NO: 810 or SEQ ID NO: 811;
h. nucleotide 8 through nucleotide 36 of a sequence that is at least 90% identical to a sequence of SEQ ID NO: 810 or SEQ ID NO: 811;
i. nucleotide 9 through nucleotide 36 of a sequence that is at least 90% identical to a sequence of SEQ ID NO: 810 or SEQ ID NO: 811;
j. nucleotide 10 through nucleotide 36 of a sequence that is at least 90% identical to a sequence of SEQ ID NO: 810 or SEQ ID NO: 811;
k. nucleotide 11 through nucleotide 36 of a sequence that is at least 90% identical to a sequence of SEQ ID NO: 810 or SEQ ID NO: 811;
l. nucleotide 12 through nucleotide 36 of a sequence that is at least 90% identical to a sequence of SEQ ID NO: 810 or SEQ ID NO: 811;
m. nucleotide 13 through nucleotide 36 of a sequence that is at least 90% identical to a sequence of SEQ ID NO: 810 or SEQ ID NO: 811;
n. nucleotide 14 through nucleotide 36 of a sequence that is at least 90% identical to a sequence of SEQ ID NO: 810 or SEQ ID NO: 811;
o. nucleotide 15 through nucleotide 36 of a sequence that is at least 90% identical to a sequence of SEQ ID NO: 810 or SEQ ID NO: 811; or
p. a sequence that is at least 90% identical to a sequence of SEQ ID NO: 812 or a portion thereof.

37. The RNA guide of any one of claims 25 to 29 or 36, wherein the direct repeat comprises:
a. nucleotide 1 through nucleotide 36 of SEQ ID NO: 810 or SEQ ID NO: 811;
b. nucleotide 2 through nucleotide 36 of SEQ ID NO: 810 or SEQ ID NO: 811;
c. nucleotide 3 through nucleotide 36 of SEQ ID NO: 810 or SEQ ID NO: 811;
d. nucleotide 4 through nucleotide 36 of SEQ ID NO: 810 or SEQ ID NO: 811;
e. nucleotide 5 through nucleotide 36 of SEQ ID NO: 810 or SEQ ID NO: 811;
f. nucleotide 6 through nucleotide 36 of SEQ ID NO: 810 or SEQ ID NO: 811;
g. nucleotide 7 through nucleotide 36 of SEQ ID NO: 810 or SEQ ID NO: 811;
h. nucleotide 8 through nucleotide 36 of SEQ ID NO: 810 or SEQ ID NO: 811;
i. nucleotide 9 through nucleotide 36 of SEQ ID NO: 810 or SEQ ID NO: 811;
j. nucleotide 10 through nucleotide 36 of SEQ ID NO: 810 or SEQ ID NO: 811;
k. nucleotide 11 through nucleotide 36 of SEQ ID NO: 810 or SEQ ID NO: 811;
l. nucleotide 12 through nucleotide 36 of SEQ ID NO: 810 or SEQ ID NO: 811;
m. nucleotide 13 through nucleotide 36 of SEQ ID NO: 810 or SEQ ID NO: 811;
n. nucleotide 14 through nucleotide 36 of SEQ ID NO: 810 or SEQ ID NO: 811;
o. nucleotide 15 through nucleotide 36 of SEQ ID NO: 810 or SEQ ID NO: 811; or
p. SEQ ID NO: 812 or a portion thereof.

38. The RNA guide of any one of claims 25 to 37, wherein the spacer sequence is substantially complementary to the complement of a sequence of any one of SEQ ID NOs: 11-390 or 819-1018.

39. The RNA guide of any one of claims 25 to 38, wherein the target sequence is adjacent to a protospacer adjacent motif (PAM) comprising the sequence 5′-NTTN-3′, wherein N is any nucleotide.

40. The RNA guide of claim 39, wherein the PAM comprises the sequence 5′-ATTA-3′, 5′-ATTT-3′, 5′-ATTG-3′, 5′-ATTC-3′, 5′-TTTA-3′, 5′-TTTT-3′, 5′-TTTG-3′, 5′-TTTC-3′, 5′-GTTA-3′, 5′-GTTT-3′, 5′-GTTG-3′, 5′-GTTC-3′, 5′-CTTA-3′, 5′-CTTT-3′, 5′-CTTG-3′, or 5′-CTTC-3′.

41. The RNA guide of claim 39 or 40, wherein the target sequence is immediately adjacent to the PAM sequence.

42. A nucleic acid encoding an RNA guide of any one of claims 25 to 41.

43. A vector comprising the nucleic acid of claim 42.

44. A vector system comprising one or more vectors encoding (i) the RNA guide as defined in any of claims 1 to 41 and (ii) a Cas12i polypeptide, optionally wherein the vector system comprises a first vector encoding the RNA guide and a second vector encoding the Cas12i polypeptide.

45. A cell comprising the composition of any one of claims 1 to 24, the RNA guide of any one of claims 25 to 41, the nucleic acid of claim 42, the vector of claim 43, or the vector of claim 44.

46. The cell of claim 45, wherein the cell is a eukaryotic cell, an animal cell, a mammalian cell, a human cell, a primary cell, a cell line, a stem cell, or a T cell.

47. A kit comprising the composition of any one of claims 1 to 24, the RNA guide of any one of claims 25 to 41, the nucleic acid of claim 42, the vector of claim 43, or the vector system of claim 44.

48. A method of editing a B2M sequence, the method comprising contacting a B2M sequence with a composition of any one of claims 1 to 24 or an RNA guide of any one of claims 25 to 41.

49. The method of claim 48, wherein the B2M sequence is in a cell.

50. The method of claim 48 or 49, wherein the composition or the RNA guide induces a deletion in the B2M sequence.

51. The method of claim 50, wherein the deletion is adjacent to a 5′-NTTN-3′ sequence, wherein N is any nucleotide.

52. The method of claim 50 or 51, wherein the deletion is downstream of the 5′-NTTN-3′ sequence.

53. The method of any one of claims 50 to 52, wherein the deletion is up to about 50 nucleotides in length.

54. The method of any one of claims 50 to 53, wherein the deletion is up to about 40 nucleotides in length.

55. The method of any one of claims 50 to 54, wherein the deletion is from about 4 nucleotides to 40 nucleotides in length.

56. The method of any one of claims 50 to 55, wherein the deletion is from about 4 nucleotides to 25 nucleotides in length.

57. The method of any one of claims 50 to 56, wherein the deletion is from about 10 nucleotides to 25 nucleotides in length.

58. The method of any one of claims 50 to 57, wherein the deletion is from about 10 nucleotides to 15 nucleotides in length.

59. The method of any one of claims 50 to 58, wherein the deletion starts within about 5 nucleotides to about 15 nucleotides of the 5′-NTTN-3′ sequence.

60. The method of any one of claims 50 to 59, wherein the deletion starts within about 5 nucleotides to about 10 nucleotides of the 5′-NTTN-3′ sequence.

61. The method of any one of claims 50 to 60, wherein the deletion starts within about 10 nucleotides to about 15 nucleotides of the 5′-NTTN-3′ sequence.

62. The method of any one of claims 50 to 61, wherein the deletion starts within about 5 nucleotides to about 15 nucleotides downstream of the 5′-NTTN-3′ sequence.

63. The method of any one of claims 50 to 62, wherein the deletion starts within about 5 nucleotides to about 10 nucleotides downstream of the 5′-NTTN-3′ sequence.

64. The method of any one of claims 50 to 63, wherein the deletion starts within about 10 nucleotides to about 15 nucleotides downstream of the 5′-NTTN-3′ sequence.

65. The method of any one of claims 50 to 64, wherein the deletion ends within about 20 nucleotides to about 30 nucleotides of the 5′-NTTN-3′ sequence.

66. The method of any one of claims 50 to 65, wherein the deletion ends within about 20 nucleotides to about 25 nucleotides of the 5′-NTTN-3′ sequence.

67. The method of any one of claims 50 to 66, wherein the deletion ends within about 25 nucleotides to about 30 nucleotides of the 5′-NTTN-3′ sequence.

68. The method of any one of claims 50 to 67, wherein the deletion ends within about 20 nucleotides to about 30 nucleotides downstream of the 5′-NTTN-3′ sequence.

69. The method of any one of claims 50 to 68, wherein the deletion ends within about 20 nucleotides to about 25 nucleotides downstream of the 5′-NTTN-3′ sequence.

70. The method of any one of claims 50 to 69, wherein the deletion ends within about 25 nucleotides to about 30 nucleotides downstream of the 5′-NTTN-3′ sequence.

71. The method of any one of claims 50 to 70, wherein the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

72. The method of any one of claims 50 to 71, wherein the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

73. The method of any one of claims 50 to 72, wherein the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

74. The method of any one of claims 50 to 73, wherein the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

75. The method of any one of claims 50 to 74, wherein the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

76. The method of any one of claims 50 to 75, wherein the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

77. The method of any one of claims 50 to 76, wherein the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

78. The method of any one of claims 50 to 77, wherein the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

79. The method of any one of claims 50 to 78, wherein the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

80. The method of any one of claims 50 to 79, wherein the 5′-NTTN-3′ sequence is 5′-CTTT-3′, 5′-CTTC-3′, 5′-GTTT-3′, 5′-GTTC-3′, 5′-TTTC-3′, 5′-GTTA-3′, or 5′-GTTG-3′.

81. The method of any one of claims 50 to 80, wherein the deletion overlaps with a mutation in the B2M sequence.

82. The method of any one of claims 50 to 81, wherein the deletion overlaps with an insertion in the B2M sequence.

83. The method of any one of claims 50 to 82, wherein the deletion removes a repeat expansion of the B2M sequence or a portion thereof.

84. The method of any one of claims 50 to 83, wherein the deletion disrupts one or both alleles of the B2M sequence.

85. The composition, RNA guide, nucleic acid, vector, cell, kit or method of any one of the previous claims, wherein the RNA guide does not consist of the sequence of:
(SEQ ID NO: 778)
AGAAAUCCGUCUUUCAUUGACGGAAUGUCGGAUGGAUGAAACC;
(SEQ ID NO: 779)
AGAAAUCCGUCUUUCAUUGACGGCUAUCUCUUGUACUACACUG;
(SEQ ID NO: 780)
AGAAAUCCGUCUUUCAUUGACGGACACGGCAGGCAUACUCAUC;
or
(SEQ ID NO: 781)
AGAAAUCCGUCUUUCAUUGACGGGUCACAGCCCAAGAUAGUUA.

86. The composition, RNA guide, nucleic acid, vector, cell, kit or method of any one of the previous claims, wherein the RNA guide comprises the sequence of any one of SEQ ID NOs: 1222-1230.
